(19) United States
(12) Patent Application Publication
(10) Pub. No.: US 2007/0065844 A1
(43) Pub. Date: Mar. 22, 2007

(54) Solution-based methods for RNA expression profiling

(75) Inventors: Todd R. Golub, Newton, MA (US); Justin Lamb, Cambridge, MA (US); David Peck, Framingham, MA (US); Jun Lu, Medford, MA (US); Eric Alexander Miska, Cambridge (GB)

Correspondence Address: Ronald I. Eisenstein;NIXON PEABODY LLP, 100 Summer Street, Boston, MA, 02110, US

(73) Assignees: Massachusetts Institute of Technology, Cambridge, MA (US); Dana-Farber Cancer Institute, Inc.,, Boston, MA (US)

(21) Appl. No.: 11/449,155

(22) Filed: Jun. 8, 2006

Related U.S. Application Data
(60) Provisional application No. 60/689,110, filed on Jun. 8, 2005.

(51) Int. Cl. C12Q 1/68; C12P 19/34

(52) U.S. Cl. 435/6; 435/91.2

(57) ABSTRACT

The present invention is directed to novel high-throughput, low-cost, and flexible solution-based methods for RNA expression profiling, including expression of microRNAs and mRNAs.

### CROSS REFERENCE TO RELATED APPLICATIONS

This application claims the benefit under 35 U.S.C. § 119(e) of U.S. Provisional Patent Application Ser. No. 60/689,110 filed Jun. 8, 2005, the contents of which are herein incorporated by reference in their entirety.

### FIELD OF THE INVENTION

The present invention is directed to methods of screening for malignancies, cellular disorders, and other physiological states as well as novel high-throughput, low-cost, and flexible solution-based methods for RNA expression profiling, including expression of microRNAs and mRNAs.

### BACKGROUND OF THE INVENTION

The availability of high-performance RNA profiling technologies is central to the elucidation of the mechanisms of action of disease genes and the identification of small molecule therapeutics by molecular signature screening (Lamb et al., Cell 114:323-34 (2003); Stegmaier et al., Nature Genetics 36:257-63 (2004)). For example, detection and quantification of differentially expressed genes in a number of conditions including malignancy, cellular disorders, etc. would be useful in the diagnosis, prognosis and treatment of these pathological conditions. Quantification of gene expression would also be useful in indicating susceptibility to a range of conditions and following up effects of pharmaceuticals or toxins on molecular level. These methods can also be used to screen for molecules that provide a desired gene profile.

The power of being able to simultaneously measure the expression level of multiple mRNA species has been of recent interest. For example, the expression of seventy and eighty-one transcripts have together been shown to outperform established clinical and histologic parameters in disease outcome prediction for breast cancer (van de Vijver et al., New Eng. J. Med. 347:1999-2009 (2002)) and follicular lymphoma (Glas et al., Blood 105:301-7 (2005)), respectively.

MicroRNAs are thought to act as post-transcriptional modulators of gene expression, and have been implicated as regulators of developmental timing, neuronal differentiation, cell proliferation, programmed cell death, and fat metabolism. Determining expression profiles of microRNAs is particularly challenging however because of their short size, typically around 21 base pairs, and high degree of sequence homology, where different microRNAs may differ by only a single base pair. It would also be highly desirable to simultaneously measure the expression level of microRNAs, a recently identified class of small non-coding RNA species.

The rapid pace of discovery of new genes generated by large-scale genomic and proteomic initiatives has required the development of high-throughput strategies to quantify the expression of a large number of genes and their alternatively spliced isoforms, as well as elucidate their biological functions, regulations and interactions. (Consortium, E. P. (2004) Science 306, 636-40; Lander et al., Nature 409, 860-921 (2001)) A number of high-throughput techniques have been developed to detect and quantify nucleic acids. Microarray-based analysis has been one widely used high-throughput technique used to study nucleic acids. Another approach for high-throughput analysis of nucleic acids involves the sequencing of a short tag of each transcript, including expressed sequence tag (EST) sequencing (Lander et al., 2001) and serial analysis of gene expression (SAGE) (Velculescu et al., Science 270, 484-7 (1995)).

However, both microarray and tag-sequencing techniques are associated with a number of significant problems. These techniques typically are not sufficiently sensitive and demand relatively high input levels of mRNA that are often unavailable, particularly when studying human diseases. In addition, the array quality is often a problem for cDNA or oligonucleotide microarrays. For example, most researchers cannot confirm the identity of what is immobilized on the surface of a microarray and generally have limited capacity to check and control possible errors in the microarray fabrication. Additionally, the high costs of microarrays have caused many investigators to perform relatively few control experiments to assess the reliability, validity, and repeatability of their findings. Moreover, given the high costs of microarray fabrication, custom designing arrays to tailor analysis to an individual expression profile is simply impractical in many instances. For the tag-sequencing analysis, a large amount of sequencing effort, generally slow and costly, is needed for tag-based analysis and the sensitivity of tag-based analyses is relatively low and high sensitivity can only be achieved by sequencing a large number of tag sequences.

Thus it would be desirable to develop simple, flexible, low-cost, high-throughput methods for the sensitive and accurate quantification of nucleic acids, which can be easily automated and scaled up to accommodate testing of large numbers of samples and overcome the problems associated with available techniques. Such a method would permit diagnostic, prognostic and therapeutic purposes, and would facilitate genomic, pharmacogenomic and proteomic applications, including the discovery of small molecule therapeutics.

### SUMMARY OF THE INVENTION

We have now discovered simple, flexible, low-cost and high-throughput solution-based methods for expression profiling nucleic acids. More specifically, the invention provides methods for detection of multiple genes in a single reaction, including for the detection of mRNAs and microRNAs.

The present invention provides a solution-based method for determining the expression level of a population of target nucleic acids, by a) providing in solution a population of target-specific bead sets, where each target-specific bead set is individually detectable and comprises a capture probe which corresponds to an individual target nucleic acid, referred to as an individual bead set; b) hybridizing in solution the population of target-specific bead sets with a population of molecules that can contain a population of detectable target molecules, where each target nucleic acid has been transformed into a corresponding detectable target molecule which will specifically bind-to-its corresponding individual target-specific bead set; and c) screening in solution for detectable target molecules hybridized to target-specific beads to determine the expression level of the population of target nucleic acids.

In one embodiment, the target-specific bead sets can have at least 5 individual bead sets that can bind with a corresponding set of target nucleic acids. The population of target-specific beads can contain at least 100 individual bead sets that bind with a corresponding set of target nucleic acids.

One preferred embodiment provides a method for detection of populations of mRNAs. In this method, mRNA is transformed into a corresponding detectable target molecule by a) reverse transcribing the mRNA to generate a cDNA; b) hybridizing an upstream probe and a downstream probe to the cDNA, where the upstream probe has a universal upstream sequence and an upstream target-specific sequence, and the downstream probe has a universal downstream sequence and a downstream target-specific sequence, such that when the upstream probe and the downstream probe are both hybridized to the cDNA the two probes are capable of being ligated; c) ligating the two probes to generate ligation complexes; and d) amplifying the ligation complexes with a universal upstream primer and a universal downstream primer, which are complementary to the universal upstream sequence and the universal downstream sequence, respectively. In this method, at least one of universal primers is detectably labeled, such that product of the amplification is delectably labeled, thereby generating a detectable target molecule which corresponds to the target nucleic acid. In this method, either the upstream probe or the downstream probe also has an amplicon tag between the universal sequence and the target-specific. The amplicon tag has a nucleic acid sequence that is unique for the mRNA to be detected, and that is complementary to the sequence of the capture probe of the corresponding bead set, allowing the detectable nucleic acid molecule to hybridize to the bead set with the complementary capture probe.

One embodiment of the invention provides the use of these multiplex mRNA detection methods to screen for the presence of a particular physiological state in a test sample, such as a malignancy, infection or a cellular disorder. In one embodiment, the genes which are specifically associated with one physiological state but not another physiological state are already determined; such a group of genes is typically referred to as an expression signature. To screen for a physiological state using the mRNA detection methods, one first determines the expression signature of a group of genes in the test sample; and then compares the expression signature between the test sample and a corresponding control sample, where a difference in the expression signature between the test sample and the control sample is indicative of the test sample comprising said malignant cells, infected cells or cellular disorder. In one embodiment, the expression signature has at least 5 genes.

One embodiment of the invention provides a method for identifying an expression signature for a physiological state, using the multiplex mRNA detection methods to rapidly screen for genes which are differentially expressed between two physiological states. In one embodiment, the expression signature has at least 5 genes. Examples of physiological states include the presence of a cancer, infection, or a cellular disorder. To identify novel expression signatures, one isolates cells from two groups of individuals, one with and one without the physiological state of interest, and then identifies those genes which are differentially expressed in the two groups of individuals. For those genes which differ at a statistically significant level, linear regression analysis can be applied to identify an expression signature of a gene group that is indicative of an individual having the physiological state of interest.

One preferred embodiment provides a method to detection of populations of microRNAs. In this method, microRNAs are transformed into corresponding detectable target molecules by first ligating at least one adaptor to each microRNA, generating an adaptor-microRNA molecule; and then detectably labeling the adaptor-microRNA molecule, thereby generating a detectable target molecule which corresponds to the target nucleic acid. In one embodiment, the adaptor-microRNA is detectably labeled by reverse transcription using the adaptor microRNA as a template for polymerase-chain reaction, wherein a pair of primers is used in said polymerase chain reaction, and wherein at least one of said primers is detectably labeled. In this method, the capture probe of the bead set which corresponds to an individual microRNA has a sequence which is complementary to the mRNA sequence, allowing the detectable target molecule to bind to the corresponding bead set.

The invention also provides the use of the multiplex microRNA detection methods to screen for the presence of a malignancy in a test sample. In one embodiment, one analyzes the level of expression of microRNAs in a test sample and a corresponding control sample, where a lower level of expression of microRNAs in the test sample relative to the control sample is indicative of the test sample containing malignant cells.

One embodiment of the invention provides a method of screening an individual at risk for cancer by obtaining at least two cell samples from the individual at different times; and determining the level of expression of microRNAs in the cell samples, where a lower level of expression of microRNAs in the later obtained cell sample compared to the earlier obtained cell sample is indicative of the individual being at risk for cancer.

Another embodiment of the invention provides methods of screening an individual at risk for cancer, by determining the level of expression for a specific group of microRNAs, sometimes referred to as a profile group of microRNAs, where lower expression of the profile group of microRNAs is associated with risk for a particular type of cancer.

One embodiment of the invention provides a method for identifying an active compound. In this embodiment, cells are contacted with a plurality of molecules including chemical compounds and biologic molecules, and the expression of a set of marker genes present in the cells is determined using the novel detection methods of the invention. To identify active compounds, the expression of the marker genes to identify a cellular phenotype is scored, the presence of a specific cellular phenotype being indicative of an active compound. In one embodiment the plurality of chemical compounds is a set of compounds selected from the group consisting of small molecule libraries, FDA approved drugs, synthetic chemical libraries, phage display libraries, dosage libraries. In another embodiment the active compound is an anti-cancer drug. In a further embodiment the active compound is a cellular differentiation factor. In certain embodiments, the set of marker genes can include genes encoding mRNAs and/or genes encoding microRNAs.

Another embodiment of the invention provides kits for determining in solution the expression level of a population of target nucleic acids. Kits can include a population of detectable bead sets, wherein each target-specific bead set is individually detectable and is capable of being coupled to a capture probe which corresponds to an individual target nucleic acid of interest; components for transforming a target nucleic acid of interest into a corresponding detectable target molecule which will specifically bind to its corresponding individual target-specific bead set; and instructions for performing the solution-based detection methods of the invention.

### BRIEF DESCRIPTION OF THE DRAWINGS

FIG. 1 shows one embodiment of the present method for multiplex detection of mRNAs. Transcripts are captured on immobilized poly-dT and reverse transcribed. Two oligonucleotide-probes are designed-against each transcript of interest. For example, the upstream probes contain in the embodiment illustrated 20 nt complementary to a universal primer (T7) site, one of one hundred different 24 nt FlexMAP barcodes, and a 20 nt sequence complementary to the 3′-end of the corresponding first-strand cDNA. The downstream probes are 5′-phosphorylated and contain a 20 nt sequence contiguous with the gene-specific fragment of the upstream probe and a 20 nt universal primer (T3) site. Probes are annealed to their targets, free probes removed, and juxtaposed probes joined by the action of Taq ligase to yield synthetic 104 nt amplification templates. PCR is performed with T3 and 5′-biotinylated T7 primers. Biotinylated barcoded amplicons are hybridized against a pool of one hundred sets of fluorescent microspheres each expressing capture probes complementary to one of the barcodes, and incubated with streptavidin-phycoerythrin (SA-PE) to fluorescently label biotin moieties. Captured labeled amplicons are quantified and beads decoded and counted by flow cytometry. This strategy is based on published methods (Elering et al., 2003; Yeakley et al., 2002).

FIG. 2 shows the reproducibility of an embodiment of the method. Mean expression levels for each transcript under each condition were computed and the deviation of each individual data point from its corresponding mean was recorded. A histogram of the fraction of data points in each of twelve bins of fold deviation values is shown. This plot represents 1,800 data points (two conditions×ninety transcripts×ten replicates).

FIG. 3 shows the results of comparison of expression levels in one embodiment. Plot of mean expression values reported by LMA-FlexMAP against IVT-GeneChip for each transcript under both conditions. Means were calculated as for FIG. 4.

FIG. 4 shows performance in a representative gene space. Total RNA from HL60 cells treated with tretinoin or vehicle (DMSO) alone were analyzed by LMA-FlexMAP in the space of ninety transcripts selected from IVT-GeneChip analysis of the same material. Plots depict log ratios of expression levels (tretinoin/DMSO) reported by both platforms for each transcript, in each of nine classes. Correlation coefficients of the log ratios between platforms within each class are shown. IVT-GeneChip, green bars; LMA-FlexMAP, yellow bars. Asterisks (*) flag failed features. Ratios were computed on the means of three parallel hybridizations of the pooled product from three amplification and labeling reactions (IVT-GeneChip) or ten parallel amplification and hybridization procedures (LMA-FlexMAP) for each condition. Basal expression categories are 20-60 (low), 60-125 (moderate) and >125 (high). Differential expression categories are 1.5-2.5×(low), 3-4.5×(moderate) and >5×(high).

FIGS. 5A-5B show schematics of target-preparation and bead detection of mRNAs. (FIG. 5A) 18 to 26-nucleotide (nt) small RNAs were purified by denaturing PAGE (polyacrylamide gel electrophoresis) from total RNAs extracted from tissues or cells. Small RNAs underwent two steps of adaptor ligation utilizing both the 5′-phosphate and 3′-hydroxyl groups, each followed by a denaturing purification. Ligation products were reverse-transcribed (RT) and PCR amplified using a common set of primers, with biotinylation on the sense primer. (FIG. 5b) Denatured targets were hybridized to beads coupled with capture probes for mRNAs. After binding to streptavidin-phycoerythrin (SAPE), the beads went through a flow cytometer that has two lasers and is capable of detecting both the bead identity and fluorescence intensity on each bead.

FIGS. 6A-6C show the specificity and accuracy of bead-based mRNA detection. (FIG. 6a) Synthetic oligonucleotides corresponding to let-7 family and mutants (see FIG. 11 for sequence similarity) were PCR-labelled and hybridized separately on beads and a glass-microarray. Synthetic targets indicated on horizontal axis, capture probes on vertical axis. Values represent proportion of signal relative to correct probe (set to 100%). (FIG. 6B) Cumulative cross-hybridization on capture probes. (FIG. 6C) Northern blot vs. bead detection (lanes 1-7: HEL, K562, TF-1, 293, MCF-7, PC-3, SKMEL-5). Bead results shown at left (averages from three (HEL, TF-1, 293, MCF-7, PC-3) or two (K562, SKMEL-5) independent experiments; error bars indicate standard deviation).

FIG. 7A-7C show hierarchical clustering of mRNA expression. (FIG. 7a) miRNA profiles of 218 samples covering multiple tissues were clustered (average linkage, correlation similarity; samples are columns, mRNAs are rows). Samples of epithelial (EP) origin or derived from the gastrointestinal tract (GI) are indicated. Supplementary FIG. 4 shows more detail. (FIG. 7B) Clustering of 73 bone marrow samples from patients with ALL. Colored bars indicate the ALL subtypes. (FIG. 7C) Comparison of mRNA data and mRNA data. For 89 epithelial samples from (FIG. 7A) that had mRNA expression data, hierarchical clustering was performed. Samples of GI origin are shown in blue. GI-derived samples largely cluster together in the space of mRNA expression, but not by mRNA expression. Abbreviations: STOM: stomach; PAN: pancreas; KID: kidney; PROST: prostate; UT: uterus; MESO: mesothelioma; BRST: breast; FCC: follicular lymphoma; MF: mycosis fungoides; LVR: liver; BLDR: bladder; MELA: melanoma; TALL: T-cell ALL; BALL: B-cell ALL; LBL: diffuse large-B cell lymphoma; AML: acute myelogenous leukemia; HYPER 47-50: hyperdiploid with 47 to 50 chromosomes; HYPER>50: hyperdiploid with over 50 chromosomes; MLL: mixed lineage leukaemia; NORMP: normal ploidy. Further details in Example 3.

FIGS. 8A-8D show comparison between normal and tumor samples reveals global changes in mRNA expression. (FIG. 8A) Markers were selected to correlate with normal vs. tumor distinction. Heatmap of mRNA expression is shown, with mRNAs sorted according to the variance-fixed t-test score. (FIG. 8B) mRNA markers of normal (norn) vs. tumor distinction in human tissues from (FIG. 8A) applied to normal lungs and lung adenocarcinomas of KRasLA1 mice. A k-nearest neighbour (kNN) classifier based on human sample-derived markers yielded a perfect classification of the mouse samples (Euclidean distance, k=3). Mouse tumor T_MLUNG_{—}5 (3rd from right) was occasionally classified as normal with other kNN parameters (Supplementary Information). (FIG. 8C) HL-60 cells were treated with ATRA (+) or vehicle (−) for the indicated days (FIG. 8D). Heatmap of mRNA expression from a representative experiment is shown.

FIG. 9 shows unsupervised analysis of miRNA expression data. miRNA profiling data of 218 samples covering multiple tissues and cancers were filtered, and centred and normalized for each feature. The data were then subjected to hierarchical clustering on both the samples (horizontally oriented) and the features (vertically oriented, with probe names on the left), with average-linkage and Pearson correlation as a similarity measure. Sample names (staggered) are indicated on the top and mRNA names on the left. Tissue types and malignancy status (MAL; N for normal, T for tumor and TCL for tumor cell line) are represented by colored bars. Samples that belong to the epithelial origin (EP) or derived from the gastrointestinal tract (GI) are also annotated below the dendrogram. STOM: stomach; PAN: pancreas; KID: kidney; PROST: prostate; UT: uterus; MESO: mesothelioma; BRST (breast); FCC: follicular lymphoma; MF: mycosis fungoides; COLON: colon; LVR: liver; BLDR: bladder; OVARY: ovary; Lung: lung; MELA: melanoma; BRAIN: brain; TALL: T-cell ALL; BALL: B-cell ALL; LBL: diffused large-B cell lymphoma; AML: acute myelogenous leukaemia.

FIG. 10 shows comparison of miRNA expression levels of poorly differentiated and more differentiated tumors. Poorly differentiated tumors (PD) with primary origins from colon, ovary, lung, breast (BRST) or lymphnode (LBL) were compared to more differentiated tumors (non-PD) of the corresponding tissue types in the miGCM collection. After filtering out non-detectible miRNAs, the remaining 173 features were centered and normalized for each tissue type separately to a mean of 0 and a standard deviation of 1. A heatmap of the data is shown. Samples with the same tissue type and PD status were sorted according to total mRNA expression readings, with higher expressing samples on the left. Features were sorted according to the variance thresholded t-test score.

FIG. 11 shows specificity and accuracy of the bead-based mRNA detection platform, probe similarity (for FIG. 6). Eleven synthetic oligonucleotides corresponding to human let-7 family of mRNAs or mutants were PCR-labelled. Each of the labelled targets was split and hybridized separately on the bead platform and on a glass microarray. The synthetic targets are indicated on the horizontal axis, and the capture probes are indicated on the vertical axis. The similarity of the capture probes are measured by the differences in nucleotides (nt) and indicated by shades of blue.

FIGS. 12A-12B show noise and linearity of bead detection of mRNAs. (FIG. 12a) The noise of target preparation and bead detection was analyzed. Multiple analyses of the same RNA samples were performed. Expression data were log2-transformed after thresholding at 1 to avoid negative numbers. The standard deviation (std) of each mRNA was plotted against the mean of that mRNA. Data were generated from independent labeling reactions and detections of five replicates of MCF-7, four replicates of PC-3, three replicates of HEL, three replicates of TF-1 and three replicates of 293 cell RNAs. Note that most mRNAs have a standard deviation below 0.75 when their mean is above 5 (in log2 scale). (FIG. 12b) Linearity of target preparation and bead detection. miRNAs were labeled and profiled from HEL cell total RNA with different starting amounts (10 ug, 5 ug, 2 ug and 0.5 ug, respectively). Data are averages of duplicate determinations, measured in median fluorescence intensity (MFI). Each line connects the readings of one mRNA with different amounts of starting material.

FIG. 13 shows hierarchical clustering analyses of miRNA data and mRNA data. For 89 epithelial samples that had successful expression data of both miRNAs and mRNAs, hierarchical clustering was performed using average linkage and correlation similarity, after gene filtering. Filtering of miRNA data eliminates genes that do not have expression values above a minimum threshold in any sample (see Supplementary Methods for details). Three different filtering methods were used for mRNA data. The first method (mRNA filt-1) uses the same criteria as used for miRNA data, resulting in 14546 genes. The second method (miRNA filt-2) employed a variation filter as described (Ramaswamy et al., 2001), and resulted in 6621 genes. The third method (mRNA filt-3) focused on transcription factors that passed the above variation filter, ending with 220 genes. Samples of gastrointestinal tract (GI) or non-GI origins are indicated. Tissue type (TT) and malignancy status (MAL) for normal (N) or tumor (T) samples are also indicated. Note that the GI-derived samples largely cluster together in the space of miRNA expression, but not by mRNA expression. Abbreviations: PAN: pancreas; KID: kidney; PROST: prostate; UT: uterus; MESO: mesothelioma; BRST: breast; COLON: colon; BLDR: bladder; OVARY: ovary; Lung: lung; MELA: melanoma.

FIGS. 14A-14D show In vitro erythroid differentiation. Purified CD34⁺ cells from human umbilical cord blood were induced to differentiate along the erythroid lineage. (FIG. 14A) Total cell counts were determined every two days. Data are averages of cell counts from a triplicate experiment and error bars represent standard deviations. (FIG. 14B) Markers of erythroid differentiation, CD71 and Glycophorin A (GlyA), were determined using flow cytometry. Percentages of cells with negative (−), low, or positive (+) marker staining are plotted. (FIG. 14C) miRNA expression profiles of differentiating erythrocytes were determined on days (FIG. 14D) indicated after induction. Data were log₂-transformed, averaged among successfully profiled same-day samples and normalized to a mean of 0 and a standard deviation of 1 for each miRNA. Data were then filtered to eliminate-miRNAs that do not have expression values higher than a minimum cut-off (7.25 on log₂ scale) in any sample. A heatmap of miRNA expression is shown, with red color indicating higher expression and blue for lower expression. Data shown are from a representative differentiation experiment of two performed.

FIG. 15 shows comparison of miRNA expression levels with an mRNA signature of proliferation. A consensus set of mRNA transcripts that positively correlate with proliferation rate was assembled based on published data (see Supplementary Data). Data for miRNA and mRNA expression in lung and breast (BRST) were centered and normalized for each gene, bringing the mean to 0 and the standard deviation to 1. The mean expression of mRNAs correlated with proliferation (on the horizontal axis) was plotted against the mean expression of miRNA markers for tumor/normal distinction (on the vertical axis). Normal samples, poorly differentiated (diff.) tumors and more differentiated tumors are represented by round, triangle and square dots, respectively. Note that the mRNA proliferation signature distinguishes normal samples from tumors, reflecting faster proliferation rates in cancer specimens; however, it does not distinguish between poorly differentiated tumors and more differentiated tumors, even though the miRNA expression levels in the latter two categories are different.

### DETAILED DESCRIPTION OF THE INVENTION

The invention is directed to the discovery and use of improved methods for expression profiling of nucleic acids. As will be discussed in detail below, we have found a simple and flexible method that permits us to rapidly and inexpensively measure gene expression of multiple genes in a single multiplex reaction, ranging from a few genes to 50, 60, 70, 90 or 200 or more genes. Using this method, we have analyzed microRNA and miRNA expression levels, and found these methods are highly efficient and as effective as commercial slide-based microarrays. However, unlike microarrays, the flexibility of the present method permits simple tailoring of the population of genes which can be analyzed in a single reaction. Thus, the present invention is particularly useful for gene expression profiling methods. In addition, using the methods of the invention, we have discovered that microRNAs are downregulated in a wide variety of cancers. Thus, the invention also provides methods for detection of cancer, using microRNA expression profiling.

In one embodiment, the method uses a population of bead sets and measures in solution the expression level of a population of target nucleic acids of interest in a sample. For each individual target nucleic acid of interest, there is a corresponding bead set which comprises a capture probe specific for its target nucleic acid and a unique detectable label, referred to as the bead signal. In this method, a target nucleic acid, such as mRNA in a cell, is first labeled with a detectable signal, referred to as the target signal, before being hybridized with the population of bead sets. Following hybridization in solution of the labeled target nucleic acids with the population of bead sets, the level of both detectable signals is determined for each hybridized bead-target complex. Thus, the bead signal indicates which target nucleic acid is present in the complex, and the level of the target signal indicates the level of expression of that target nucleic acid in the sample. The method can be used to detect tens, or hundreds, or thousands of different target nucleic acids in a single sample.

Accordingly, the invention provides simple, flexible, low-cost, high-throughput methods for simultaneously measuring the expression level of multiple nucleic acids, including mRNAs and microRNAs. In terms of multiplicity, the methods allow the expression level of a few to hundreds, and even thousands, of different target nucleic acids to be measured simultaneously in a single reaction (e.g. 5, 10, 50, 100, 500, or even 1,000 different target nucleic acids). In terms of throughput, the methods allow high numbers of the multiplexed samples to be processed simultaneously, allowing thousands of samples to be rapidly processed. The simplicity of the methods allows the entire procedure to be readily automated. The low cost aspect of the method is reflected for example in a typical unit cost of only several dollars to analyze the expression of 100 nucleic acids in a single sample. As exemplified herein, the performance of the present methods is at least comparable to the current industry-standard oligonucleotide microarrays.

One particularly important advantage of the present method is the high degree of flexibility it provides regarding the population of target nucleic acids to be analyzed. Because the population of bead sets is not fixed, as opposed to the probes on a microarray, the bead population can be readily changed by adding or removing one of the individual bead sets, without altering the other bead sets in the total population. Thus, unlike a slide-based microarray, the population of target nucleic acids to be analyzed can be readily tailored to specific needs, without refabrication of the entire population of bead sets.

The detection methods of the invention can be used in a wide variety of applications as described in detail below, including but not limited to gene expression profiling, screening assays, diagnostic and prognostic assays, for example for gene expression signatures, small molecule or genetic library screening, such as screening cDNA/ORFs, shRNAs, and microRNAs, pharmacogenomics, and the classification of induced biological states.

The invention provides a solution-based method for determining the expression level of a population of target nucleic acids. The method comprises the steps of (a) providing in solution a population of target-specific bead sets, wherein each target-specific bead set is individually detectable and comprises a capture probe which corresponds to an individual target nucleic acid referred to as an individual bead set; (b) hybridizing in solution the population of target-specific bead sets with a population of molecules that can contain a population of detectable target molecules, wherein each target nucleic acid has been transformed into a corresponding detectable target molecule which will specifically bind to its corresponding individual target-specific bead set; and (c) screening in solution for detectable target molecules hybridized to target-specific beads to determine the expression level of the population of target nucleic acids.

In one embodiment, the population of target-specific bead sets comprises at least 5 individual bead sets that can bind with a corresponding set of target nucleic acids. In one embodiment, the population of target-specific beads comprises at least 100 individual bead sets that can bind with a corresponding set of target nucleic acids.

In one embodiment, the population of target nucleic acids is a population of mRNAs. In one embodiment, the population of target nucleic acids is a population of microRNAs.

In one embodiment, each target nucleic acid is an mRNA which has been transformed into a corresponding detectable target molecule. The mRNA is transformed into a corresponding detectable target molecule by a process comprising the steps of (a) reverse transcribing the mRNA target nucleic acid to generate a cDNA; (b) contacting the cDNA with an upstream probe and a downstream probe, wherein the upstream probe comprises a universal upstream sequence and an upstream target-specific sequence, and the downstream probe comprises a universal downstream sequence and a downstream target-specific sequence, such that when the upstream probe and the downstream probe are both hybridized to the cDNA the two probes are capable of being ligated; (c) ligating said cDNA contacted with said upstream and downstream probes to generate ligation complexes; and (d) amplifying said ligation complexes with a pair of universal primers comprising a universal upstream primer and a universal downstream primer. The universal upstream primer is complementary to the universal upstream sequence and the universal downstream primer is complementary to the universal downstream sequence. At least one of the pair of universal primers is detectably labeled. The product of the amplification is detectably labeled. Accordingly, a detectable target molecule is generated which corresponds to the target nucleic acid.

In one embodiment, in the process of transforming the mRNA into a corresponding detectable target molecule, either the upstream probe further comprises an amplicon tag between the universal sequence and the target-specific sequence or the downstream probe further comprises an amplicon tag between the universal sequence and the target-specific sequence. The amplicon tag comprises a nucleic acid sequence that is complementary to the sequence of the capture probe of the bead set.

In one embodiment, each target nucleic acid is a microRNA which has been transformed into a corresponding detectable target molecule. The process of transforming the microRNA into a corresponding detectable target molecule comprises the steps of (a) ligating at least one adaptor to the microRNA, generating an adaptor-microRNA molecule; (b) detectably labeling said adaptor-microRNA molecule. Accordingly, a detectable target molecule is generated which corresponds to the target nucleic acid.

In one embodiment, the adaptor-microRNA is detectably labeled by reverse transcription using the adaptor-microRNA as a template for polymerase chain reaction. In one embodiment, a pair of primers is used in said polymerase chain reaction, and at least one of said primers is detectably labeled.

The present invention further provides a method of screening for the presence of malignancy, infection, cellular disorder, or response to a treatment in a test sample. The method comprises the steps of (a) determining the expression signature of a group of genes in the test sample; and (b) comparing the expression signature between the test sample and a reference sample. A similarity or difference in the expression signature between the test sample and the reference sample is indicative of the presence of malignant cells, infected cells, cellular disorder, or response to a treatment in the test sample. In one embodiment, the solution-based method for determining the expression level of target nucleic acids is used for determination of the expression signature in the test sample and the target nucleic acids are mRNAs. In one embodiment, the expression signature comprises at least 5 genes.

In one embodiment, the reference sample is known to express a predetermined expression signature indicative of the presence of malignancy, infection, or cellular disorder, and the similarity of the expression signature of the test sample to the predetermined expression signature of the reference sample indicates the presence of malignant cells, infected cells, or cellular disorder, in the test sample.

In one embodiment, the reference sample is known to express a predetermined expression signature indicative of a response to treatment, and the similarity of the expression signature of the test sample to the predetermined expression signature of the reference sample indicates the presence of malignant the response to a treatment in the test sample. In one embodiment, the response to treatment is an adverse response to treatment. In one embodiment, the response to treatment is a therapeutic response to treatment.

The invention further provides a method of identifying an expression signature associated with the presence or risk of cancer, infection, cellular disorder, or response to treatment. The method comprises the steps of (a) isolating cells from a group of individuals with said cancer, infection, cellular disorder, or response to treatment, and determining the expression levels of a group of genes; (b) isolating cells from a group of individuals without said cancer, infection, cellular disorder, or response to treatment, and determining the expression levels of said group of genes; and (c) identifying differentially expressed genes from said group of genes which are together indicative of the presence or risk of cancer, infection, cellular disorder, or response to treatment in an individual. Accordingly, an expression signature is identified associated with the presence or risk of cancer, infection, cellular disorder, or response to treatment. In one embodiment, the expression levels of the group of genes is determined using the solution-based method of determining expression level of target nucleic acids.

The invention further provides a method of screening for the presence of malignant cells in a test sample. The method comprises the steps of (a) determining the level of expression of a group of microRNAs in the test sample, and (b) comparing the level of expression of a group of microRNAs between the test sample and a reference sample. In one embodiment, a lower level of expression of the group of microRNAs in the test sample compared to the reference sample is indicative of the test sample containing malignant cells. In one embodiment, a similarity or difference in the level of expression of the group of microRNAs in the test sample compared to the reference sample is indicative of the test sample containing malignant cells. In one embodiment, the microRNAs are transformed into a corresponding detectable target molecule by the process of the present invention. In one embodiment, the determination of the level of microRNA in the sample is determined by the solution-based method of the present invention for determining the expression level of a population of target nucleic acids. In one embodiment, the group of microRNAs comprises at least 5 microRNAs. In one embodiment, the test sample is isolated from an individual at risk of or suspected of having cancer.

The invention further provides a method of screening an individual at risk for cancer. The method comprises the steps of (a) obtaining at least two cell samples from the individual at different times; (b) determining the level of expression of a group of microRNAs in the cell samples, and (c) comparing the level of expression of a group of microRNAs between the cell samples obtained at different times. A lower level of expression of the group of microRNAs in the later obtained cell sample compared to the earlier obtained cell sample is indicative of the individual being at risk for cancer. In one embodiment, the microRNAs are transformed into a corresponding detectable target molecule by the process of the present invention. In one embodiment, the determination of the level of microRNA in the sample is determined by the solution-based method of the present invention for determining the expression level of a population of target nucleic acids.

The invention further provides a method of identifying a microRNA expression signature associated with the presence or risk of cancer, infection, cellular disorder, or response to treatment. The method comprises the steps of (a) isolating cells from a group of individuals with said cancer, infection, cellular disorder, or response to treatment, and determining the expression levels of a group of microRNAs; (b) isolating cells from a group of individuals without said cancer, infection, cellular disorder, or response to treatment, and determining the expression levels of said group of microRNAs; and (c) identifying differentially expressed microRNAs from said group of microRNAs which are together indicative of the presence or risk of cancer, infection, cellular disorder, or response to treatment in an individual. Accordingly, a microRNA expression signature is identified associated with the presence or risk of cancer, infection, cellular disorder, or response to treatment. In one embodiment, the microRNAs are transformed into a corresponding detectable target molecule by the process of the present invention. In one embodiment, the determination of the level of microRNA in the sample is determined by the solution-based method of the present invention for determining the expression level of a population of target nucleic acids.

The invention further provides a method of classifying a tumor sample. The method comprises (a) determining the expression pattern of a group of microRNAs in a tumor sample of unknown tissue origin, generating a tumor sample profile; (b) providing a model of tumor origin microRNA-expression patterns based on a dataset of the expression of microRNAs of tumors of known origin; and (c) comparing the tumor sample profile to the model to determine which tumors of known origin the sample most closely resembles. Accordingly, the tissue origin of the tumor sample is classified. In one embodiment, the determination of the level of microRNA in the sample is determined by the solution-based method of the present invention for determining the expression level of a population of target nucleic acids.

The invention further provides a method of classifying a sample from an unknown mammalian species. The method comprises the steps of (a) determining the expression pattern of a group of microRNAs in a sample of an unknown mammalian species, generating a sample profile; (b) providing a model of known mammalian species microRNA expression patterns based on a dataset of the expression of microRNAs of known mammalian species; and (c) comparing the sample profile to the model of known species to determine which known mammalian species the sample profile most closely resembles. Accordingly, the mammalian species of the sample is classified. In one embodiment, the determination of the level of microRNA in the sample is determined by the solution-based method of the present invention for determining the expression level of a population of target nucleic acids.

The invention further provides a method for identifying an active compound or molecule. The method comprises the steps of (a) contacting cells with a plurality of compounds or molecules, (b) determining the expression of a set of marker genes present in the cells using the solution-based method of the present invention for determining the expression level of a population of target nucleic acids, and (c) scoring the expression of the marker genes to identify a cellular phenotype. The presence of a specific cellular phenotype is indicative of an active compound or molecule. In one embodiment, the plurality of chemical compounds or molecules is a set of compounds or molecules selected from the group consisting of small molecule libraries, FDA approved drugs, synthetic chemical libraries, phage display libraries, dosage libraries. In one embodiment, the set of marker genes comprises genes which encode microRNAs and/or messenger RNAs. In one embodiment, the active compound is an anti-cancer drug. In one embodiment, the cellular phenotype is a tumorigenic status of the cell. In one embodiment, the cellular phenotype is a metastatic status of the cell. In one embodiment, the set of marker genes is a cancer versus non-cancer marker gene set. In one embodiment, the set of marker genes is a metastatic versus non-metastatic marker gene set. In one embodiment, the set of marker genes is a radiation resistant versus radiation sensitive marker gene set. In one embodiment, the set of marker genes is a chemotherapy resistant versus chemotherapy sensitive marker gene set. In one embodiment, the active compound is a cellular differentiation factor. In one embodiment, the cellular phenotype is a cellular differentiation status.

The invention further provides a kit for determining in solution the expression level of a population of target nucleic acids. The kit comprises: (a) a population of detectable bead sets, wherein each target-specific bead set is individually detectable and is capable of being coupled to a capture probe which corresponds to an individual target nucleic acid of interest; (b) components for transforming a target nucleic acid of interest into a corresponding detectable target molecule which will specifically bind to its corresponding individual target-specific bead set; and (c) instructions for performing the solution-based method of the present invention for determining the expression level of a population of target nucleic acids. In one embodiment, the population of target nucleic acids comprises mRNAs and the kit further comprises components for performing the method of the present invention for transforming mRNA into a corresponding detectable target molecule. In one embodiment, the population of target nucleic acids comprises microRNAs, and the kit further comprises components for performing the method of the present invention or transforming microRNA into a corresponding detectable target molecule. In one embodiment, the kit further comprises a polymerase and nucleotide bases. In one embodiment, the kit further comprises a plurality of detectable labels. In one embodiment, the kit further comprises capture probes capable of specifically hybridizing to at least 10 different microRNAs, at least 30 different microRNAs, at least 100 different microRNAs, at least 200 different target microRNAs. In one embodiment, the kit further comprises oligonucleotides for use as capture probes or oligonucleotide sequence information to design target specific probes capable of specifically hybridizing to at least 10 different target mRNAs, at least 30 different target mRNAs, at least 100 different target mRNAs, at least 200 different target mRNAs. In one embodiment, the population of target nucleic acids comprises a set of marker genes associated with the presence or risk of cancer, infection, cellular disorder, or response to treatment. In one embodiment, the sample comprises or is suspected of comprising malignant cells.

Samples

The target nucleic acid can be only a minor fraction of a complex mixture such as a biological sample. As used herein, the term “biological sample” refers to any biological material obtained from any source (e.g. human, animal, plant, bacteria, fungi, protist, virus). For use in the invention, the biological sample should contain a nucleic acid molecule. Examples of appropriate biological samples for use in the instant invention include: solid materials (e.g tissue, cell pellets, biopsies) and biological fluids (e.g. urine, blood, saliva, amniotic fluid, mouth wash).

Nucleic acid molecules can be isolated from a particular biological sample using any of a number of procedures, which are well-known in the art, the particular isolation procedure chosen being appropriate for the particular biological sample.

Solution-Based Method to Determine Expression Levels of Nucleic Acids

The invention provides a solution-based method for highly multiplexed determination of the expression levels of a population of target nucleic acids. The population of target nucleic acids can be a collection of individual target nucleic acids of interest, such as a member of a gene expression signature or just a particular gene of interest. Each individual target nucleic acid of interest is first transformed into a detectable target molecule in a quantitative or semi-quantitative manner, such that the level of each target nucleic acid is reflected by the level of the corresponding detectable target molecule, which is labeled with a detectable signal such as a fluorescent marker. The detectable signal of the target molecule is sometimes referred to as the target molecule signal or simply as the target signal. The method also involves a population of target-specific bead sets, where each target-specific bead set is individually detectable and has a capture probe which corresponds to an individual target nucleic acid. The population of bead sets is hybridized in solution with the population of detectable target molecules to form a hybridized bead-target complex. To determine the expression level of the population of target nucleic acids present, one detects both the target signal and the bead signal for each hybridized bead-target complex, such that the level of the target signal indicates the level of expression of the target nucleic acid, and the bead signal indicates the identity of the target nucleic acid being detected. In one embodiment, the beads can be Luminex™ beads, which are polystyrene microspheres that are internally labeled with two spectrally distinct fluorochromes, such that each set of Luminex™ beads can be distinguished by its spectral address.

The methods of the invention can be used to detect any population of target nucleic acids of interest, including but not limited to DNAs and RNAs. In one preferred embodiment the target nucleic acids are messenger RNAs (mRNAs). In another preferred embodiment the target nucleic acids are microRNAs (microRNAs).

The present invention provides multiplex detection of target nucleic acids in a sample. As used herein, the phrase multiplex or grammatical equivalents refers to the detection of more than one target nucleic acid of interest within a single reaction. In one embodiment of the invention, multiplex refers to the detection of between 2-10,000 different target nucleic acids in a single reaction. As used herein, multiplex refers to the detection of any range between 2-10,000, e.g., between 5-500 different target nucleic acids in a single reaction, 25-1000 different target nucleic acids, 10-100 different target nucleic acids in a single reaction etc.

The present invention also provides high throughput detection and analysis of target nucleic acids in a sample. As used herein, the phrase “high throughput” refers to the detection or analysis of more than one reaction in a single process, where each reaction is itself a multiplex reaction, detecting more than one target nucleic acid of interest. In one preferred embodiment, 2-10,000 multiplex reactions can be processed simultaneously.

Detectable Bead Sets

The solution-based methods of the invention use detectable target-specific bead sets which comprise a capture probe coupled to a detectable bead, where the capture probe corresponds to an individual target nucleic acid. As used herein, beads, sometimes referred to as microspheres, particles, or grammatical equivalents, are small discrete particles.

Each population of bead sets is a collection of individual bead sets, each of which has a unique detectable label which allows it to be distinguished from the other bead sets within the population of bead sets. In one embodiment, the population comprises at least 5 different individual bead sets. In another embodiment, the population comprises at least 20 different individual bead sets. The population can comprise any number of bead sets as long as there is a unique detectable signal for each bead set. For example, at least 10, 20, 30, 50, 70, 100, 200, 500 or even more different individual bead sets. In a further embodiment, the population comprises at least 1000 different individual bead sets.

Any labels or signals can be used to detect the bead sets as long as they provide unique detectable signals for each bead set within the population of bead sets to be processed in a single reaction. Detectable labels include but are not limited to fluorescent labels and enzymatic labels, as well as magnetic or paramagnetic particles (see, e.g., Dynabeads® (Dynal, Oslo, Norway)). The detectable label may be on the surface of the bead or within the interior of the bead. Detectable labels for use in the invention are described in greater detail below.

The composition of the beads can vary. Suitable materials include any materials used as affinity matrices or supports for chemical and biological molecule syntheses and analyses, including but not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications.

Typically the beads have at least one dimension in the 5-10 mm range or smaller. The beads can have any shape and dimensions, but typically have at least one dimension that is 100 mm or less, for example, 50 mm or less, 10 mm or less, 1 mm or less, 100 μm or less, 50 μm or less, and typically have a size that is 10 μm or less such as, 1 μm or less, 100 nm or less, and 10 nm or less. In one embodiment, the beads have at least one dimension between 2-20 μm. Such beads are often, but not necessarily, spherical e.g. elliptical. Such reference, however, does not constrain the geometry of the matrix, which can be any shape, including random shapes, needles, fibers, and elongated. Roughly spherical, particularly microspheres that can be used in the liquid phase, also are contemplated. The beads can include additional components, as long as the additional components do not interfere with the methods and analyses herein.

Commercially available beads which can be used in the methods of the invention include but are not limited to bead-based technologies available from Luminex, Illumina, and Lynx. In one embodiment provides microbeads labeled with different spectral property and/or fluorescent (or colorimetric) intensity. For example, polystyrene microspheres are provided by Luminex Corp, Austin, Tex. that are internally dyed with two spectrally distinct fluorochromes. Using precise ratios of these fluorochromes, a large number of different fluorescent bead sets (e.g., 100 sets) can be produced. Each set of the beads can be distinguished by its spectral address, a combination of which allows for measurement of a large number of analytes in a single reaction vessel. In this embodiment, the detectable target molecule is labeled with a third fluorochrome. Because each of the different bead sets is uniquely labeled with a distinguishable spectral address, the resulting hybridized bead-target complexes will be distinguishable for each different target nucleic acid, which can be detected by passing the hybridized bead-target complexes through a rapidly flowing fluid stream. In the stream, the beads are interrogated individually as they pass two separate lasers. High speed digital signal processing classifies each of the beads based on its spectral address and quantifies the reaction on the surface. Thousands of beads can interrogated per second, resulting a high speed, high throughput and accurate detection of multiple different target nucleic acids in a single reaction.

In addition to a detectable label, the bead sets also contain a capture probe which corresponds to an individual target nucleic acid. Typically, the capture probes are short unique DNA sequences with uniform hybridization characteristics. Useful capture probes of the invention are described in detail below.

The capture probe can be coupled to the beads using any suitable method which generates a stable linkage between probe and the bead, and permits handling of the bead without compromising the linkage using further methods of the invention. Coupling reactions include but are not limited to the use capture probes modified with a 5′ amine for coupling to carboxylated microsphere or bead.

Methods to Transform a Target mRNA into a Detectable Target Molecule

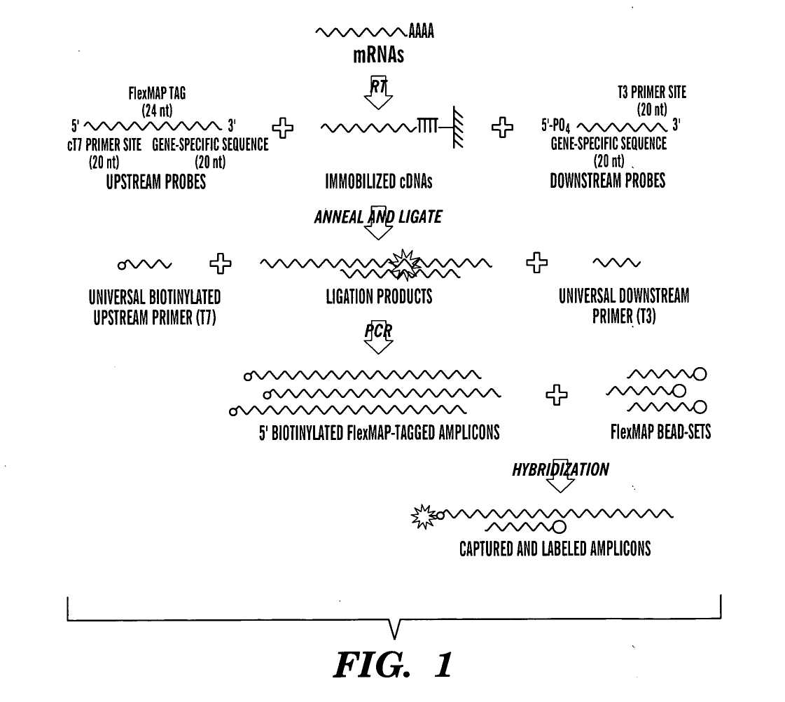

In one preferred embodiment, the present invention provides methods to detect a population of target nucleic acids, where the target nucleic acids are mRNAs, as illustrated in FIG. 1.

To detect a nucleic acid, for example, mRNAs, the invention provides methods to transform a mRNA into a corresponding detectable target molecule. However, any nucleic acid can be used, e.g., DNA, microRNA, etc. In this example, the mRNA target nucleic acid is first reverse transcribed to generate a cDNA, which is then amplified. During the amplification reaction, a detectable signal is also introduced to create a detectable target molecule, sometimes referred to as a tagged or detectable amplicon. In this process, an upstream probe and a downstream probe are first hybridized to the cDNA. The upstream probe comprises a universal upstream sequence and an upstream target-specific sequence, and the downstream probe comprises a universal downstream sequence and a downstream target-specific sequence, such that when the upstream probe and the downstream probe are both hybridized to the cDNA, the two probes are capable of being ligated, as illustrated in FIG. 1. Next, the upstream and downstream probes hybridized to the cDNA are ligated, to generate a ligation complex. For each mRNA present in the starting sample, a single ligation complex is created. Thus, the number of ligation complexes present is a function of the number of individual mRNA molecules present in the starting sample. Finally, the population of ligation complexes is amplified using a pair of universal primers, a universal upstream primer and a universal downstream primer. The universal upstream primer is complementary to the universal upstream sequence, and the universal downstream primer is complementary to the universal downstream sequence. Typically, the universal upstream sequence and the universal downstream sequence are common between all upstream and downstream probes, respectively, so that within a single multiplex reaction, only two universal primers are required to amplify all of the different target nucleic acids being detected. At least one of the pair of universal primers is detectably labeled, such that the product of the amplification is detectably labeled. Accordingly, this process generates a detectable target molecule which corresponds to the target nucleic acid. Detectable labels are discussed in detail below.

The target-specific sequences of the upstream and the downstream probes comprise polynucleotide sequences that are complementary to a portion of the polynucleotide sequence of the target nucleic acid of interest. Preferably, the target-specific sequences of the present invention are completely complimentary to their corresponding target sequence in the nucleic acid of interest. However, the target-specific sequences used in the present invention can have less than exact complementarity with their target sequences, as long as the upstream and downstream probes hybridized to the target sequence can be ligated by a DNA ligase.

To allow hybridization to the capture probe of the corresponding bead set, a sequence which is complementary to the capture probe must be present in the detectable target molecule. For the detection and analysis of mRNA, this sequence is sometimes referred to as the amplicon tag. The amplicon tag may be a sequence within the target nucleic acid-specific sequence, i.e. part of the upstream or downstream target specific sequences. Alternatively, either the upstream probe or the downstream probe may additionally contain an amplicon tag, which lies between the universal sequence and the target specific sequence of the probe. For example, if the amplicon tag resides within the upstream probe, then it is between the upstream universal sequence and the upstream target specific sequence.

Methods to Transform a microRNA into a Detectable Target Molecule

The present invention also provides methods to detect other nucleic acid, such as a population of microRNAs. The detection of microRNAs represents a significant problem in the art because of their size and sequence similarities. microRNAs are a recently identified class of small non-coding RNAs, which are typically around 21 nucleotides and may differ in sequence by only one or a few nucleotides. At present, hundreds of distinct microRNAs have been identified; however, new microRNAs continue to be described.

Mature microRNAs are excised from a stem-loop precursor that itself can be transcribed as part of a longer primary RNA, sometimes referred to as pri-microRNA. The pri-microRNA is then processed by a nuclear RNAse, cleaving the base of the stem-loop and defining one end of the microRNA. Following export to the cytoplasm, the precursor microRNA is further processed by a second RNAse which cleaves both strands of the RNA, typically about 22 nucleotides from the base of the stem. The two strands of the resulting double-stranded RNA are differentially stable, and the mature microRNA resides on the more stable strand. See Lee, EMBO J. 21:4663-70 (2002); Lee, Nature 425:415-19 (2003); Yi, Genes Dev. 17:17:3011-16 (2003); Lund, Science 303:95-8 (2004); Khvorova, Cell 115:209-16 (2003); and Schwarz, Cell 115:199-208 (2003).

To detect a population of microRNAs, the invention provides methods to transform a microRNA into a corresponding detectable target molecule using essentially the method previously described in Miska et al., Genome Biology 5:R68 (2004). In this method, one first ligates at least one adaptor to the population of microRNAs, generating a population of ligated adaptor-microRNA molecules. These ligated molecules are then detectably labeled, thereby generating a detectable target molecule which corresponds to the specific microRNA. In one embodiment, the adaptor-microRNA is detectably labeled by reverse transcription using the adaptor-microRNA as a template for polymerase chain reaction. At least one of the primers used in said polymerase chain reaction is detectably labeled. Detectable labels are described in detail below.

More particularly, the method involves first size selecting 18-26 nucleotide RNAs from total RNA, for example using denaturing polyacrylamide gel electrophoresis (PAGE). Oligonucleotides are then attached to the 5′ and 3′ ends of the small RNAs to generate ligated small RNAs. The ligated small RNAs are then used as templates for reverse transcription PCR, as previously described for microRNA cloning. See Lee, Science 294:862-4 (2001); Lagos-Quintana, Science 294:853-8 (2001); Lau, Science 294:858-62 (2001). The RT-PCR can include for example 10 cycles of amplification. To detectably label the resulting amplification product, either of the primers used for the RT-PCR reaction can have a detectable label, such as a fluorophore such as Cy3. Preferably, the detectable label is attached to the 5′ end of the primer.

The adaptors of the present invention are comprised of nucleic acid sequences typically not found in the population of microRNAs. Preferably, there is less than 35% identity (homology) between the adaptor sequence and the template, more preferably less than 30% identity, still more preferably less than 25% identity. The sequence analysis programs used to determine homology are run at the default setting.

To specifically identify individual microRNAs, the invention provides a population of bead sets where the capture probes are complementary to the microRNA sequences themselves, rather than the adaptor sequences. Thus, the invention provides in certain embodiments a populations of bead sets which are specific to all known microRNAs. As microRNAs continue to be discovered, the invention allows ready addition of new bead sets corresponding to the newly discovered microRNAs to be added. As discussed in detail below, the invention also provides specific sets of populations of bead sets for the expression profiling of signature microRNAs.

Primers, Probes, and Adaptors

As described above, the probes, primers, and adaptors of the invention comprise include but are not limited to the capture probes of the bead sets, universal primers for amplification of the ligation complexes for nucleic acid detection such as mRNA detection, adaptors for the detection of different nucleic acids such as microRNAs, and amplicon tags for hybridization of the detectable target molecules to the capture probes of the bead sets. The invention also provides additional primers, probes, and adaptors for use in various nucleic acid manipulations. The probes, primers and adaptors are sometimes referred to simply as primers.

The probes, primers, and adaptors used in the methods of the invention can be readily prepared by the skilled artisan using a variety of techniques and procedures. For example, such probes, primers, and adaptors can be synthesized using a DNA or RNA synthesizer. In addition, probes, primers, and adaptors may be obtained from a biological source, such as through a restriction enzyme digestion of isolated DNA. Preferably, the primers are single-stranded.

As used herein, the term “primer” has the conventional meaning associated with it in standard PCR procedures, i.e., an oligonucleotide that can hybridize to a polynucleotide template and act as a point of initiation for the synthesis of a primer extension product that is complementary to the template strand.

Preferably, the primers of the present invention have exact complementarity with its target sequence. However, primers used in the present invention can have less than exact complementarity with their target sequence as long as the primer can hybridize sufficiently with the target sequence so as to function as described; for example to be extendible by a DNA polymerase or for hybridization with the capture probe of the bead set.

For use in a given multiplex reaction, the universal primer sequences are typically analyzed as a group to evaluate the potential for fortuitous dimer formation between different primers. This evaluation may be achieved using commercially available computer programs for sequence analysis, such as Gene Runner, Hastings Software Inc. Other variables, such as the preferred concentrations of Mg⁺², dNTPs, polymerase, and primers, are optimized using methods well-known in the art (Edwards et al., PCR Methods and Applications 3:565 (1994)).

Detectable Labels

Any labels or signals which allow detection of the bead set and the detectable target molecules can be used in the methods of the invention. Such detectable labels are well known in the art.

According to the invention, there is a target-specific bead set which corresponds to each target nucleic acid of interest. For each bead set there is a detectable signal, and for the corresponding target nucleic acid there is a distinct detectable signal. Thus, detection of an individual target nucleic interest requires two distinguishable detectable signals.

The detectable labels of the invention may be added to the target nucleic acid and/or the bead sets using various methods. The detectable label may be covalently conjugated with the nucleic acid or non-covalently attached to the nucleic through sequence-specific or non-sequence-specific binding. Examples of the detectable labels include, but are not limited to biotin, digoxigenin, fluorescent molecule (e.g., fluorescin and rhodamine), chemiluminescent moiety (e.g., luminol), coenzyme, enzyme substrate, radio isotopes, a particle such as latex or carbon particle, nucleic acid-binding protein, polynucleotide that specifically hybridizes with either the target or reference nucleic acid strand. Detection of the presence of the label can be achieved by observation or measurement of signals emitted from the label. The production of the signal may be facilitated by binding of the label to its counter-part molecule, which triggers a reaction directly or indirectly. For example, the target nucleic acid may be labeled with biotin; upon binding of streptavidin-HRP (horse radish peroxidase) and addition of the substrate for HRP (e.g., ABTS), the presence of the biotin-labeled target molecule can be detected by observing or measuring color changes in the mixture.

In certain preferred embodiments, the labels are fluorescent and the hybridized bead-target complexes are detected using fluorescence polarization machine, also referred to as a flow cytometer. Fluorescent dyes with diverse spectral properties (e.g., as supplied by Molecular Probes, Eugene, Oreg.) may be used to simultaneously detect multiple detectable target molecules. In this assay, each target molecules may be labeled with a fluorescent dye having different spectral property than that for another target molecule. In another preferred embodiment, the detectable target molecule is labeled with a biotin, and the final hybridized bead-target complexes are further reacted with a signal such as streptavadin-phycoerythrin.

Target Nucleic Acids

In the present invention, a target nucleic acid refers to a sequence of nucleotides to be studied either for the presence of a difference from a reference sequence or for the determination of its presence or absence. The target nucleic acid sequence may be double stranded or single stranded and from a natural or synthetic source. When the target nucleic acid sequence is single stranded, a nucleic acid duplex comprising the single stranded target nucleic acid sequence may be produced by primer-extension and/or amplification.

The present invention is preferably used with at least 5 targets in a single reaction, more preferably at least 10 targets, still more preferably with at least 14 targets, even more preferably with at least 20 targets, yet more preferably with at least 30 targets, still more preferably with at least 50 targets, and even more preferably with at least 100 targets in a single reaction, although one can target any number from 5-1000 as long as a uniquely detectable signal is used. Multiplex detection as used herein refers to the simultaneous detection of multiple nucleic acid targets in a single reaction mixture.

High-throughput denotes the ability to simultaneously process and screen a large number of individual reaction mixtures such as multiplexed nucleic acid samples (e.g. in excess of 100 RNAs) in a rapid and economical manner, as well as to simultaneously screen large numbers of different target nucleic acids within a single multiplexed nucleic acid sample.

Any nucleic acid sample of interest may be used in practicing the present invention, including without limitation eukaryotic, prokaryotic and viral DNA or RNA. In a preferred embodiment, the target nucleic acids represents a sample of total RNA, including mRNA and microRNA, isolated from an individual. This DNA may be obtained from any cell source or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, semen and tissue exudates at the site of infection or inflammation. Nucleic acid such as RNA is extracted from the cell source or body fluid using any of the numerous methods that are standard in the art. It will be understood that the particular method used to extract the nucleic acid will depend on the nature of the source and the type of nucleic acid to be extracted.

The present method can be used with polynucleotides comprising either full-length RNA or DNA, or their fragments. The RNA or DNA can be either double-stranded or single-stranded, and can be in a purified or unpurified form. Preferably, the polynucleotides are comprised of RNA. In certain embodiments, the present invention can be used with full-size cDNA polynucleotide sequences, such as can be obtained by reverse transcription of RNA. The DNA fragments used in the present invention can be obtained by digestion of cDNA with restriction endonucleases, or by amplification of cDNA fractions from cDNA using arbitrary or sequence-specific PCR primers. The nucleic acid can be obtained from a variety of sources, including both natural and synthetic sources. The nucleic acid can be from any natural source including viruses, bacteria, yeast, plants, insects and animals.

Certain embodiments of the invention provide amplification of a nucleic acid using polymerase chain reaction (PCR). “Amplification” of DNA as used herein denotes the use of polymerase chain reaction (PCR) to increase the concentration of a particular DNA sequence within a mixture of DNA sequences. In practicing the present invention, a nucleic acid sample is contacted with pairs of oligonucleotide primers under conditions suitable for polymerase chain reaction. Conditions for performing PCR are well known in the art. Standard PCR reaction conditions may be used, e.g., 1.5 mM MgCl.sub.2, 50 mM KCl, 10 mM Tris-HCl, pH 8.3, 200 μM deoxynucleotide triphosphates (dNTPs), and 25-100 U/ml Taq polymerase (Perkin-Elmer, Norwalk, Conn.). The concentration of each primer in the reaction mixture can range from about 0.05 to about 4 μM. Each potential primer can be evaluated by performing single PCR reactions using each primer pair (e.g. a universal upstream primer and a universal downstream primer) individually. Similarly, each primer pair can be evaluated independently to confirm that all primer pairs to be included in a single multiplex PCR reaction generate a product of the expected size. As the number of targets in a single reaction increases, certain targets may not be amplified as efficiently as other targets. The concentration of the primers for such underrepresented targets may be increased to increase their yield. For example, when multiplying 15 or more targets; more preferably, when multiplying 30 or more targets.

Multiplex PCR reactions are typically carried out using manual or automatic thermal cycling. Any commercially available thermal cycler may be used, such as, e.g., Perkin-Elmer 9600 cycler.

A variety of DNA polymerases can be used during PCR with the present invention. Preferably, the polymerase is a thermostable DNA polymerase such as may be obtained from a variety of bacterial species, including Thermus aquaticus (Taq), Thermus thermophilus (Tth), Thermus filiformis, Thermus flavus, Thermococcus literalis, and Pyrococcus furiosus (Pfu). Many of these polymerases may be isolated from the bacterium itself or obtained commercially. Polymerases to be used with the present invention can also be obtained from cells which express high levels of the cloned genes encoding the polymerase. Preferably, a combination of several thermostable polymerases can be used.

The PCR conditions used to amplify the targets are standard PCR conditions which are well known in the art. Typical conditions use 35-40 cycles, with each cycle comprising a denaturing step (e.g. 10 seconds at 94° C.), an annealing step (e.g. 15 sec at 68° C.), and an extension step (e.g. 1 minute at 72° C.). As the number of targets in a single reaction increases, the length of the extension time may be increased. For example, when amplifying 30 or more targets, the extension time may be three times as longer than when amplifying 10-15 targets (e.g. 3 minutes instead of 1 minute).

In addition to the detection methods specific to the present invention, the reaction products can be analyzed using any of several methods that are well-known in the art, for example to confirm isolated steps of the methods. For example, agarose gel electrophoresis can be used to rapidly resolve and identify each of the amplified sequences. In a multiplex reaction, different amplified sequences are preferably of distinct sizes and thus can be resolved in a single gel. In one embodiment, the reaction mixture is treated with one or more restriction endonucleases prior to electrophoresis. Alternative methods of product analysis include without limitation dot-blot hybridization with allele-specific oligonucleotides and SSCP.

Applications

The methods of the invention can be used in any application or method in which it is desirable to measure or detect the presence of a population of target nucleic acids, such as for gene expression profiling or microRNAs profiling. While several preferred applications are described in detail here, the invention is in no way limited to these embodiments. Other applications would become apparent to one skilled in the art having the benefit of this disclosure.

As described in detail below, the invention can be used in methods for gene expression profiling assays such as, diagnostic and prognostic assays, for example for gene expression signatures, molecule or genetic library screening, such as screening cDNA/ORFs, shRNAs, and microRNAs, pharmacogenomics, and the classification of induced biological states.

Expression Profiling Applications

The methods of the invention are useful for a variety of gene expression profiling applications. More particularly, the invention encompasses methods for high-throughput genetic screening. The method allows the rapid and simultaneous detection of multiple defined target nucleic acids such as mRNA or microRNA sequences in nucleic samples obtained from a multiplicity of individuals. It can be carried out by simultaneously amplifying many different target sequences from a large number of desired samples, such as patient nucleic acid samples, using the methods described above.

In general, as used herein, an expression signature is a set of genes, where the expression level of the individual genes differs between a first physiological state or condition relative to their expression level in a second physiological state or condition, i.e. state A and state B. For example, between cancerous cells and non-cancerous cells, or cells infected with a pathogen and uninfected cells, or cells in different states of development.

The terms “differentially expressed gene,” “differential gene express” and their synonyms, which are used interchangeably, refer to a gene whose expression is activated to a higher or lower level in one physiological state relative to a second physiological subject suffering from a disease, such as cancer, relative to its expression in a normal or control subject. As used herein, “gene” specifically includes nucleic acids which do not encode proteins, such as microRNAs. The terms also include genes whose expression is activated to a higher or lower level at different states of the same disease. A differentially expressed gene may be either activated or inhibited at the nucleic acid level or protein level, or may be subject to alternative splicing to result in a different polypeptide product. Such differences may be evidenced by a change in mRNA levels or microRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. Differential gene expression is considered to be present when there is at least an about two-fold, preferably at least about four-fold, more preferably at least about six-fold, more preferably at least about ten-fold difference between the expression of a given gene between two different physiological states, such as in various stages of disease development in a diseased individual.

An expression signature is sometimes referred to herein as a set of marker genes. An expression signature, or set of marker genes, is a minimum number of genes that is capable of identifying a phenotypic state of a cell. A set of marker genes that is representative of a cellular phenotype is one which includes a minimum number of genes that identify markers to demonstrate that a cell has a particular phenotype. In general, two discrete cell populations in different physiological states having the desired phenotypes may be examined by the methods of the invention. The minimum number of genes in a set of marker genes will depend on the particular phenotype being examined. In some embodiments the minimum number of genes is 2 or, more preferably, 5 genes. In other embodiments, the minimum number of genes is 10, 15, 20, 25, 30, 35, 40, 45, 50, 55, 60, 65, 70, 75, 80, 85, 90, 95, or 1000 genes.

Screening for Expression Signatures

One embodiment of the invention provides highly practical, i.e. low cost, high throughput, and highly flexible routine miRNA expression analysis, for example for clinical testing. The invention provides methods to analyze the expression signature for a cellular phenotype of interest by determining the expression level of a set of marker genes in a test sample. A “phenotype” as used herein refers to a physiological state of a cell under a specific set of conditions, including but not limited to malignancy, infection or a cellular disorder.

In general, analysis of an expression signature involves first determining the expression profile of a gene group, also known as the expression signature, in the test sample, and comparing the expression profile between the test sample and a corresponding control sample, where a difference in the expression profile between the test sample and the control sample is indicative of the test sample expressing the physiological state or cellular phenotype associated with the signature profile. There can be a range of differences in gene expression in the expression profile between the control sample and the profile of interest. Preferably, there are differences from the control profile in at least 25% of the genes being looked at. This can range from a sample showing a 25% change to 100% change from the control sample pattern to the condition of interest and all points in between at least 30%, at least 40%, at least 50%, at least 75%, at least 90%.

The methods of the invention can be used to analyze any expression signature for a cellular phenotype of interest. The identification of expression signatures is the subject of intense study. The invention contemplates the analysis of any expression signature of interest and is in no way limited to the specific embodiments described herein.

In one embodiment, the present invention provides methods to measure gene expression signatures in a sample, where the expression signature is indicative of a malignancy. For example, van de Vivjer et al. New Engl. J. Med. 347:1999-2009 (2002) described a 70 member expression signature associated with breast cancer malignancy or metastasis, and is a predictor of survival. U.S. Patent Application Publication No. 2004/0018527 discloses a group of 91 genes associated with docetaxel chemosensitivity in breast cancer. Additional breast cancer expression signatures are described in detail in U.S. Patent Application Publication No. 2004/0058340 as well as Abba et al., BMC Genomics 6:37 (2005). Glas et al. (2005) described an 81 member expression signature associated with follicular lymphoma, particularly the aggressiveness of the lymphoma. Stegmaier et al. (2004) described a 5 member expression signature which was used in a cell-based small molecule screen for agents inducing the differentiation of human leukemia cells. U.S. Patent Application Publication No. 2004/0009523 discloses 14 genes associated with a diagnosis of multiple mycloma, as well as four subgroups of 24-genes associated with a prognosis of multiple myeloma. U.S. Patent Application Publication No. 2005/0089895 discloses 26 genes associated with the likelihood of recurrence in hepatocellular carcinoma. O'Donnell et al., 2005, Oncogene 24:1244-51, described a group of 116 genes associated with squamous cell carcinoma of the oral cavity. Beer et al. 2002, Nat Med 8:816-824 discloses 50 gene risk index associated with lung adenocarcinoma survival. Classification of human lung cancer by gene expression profiling has been described in several recent publications (M. Garber, PNAS, 98(24): 13784-13789 (2001); A. Bhattacharjee, PNAS, 98(24):13790-13795 (2001). Ramaswamy et al., 2002, Nat Gen 33:49-54 discloses 128 genes whose relative expression levels distinguish between primary and metastatic tumors. Glinsky et al., 2005, J. Clin. Invest. 115:1503-21, discloses 11 genes associated with highly aggressive disease outcomes for several different cancers.

Other disease conditions have also been found to be associated with expression signatures. For example, U.S. Patent Application Publication No. 20040220125 discloses 40 cardioprotective genes, which are useful as a means to diagnose cardiopathology. Baechier et al. 2003, PNAS 100:2610-15 disclose a group of 161 genes associated with severe lupus; see also U.S. Patent Application Publication No. 2004/0033498.

Other cellular states for which expression signatures have been reported include apoptosis, for which a set of 35 regulator genes has been reported (Eldering et al., Nuc. Acid Res. 31:e153 (2003), as well as inflammation, which was associated with a group of 30 genes (Id.).

The present invention also provides methods for diagnosis of infection by gene expression profiling using the methods of the invention. In one embodiment, the expression signature is comprised of cellular host genes whose expression is altered in the presence of an infectious agent. For example, U.S. Patent Application Publication No. 20040038201 discloses expression signatures of cellular host genes associated with infection with a variety of infectious agents, including E. coli, the enterohemorrhagic pathogen E. coli 0157:H7, Salmonella spp. Staphylococcus aureus, Listeria monocytogenes, M. tuberculosis, and M. bovis bacilli Calmette-Gurin (BCG).

In another embodiment, the expression signature is comprised of genes of the infectious agent. The expression signature can also comprise a combination of host and infectious agent genes.

Another preferred embodiment of the invention provides methods for screening for the presence of an infection in a sample by detecting the presence of multiple genes associated with the infectious agent. Viruses, bacteria, fungi and other infectious organisms contain distinct nucleic acid sequences, which are different from the sequences contained in the host cell. Detecting or quantifying nucleic acid sequences that are specific to the infectious organism is important for diagnosing or monitoring infection. Examples of disease causing viruses that infect humans and animals and which may be detected by the disclosed processes include but are not limited to: Retroviridae (e.g., human immunodeficiency viruses, such as HIV-1 (also referred to as HTLV-III, LAV or HTLV-III/LAV, See Ratner, L. et al., Nature, Vol. 313, Pp. 227-284 (1985); Wain Hobson, S. et al, Cell, Vol. 40: Pp. 9-17 (1985)); HIV-2 (See Guyader et al., Nature, Vol. 328, Pp. 662-669 (1987); European Patent Publication No. 0 269 520; Chakraborti et al., Nature, Vol. 328, Pp. 543-547 (1987); and European Patent Application No. 0 655 501); and other isolates, such as HIV-LP (International Publication No. WO 94/00562 entitled “A Novel Human Immunodeficiency Virus”; Picornaviridae (e.g., polio viruses, hepatitis A virus, (Gust, I. D., et al., Intervirology, Vol. 20, Pp. 1-7 (1983); entero viruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Bimaviridae, Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses); Poxyiridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Examples of infectious bacteria include but are not limited to: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus monilifonmis, Treponema pallidium, Treponema pertenue, Leptospira, and Actinomyces israelli.

Examples of parasitic protozoan infections include but are not limited to: Plasmodium vivax, Plasmodium ovale, Plasmodium malariae, Plasmodium falciparum, Toxoplasma gondii, Pneumocystis carinii, Trypanosoma cruzi, Trypanasoma brucei gambiense, Trypanasoma brucei rhodesiense, Leishmania species, including Leishmania donovani, Leishmania mexicana, Naegleria, Acanthamoeba, Trichomonas vaginalis, Cryptosporidium species, Isospora species, Balantidium coli, Giardia lamblia, Entamoeba histolytica, and Dientamoeba fragilis. See generally, Robbins et al, Pathologic Basis of Disease (Saunders, 1984) 273-75, 360-83.

microRNA Expression Profiles

We have also found that one can screen for the presence of malignant cells in a test sample by determining the level of expression of total microRNAs in a test sample; and comparing the levels of expression of microRNAs of the test sample and a control sample. A lower level of expression of microRNAs in the test sample compared to the control sample is indicative of the test sample containing malignant cells. One can use any screening method including the solution base method described herein, or other known methods such as micorarrays for microRNAs, such as that described in Miska et al., 2004.

Another embodiment of the invention provides methods of screening an individual at risk for cancer by obtaining at least two cell samples from the individual at different times; and comparing the level of expression of microRNAs in the cell samples, where a lower level of expression of microRNAs in the later obtained cell sample compared to the earlier obtained cell sample indicates that the individual is at risk for cancer.

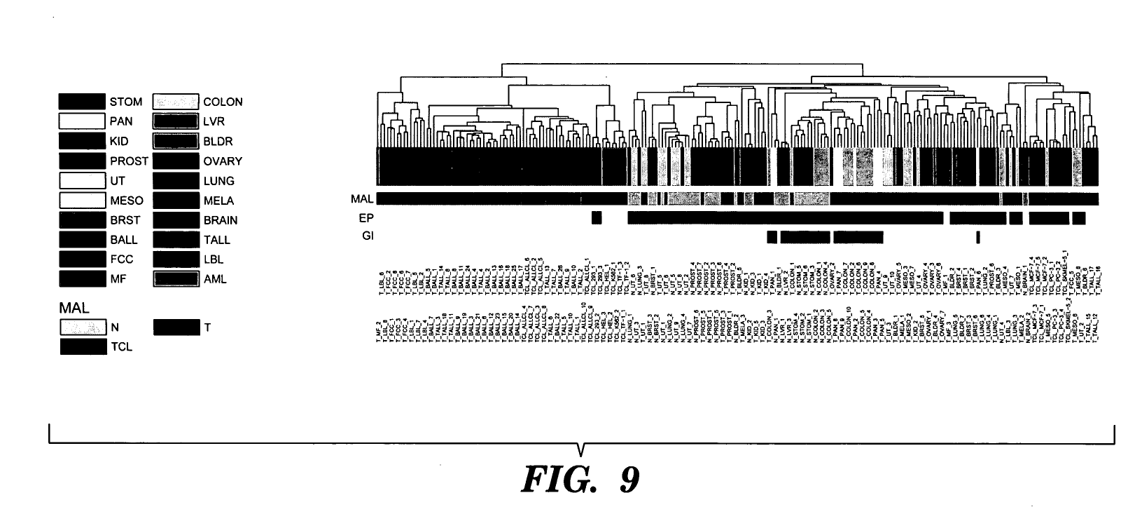

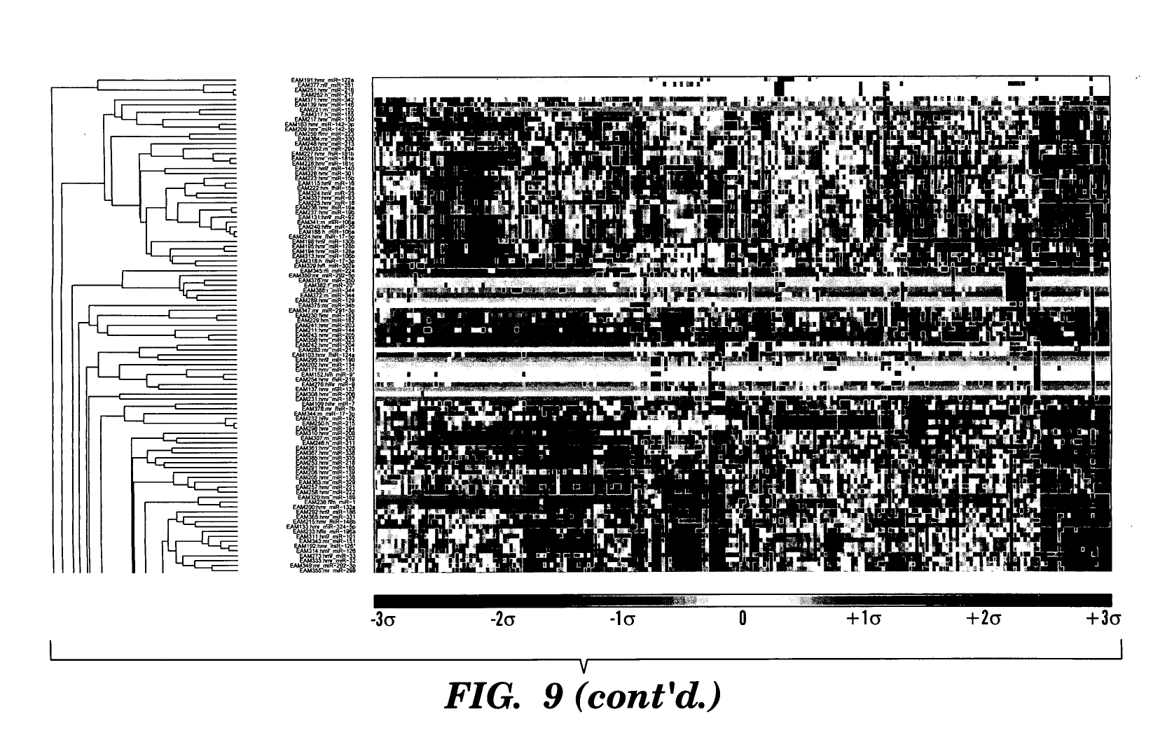

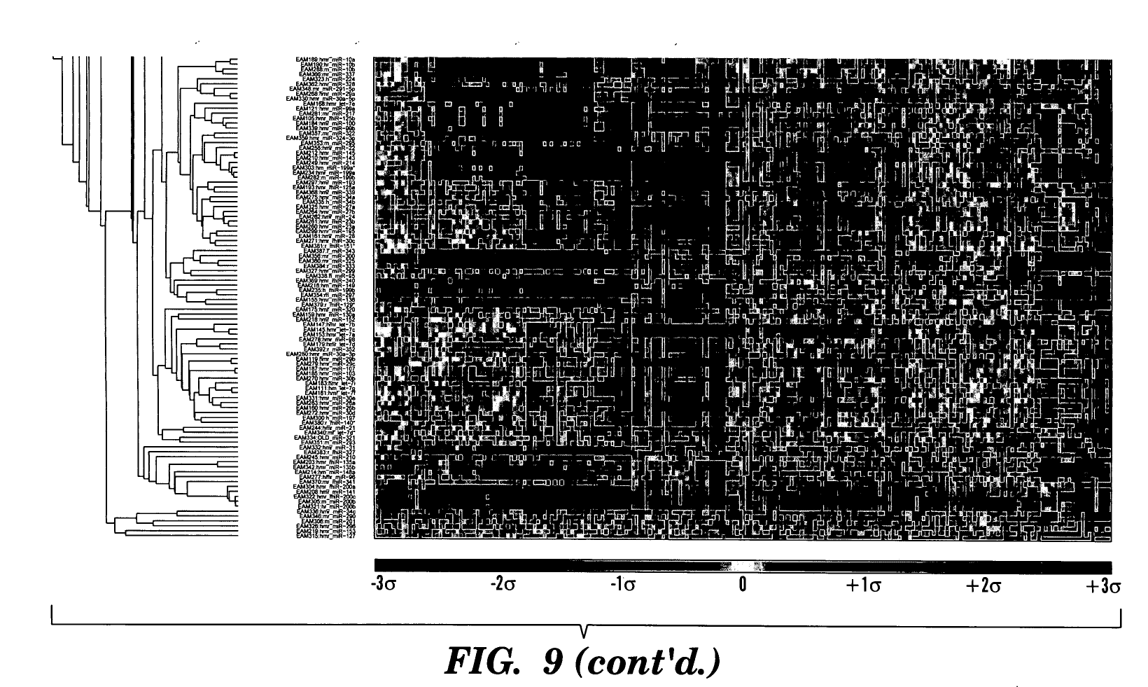

In one preferred embodiment, the methods of the present invention are useful for characterizing poorly differentiated tumors. As exemplified herein, microRNA expression distinguishes tumors from normal tissues, even for poorly differentiated tumors. As shown in FIG. 9, the majority of microRNAs analyzed were expressed in lower levels in tumors compared to normal tissues, irrespective of cell type.

The methods of detecting microRNAs are particularly useful for detecting tumors of histologically uncertain cellular origin, which account for 2-4% of all cancer diagnoses. In this embodiment, the expression profile of microRNAs in a tumor of uncertain cellular origin is compared to a set of microRNA expression profiles for a set of tumors of known origin, allowing classification of the test samples to be assessed based on the comparison.

In another embodiment, the level of expression for a specific group of microRNAs, sometimes referred to a profile group of microRNAs, is determined, where lower expression of said profile group of microRNAs is associated with risk for a particular type of cancer. In particular, microRNAs can be used to classify acute lymphoblastic leukemias into the following subclassifications: t(9;22) BCR/ABL ALLs; t(12;21) TEL/AML1 ALLs; and T-cell ALLs.

Identification of Novel Expression Signatures

We have also discovered methods for identifying an expression profile of a gene group associated with risk of a cellular disorder. It can be any type of nucleic acid that is viewed. In certain embodiments, the genes encode mRNAs. In other preferred embodiments, the genes encode microRNAs.

In one embodiment, the methods involve the establishment of two or more sets of gene expression profiles. The gene expression profiles are utilized to develop marker gene sets which identify a phenotype. Thus, the methods of the invention involve the identification of a cell signature which is useful for identifying a phenotype of a cell.

As used herein, a control gene or set of control genes is selected that are common between the two physiological states in similar or equivalent degrees of gene expression. Additionally, a common housekeeping gene(s) may be used as an “internal” reference or control to normalize the readout for relative differences in cell populations in the screening assay. One example of a common gene useful in the invention is glyceraldehyde 3-phosphate dehydrogenase (GAPDH) (M33197). The expression level of the marker genes will define the phentypic state when taken in ratio to the common gene(s). Hence, quantitation of the expression levels for 2 or more marker genes will be adequate to identify a new phenotypic state.

In this method, one isolates cells from a group of individuals with a cancer, infection, or cellular disorder, and determining the expression level of multiple genes; isolating cells from a group of individuals without said cancer, infection, or cellular disorder, and determining the expression level of said multiple genes; and identifying differential gene expression patterns that are statistically significant; and applying linear regression analysis to identify an expression profile of a gene group that is indicative of an individual having risk of said cancer, infection, or cellular disorder. One can use any screening technique to identify the expression profile. The method described herein is particularly useful because of the flexibility it provides in selecting beads that suit a specific profile.

Small Molecule Screening Methods

The present invention also provides methods to screen a library to identify molecules that change the profile of a cell to result in a desired result. The methods of multiplex target nucleic acid detection are particularly useful in methods for drug screening, such as those disclosed in U.S. Published Patent Application No. 2004/0009495, which is hereby incorporated herein in its entirety.

In this method, the effect of a molecule such as a small molecule protein, etc. on the expression profile signature is used to identify small molecules of interest. For example, one can screen for molecules which alter an expression signature associated with a biological state, such as cancer, such that the expression signature of a sample exposed to the small molecule is altered to more closely resemble the healthy state, i.e. a non-cancerous state. One would look for molecules that change the profile of at least 25% of the genes in the profiling to a profile of the healthy cell. In other embodiments, one looks for molecules or groups of molecules that result in a change of the expression profile of at least 30$, at least 40%, at least 50%, at least 60%, at least 75%, at least 80%, at least 90% until one gets virtual identity with the desired state.

In another embodiment, one can also screen from molecules that cause an undesired condition by looking at how an expression profile is changes from the desired profile to an undesired profile. The present methods can also be used to monitor when a patient should get therapy, what therapy and the effect of that therapy. For example, in pharmacogenomics applications and methods, including the use of gene expression signatures to predict response to therapy. Such applications can be deployed on this platform providing a practical (i.e. low cost, high throughput) mRNA expression based tool to inform treatment decisions or enrollment in clinical trials.

The screening methods may be used for identifying therapeutic agents or validating the efficacy of agents. Agents of either known or unknown identity can be analyzed for their effects on gene expression in cells using methods such as those described herein. Briefly, purified populations of cells are exposed to the plurality of chemical compounds, preferably in an in vitro culture high throughput setting, and optionally after set periods of time, the entire cell population or a fraction thereof is removed and mRNA is harvested therefrom. Any target nucleic acids, such as mRNAs or microRNAs, are then analyzed for expression of marker genes using methods such as those described herein. Hybridization or other expression level readouts may be then compared to the marker gene data. These methods can be used for identifying novel agents, as well as confirming the identity of agents that are suspected of playing a role in regulation of cellular phenotype.

The methods of the invention allows for subjects to be screened and potentially characterized according to their ability to respond to a plurality of drugs. For instance, cells of a subject, e.g., cancer cells, may be removed and exposed to a plurality of putative therapeutic compounds, e.g., anti-cancer drugs, in a high throughput manner. The nucleic acids of the cells may then be screened using the methods described herein to determine whether marker genes indicative of a particular phenotype are expressed in the cells. These techniques can be used to optimize therapies for a particular subject. For instance, a particular anti-cancer therapy may be more effective against a particular cancer cell from a subject. This could be determined by analyzing the genes expressed in response to the plurality of compounds. Likewise a therapeutic agent with minimal side effects may be identified by comparing the genes expressed in the different cells with a marker gene set that is indicative of a phenotype not associated with a particular side effect. Additionally, this type of analysis can be used to identify subjects for less aggressive, more aggressive, and generally more tailored therapy to treat a disorder.

The methods are also useful for determining the effect of multiple drugs or groups of drugs on a cellular phenotype. For instance it is possible to perform combined chemical genomic screens to identify a synergistic or other combined effect arising from combinations of drugs. One set of drugs that induces a first set of marker genes indicative of a phenotype, while another drug induces an second set of marker genes. When the two sets of drugs are combined they may act to achieve a collective phenotypic change, exemplified by a third set of marker genes. Additionally the methods could be used to assess complex multidrug effects on cell types. For instance, some drugs when used in combination produce a combined toxic effect. It is possible to perform the screen to identify marker genes associated with the toxic phenotype. Existing compounds could be screened for there ability to “trip” the signal signature of toxic effect, by monitoring the marker genes associated with the toxic phenotype.

The methods may also be used to enhance therapeutic strategies. For instance, oncolytic therapy involves the use of viruses to selectively lyse cancer cells. A set of marker genes which identify a gene expression signature favorable to selective viral infection can be identified. Using this set of marker genes, drugs can be found which favor or enable selective viral infectivity in order to enhance the therapeutic benefit.

Thus, the methods of the invention are useful for screening multiple compounds. For instance, the methods are useful for screening libraries of molecules, FDA approved drugs, and any other sets of compounds. Preferably the methods are used to screen at least 20 or 30 compounds, and more preferably, at least 50 compounds. In some embodiments, the methods are used to screen more than 96, 384, or 1536 compounds at a time.

In one embodiment, the methods of the invention are useful for screening FDA approved drugs. An FDA approved drug is any drug which has been approved for use in humans by the FDA for any purpose. This is a particularly useful class of compounds to screen because it represents a set of compounds which are believed to be safe and therapeutic for at least one purpose. Thus, there is a high likelihood that these drugs will at least be safe and possibly be useful for other purposes. FDA approved drugs are also readily commercially available from a variety of sources.

A “library of molecules” as used herein is a series of molecules displayed such that the compounds can be identified in a screening assay. The library may be composed of molecules having common structural features which differ in the number or type of group attached to the main structure or may be completely random. Libraries are meant to include but are not limited to, for example, phage display libraries, peptides-on-plasmids libraries, polysome libraries, aptamer libraries, synthetic peptide libraries, synthetic small molecule libraries and chemical libraries. Methods for preparing libraries of molecules are well known in the art and many libraries are commercially available. Libraries of interest include synthetic organic combinatorial libraries. Libraries, such as, synthetic small molecule libraries and chemical libraries. The libraries can also comprise cyclic carbon or heterocyclic structure and/or aromatic or polyaromatic structures substituted with one or more functional groups. Libraries of interest also include peptide libraries, randomized oligonucleotide libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties which are less subject to enzymatic degradation compared to their naturally-occurring counterparts.

Small molecule combinatorial libraries may also be generated. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A “compound array” as used herein is a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Pat. No. 5,712,171 issued Jan. 27, 1998.

One type of library, which is known as a phage display library, includes filamentous bacteriophage which present a library of peptides or proteins on their surface. Phage display libraries can be particularly effective in identifying compounds which induce a desired effect in cells. Briefly, one prepares a phage library (using e.g. m13, fd, lambda or T7 phage), displaying inserts from 4 to about 80 amino acid residues using conventional procedures. The inserts may represent, for example, a completely degenerate or biased array. DNA sequence analysis can be conducted to identify the sequences of the expressed polypeptides. The minimal linear peptide or amino acid sequence that have the desired effect on the cells can be determined. One can repeat the procedure using a biased library containing inserts containing part or all of the minimal linear portion plus one or more additional degenerate residues upstream or downstream thereof.

For certain embodiments of this invention, e.g., where phage display libraries are employed, a preferred vector is filamentous phage, though other vectors can be used. Vectors are meant to include, e.g., phage, viruses, plasmids, cosmids, or any other suitable vector known to those skilled in the art. The vector has a gene, native or foreign, the product of which is able to tolerate insertion of a foreign peptide. By gene is meant an intact gene or fragment thereof. Filamentous phage are single-stranded DNA phage having coat proteins. Preferably, the gene that the foreign nucleic acid molecule is inserted into is a coat protein gene of the filamentous phage. Examples of coat proteins are gene III or gene VIII coat proteins. Insertion of a foreign nucleic acid molecule or DNA into a coat protein gene results in the display of a foreign peptide on the surface of the phage. Examples of filamentous phage vectors which can be used in the libraries are fUSE vectors, e.g., fUSE1 fUSE2, fUSE3 and fUSE5, in which the insertion is just downstream of the pill signal peptide. Smith and Scott, Methods in Enzymology 217:228-257 (1993).

By recombinant vector it is meant a vector having a nucleic acid sequence which is not normally present in the vector. The foreign nucleic acid molecule or DNA is inserted into a gene present on the vector. Insertion of a foreign nucleic acid into a phage gene is meant to include insertion within the gene or immediately 5′ or 3′ to, respectively, the beginning or end of the gene, such that when expressed, a fusion gene product is made. The foreign nucleic acid molecule that is inserted includes, e.g., a synthetic nucleic acid molecule or a fragment of another nucleic acid molecule. The nucleic acid molecule encodes a displayed peptide sequence. A displayed peptide sequence is a peptide sequence that is on the surface of, e.g. a phage or virus, a cell, a spore, or an expressed gene product.

In certain embodiments, the libraries may have at least one constraint imposed upon their members. A constraint includes, e.g., a positive or negative charge, hydrophobicity, hydrophilicity, a cleavable bond and the necessary residues surrounding that bond, and combinations thereof. In certain embodiments, more than one constraint is present in each of the broader sequences of the library.

In addition to the basic libraries, the methods can also be used to screen combinations of drugs. Thus, more than one type of drug can be contacted with each cell.

In other aspects of the invention, the cells do not necessarily need to be contacted with any compounds. The cells may be analyzed for phenotypic status based on environmental condition, such as in vivo or in vitro conditions. It is possible to analyze the differentiation state or tumorigenic state of a cell using the marker gene sets or metagenes of the invention. Thus, a cell may be subjected to conditions in vitro or in vivo and then analyzed for differentiation status.

Additionally, it is possible to screen sets of compounds to identify particular dosages effective at producing a phenotypic state in a cell. For instance, one or more drugs could be contacted with the cells at a variety of dosages over a large range. When the level of marker genes expressed in each of the cells is assessed, it will be possible to identify an optimum dosage for producing a particular phenotypic state of the cell. Additionally, if some markers are associated with the production of undesirable side effects, such as production of cytotoxic factors, then an optimum drug, combination of drug or dosage of drug can be identified using the methods of the invention.

The methods of the invention are useful for assaying the effect of compounds on cells or for analyzing the phenotypic status of a cell. The methods may be used on any type of cell known in the art. For instance the cell may be a cultured cell line or a cell isolated from a subject (i.e. in vivo cell population). The cell may have any phenotypic property, status or trait. For instance, the cell may be a normal cell, a cancer cell, a genetically altered cell, etc.

Cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; liver cancer; lung cancer (e.g., small cell and non-small cell); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas. Some cancer cells are metastatic cancer cells.

“Normal cells” as used herein refers any cell, including but not limited to mammalian, bacterial, plant cells, that is a non-cancer cell, non-diseased, or a non-genetically engineered cell. Mammalian cells include but are not limited to mesenchymal, parenchymal, neuronal, endothelial, and epithelial cells.

A “genetically altered cell” as used herein refers to a cell which has been transformed with an exogenous nucleic acid.

Kits

The present invention further concerns kits which contain, in separate packaging or compartments, the reagents such as adaptors and primers required for practicing the detection methods of the invention. Such kits typically include at least a population of detectable bead sets and preferably several different primers to generate a population of delectably labeled target molecules for detection. Such kits may optionally include the reagents required for performing ligation reactions, such as DNA or RNA ligases, PCR reactions, such as DNA polymerase, DNA polymerase cofactors, and deoxyribonucleotide-5′-triphosphates. Optionally, the kit may also include various polynucleotide molecules, restriction endonucleases, reverse transcriptases, terminal transferases, various buffers and reagents. Optimal amounts of reagents to be used in a given reaction can be readily determined by the skilled artisan having the benefit of the current disclosure.

The kits may also include reagents necessary for performing positive and negative control reactions. Preferably the kits include several target nucleic acids, in separate vials or tubes, or preferably, a set of combined standards comprising at least two different standards in the same vial or tube with known amount of dried standard nucleic acid(s) with instructions to dilute the sample in a suitable buffer, such as PBS, to a known concentration for use in the quantification reaction. Alternatively, the standard is pre-diluted at a known concentration in a suitable buffer, such as PBS. Suitable buffer can be either suitable for both for storing nucleic acids and for, e.g., PCR or direct enhancement reactions to enhance the difference between the standard and a corresponding target nucleic acid as described above, or the buffer is just for storing the sample and a separate dilution buffer is provided which is more suitable for the consequent PCR, enhancement and quantification reactions. In a preferred embodiment, all the standard nucleic acids are combined in one tube or vial in a buffer, so that only one standard mix can be added to a nucleic acid sample containing the target nucleic acid.

The kit also preferably comprises a manual explaining the reaction conditions and the measurement of the amount of target nucleic acid(s) using the standard nucleic acid(s) or a mixture of them and gives detailed concentrations of all the standards and of the type of buffer. Kits contemplated by the invention include, but are not limited to kits for determining the amount of target nucleic acids in a biological sample, and kits determining the amount of one or more transcripts that is expected to be increased or decreased after administration of a medicament or a drug, or as a result of a disease condition such as cancer.

The present invention also provides kits specific for the detection of particular gene expression signatures, as described above. For example, a kit containing target specific bead sets for detecting microRNA for use in determining microRNA expression profiles in samples, including for example diagnostic screening kits.

### EXAMPLES

### Example 1

### A Bead-Based Gene Expression Signature Analysis Method

Materials and Methods

Cell Culture and RNA Isolation:

HL60 (human promyelocytic leukemia) cells were cultured in RPMI supplemented with 10% fetal bovine serum and antibiotics. Cells were treated with 1 μM tretinoin (all-trans-retinoic acid; Sigma-Aldrich) in dimethylsulfoxide (DMSO; final concentration 0.1%) or DMSO alone for five days. Total RNA was isolated from bulk cultures with TRIzol Reagent (Invitrogen) in accordance with the manufacturer's directions. Cells cultured in microtiter plates were treated with 200 nM tretinoin or DMSO for two days and prepared for mRNA capture by the addition of Lysis Buffer (RNAture).

Microarrays:

Total RNA was amplified and labeled using a modified Eberwine method, the resulting cRNA hybridized to Affymetrix GeneChip HG-U133A oligonucleotide microarrays, and the arrays scanned in accordance with the manufacturer's directions. Intensity values were scaled such that the overall fluorescence intensity of each microarray was equivalent. Expression values below an arbitrary baseline (20) were set to 20. These data are provided as Tables 5-8.

Gene Selection:

The 9466 probe-sets reporting above baseline were first divided into up- and down-regulated groups by differences in mean expression levels between tretinoin and vehicle treatments. Each of these groups was further divided into three sets of approximately equal size on the basis of the lower mean expression level. The selected basal expression categories were 20-60 (low), 60-125 (moderate) and >125 (high). Probe-sets reporting small (1.5-2.5×), medium (3-4.5×) or large (>5×) changes in mean expression level within each basal expression category were extracted and ranked by signal to noise ratio. The top five probes mapping to unique RefSeq identifiers according to NetAffx (www.affyinetrix.com) in each of the eighteen categories were selected, populating nine sets of ten genes (Table 2).

Probes and Primers:

Upstream LMA probes were composed (5′ to 3′) of the complement of the T7 primer site (TAA TAC GAC TCA CTA TAG GG), a 24 nt barcode, and a 20 nt gene-specific sequence. Downstream LMA probes were 5′-phosphorylated and contained a 20 nt gene-specific sequence and the T3 primer site (TCC CTT TAG TGA GGG TTA AT). Barcode sequences were developed by Tm Bioscience (www.universalarray.com) and detailed in the FlexMAP Microspheres Product Information Sheet (Luminex). Gene-specific fragments of LMA probes were designed against the Oligator Human Genome RefSet (sequences available for download at www.illumia.com) keyed by RefSeq identifier. A 40 nt region was manually selected from within these 70 nt sequences to yield two fragments of equal length with roughly similar base composition and juxtaposing nucleotides being C-G or G-C, where possible. Probe sequences are provided as Table 3. Capture probes contained the complement of the barcode sequences and had 5′-amino modification and a C12 linker. The T7 primer (5′-TAA TAC GAC TCA CTA TAG GG-3′) was 5′-biotinylated. The T3 primer has the sequence 5′-ATT AAC CCT CAC TAA AGG GA-3′. Oligonucleotides (all with standard desalting) were from Integrated DNA Technologies.

Beads and Bead Coupling:

xMAP Multi-Analyte COOH Microspheres (Luminex) were coupled to capture probes in a semi-automated microtiter plate format. Approximately 2.5×10⁶ microspheres were dispensed to the wells of a V-bottomed microtiter plate, pelleted by centrifugation at 1800 g for 3 min, and the supernatant removed. Beads were resuspended in 25 μl of binding buffer [0.1M 2-(N-morpholino)ethansulfonic acid, pH 4.5] by sonication and pipeting, and 100 pmol of capture probe added. Two and a half lp of a freshly prepared 10 mg/ml aqueous solution of 1-ethyl-3-[3-dimethylaminopropyl] carbodiimide hydrochloride (Pierce) was added, and the plate incubated at room temperature in the dark for 30 min. This addition and incubation step was repeated, and 180 μl 0.02% Tween-20 added with mixing. Beads were pelleted by centrifugation, as before, and washed sequentially in 180 μl 0.1% SDS and 180 μl TE (pH 8.0) with intervening spins. Coupled microspheres were resuspended in 50 μl TE (pH 8.0) and stored in the dark at 4° for up to one month. Bead mixes were freshly prepared and contained ˜1.5×10⁵/ml of each microsphere in 1.5×TMAC buffer [4.5 M tetrametlylammonium chloride; 0.15% N-lauryl sarcosine, 75 mM tris-HCl, pH 8.0; 6 mM EDTA, pH 8.0]. The mapping of bead number to capture probe sequence is provided as Table 4.

Ligation-Mediated Amplification (LMA):

Transcripts were captured in oligo-dT coated 384 well plates (GenePlateHT; RNAture) from total RNA (500 ng) in Lysis Buffer (RNAture) or whole cell lysates (20 μl). Plates were covered and centrifuged at 500 g for 1 min, and incubated at room temperature for 1 h. Unbound material was removed by inverting the plate onto an absorbent towel and spinning as before. Five μl of an M-MLV reverse transcriptase reaction mix (Promega) containing 125 μM of each dNTP (Invitrogen) was added. The plate was covered, spun as before, and incubated at 37° for 90 min. Wells were emptied by centrifugation, as before. Ten fmol of each probe was added in 1×Taq Ligase Buffer (New England Biolabs) (5 μl), the plate covered, spun as before, heated at 95° for 2 min and maintained at 50° for 6 h. Unannealed probes were removed by centrifugation, as before. Five μl of 1×Taq Ligase Buffer containing 2.5 U Taq DNA ligase (New England Biolabs) was added, the plate covered, spun as before and incubated at 45° for 1 h followed by 65° for 10 min. Wells were emptied by centrifugation, as before. Fifteen μl of a HotStarTaq DNA Polymerase mix (Qiagen) containing 16 μM of each dNTP (Invitrogen) and 100 nM of T3 primer and biotinylated-T7 primer was added. The plate was covered, spun as before, and PCR performed in a Thermo Electron MBS 384 Satellite Thermal Cycler (initial denaturation of 92° for 9 min; 92° for 30 s, 60° for 30 s, 72° for 30 s for 39 cycles; final extension at 72° for 5 min).

Hybridization and Detection:

Fifteen μl of LMA reaction product was mixed with 5 μl TE (pH 8.0) and 30 μl of bead mix (˜4500 of each microsphere) in the wells of a Thermowell P microtiter plate (Costar). The plate was covered and incubated at 95° for 2 min and maintained at 45° for 60 min. Twenty μl of a reporter mix containing 10 ng/μl streptavidin R-phycoerythrin conjugate (Molecular Probes) in 1×TMAC buffer [3 M tetramethylammonium chloride; 0.1% N-lauryl sarcosine; 50 mM tris-HCl, pH 8.0; 4 mM EDTA, pH 8.0] was added with mixing and incubation continued at 45° for 5 min. Beads were analyzed with a Luminex 100 instrument. Sample volume was set at 50 μl and flow rate was 60 μl/min. A minimum of 100 events were recorded for each bead set and median fluorescence intensities (MFI) computed. Expression values for each transcript were corrected for background signal by subtracting the MFI of corresponding bead sets from blank (ie TE only) wells. Values below an arbitrary baseline (5) were set to 5, and all were normalized against an internal control feature (GAPDH-3′).

k-nearest-neighbor (KNN) Classifier:

The IVT-GeneChip data from long duration high dose tretinoin or vehicle treatments were used to train a series of KNN classifiers in the spaces of the full ninety member gene set and each of the nine ten member gene categories. These were applied to the corresponding data from the eighty-eight LMA-FlexMAP test samples whose internal reference feature (GAPDH-3′) was within two standard deviations from the mean. To permit the cross-platform analysis, both the train and test data sets were normalized so that each gene had a mean of zero and a standard deviation of one. The KNN algorithm classifies a sample by assigning it the label most frequently represented among the k nearest samples. In this case k was set to 3. The votes of the nearest neighbors were weighted by one minus the cosine distance. This analysis was performed with the GenePattern software package (http://www.broad.mit.edu/cancer/software/genevattern/index.html).

13, 21-27 (1967).

Results

Measurement of seventy and eight-one transcripts has been shown to outperform established clinical and histologic parameters in disease outcome prediction for breast cancer (van de Vijver et al., 2002) and follicular lymphoma (Glas et al., 2005), respectively. Signatures of similar size and comparable prognostic power are sure to follow for a wide variety of diseases. A five member gene expression signature has also been used successfully in a cell-based small molecule screen for agents inducing the differentiation of human leukemia cells (Stegmaier et al., 2004). The absence of reliance upon prior target identification makes gene expression signature screening a powerful new strategy in drug discovery. However, immediate implementation of these and other important medical and pharmaceutical applications of genomics research is now blocked simply by the absence of a cost-effective gene expression profiling solution tailored specifically for the analysis of any feature-set of up to one hundred transcripts.

High-density oligonucleotide microarrays (Lockhart et al., 1996) coupled with RNA amplification and labeling based on in vitro transcription (Van Gelder et al., 1990) provide the solution of choice for unbiased transcriptome analysis. However, the number and complexity of manipulations required, together with the cost of reagents, instrumentation, and the arrays themselves preclude its use for routine clinical and high-throughput applications. Fluorescence mediated real-time RT-PCR integrates amplification, labeling and detection Gibson et al., 1996; Morrison et al., 1998; Tyagi and Fr, 1996) and is ideal for quantitative assessment of individual transcripts. But the absence of a stable multiplex implementation makes this approach equally unsuitable for signature analysis. Conventional multiplex RT-PCR is simple and cheap but suffers from low amplification fidelity, not to mention the absence of a convenient way to detect, identify and quantify multiple amplicons.

Ligation-mediated amplification (LMA), in which two oligonucleotide probes are annealed immediately adjacent to each other on a complementary target DNA or RNA molecule and fused together by a DNA ligase (Landegren et al., 1988; Nilsson et al., 2000) to yield an synthetic amplification template (Hsuih et al., 1996), provides high targeting specificity and, by incorporating universal primer recognition sequences in fixed length ligation products, maintains target representation during multiplex PCR. Further, the ability to include distinct sequence addresses in one of the paired probes allows each of the resulting amplicons to be uniquely identified. Two gene expression profiling solutions based upon these principles-known as RASL (Yeakley et al., 2002) and RT-MLPA (Eldering et al., 2003)—each allowing the simultaneous analysis of around fifty transcripts, have been described.

The Luminex xMAP technology platform is composed of a basic auto-injecting bench-top two laser flow cytometer and a panel of one hundred sets of carboxylated polystyrene microspheres, each set being impregnated with different proportions of two fluorophores, allowing each bead to be classified on its passage through the flow cell (www.luminexcorp.com). Furnishing bead sets with so-called molecular barcodes (Shoemaker et al., 1996)—short unique DNA sequences with uniform hybridization characteristics—delivers an optimized universal detection solution for amplicons designed to contain complementary sequences (lannone et al., 2000). The simplicity, flexibility, throughput and modest capital and operating costs of the Luminex system compares very favorably with the self-assembled bead fiber-optic bundle array and capillary electophoresis detection pieces intrinsic to the RASL and RT-RLPA procedures (Eldering et al., 2003; Yeakley et al., 2002). This motivated evaluation of an integrated LMA-FlexMAP gene expression signature analysis solution (FIG. 1). A detailed description of our method is also available online (www.broad.mit.edu/cancer).

A ninety member gene expression signature was derived from an unbiased genome-wide transcriptional analysis of a cell culture model of differentiation. Total RNA was isolated from HL60 cells following treatment with tretinoin or vehicle (DMSO) alone, amplified and labeled by in vitro transcription (IVT), and target hybridized to Affymetrix GeneChip microarrays. Features reporting above threshold were binned into three groups of equal size on the basis of expression level. Ten transcripts exhibiting low, moderate and high differential expression between the two conditions were then selected from each bin, populating a matrix of nine classes (Table 2) representing the diversity of expression characteristics.

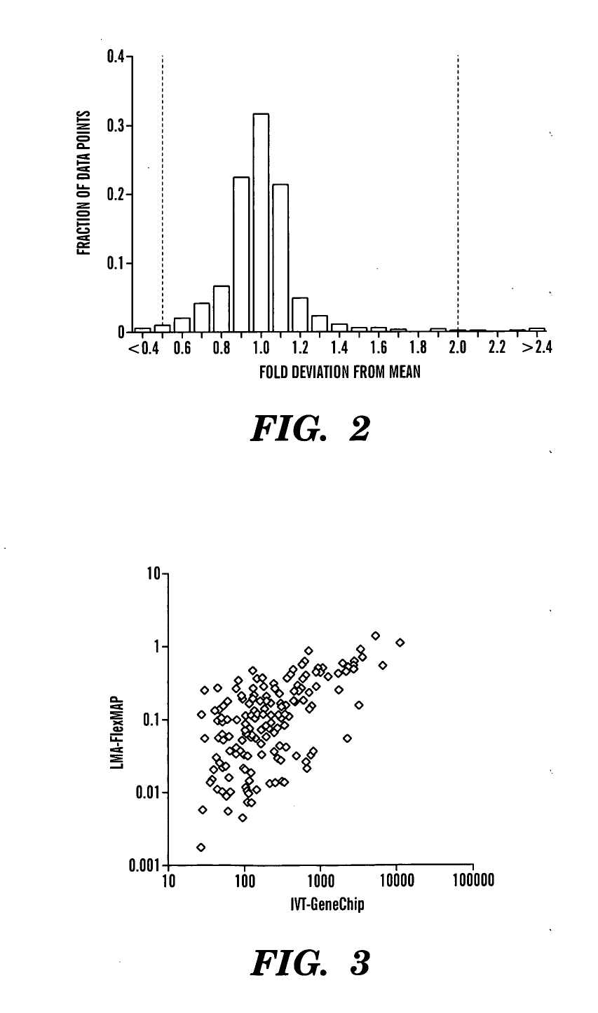

Probe pairs incorporating unique FlexMAP barcode sequences were designed against each of the ninety transcripts (Table 3) and ten aliquots of the two original RNA samples were analyzed in this space by LMA-FlexMAP. Following subtraction of background signals, thresholding and normalization against an internal reference control feature (ie GAPDH), 98.5% of data points fell within two fold of their corresponding means (FIG. 2). This compares well with a similar assessment of variability for RASL (Yeakley et al., 2002) and demonstrates the high reproducibility of the method. Most of the variability was accounted for by a single feature (13/38 failures) and two wells (17/38).

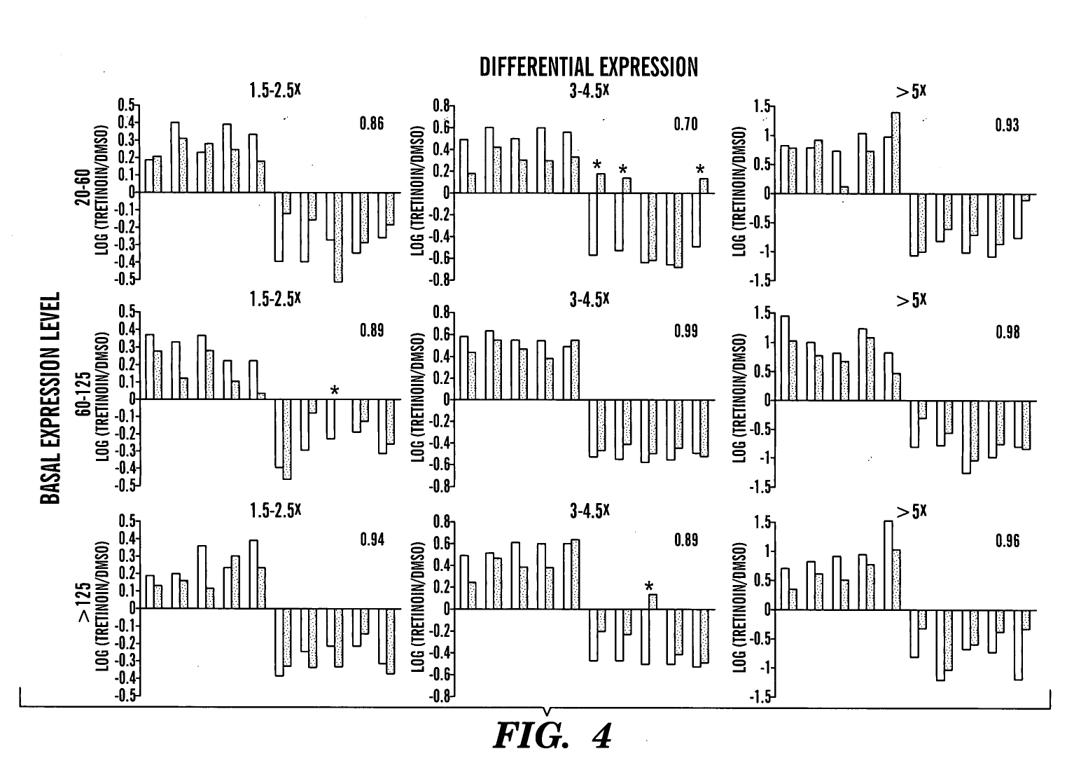

There was a poor overall correlation between the mean expression levels reported by the two platforms (correlation coefficient=0.714). LMA-FlexMAP appears to overestimate transcript levels relative to IVT-GeneChip but to a degree inversely related to absolute level (FIG. 3). Estimates of the extent of differential expression reported by our solution were correspondingly less across the entire feature space, but there was broad qualitative agreement in this parameter even in the low basal and low differential expression classes (FIG. 4). Five probe pairs produced gross errors, in line with our typical first-pass probe failure rate of 5%. One failure is attributable to ambiguous annotation of the microarray and another to high background signal. All failure modes can generally be remedied by probe redesign. Irrespective, the overall correlation of log ratios between the platforms was 0.924, somewhat higher than that reported for a similar comparison between oligonucleotide and cDNA microarrays (Yuen et al., 2002). We repeated this entire LMA-FlexMAP analysis on two separate occasions with similar results. The coefficient of variation of mean expression level for each of the ninety features across all three independent evaluations had a mean of 13.8% (maximum of 49.8%), indicating high stability of the platform.

Next, we applied our method to all idealized gene expression signature analysis problem, requiring the ability to diagnose the presence of a predefined biological state in each of a large number of samples. Data were collected for our ninety gene feature set from ninety-four microtiter well cultures of HL60 cells each treated with either tretinoin or vehicle alone. Drug concentration and treatment duration were reduced by 80% and 60%, respectively, to model the sub-maximal signatures encountered in a small molecule screen. Process time from the additional of cell lysis buffer to data delivery was sixteen hours, and overall unit cost was approximately $2. Six wells (6.4%) had internal control features signals more than two standard deviations from the mean and were discarded. This throughput and overall drop out rate is typical.

Although the feature set was designed to represent the diversity of expression characteristics rather than to contain the transcripts most highly correlated with the distinction, a k-nearest-neighbor (KNN) classifier (Cover and Hart, 1967) trained on the original high dose long duration IVT-GeneChip data delivered 100% classification accuracy for these low dose short duration samples in the full ninety gene feature space. Classifiers built in the space of each of the nine ten member gene categories had error rates between 14.8% (medium level, low differential expression) and 0% (high level, high differential expression) (Table 1). These results demonstrate both the successful deployment of our solution and the advantage of a method with higher level multiplexing capability.

Our solution underestimates changes in expression level relative to the industry-standard high-end state-of-the-art gene expression profiling platform. However, its impressive classification accuracy in an idealized application indicates that performance can easily be sacrificed for throughput in pursuit of a practical gene expression signature analysis solution, and bodes well for the rapid deployment of any legacy signature with minimal or even no optimization. The assessments reported here also suggest that new signatures designed specifically for this platform should exploit the full content capacity and avoid transcripts expressed at low or moderate levels with low degrees of differential expression. With its simplicity, flexibility, throughput and cost-effectiveness the LMA-FlexMAP method has been a transformative tool in our laboratories whose exploitation for biological discovery shall be reported elsewhere.

### Example 2

### A Bead-Based microRNA-Expression Profiling Method

Materials and Methods

Samples

Details of sample information are available in Table 9. Total RNAs were prepared from tissues or cell lines using TRIzol (Invitrogen, Carlsbad, Calif.), as described (Ramaswamy et al., 2001), and in compliance with IRB protocols. Leukemia bone marrow mononuclear cells were collected from patients treated at St. Jude Children's Research Hospital and at Dana-Farber Cancer Institute and their immunophenotype and genotype determined as previously described (Ferrando et al., 2002; Yeoh et al., 2002). Normal mouse lung and mouse lung cancer samples were collected from KRasLA1 mice, and genotyped as described (Johnson et al., 2001). Lungs from four- to five-month old mice were inflated with phosphate-buffered saline prior to removal. Individual lung tumors and normal lungs were dissected and immediately frozen on dry ice before RNA preparation. HL-60 cells were plated at 1.5×10⁵ cell/ml and induced to differentiate by 1 μM all-trans retinoic acid (Sigma, St. Louis, Mo.; in ethanol). Cells were harvested after 1, 3 and 5 days. Culturing conditions for other cells are detailed in Example 3.

miRNA Labelling

Target preparation from total RNA follows the described procedure (Miska et al., 2004), with modifications. Briefly, two synthetic pre-labeling-control RNA oligonucleotides (5′-pCAGUCAGUCAGUCAGUCAGUCAG-3′ (Seq ID No: 872), and 5′-pGACCUCCAUGUAAACGUACAA-3′ (Seq ID No: 873), Dharmacon, Lafayette, Colo.) were used to control for target preparation efficiency. They were each spiked at 3 fmoles per μg total RNA. Small RNAs (18- to 26-nucleotide) were recovered from 1 to 10 μg total RNA through denaturing polyacrylamide gel purification. Small RNAs were adaptor-ligated sequentially on the 3′-end and 5′-end using T4 RNA ligase (Amersham Biosciences, Piscataway, N.J.). After reverse-transcription using adaptor-specific primer, products were PCR amplified (95° C. 40 see, 50° C. 30 sec. 72° C. 30 sec, 18 cycles for 10 μg starting total RNA; 3′-primer: 5′-tactggaattcgcggtta-3′ (Seq ID No: 874), 5′ primer: 5′-biotin-caacggaattcctcactaaa-3′. (Seq ID No: 875), IDT, Coralville, Iowa). For side-by-side comparison of the bead-detection and the glass-microarray, a 5′-Alexa-532-modified primer was used for compatibility with the glass-microarray. PCR products were precipitated and dissolved in 66 μl TE buffer (10 mM Tris HCl, pH8.0, 1 mM EDTA) containing two biotinylated post-labeling-control oligonucleotides (100 fmoles of FVR506, and 25 fmoles PTG20210, see Table 10).

Bead-Based Detection

miRNA capture probes were 5′-amino-modified oligonucleotides with a 6-carbon linker (IDT). Capture probes for miRNAs and controls were divided into three sets (see Table 10), and each sample was profiled in 3 assays on these three probe sets separately. Probes were conjugated to carboxylated xMAP beads (Luminex Corporation, Austin, Tex.) in 96-well plates, following the manufacturer's protocol. For each probe set, 3 μl of every probe-bead conjugate were mixed into 1 ml of 1.5×TMAC (4.5 M tetramethylammonium chloride, 0.15% sarkosyl, 75 mM Tris-HCl, pH 8.0, 6 mM EDTA). Samples were hybridized in a 96-well plate, with two mock PCR samples (using water as template) in each plate for background control. Hybridization was carried out with 33 μl of the bead mixture and 15 μl of labelled material, at 50° C. overnight. Beads were spun down, resuspended in 1×TMAC containing 10 μg/ml streptavidin-phycoerythrin (Molecular Probes, Eugene, Oreg.) and incubated at 50° C. for 10 minutes before data acquisition on a Luminex 100IS machine. Median fluorescence intensity values were measured.

Computational Analyses

Profiling data were first scaled according to the post-labeling-controls and then the pre-labeling-controls, in order to normalize readings from different probe/bead sets for the same sample, and to normalize for the labeling efficiency, as detailed in Materials and Methods of Example 3. Data were thresholded at 32 and log₂-transformed. Hierarchical clustering was performed with average linkage and Pearson correlation. Prior to clustering, data were filtered to eliminate genes with expression lower than 7.25 (on log₂ scale) in all samples. Next, all features were centered and normalized to a mean of 0 and a standard deviation of 1. k-Nearest-Neighbor classification of normal vs. tumor was performed with k=3 in the selected feature space using Euclidean distance measure. Note that different metrics were used for clustering and normal/tumor classification. Features were selected for the distinction between all normal samples vs. all-tumors (for colon, kidney, prostate, uterus, lung and breast; P<0.05 after Bonferroni-correction). P values were calculated using a variance-fixed t-test with a minimal standard deviation of 0.75, after confounding the tissue types. Multi-class predictions of poorly differentiated tumors were performed using the probabilistic neural network algorithm, a Gaussian-weighted nearest neighbor method. For each test sample, the tissue type that had the highest probability in multiple one-tissue-versus-the-rest predictions was assigned. Feature number and the Gaussian width were optimized based on leave-one-out cross-validations on the training data set. Features were selected based on the variance-fixed t-test score, requiring equal number of up- and down-regulated features. Distances were based on the cosine in the selected feature space.

Expression Data

miRNA expression data have been submitted to GEO (http://www.ncbi.nlm.nih.gov/geo), with a series accession number of GSE2564. mRNA expression data were published previously (Ramaswamy et al., 2001), and are available together with miRNA expression data at http://www.broad.mit.edu/cancer/pub/miGCM.

Results and Discussion

Much progress has been made over the past decade in developing a molecular taxonomy of cancer (see review Chung et al., 2002). In particular, it has become clear that among the ˜22,000 protein-coding transcripts are mRNAs capable of classifying a wide variety of human cancers (Ramaswamy et al., 2001). Recently, hundreds of small, non-coding miRNAs have been discovered (see review-Bartel, 2004). The first identified miRNAs, the products of the C. elegans genes lin-4 and let-7, play important roles in controlling developmental timing and probably act by regulating miRNA translation (Ambros and Horvitz, 1984; Lee et al., 1993; Reinhart et al., 200). When lin-4 or let-7 is inactivated, specific epithelial cells undergo additional cell divisions as opposed to their normal differentiation. Since abnormal proliferation is a hallmark of human cancers, it seemed possible that miRNA expression patterns might denote the malignant state. Furthermore, altered expression of a few miRNAs has been found in some tumor types (Calin et al., 2002; Eis et al., 2005; Johnson et al., 2005; Michael et al., 2003). However, the potential for miRNA expression to inform cancer diagnosis has not been systematically explored.

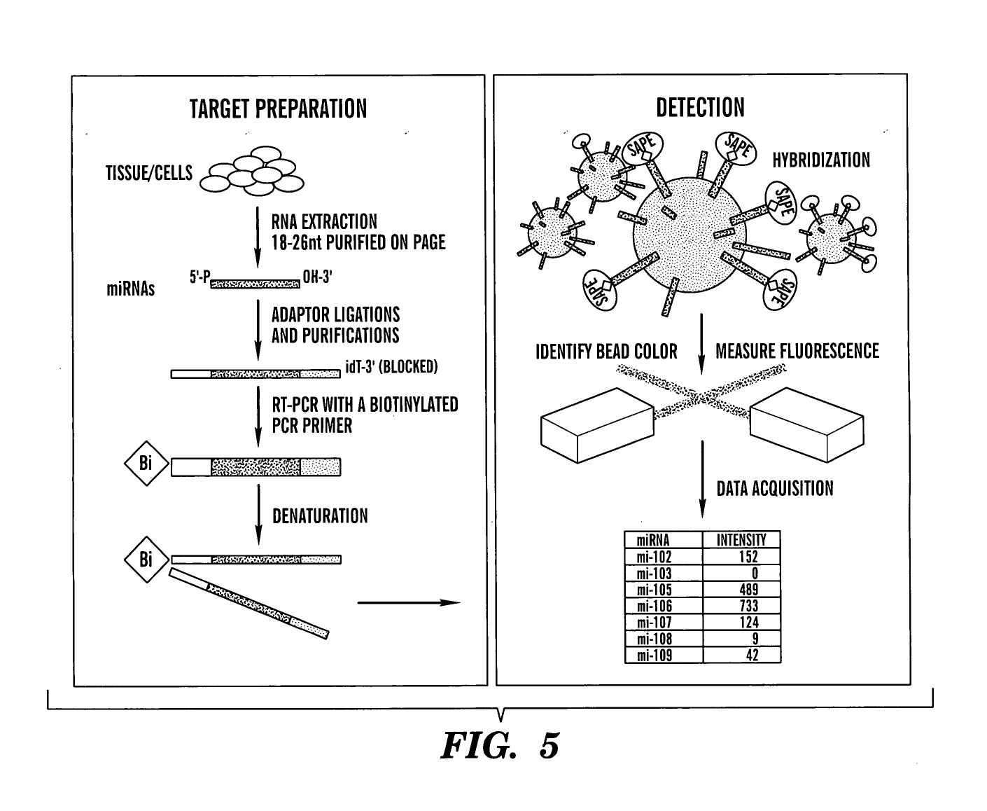

To determine the expression pattern of all known miRNAs, we first needed to develop an accurate and inexpensive profiling method. This goal is challenging, because of the miRNAs' short size (around 21 nucleotides) and the sequence similarity of members of miRNA families. Glass-slide microarrays have been used for miRNA profiling (Babak et al., 2004; Barad et al. 2004; Liu et al., 2004; Miska et al., 2004; Nelson et al., 2004; Thomson et al., 2004; Sun et al., 2004), but cross-hybridization of related miRNAs has been problematic. We therefore developed a bead-based profiling method. Oligonucleotide-capture probes complementary to miRNAs of interest were coupled to carboxylated 5-micron polystyrene beads impregnated with variable mixtures of two fluorescent dyes that yield up to 100 colors, each representing a miRNA. Following adaptor ligations utilizing both the 5′-phosphate and the 3′-hydroxyl groups of miRNAs (Miska et al., 2004), reverse-transcribed miRNAs were PCR-amplified using a common biotinylated primer, hybridized to the capture beads, and stained with streptavidin-phycoerythrin. The beads were then analyzed on a flow cytometer capable of measuring bead color (denoting miRNA identity) and phycoerythrin intensity (denoting miRNA abundance) (FIG. 5).

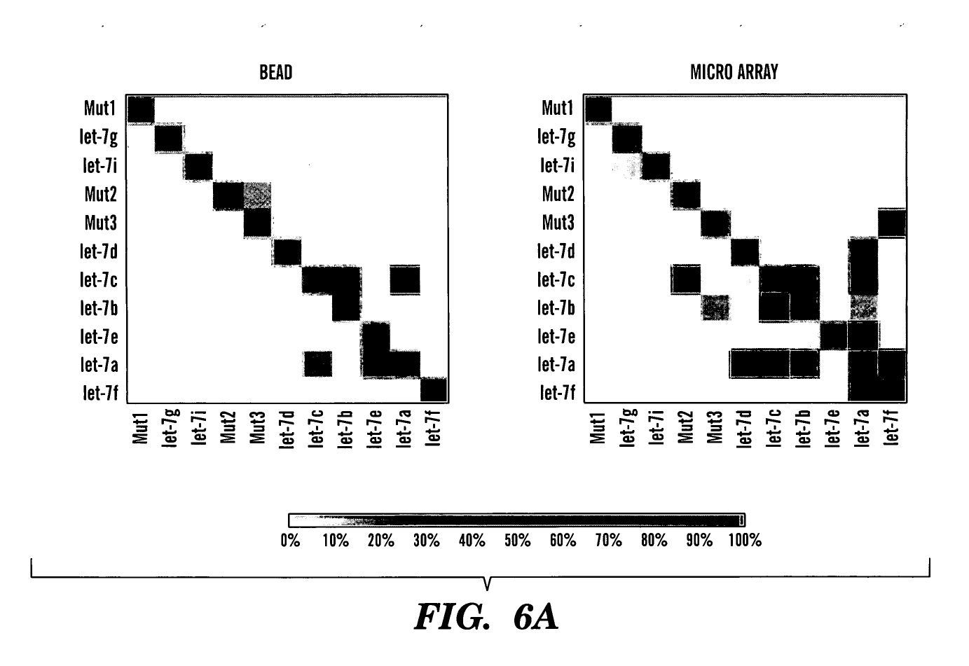

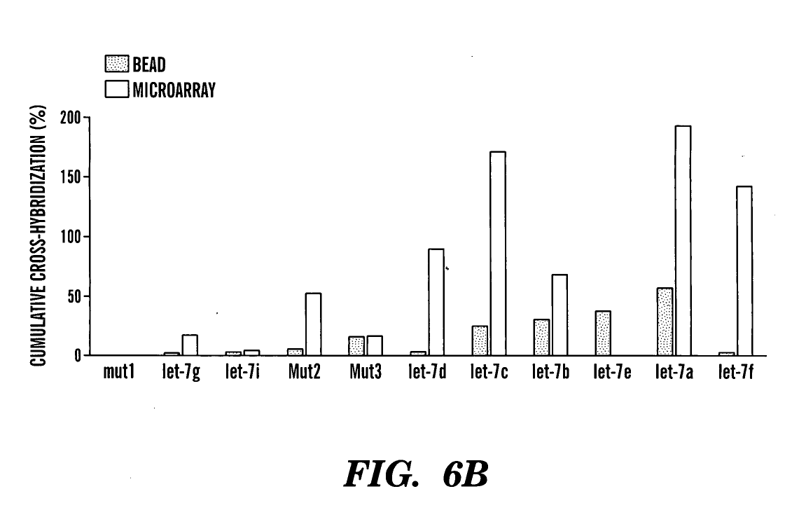

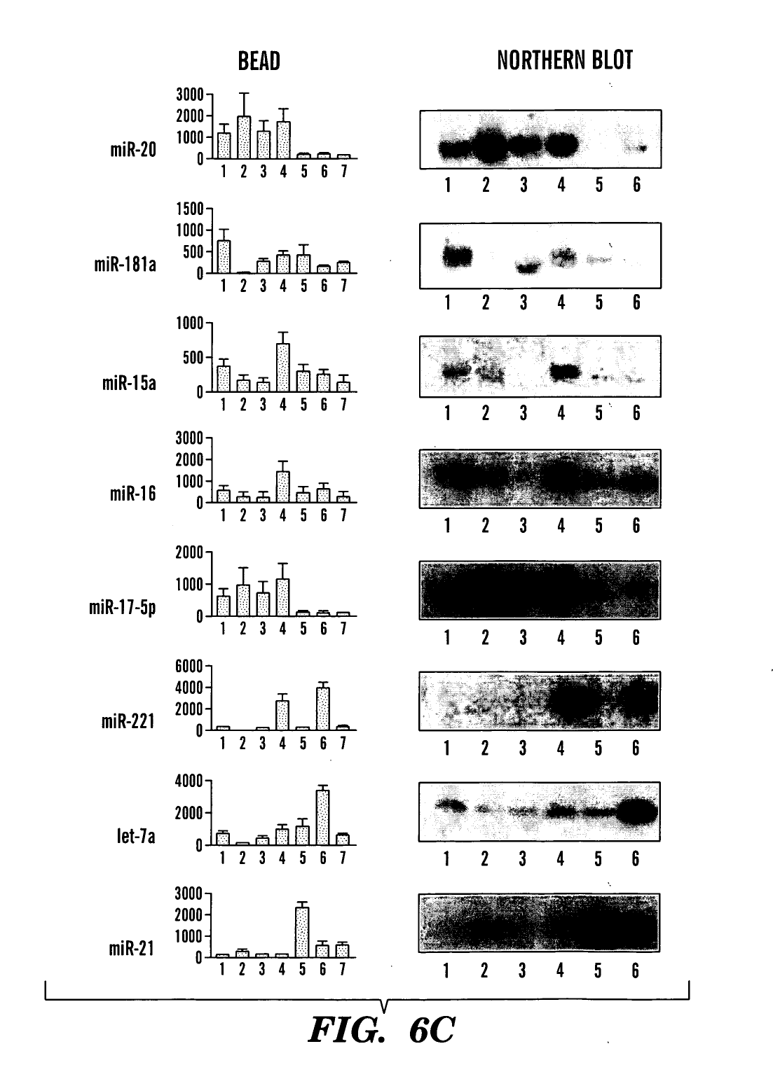

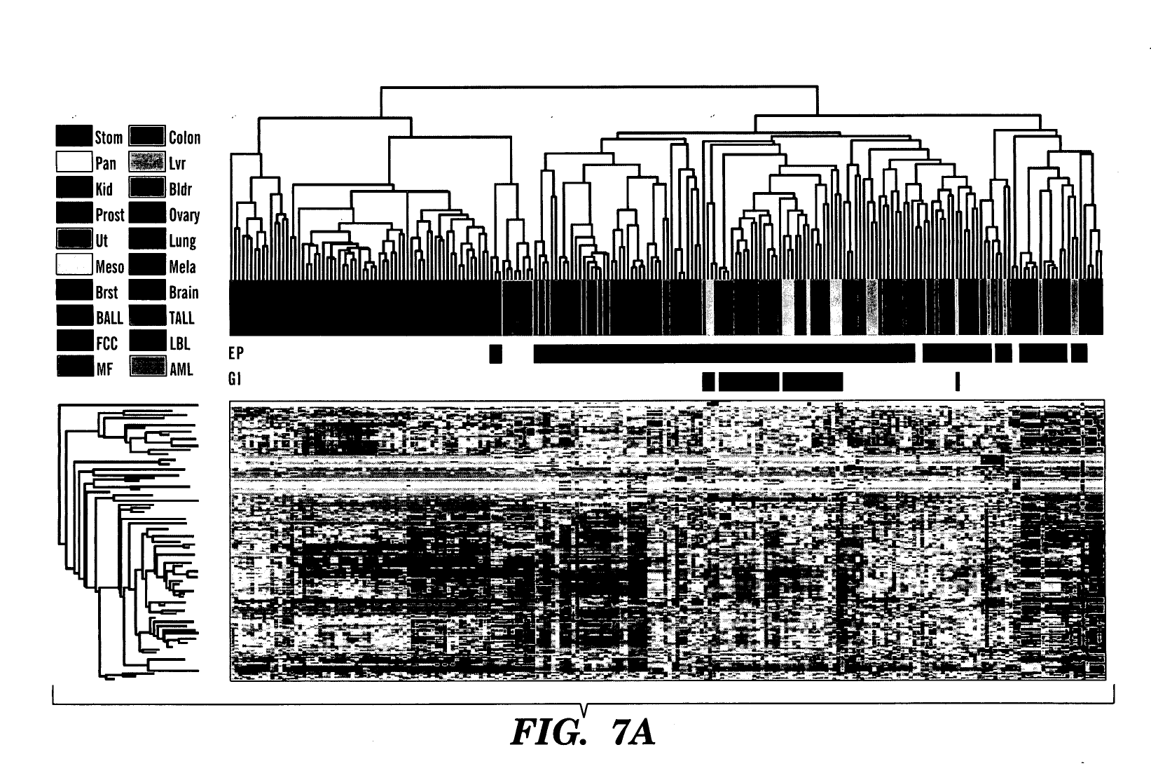

Bead-based hybridization has the theoretical advantage that it may more closely approximate hybridization in solution and as such the specificity might be expected to be superior to glass microarray hybridization. Indeed, a spiking experiment involving 11 related sequences comparing bead-based detection to microarray-based detection demonstrated increased specificity of beads compared to microarrays, even for single base-pair mismatches (FIG. 6a, 6b). In addition, the bead method exhibited linear detection over two logs of expression (Example 3). Eight miRNAs were validated by northern blotting in seven cell lines. In all cases, bead-based detection paralleled the northern data (FIG. 6c). These results demonstrate that bead-based miRNA detection is feasible, having the attractive properties of improved accuracy, high speed and low cost. The bead-based detection platform also provides flexibility in that additional miRNA capture beads can be added to the mixture, thereby detecting newly discovered miRNAs.

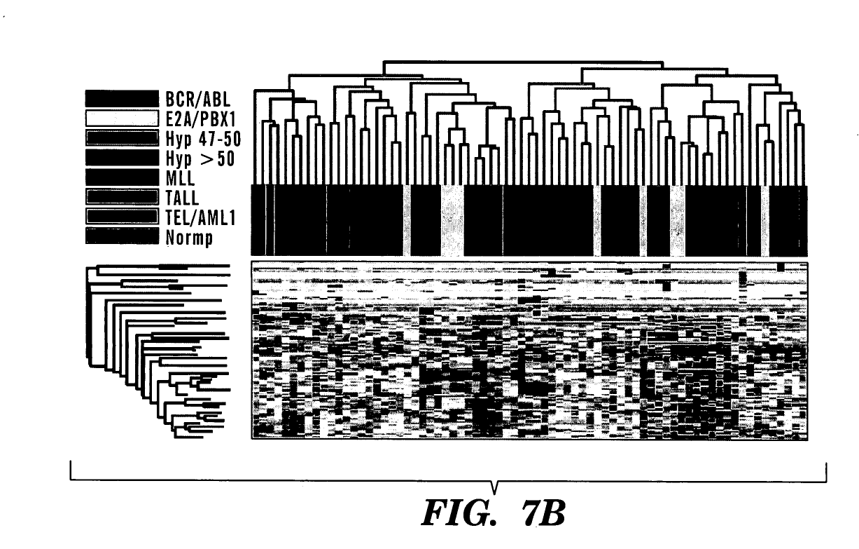

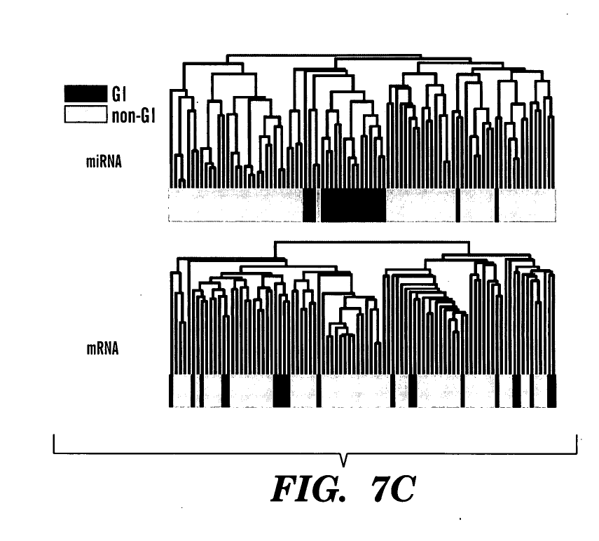

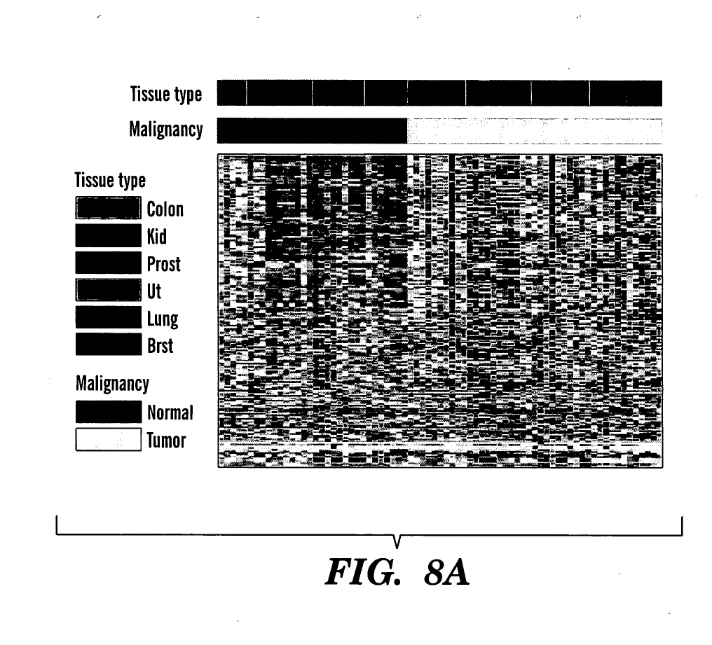

We then set out to determine the expression pattern of all known miRNAs across a large panel of samples representing a diversity of human tissues and tumor types. While miRNA expression has been previously explored in small sets of tissues (Babak et al., 2004; Barad et al., 2004; Liu et al., 2004; Nelson et al., 2004; Thomson et al., 2004; Sun et al., 2004) or isolated cell types (e.g. chronic lymphocytic leukemia in Calin et al., 2001), the extent of differential expression of miRNAs across cancers has not been previously determined. Indeed, one might not have expected that miRNA expression patterns would be informative with respect to cancer diagnosis, because of the relatively small number of miRNAs encoded in the genome. Remarkably, we observed differential expression of nearly all miRNAs across cancer types (FIG. 7a). Moreover, hierarchical clustering of the samples in the space of miRNAs recapitulated the developmental origin of the tissues. For example, samples of epithelial origin fell on a single branch of the dendrogram, whereas the other major branch was predominantly populated with hematopoietic malignancies.

Furthermore, the miRNAs partitioned tumors within a single lineage. For example, we examined the miRNA profiles of 73 bone marrow samples obtained from patients with acute lymphoblastic leukemia (ALL). As shown in FIG. 7b, hierarchical clustering revealed non-random partitioning of the samples into three major branches: one containing all 5 t(9;22) BCR/ABL positive ALLs and 10 of 11 t(12;21) TEL/AML1 cases, a second branch containing 15/19 T-cell ALLs, and a third containing all but one of the samples with MLL gene rearrangement. These experiments demonstrate that even within a single developmental lineage, distinct patterns of miRNA expression reflecting mechanism of transformation are observable and further support the notion that miRNA expression patterns encode the developmental history of human cancers.

Among the epithelial samples, those of the gastrointestinal tract were of particular interest. Samples from colon, liver, pancreas and stomach all clustered together (FIG. 7a), reflecting their common derivation from tissues of embryonic endoderm. That is, the dominant structure in the space of miRNAs was one of developmental history. In contrast, when these samples were profiled in the space of ≠16,000 miRNAs, the coherence of gut-derived samples was not recovered (FIG. 7c). This observation may result from the large amount of noise and unrelated signals that are embedded in the high dimensional miRNA data. Whether or not the miRNAs that are highly expressed in the gut-associated cluster (miR-192, miR-194, miR-215) play a functional role in the specification of gut development or gut-derived tumors remains to be investigated.

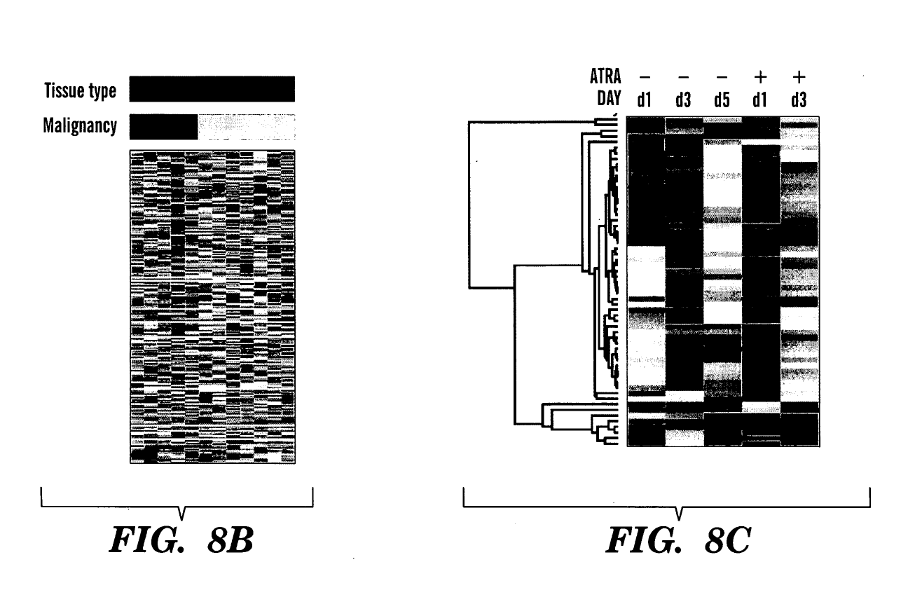

Having determined that miRNA expression distinguishes tumors of different developmental origin, we next asked whether miRNAs could be used to distinguish tumors from normal tissues. We previously reported that there exist no robust mRNA markers that are uniformly differentially expressed across tumors and normal tissues of different lineages (Ramaswamy et al., 2001). It was therefore striking to observe that despite the fact that some mRNAs are upregulated or unchanged, the majority of the miRNAs (129/217, p<0.05, after correction for multiple hypothesis testing) had lower expression in tumors compared to normal tissues, irrespective of cell type (FIG. 8a). Importantly, the cancer cell lines also showed low miRNA expression relative to normal tissues (FIG. 9).

To exclude any possibility that the differential miRNA expression might be related to differences in collection of tumor vs. normal samples, we studied a mouse model of KRas-induced lung cancer (Johnson et al., 2001). We isolated miRNAs from normal lung or lung adenocarcinomas from individual mice, thereby precluding any differences in collection procedure. Notably, because of miRNA sequence conservation between human and mouse, the same miRNA capture beads could be used to profile the murine samples. As shown in FIG. 8b, the same tumor vs. normal distinction is seen in the mouse. Accordingly, a tumor-normal classifier built on human samples had 100% accuracy when tested in the mouse. Taken together, these studies indicate that miRNAs are unexpectedly rich in information content with respect to cancer.

Our observation that miRNA expression appeared globally higher in normal tissues compared to tumors led to the hypothesis that global miRNA expression reflects the state of cellular differentiation. To test this hypothesis, we explored an experimental model in which we treated the myeloid leukemia cell line HL-60 with all-trans retinoic acid, a potent inducer of neutrophilic differentiation-(Stegmaier et al., 2004). As predicted, miRNA profiling demonstrated the induction of many miRNAs coincident with differentiation (FIG. 8c). In primary human hematopoietic progenitor cells undergoing erythroid differentiation in vitro, we observed a similar increase in miRNA expression occurring at a stage in differentiation when the cells continued to proliferate (see Example 3). These experiments support the hypothesis that global changes in miRNA expression are associated with differentiation, the abrogation of which is a hallmark of all human cancers. These findings are also consistent with the recent observation that mouse embryonic stem cells lacking Dicer, an enzyme required for miRNA maturation, fail to differentiate normally (Kanellopoulou et al., 2005).

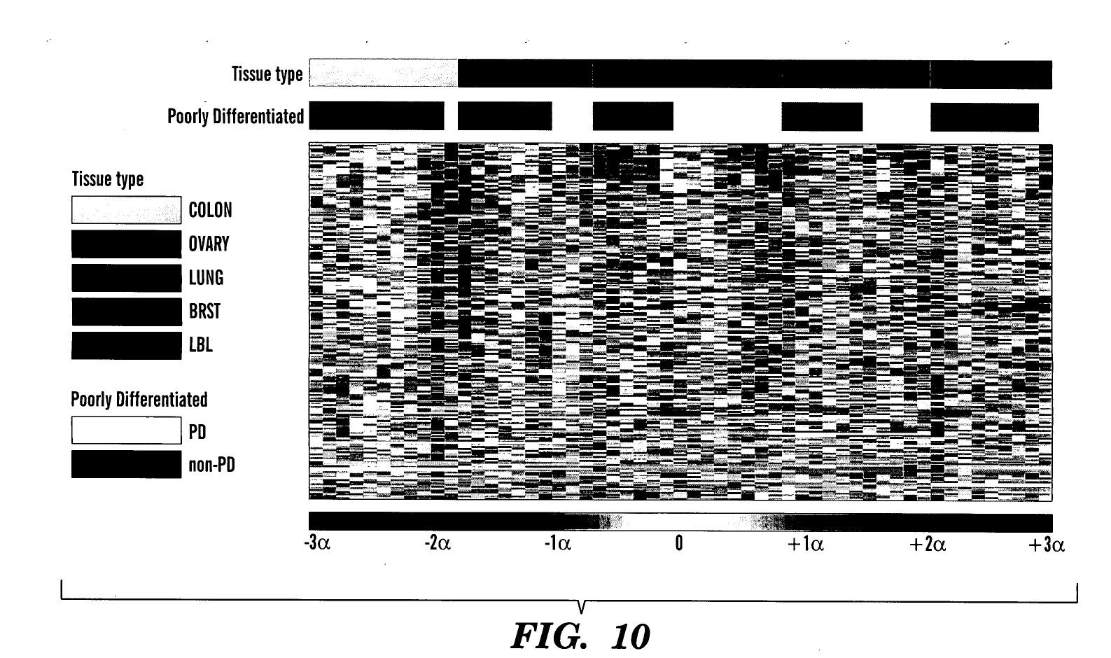

We next turned to a more challenging diagnostic distinction: that of tumors of histologically uncertain cellular origin. It is estimated that 2%-4% of all cancer diagnoses represent cancers of unknown origin or diagnostic uncertainty (see review Pavlidis et al., 2003). To address this, we analyzed 17 poorly differentiated tumors whose histological appearance alone was non-diagnostic, but whose clinical diagnosis was established by anatomical context, either directly (e.g. a primary tumor arising in the colon) or indirectly (a metastasis of a previously identified primary). A training set of 68 more differentiated tumors representing 11 tumor types for which both mRNA and miRNA profiles were available was used to generate a classifier. This classifier was then used without modification to classify the 17 poorly-differentiated test samples. As a group, poorly differentiated tumors had lower global levels of miRNA expression compared to the more-differentiated training set samples (FIG. 10), consistent with the notion that miRNA expression is closely linked to differentiation. Despite this overall low level of miRNA expression, the miRNA-based classifier established the correct diagnosis of the poorly differentiated samples far beyond what would be expected by chance for an 11-class classifier (12/17 correct; p<5×10⁻¹¹). In contrast, the mRNA-based classifier was highly inaccurate (1/17 correct; p=0.47), as we previously reported (Ramaswamy et al., 2001).

The experiments reported here demonstrate the feasibility and utility of monitoring the expression of miRNAs in human cancer. The unexpected findings are the extraordinary level of diversity of miRNA expression across cancers and the large amount of diagnostic information encoded in a relatively small number of miRNAs. The implication is that, unlike with mRNA expression, a modest number of miRNAs (˜200 in total) might be sufficient to classify human cancers. Moreover, the bead-based miRNA detection method has the attractive property of being not only accurate and specific but also being easily implementable in a routine clinical setting. In addition, unlike mRNAs, mRNAs remain largely intact in routinely collected, formalin-fixed paraffin-embedded clinical tissues (Nelson et al., 2004). More work is required to establish the clinical utility of miRNA expression in cancer diagnosis, but the work described here indicates that miRNA profiling has unexpected diagnostic potential. The mechanism by which miRNAs are under-expressed in cancer remains unknown. We did not observe substantive decreases of miRNAs encoding components of the miRNA processing machinery (Dicer, Drosha, Argonaute2, DGCR8 (Cullen, 2004), Example 3), but clearly other mechanisms of regulating miRNAs are possible.

The findings reported here are consistent with the hypothesis that in mammals, as in C. eleganis, miRNAs can function to prevent cell division and drive terminal differentiation. An implication of this hypothesis is that down-regulation of some miRNAs might play a causal role in the generation or maintenance of tumors. Epithelial cells affected in C. elegans lin-4 and let-7 miRNA mutants generate a stem-cell-like lineage, dividing to produce daughters that, like them selves, divide rather than differentiate (Ambros and Horvitz, 1984; Reinhart et al., 2000). We speculate that aberrant miRNA expression might similarly contribute to the generation or maintenance of “cancer stem cells” recently proposed to be responsible for cancerous growth in both leukemias and solid tumors (Al-Hajj et al., 2003; Lapidot et al., 1994; Reya et al., 2001; Singh et al., 2004).

### Example 3

### MicroRNA Expression Profiles Classify Human Cancers

Additional information about the paper and a frequently-asked-questions (FAQ) page are available at http://www.broad.mit.edu/cancer/pub/miGCM.

Materials and Methods

Cell Culture

HEL, TF-1, PC-3, MCF-7, HL-60, SKMEL-5, 293 and K562 cells were obtained from the American Type Culture Collection (ATCC, Manassas, Va.), and cultured according to ATCC instructions. All T-cell ALL cell lines were cultured in RPMI medium supplemented with 10% fetal bovine serum. CCRF-CEM and LOUCY cells were obtained from ATCC. ALL-SIL, HPB-ALL, PEER, TALL1, P12-ICHIKAWA cells were obtained from the German Collection of Microorganisms and Cell Cultures (DSMZ, Braunschweig, Genmany). SUPT11 cells were a kind gift of Dr. Michael Cleary at Stanford University.

Umbilical cord blood was obtained under an IRB approved protocol from the Brigham and Women's Hospital. Light-density mononuclear cells were separated by Ficoll-Hypaque centrifugation, and CD34⁺ cells (85-90% purity) were enriched using Midi-MACS columns (Miltenyi Biotec, Auburn, Calif.). Erythroid differentiation of the CD34⁺ cells was induced in two stages in liquid culture (Ebert et al., 2005). For the first seven days, cells were cultured in Serum Free Expansion Medium (SFEM, Stem Cell Technologies, Tukwila, Wash.) supplemented with penicillin/streptomycin, glutamine, 100 ng/mL stem cell factor (SCF), 10 ng/mL interleukin-3 (IL-3), 1 μM dexamethasone (Sigma), 40 μg/ml lipids (Sigma), and 3 IU/ml erythropoietin (Epo). After 7 days, cells were cultured in the same medium without dexamethasone and supplemented with 10 IU/ml Epo. For flow cytometry analyses, approximately 1 to 5×10⁵ cells were labeled with a phycoberythrin-conjugated antibody against glycophorin-A (CD235a, Clone GA-R2, BD-Pharmingen, San Jose, Calif.) and a FITC-conjugated antibody against CD71 (Clone M-A712, BD-Phanningen). Flow cytometry analyses were performed using a FACScan flow cytometer (Becton Dickinson).

Glass-Slide Detection of miRNAs

Glass slide microarrays were spotted oligonucleotide arrays and hybridized as described previously (Miska et al., 2004). Briefly, 5′-amino-modified oligonucleotide probes (the same ones as used on the bead platform) were printed onto amide-binding slides (CodeLink, Amersham Biosciences). Printing and hybridization were done following the slides manufacturer's protocols with the following modifications: oligonucleotide concentration for printing was 20 μM in 150 mM sodium phosphate, pH 8.5. Printing was done on a MicroGrid TAS II arrayer (BioRobotics) at 50% humidity. Labeled PCR product was resuspended in hybridization buffer (5×SSC, 0.1% SDS, 0.1 mg/ml salmon sperm DNA) and hybridized at 50° C. for 10 hours. Microarray slides were scanned using an arrayWoRx^{e} biochip reader (Applied Precision) and primary data were analyzed using the Digital Genome System suite (Molecularware).

Northern Blot Analysis

Northern blot analyses were carried out as described (Lau et al., 2001). Total RNAs from cell lines were loaded at 10 μg per lane. Blots were detected with DNA probes complementary for human miR-20, miR-181a, miR-15a, miR-16, miR-17-5p, miR-221, let-7a, and miR-21.

Quantitative RT-PCR

Reverse transcription (RT) reactions were carried out on 50 to 200 ng total RNA in 10 μl reaction volumes, using the TaqMan reverse transcription kit (Applied Biosystems, Foster City, Calif.) and random hexamers, following the manufacturer's protocol. RT products were diluted 5-fold in water and assayed using TaqMan Gene Expression Assays (Applied Biosystems) in triplicates, on an ABI PRISM 7900HT real-time PCR machine. Efficiency of PCR amplification was determined by 5 two-fold-serial-diluted samples from HL-60 cDNA. The TaqMan Gene Expression Assays used are listed in the parentheses. (Dicer1: Hs00998566_ml; Ago2/EIF2C2: Hs00293044_ml; Drosha/RNase3L: Hs00203008_ml; DGCR8: Hs00256062_ml; and eukaryotic 18S rRNA endogenous control)

Data Preprocessing and Quality Control

To eliminate bead-specific background, the reading of every bead for every sample was first processed by subtracting the average readings of that particular bead in the two-embedded mock-PCR samples in each plate. As stated in the Methods, every sample was assayed in three wells. Each of the three wells contained 94-probes (19 common probes and 75 unique ones). Out of the 19 common probes are the two pre-labeling controls and the two post-labeling controls. Quality control was performed as part of the preprocessing by requiring that the reading from each control probe exceeds some minimal probe-specific threshold. These thresholds were determined by identifying a natural lower cutoff, i.e. a dip, in the distribution of each control probe. The cutoff values were chosen based on a set of samples in a pilot study. The lower post-control should be greater than 500 and the higher post-control must exceed 2450. The lower and higher pre-controls should exceed 1400 and 2000 respectively (after well-to-well scaling). In this study, about 70% of the samples passed the quality control. Note that the above specifications were used on version 1 of the platform. A similar preprocessing was performed on version 2 of the platform.

Preprocessing was done in four steps: (i) well-to-well scaling—the reading from each well were scaled such that the total of the two post-labeling controls, in that well, became 4500 (a median value based on a pilot study); (ii) sample scaling—the normalized readings were scaled such that total of the 6 pre-labeling controls in each sample reached 27,000 (a median value based on a pilot study); (iii) thresholding at 32 (see below); and (iv) log₂ transformation. All control probes, as well as a probe (EAM296) which had a high background in the absence of any prepared target, were removed before any further analysis. After eliminating these probes, 217 (255 for version 2 of the platform) features were left and these were used throughout the analysis.

Hierarchical Clustering

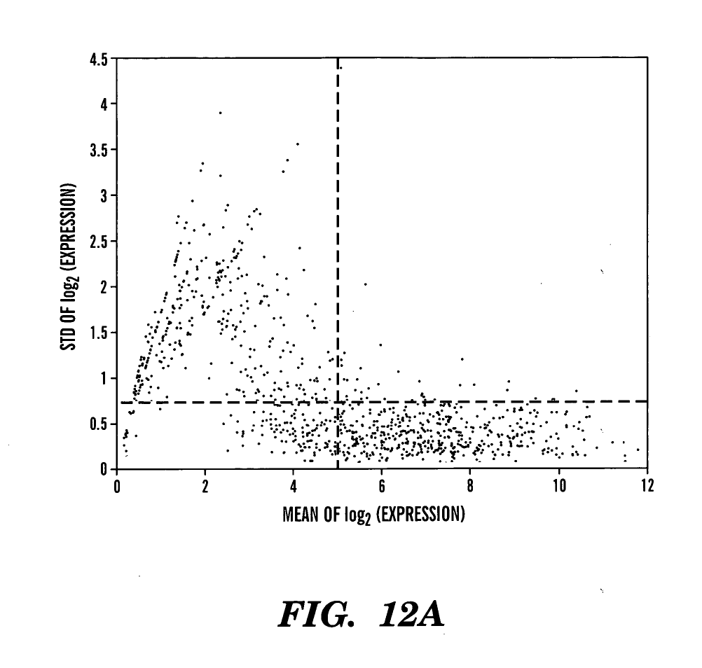

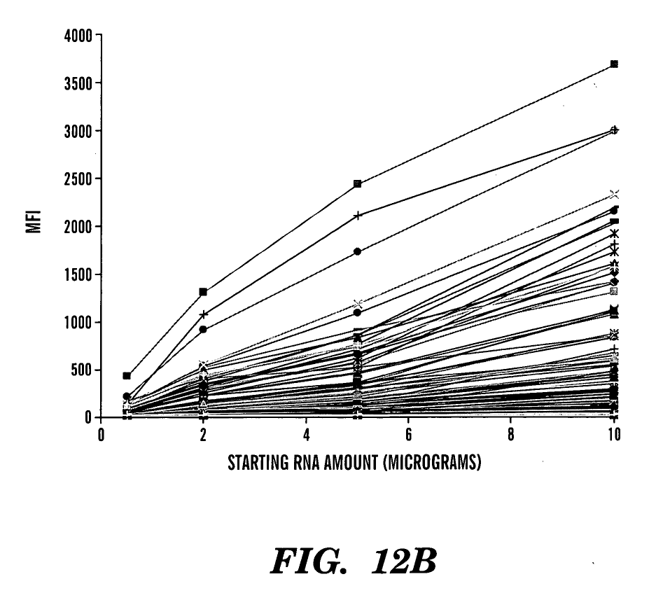

miRNA expression data first underwent filtering. The purpose of this filtering is to remove features which have no detectable expression and thus are uninformative but may introduce noise to the clustering. A miRNA was regarded as “not expressed” or “not detectible”, if in none of the samples, that particular miRNA has an expression value above a minimal cutoff. We applied a cutoff of 7.25 (after data were log₂-transformed). This cutoff value was determined based on noise analyses of target preparation and bead detection (see below and FIG. 12a). In that experiment, the majority of features had a standard deviation below 0.75 when their mean was over 5 in log₂-transformed data. Thus we used a cutoff of 3 standard deviations above the minimal expression level (5+3×0.75=7.25). Any feature that is not expressed under this criterion was filtered out before clustering. Data were then centered and normalized for each feature, bringing the mean to 0 and the standard deviation to 1. This equalizes the contributions of all features. For hierarchical clustering, we used Pearson correlation as a similarity measure, and used the average-linkage algorithm (Jain et al., 1988) for both the samples and the features.

k-Nearest Neighbor (kNN) Prediction

After feature filtration (described in the hierarchical clustering), marker selection was performed on 187 features. The variance-thresholded t-test score was used as a measure to score features. A minimal standard deviation of 0.75 was applied. Markers were searched among the filtered miRNAs. Nominal P-value was calculated for each feature, by permuting the class labels of the samples. In order to select features that best distinguish tumors from normal samples on all tissue types, i.e. taking into account the confounding tissue-type phenotype, restricted permutations were performed (Good, 2004). In restricted permutations, one shuffles the tumor/normal labels only within each tissue type to get the distribution under the desired null hypothesis. To achieve accurate estimates for the p-values, 400 times the number of features (400×187=74,800) of iterations were performed. To correct for multiple-hypotheses testing, markers were selected requiring the Bonferroni-corrected P-values to be less than 0.05. kNN prediction was performed using the kNN module in the GenePattern software, with k=3 and a Euclidean distance measure (GenePattern at http://www.broad.mit.edu/cancer/software/genepattern/index.html).

Probabilistic Neural Network (PNN) Prediction

A two-class PNN (Specht, 1990) prediction was calculated based on the following class posterior probability:

$$P ( c \vert x ) = \frac{P ( x \vert c ) P ( c )}{{\sum }_{{c}^{′}}^{} P ( x \vert {c}^{′} ) P ( {c}^{′} )} = \frac{\frac{P ( c )}{{n}_{c}} {\sum }_{i : {\overline{y}}_{i} ∈ c}^{} exp ( - {D ( x , {y}_{i} )}^{2} / 2 {σ}^{2} )}{{\sum }_{{c}^{′}}^{} [ \frac{P ( {c}^{′} )}{{n}_{{c}^{′}}} {\sum }_{i : {\overline{y}}_{i} ∈ {c}^{′}}^{} exp ( - {D ( x , {y}_{i} )}^{2} / 2 {σ}^{2} ) ]} ,$$

where x is the predicted sample and c is the class for which the posterior probability is calculated. The training set samples are yᵢ, n_{c} is the number of samples of class c in the training set, and D(x,yᵢ) is the distance between the predicted sample and training sample i. In our case, the sum in the denominator (of c′) is over two class values, since we predict a sample either to belong or not to belong to a specific tissue-type. Note that the first step is derived using Bayes rule which allows to incorporate a prior probability for each class, P(c). We used a uniform prior over all 11 tissue-types which translated to 1/11 for being in a certain type and 10/11 for not being in that type. We did not use the tissue-type frequencies in the training set since they likely do not represent the frequencies of different tumors in the general population.

Multi-class prediction using PNN was achieved by breaking down the question into multiple one vs. the rest (OVR) predictions. To perform PNN OVR two-class classification, we built a model based on the training set. This model has two parameters: the number of features used, and σ (the standard deviation of the Gaussian kernel which is used to calculate the contribution of each training sample to the classification). The optimal parameters (for each OVR classifier) were selected using a leave-one-out cross-validation procedure from all possible parameter-pairs in which the number of features ranges from 2 to 30 in steps of 2 and σ takes the values from 1 to 4 times the median nearest neighbor distance, in steps of 0.5 (a total number of 105 combinations). The best model was determined by (i) the fewest number of leave-one-out errors on the training set, which include both false-positive and false-negative errors with the same weight, and (ii) among all conditions with the same error rate, the parameters that gave rise to the maximal mean log-likelihood of the training set were selected. The mean log-likelihood is defined as

$$L [ \{ {x}_{i} \} ; M ] = \frac{1}{\# of training examples} {\sum }_{i}^{} log ( {P}_{m} ( {c}_{i} \vert {x}_{i} ) )$$

where cᵢ is the true class of sample xᵢ and the probability is evaluated using the model M. The top n features were selected using the variance-thresholded t-test score in a balanced manner; n/2 features with the top positive scores and n/2 features with most negative scores. The cosine distance measure was used; D(x,yᵢ)=1−cosine(x,yᵢ).
P-Value Calculation for the Numiber of Correct Classifications

A Binomial distribution was used to calculate the probability to obtain at least the number of correct classifications (on the test set) as we observed. Assuming a random classifier would predict the tissue-type randomly with a uniform distribution over the 11 possible outcomes, the probability of a correct classification is 1/11. This is applicable to the PNN prediction, in which the background frequency of each tissue type was assumed to be 1/11. The p-value is, therefore, the tail of the Binomial distribution from the observed number of correct classifications, s, to the total number of samples in the test set, n:

$$P \text{-} value = {\sum }_{t = s}^{n} ( \begin{matrix}n \\ t\end{matrix} ) {{p}^{t} ( 1 - p )}^{n - t}$$

where p is one over the number of tissue-types (1/11, in our case) and t is the number of correct classification which goes from the observed number, s, to the maximum of possible correct samples n.
Results and Discussion
Development of a Bead-Based miRNA Profiling Platform

Compared with glass-based microarrays, bead-based profiling solutions have the advantages of higher sample throughput and liquid phase hybridization kinetics, while having the disadvantage of lower feature throughput. For the genomic analysis of miRNA expression, this disadvantage is negligible because of the relative small number of identified miRNAs. Since new miRNAs are still being discovered, the flexibility and ease of these “liquid chips” to introduce new features is of particular value.

We developed a bead-based miRNA profiling platform, as detailed in the Methods section. Version 1 of this platform (used for most samples in this study) covers 164 human, 185 mouse, and 174 rat miRNAs, according to Rfam 5.0 miRNA registry database (Ambros et al., 2003; Griffiths-Jones, 2004) (http://www.sanger.ac.uk/Software/Rfam/mirna/index.shtml). Version 2 of this platform (used, for the acute lymphoblastic leukemia study and the erythroid differentiation study) covers additional 24 human, 13 mouse and 2 rat miRNAs (refer to Table 10 for details).

This profiling platform is compatible in theory with any miRNA labeling method that labels the sense strand. For our study, we followed one described by Miska et al., 2004 that labels mature miRNAs through adaptor ligation, reverse-transcription and PCR amplification. We reasoned that the amplification step will allow future use of these labeled materials, which were from precious clinical samples. Defined amounts of synthetic artificial miRNAs were added into each sample of total RNAs as pre-labeling controls. This allows us to normalize the profiling data according to the starting amount of total RNA, using readings from capture probes for these synthetic miRNAs (see Methods for details). This contrasts the use of total feature intensity to normalize the readings of different samples; the hidden assumption of the latter is that the total miRNA expression is the same in all samples, which may not be true considering the small known number of miRNAs.

We analyzed the variation caused by labeling and detection using repetitive assays of the same RNA samples of a few cell lines originated from different tissues; these cell lines have different miRNA profiles; We plotted the standard deviation of each probe versus its means, after the data were log₂-transformed (FIG. 12a). The variations are large for low means, and decrease and stabilize with increasing means. For most measured features with mean above 5 (32 before log₂-transformation), the standard deviation is below 0.75. This value of mean provides a good cutoff for a lower threshold of the data, which was thus used in this study.

We compared the data from expression profiles and northern blots on a panel of 7 cell lines; the same quantities of the same starting total RNAs were used for both analyses. We picked eight miRNAs that are expressed in any of these cell lines and that show differential expression according to the expression profiles, and probed them with northern blots. All eight display good concordance between the two assays (FIG. 6c), indicating that our profiling platform has good accuracy.

We next examined the linearity of profiling (both labeling and detection) by measuring a series of starting materials, covering 0.5 μg to 10 μg of total RNAs from HEL cells. Most miRNAs report good linearity up to 3500 median fluorescence intensity readings (after normalization with pre-labeling-controls. FIG. 12b). Taken together with the threshold level of 32, the profiling method has roughly 100-fold of dynamic range.

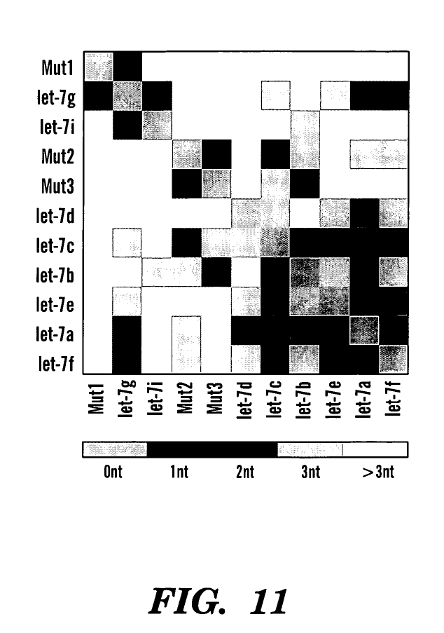

One common issue that affects hybridization-based analyses for miRNAs is the specificity of detection, since many miRNAs are closely-related on the sequence level. To assess the specificity of detection, we synthesized oligonucleotides corresponding to the reverse-transcription products of adaptor-ligated miRNAs, in this case the human let-7 family of miRNAs and a few artificial mutants. The sequences for these oligonucleotides are in Table 11, and the alignment of human let-7 miRNAs and mutant sequences are listed in Table 12. They were then labeled through PCR using the same primer sets. This provides a collection of sequence-pairs that differ by one, two, or a few nucleotides (FIG. 11 and Table 12). Results are presented in Example 2 and in FIG. 6a,b.

Hierarchical Clustering of Multiple Cancer and Normal Samples

We applied this miRNA profiling platform for 140 human cancer specimens, 46 normal human tissues, and various cell lines. The collection of samples covers more than ten tissues and cancer types. This collection was referred to as miGCM (for miRNA Global Cancer Map). We first examined the miRNA expression profiles to see whether we can detect previously reported tissue-restricted expression of miRNAs. Indeed, we observed tissue-restricted expression patterns. For example, miR-122a, a reported liver-specific miRNA (Lagos-Quintana et al., 2002), is exclusively expressed in the liver samples, whereas miR-124a, a brain-specific miRNA (Lagos-Quintana et al., 2002), is abundantly expressed in the brain samples.

We performed hierarchical clustering on this data set, as described in the Methods. Hierarchical clustering is an unsupervised analysis tool that captures internal relationship between the samples. It organizes the samples (or features) into a tree structure (a dendrogram) according to the similarity between the samples (or the features). Close pairs of samples (ones with similar expression profiles) will generally be connected in the dendrogram at an earlier phase, while samples with larger distances (with less similar expression profiles) will be connected at a later phase (details can be found in Duda et al., 2000). The detailed result of hierarchical clustering on both the samples and features using correlation metrics is presented in FIG. 7a and FIG. 9.

Comparison of miRNA and miRNA Clustering in Regard to GI Samples

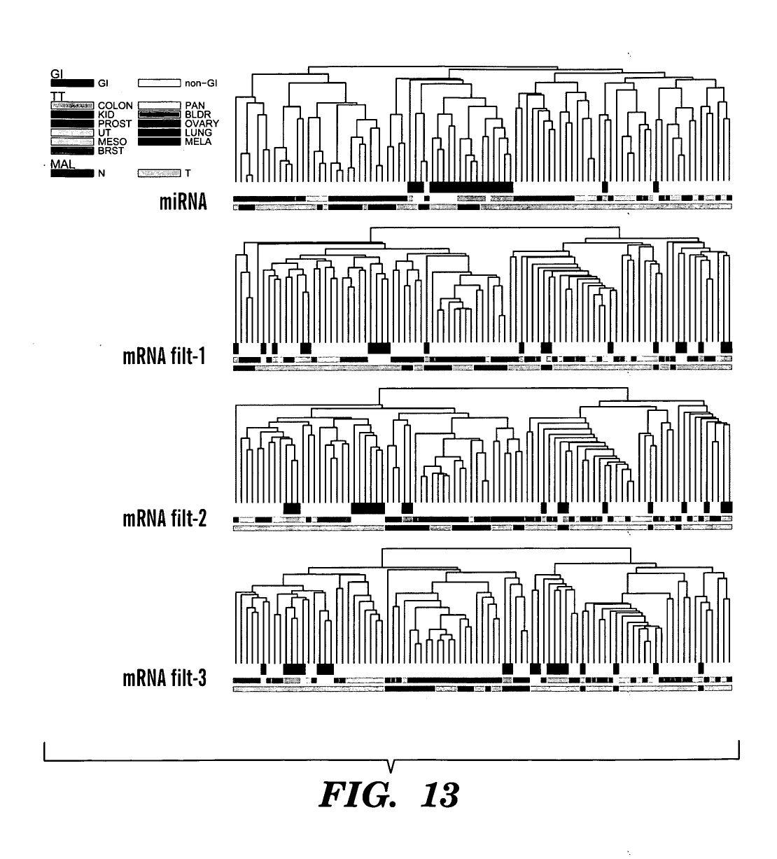

After finding that the gastrointestinal tract samples were clustered together (Example 2 and FIG. 7a), we asked whether or not this structure is similarly displayed by clustering in the mRNA space. We took 89 epithelial samples that have both successful mRNA and miRNA profiling data, and subjected them to hierarchical clustering. Both data underwent identical gene filtering, i.e. a lower threshold filter to eliminate genes that do not have expression values over 7.25 (on 10g2 scale) in any sample, and underwent the same clustering procedure. This gene filtering resulted in 195 miRNAs and 14546 mRNAs. Data were presented in the main text, FIG. 7c and FIG. 13. Results show that the mRNA clustering does not recover the coherence of GI samples, as identified in the miRNA expression space. Of note, the exact outcome of hierarchical clustering is dependent on the collection of samples present for analysis. Consequently, the cluster of the GI samples in miRNA clustering in FIG. 7c is slightly different from that of FIG. 7a, since the latter comprises of many more samples.

In order to test whether the lack of coherence of GI samples in the mRNA clustering is sensitive to the choice of genes that were used to represent each sample, we tested two additional gene filtering methods. First, we used a variation filter as was performed in Ramaswamy et al., 2001 (lower threshold of 20, upper threshold of 16000, the maximum value is at least 5 fold greater than the minimum value, and the maximum value is more than 500 greater than the minimum value), which yielded 6621 genes. Second, we examined only transcription factors, a set of gene regulators as are miRNAs. We took the genes that passed the above variation filter and that are also annotated with transcription factor activity in the Gene Ontology (www.geneontology.org, GO:0003700). This resulted in 220 transcription factors as listed in the Table 13. Similar to the minimum-expression filter on the mRNA data, these two gene selection methods yielded clustering by tissue types to a certain degree. However, none recovered the gut coherence (FIG. 13). This indicated either that the miRNA space contains some different information from the mRNA space or that in the mRNA space, the gut signal is masked by other signals or noise. Importantly, a set of transcription factors did not mimic miRNAs in this test, suggesting the difference is not solely due to the gene regulator nature of miRNAs.

Normal/Tumor Classifier and kNN Prediction of Mouse Lung Samples

In order to build a classifier of normal samples vs. tumor samples based on the miGCM collection, we first picked tissues that have enough normal and tumor samples (at least 3 in each class). Table 14 summarizes the tissues for this analysis.

kNN (Duda et al., 2000) is a predicting algorithm that learns from a training data set (in this case, the above samples from the miGCM data set) and predicts samples in a test data set (in this case, the mouse lung sample set). A set of markers (features that best distinguishes two classes of samples, in this case, normal vs. tumor) was selected using the training data set. Distances between the samples were measured in the space of the selected markers. Prediction is performed, one test sample at a time, by: (i), identifying the k nearest samples (neighbors) of the test sample among the training data set; and (ii) assigning the test sample to the majority class of these k samples.

We first selected markers that best differentiate the normal and tumor samples (see Materials and Methods above) out of the 187 features that passed the filter (which was applied on the training set alone). This generated a list of 131 markers that each has a p-value <0.05 after Bonferroni correction; 129/131 markers are over-expressed in normal samples, whereas 2/131 are over-expressed in the tumor samples. Table 15 lists these markers.

These 131 markers were used without modification to predict the 12 mouse lung samples using the k-nearest neighbour algorithm. Each mouse sample was predicted separately, using log₂ transformed mouse and human expression data. The tumor/normal phenotype prediction of a mouse sample was based on the majority type of the k nearest human samples using the chosen metric in the selected feature space. Since the tumor/normal distinction was observed at the raw miRNA expression levels, we decided to use Euclidean distance to measure the distances between samples. Thus, we performed kNN with the Euclidean distance measure and k=3, resulting in 100% accuracy. The detailed prediction results are available in Table 16. Similar classification results were obtained with other kNN parameters, with the exception of one mouse tumor T_MLUNG_{—}5 (3rd column from right in FIG. 12b). This sample was occasionally classified as normal, for example, when using cosine distance measure (k=3). It should be pointed out that cosine distance captures less an overall shift in expression levels compared to Euclidean distance. It rather focuses on comparing the relationships among the different miRNAs So it appears that the same miRNA data capture different information with different distance metrics; Pearson correlation captures information about the lineage (as seen in clustering results), and Euclidean distance captures the normal/tumor distinction.

Differentiation of HL-60 Cells

One hypothesis for the global decrease of miRNA expression in tumors (FIG. 7a, FIG. 8a,b) is that many miRNAs are upregulated during differentiation. We examined an in vitro differentiation system, the differentiation of HL-60 acute myeloblastic leukemia cells. HL-60 cells differentiate with increasing neutrophil-characteristics upon treatment with all-trans retinoic acid (ATRA) during a course of 5 days (Stegmaier et al., 2004). We found 59 miRNAs commonly expressed (see Materials and Methods for the definition of “expressed”) in three independent experiments of HL-60 cells with or without ATRA treatment. These 59 miRNAs are shown in Table 17. A heatmap is shown in FIG. 8c, reflecting averages of successfully profiled same condition samples. Results indicate increased expression of many miRNAs after 5 days of ATRA-induced differentiation (5d+). Since HL-60 is a cancerous cell line, this result supports the hypothesis that the global miRNA downregulation in cancer is related to differentiation. Whether or not the observed global miRNA expression change is associated with certain windows of differentiation needs further investigation.

Erythroid Differentiation of Primary Hematopoietic Cells in Vitro

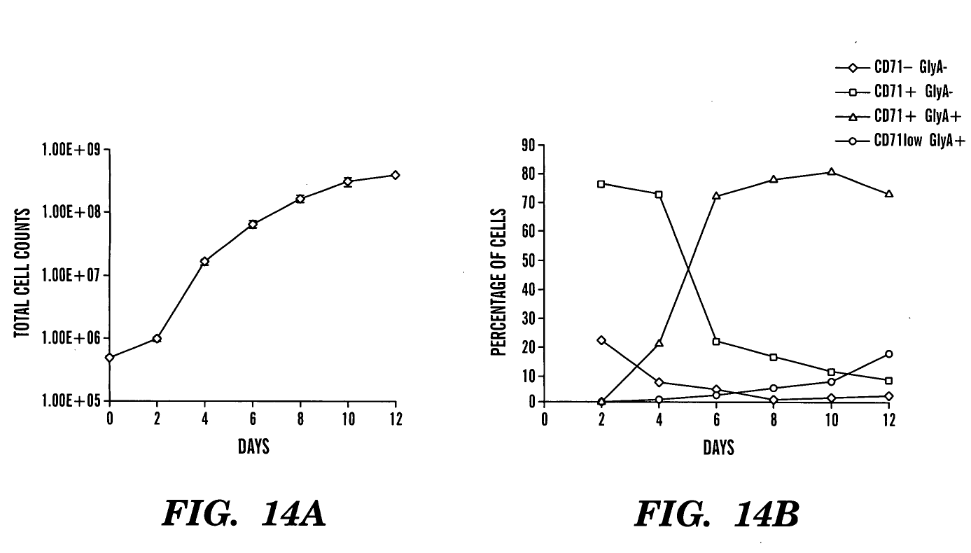

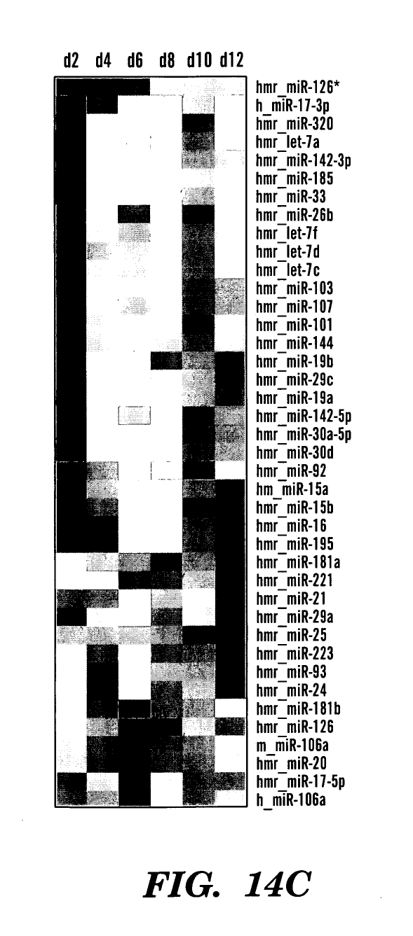

We profiled the expression of miRNAs during erythroid differentiation in vitro to ask whether the increase in miRNA expression observed in the differentiation of HL-60 cells also occurs in primary cells. The accessibility of normal hematopoietic progenitor cells and the ability to recapitulate erythropoiesis in vitro provide a model to study normal differentiation. We purified CD34⁺ hematopoietic progenitor cells from umbilical cord blood. Erythroid differentiation was induced in vitro using a two phase liquid culture system. The state of differentiation of cultured cells was monitored every other day by evaluating expression of CD71 and glycophorin A (Gly-A) (FIG. 14b). CD71 expression increases early in erythroid differentiation and gradually decreases in terminal erythroid differentiation. Gly-A expression increases later in erythropoiesis and remains elevated through terminal differentiation. As in HL60 cells, the expression of many miRNAs increased during differentiation (FIG. 14c). Unlike HL-60 cells, the erythroid cells continued to proliferate at the time points when miRNA expression increased (FIG. 14a). This suggests that proliferation itself, which is often integrally linked to differentiation, cannot account completely for the increased miRNA expression during differentiation.

Analyzing Tissue Samples Using an miRNA Proliferation Signature

It is conceivable that differences in cellular proliferation, often integrally linked to differentiation, may contribute to the global miRNA signals. We asked whether the miRNA global expression differences among samples are merely a consequence of their differences in proliferation rates. To estimate the proliferation rates in tissue samples, we assembled a consensus miRNA signature of proliferation, reported to positively correlate with proliferation or mitotic index in breast tumors, lymphomas and HeLa cells (Alizadeh et al., 2000; Perou et al., 2000; Whitfield, et al., 2002). Table 18 summarizes this list.

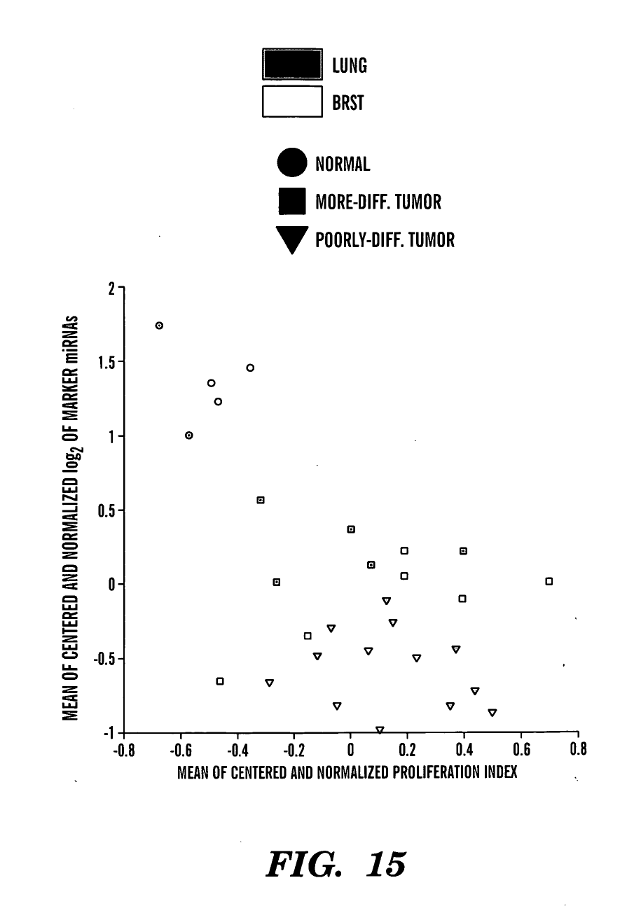

We first asked whether the miRNA proliferation signature reflects proliferation rates in our samples. Indeed, we noticed that the mean expression of these miRNAs is higher in tumors than normal tissues (FIG. 15), reflecting faster proliferation rates in tumor samples.

Next, we examined in the tumor samples the expression of the miRNA proliferation signature. We focused on lung and breast, two tissues that we have sufficient numbers of poorly differentiated tumors and more differentiated tumors. It is important to point out that poorly differentiated tumors have globally lower miRNA expression than more differentiated tumors. However, we did not observe any difference in the mRNA proliferation signature between these two categories of samples (FIG. 15). This result also suggests that the global miRNA expression is unlikely to be solely dependent on proliferation rates.

RT-PCR Analyses of Genes Involved in miRNA Machinery

One possible mechanism of the observed global miRNA expression difference between normal samples and tumors is changes in expression levels of miRNA processing enzymes. In lung cancer, Dicer levels were reported to correlate with prognosis (Karube et al., 2005). We decided to examine Dicer1, Drosha, DGCR8 and Argonaute 2 (Ago2), which are critical in miRNA processing (Tomari et al., 2005). Lacking probe sets representing these genes in our mRNA data, we used quantitative RT-PCR and analyzed 79 samples (32 normal samples and 47 tumors, covering 8 tissues, including colon, breast, uterus, lung, kidney, pancreas, prostate and bladder). We normalized the quantitative PCR data with 18S rRNA levels. We performed Student's t-test (two-tail, unequal variance) for normal/tumor phenotypes on all samples examined (P=0.3 for Dicer1, P=0.11 for Drosha, P=0.0011 for DGCR8, P=0.0138 for Ago2). DGCR8 and Ago2 have significant nominal p-values under the above test. However, the fold differences of DGCR8 and Ago2 are small between tumors and normal samples (tumor samples have higher mean threshold cycle (Ct) values for these two genes; the mean Ct differences between normal and tumor samples are: 0.776 for DGCR8 and 0.798 for Ago2, corresponding to 1.7-fold and 1.5-fold absolute level differences respectively, after correction for PCR amplification efficiency). Whether or not the observed weak decreases on the transcript level may account for the differences in miRNA expression needs further investigation. It is also important to note that these results do not exclude the possibility that these miRNA machinery genes are involved in regulating tumor/normal miRNA expression in certain cancer types, or are regulated on the protein and activity levels.

Analyses of Poorly Differentiated Tumors

We first set out to determine whether poorly differentiated tumors show a globally weaker miRNA expression than tumor samples in the miGCM collection, which represent more differentiated states. To this end, we made a comparison of poorly differentiated tumors to more differentiated tumors of the corresponding tissue types. The analysis was performed on 180 features, after the data were filtered to eliminate non-expressing miRNAs on the 55 samples which belong to tissue types that have both more differentiated and poorly-differentiated samples (see the hierarchical clustering section in Supplementary Methods for data filtration). FIG. 10 shows that poorly differentiated tumors indeed have globally lower miRNA expression. Out of the 180 features, 95 miRNAs display lower mean expression levels in poorly differentiated tumors (p<0.05 with a variance-thresholded t-test).

We used PNN for prediction of tissue origin of poorly differentiated tumors. PNN is a probability based prediction algorithm and can be considered as a smooth version of kNN. For a multi-class prediction, PNN avoids the ambiguity often encountered with kNN, when multiple training classes are equally presented in the k nearest neighbours of a test sample. For a two-class classification problem, PNN assigns a probability for a test sample to be classified into one of the two classes. The contribution of each training sample to the classification of a test sample is related to their distance and follows the Gaussian distribution: the closer the test sample, the larger the contribution. The probability for a test sample to belong to a certain class is the total contribution from every training sample belonging to that class, divided by the total contributions of all training samples (see Materials and Methods for more details).

For the prediction of poorly differentiated tumors, the training sample set consists of 68 tumor samples with both miRNA and mRNA profiling data, covering 11 tissue types. The test set contains 17 poorly differentiated tumors. Table 19 summarizes the information on the 17 poorly differentiated tumors. To solve this multi-class prediction problem, we broke down the task into 11 two-class predictions. Each two-class prediction assigns a probability for a test sample to belong to a certain tissue-type vs. the rest of the tissue-types (one vs. the rest, OVR), for example, colon vs. non-colon. After performing OVR classifications for all 11 tissues, the one tissue-type that receives the highest probability marks the predicted tissue type. The prediction results are summarized in Table 20.

### REFERENCES

van de Vijver, M. J. et al. A gene-expression signature as a predictor of survival in breast cancer. New Engl. J. Med. 347, 1999-2009 (2002).
Glas, A. M. et al. Gene expression profiling in follicular lymphoma to assess clinical aggressiveness and to guide the choice of treatment. Blood 105, 301-307 (2005).
Stegmaier, K. et al. Gene expression-based high-throughput screening (GE-HTS) and application to leukemia differentiation. Nat. Genet. 36, 257-263 (2004).
Lockhart, D. J. et al. Expression monitoring by hybridization to high-density oligonucleotide arrays. Nat. Biotechnol. 14, 1675-1680 (1996).
Van Gelder, R. N. et al. Amplified RNA synthesized from limited quantities of heterogeneous cDNA. Proc. Natl. Acad. Sci. USA 87, 1663-1667 (1990).
Tyagi, S. & FR, K. Molecular beacons: probes that fluoresce upon hybridization. Nat. Biotechnol. 14, 303-308 (1996).
Gibson, U. E., Heid, C. A. & Williams, P. M. A novel method for real time quantitative RT-PCR. Genome Res. 6, 995-1001 (1996).
Morrison, T. B., Weis, J. J. & Wittwer, C. T. Quantification of low-copy transcripts by continuous SYBR Green I monitoring during amplification. Biotechniques 24, 954-962 (1998).
Landegren, U., Kaiser, R., Sanders, J. & Hood, L. A ligase-mediated gene detection technique. Science 241, 1077-1080 (1988).
Nilsson, M., Barbany, G., Antson, D. O., Gertow, K. & Landegren, U. Enhanced detection and distinction of RNA by enzymatic probe ligation. Nat. Biotechnol. 18, 791-793 (2000).
Hsuih, T. C. et al. Novel, ligation-dependent PCR assay for detection of hepatitis C in serum. J. Clin. Microbio. 34, 501-507 (1996).
Yeakley, J. M. et al. Profiling alternative splicing on fiber-optic arrays. Nat. Biotechnol. 20, 353-358 (2002).
Eldering, E. et al. Expression profiling via novel multiplex assay allows rapid assessment of gene regulation in defined signalling pathways. Nucleic Acids Res. 31, e153 (2003).
Shoemaker, D. D., Lashkari, D. A., Morris, D., Mittmann, M. & Davis, R. W. Quantitative phenotypic analysis of yeast deletion mutants using a highly parallel molecular bar-coding strategy. Nat. Genet. 14, 450-456 (1996).
Iannone, M. A. et al. Multiplexed single nucleotide polymorphism genotyping by oligonucleotide ligation and flow cytometry. Cytometry 39, 131-140 (2000).
Yuen, T., Wun-bach, E., Pfeffer, R. L., Ebersole, B. J. & Sealfon, S. C. Accuracy and calibration of commercial oligonucleotide and custom cDNA microarrays. Nucleic Acids Res. 30, e48 (2002).
Cover, T. M. & Hart, P. E. Nearest neighbor pattern classification. IEEE Trans. Info. Theory IT-13, 21-27-(1967).
Bartel, D. P. MicroRNAs: genomics, biogenesis, mechanism, and function. Cell 116, 281-97 (2004).
Ambros, V. The functions of animal microRNAs. Nature 431, 350-5 (2004).
Chung, C. H., Bernard, P. S. & Perou, C. M. Molecular portraits and the family tree of cancer. Nat Genet 32 Suppl, 533-40 (2002).
RanaswAamy, S. et al. Multiclass cancer diagnosis using tumor gene expression signatures. Proc Natl Acad Sci USA 98, 15149-54 (2001).
Ambros, V. & Horvitz, H. R. Heterochronic mutants of the nematode Caenorhabditis elegans. Science 226, 409-16 (1984).
Lee, R. C., Feinbaum, R. L. & Ambros, V. The C. elegans heterochronic gene lin-4 encodes small RNAs with antisense complementarity to lin-14. Cell 75, 843-54 (1993).
Reinhart, B. J. et al. The 21-nucleotide let-7 RNA regulates developmental timing in Caenorhabditis elegans. Nature 403, 901-6 (2000).
Michael, M. Z., SM, O. C., van Holst Pellekaan, N. G., Young, G. P. & James, R. J. Reduced accumulation of specific microRNAs in colorectal neoplasia. Mol Cancer Res 1, 882-91 (2003).
Calin, G. A. et al. Frequent deletions and down-regulation of micro-RNA genes miR15 and miR16 at 13q14 in chronic lyinphocytic leukemia. Proc Natl Acad Sci USA 99, 15524-9 (2002).
Eis, P. S. et al. Accumulation of miR 155 and BIC RNA in human B cell lymphomas. Proc Natl Acad Sci USA 102, 3627-32 (2005).
Johnson, S. M. et al. RAS is regulated by the let-7 microRNA family. Cell 120, 635-47 (2005).
Liu, C. G. et al. An oligonucleotide microchip for genome-wide microRNA profiling in human and mouse tissues. Proc Natl Acad Sci USA 101, 9740-4 (2004).
Miska, E. A. et al. Microarray analysis of microRNA expression in the developing mammalian brain. Genome Biol 5, R68 (2004).
Thomson, J. M., Parker, J., Perou, C. M. & Hammond, S. M. A custom microarray platform for analysis of microRNA gene expression. Nature Methods 1, 47-53 (2004).
Nelson, P. T. et al. Microarray-based, high-throughput gene expression profiling of microRNAs. Nature Methods 1, 155-161 (2004).
Babak, T., Zhang, W., Morris, Q., Blencowe, B. J. & Hughes, T. R. Probing microRNAs with microarrays: tissue specificity and functional inference. RNA 10, 1813-9 (2004).
Sun, Y. et al. Development of a micro-array to detect human and mouse microRNAs and characterization of expression in human organs. Nucleic Acids Res 32, el 88 (2004).
Barad, O. et al. MicroRNA expression detected by oligonucleotide microarrays: system establishment and expression profiling in human tissues. Genome Res 14, 2486-94 (2004).
Calin, G. A. et al. MicroRNA profiling reveals distinct signatures in B cell chronic lymphocytic leukemias. Proc Natl Acad Sci USA 101, 11755-60 (2004).
Johnson, L. et al. Somatic activation of the K-ras oncogene causes early onset lung cancer in mice. Nature 410, 1111-6 (2001).
Kanellopoulou, C. et al. Dicer-deficient mouse embryonic stem cells are defective in differentiation and centromeric silencing. Genes Dev 19, 489-501 (2005).
Pavlidis, N., Briasoulis, E., Hainsworth, J. & Greco, F. A. Diagnostic and therapeutic management of cancer of an unknown primary. Eur J Cancer 39, 1990-2005 (2003).
Cullen, B. R. Transcription and processing of human microRNA precursors. Mol Cell 16, 861-5 (2004).
Lapidot, T. et al. A cell initiating human acute myeloid leukaemia after transplantation into SCID mice. Nature 367, 645-8 (1994).
Reya, T., Morrison, S. J., Clarke, M. F. & Weissman, I. L. Stem cells, cancer, and cancer stem cells. Nature 414, 105-11 (2001).
Al-Hajj, M., Wicha, M. S., Benito-Hernandez, A., Morrison, S. J. & Clarke, M. F. Prospective identification of tumorigenic breast cancer cells. Proc Natl Acad Sci USA 100, 3983-8 (2003).
Singh, S. K. et al. Identification of human brain tumour initiating cells. Nature 432, 396-401 (2004).
Yeoh, E. J. et al. Classification, subtype discovery, and prediction of outcome in pediatric acute lymphoblastic leukemia by gene expression profiling. Cancer Cell 1, 133-43 (2002).
Ferrando, A. A. et al. Gene expression signatures define novel oncogenic pathways in T cell acute lymphoblastic leukemia. Cancer Cell 1, 75-87 (2002).
Ebert, B. L. et al. An RNA interference model of RPS19 deficiency in Diamond Blackfan Anemia recapitulates defective hematopoiesis and rescue by dexamethasone: identification of dexamethasone responsive genes by microarray. Blood (2005).
Lau, N. C., Lim., L. P., Weinstein, E. G. & Bartel, D. P. An abundant class of tiny RNAs with probable regulatory roles in Caenorhabditis elegans. Science 294, 858-62 (2001).
Jain, A. K. & Dubes, R. C. Algorithms for clustering data. Prentice-Hall Inc., Upper Saddle River (1988).
Good, P. I. Permutation Tests: A Practical Guide to Resampling Methods for Testing Hypotheses, 2nd Ed. Springer-Verlag, New York (2000).
Specht, D. F. Probabilistic Neural Networks, Neural Networks. Elsevier Science Ltd., St. Louis 3, 109-118 (1990).
Griffiths-Jones, S. The microRNA Registry. Nucleic Acids Res 32 Database issue, D109-11 (2004).
Ambros, V. et al. A uniform system for microRNA annotation. RNA 9, 277-9 (2003).
Lagos-Quintana, M. et al. Identification of tissue-specific microRNAs from mouse. Curr Biol 12, 735-9 (2002).
Duda, R. O., Hart, P. E. & Stork, D. G. Pattern Classification, 2nd Ed. Wiley-Interscience, Hoboken (2000).
Alizadeh, A. A. et al. Distinct types of diffuse large B-cell lymphoma identified by gene expression profiling. Nature 403, 503-11 (2000).
Perou, C. M. et al. Molecular portraits of human breast tumours. Nature 406, 747-52 (2000).
Whitfield, M. L. et al. Identification of genes periodically expressed in the human cell cycle and their expression in tumors. Mol Biol Cell 13, 1977-2000 (2002).
Karube, Y. et al. Reduced expression of Dicer associated with poor prognosis in lung cancer patients. Cancer Sci 96, 111-5 (2005).
Tomari, Y. & Zamore, P. D. MicroRNA biogenesis: drosha can't cut it without a partner. Curr Biol 15, R61-4 (2005).

All references described herein are incorporated by reference.

TABLE 1
Classification Accuracy.

| | | differential expression | | |
|---|---|---|---|---|
| | | 1.5-2.5x | 3-4.5x | >5x |
| basal | 20-60 | 12.5 | 2.3 | 2.3 |
| expression | 60-125 | 14.8 | 1.1 | 5.7 |
| level | >125 | 1.1 | 1.1 | 0 |

Error rates (%) of a k-nearest-neighbor classifier trained on IVT-GeneChip data to predict the true identity (tretinoin or DMSO) of eighty-eight test samples in the space of each of the nine gene classes from FIG. 4.

TABLE 2
Gene Selection

| | | mean expression | | | | standard | | |
|---|---|---|---|---|---|---|---|---|
| | | level | | fold | log10 | deviation | | signal to |
| Affymetrix ID | RefSeq ID(s) | DMSO | tretinoin | change | (fold change) | DMSO | tretinoin | noise ratio |

basal expression level 20-60 units
fold change 1.5-2.5

| | | | | | | | | |
|---|---|---|---|---|---|---|---|---|
| 200721_s_at | NM_005736 | 51.20 | 81.30 | 1.59 | 0.20 | 1.05 | 1.37 | 12.47 |
| 210944_s_at | NM_000070 | 52.48 | 130.88 | 2.49 | 0.40 | 3.88 | 3.84 | 10.15 |
| | NM_024344 | | | | | | | |
| | NM_173087 | | | | | | | |
| | NM_173088 | | | | | | | |
| | NM_173089 | | | | | | | |
| | NM_173090 | | | | | | | |
| | NM_212464 | | | | | | | |
| | NM_212465 | | | | | | | |
| | NM_212467 | | | | | | | |
| 218282_at | NM_018217 | 46.40 | 78.77 | 1.70 | 0.23 | 2.78 | 0.52 | 9.79 |
| 218327_s_at | NM_004782 | 52.94 | 128.96 | 2.44 | 0.39 | 5.00 | 3.26 | 9.20 |
| 202946_s_at | NM_014962 | 27.21 | 59.36 | 2.18 | 0.34 | 2.50 | 1.58 | 7.87 |
| | NM_181443 | | | | | | | |
| 203064_s_at | NM_004514 | 124.55 | 50.66 | 2.46 | 0.39 | 4.95 | 1.00 | 12.43 |
| | NM_181430 | | | | | | | |
| | NM_181431 | | | | | | | |
| 208896_at | NM_006773 | 114.16 | 46.90 | 2.43 | 0.39 | 4.71 | 2.17 | 9.77 |
| 205176_s_at | NM_014288 | 110.04 | 58.77 | 1.87 | 0.27 | 4.05 | 1.88 | 8.65 |
| 213761_at | NM_017440 | 97.62 | 43.75 | 2.23 | 0.35 | 6.15 | 1.37 | 7.17 |
| | NM_020128 | | | | | | | |
| 209054_s_at | NM_007331 | 103.36 | 58.15 | 1.78 | 0.25 | 3.70 | 2.78 | 6.97 |
| | NM_014919 | | | | | | | |
| | NM_133330 | | | | | | | |
| | NM_133331 | | | | | | | |
| | NM_133332 | | | | | | | |
| | NM_133333 | | | | | | | |
| | NM_133334 | | | | | | | |
| | NM_133335 | | | | | | | |
| | NM_133336 | | | | | | | |

fold change 3-4.5

| | | | | | | | | |
|---|---|---|---|---|---|---|---|---|
| 212467_at | NM_173823 | 40.63 | 125.08 | 3.08 | 0.49 | 0.69 | 3.21 | 21.68 |
| 205128_x_at | NM_000962 | 58.26 | 249.54 | 4.28 | 0.63 | 11.31 | 2.21 | 14.14 |
| | NM_080591 | | | | | | | |
| 214544_s_at | NM_003825 | 43.98 | 136.04 | 3.09 | 0.49 | 6.06 | 1.59 | 12.03 |
| | NM_130798 | | | | | | | |
| 217783_s_at | NM_016061 | 51.52 | 214.96 | 4.17 | 0.62 | 6.70 | 7.03 | 11.90 |
| 204417_at | NM_000153 | 46.08 | 163.45 | 3.55 | 0.55 | 4.18 | 7.57 | 9.98 |
| 202557_at | NM_006948 | 113.75 | 30.10 | 3.78 | 0.58 | 5.27 | 1.27 | 12.79 |
| 208433_s_at | NM_004631 | 168.09 | 49.49 | 3.40 | 0.53 | 9.79 | 3.58 | 8.87 |
| | NM_017522 | | | | | | | |
| | NM_033300 | | | | | | | |
| 203362_s_at | NM_002358 | 218.12 | 52.85 | 4.13 | 0.62 | 15.89 | 3.67 | 8.45 |
| 208962_s_at | NM_013402 | 165.07 | 37.06 | 4.45 | 0.65 | 8.70 | 7.42 | 7.94 |
| 203627_at | NM_000875 | 111.98 | 35.96 | 3.11 | 0.49 | 6.82 | 3.90 | 7.09 |
| | NM_015883 | | | | | | | |

fold change >5

| | | | | | | | | |
|---|---|---|---|---|---|---|---|---|
| 207111_at | NM_001974 | 39.97 | 287.27 | 7.19 | 0.86 | 2.28 | 4.89 | 34.51 |
| 205786_s_at | NM_000632 | 51.38 | 331.91 | 6.46 | 0.81 | 7.15 | 4.53 | 24.01 |
| 212412_at | NM_006457 | 47.38 | 242.16 | 5.11 | 0.71 | 6.38 | 4.85 | 17.34 |
| 204446_s_at | NM_000698 | 50.70 | 563.72 | 11.12 | 1.05 | 5.18 | 26.90 | 15.99 |
| 210724_at | NM_032571 | 26.85 | 278.89 | 10.39 | 1.02 | 1.98 | 17.05 | 13.24 |
| | NM_152939 | | | | | | | |
| 210254_at | NM_006138 | 500.13 | 43.80 | 11.42 | 1.06 | 11.55 | 3.22 | 30.90 |
| 212563_at | NM_015201 | 189.55 | 30.71 | 6.17 | 0.79 | 1.90 | 3.97 | 27.08 |
| 204538_x_at | NM_006985 | 298.36 | 28.02 | 10.65 | 1.03 | 12.03 | 4.11 | 16.76 |
| 221539_at | NM_004095 | 622.12 | 51.77 | 12.02 | 1.08 | 18.14 | 20.13 | 14.90 |
| 222036_s_at | NM_005914 | 243.17 | 44.11 | 5.51 | 0.74 | 18.70 | 5.26 | 8.31 |
| | NM_182746 | | | | | | | |

basal expression level 60-125 units
fold change 1.5-2.5

| | | | | | | | | |
|---|---|---|---|---|---|---|---|---|
| 201779_s_at | NM_007282 | 121.10 | 297.22 | 2.45 | 0.39 | 2.64 | 11.71 | 12.27 |
| | NM_183381 | | | | | | | |
| | NM_183382 | | | | | | | |
| | NM_183383 | | | | | | | |
| | NM_183384 | | | | | | | |
| 211067_s_at | NM_003644 | 122.85 | 267.79 | 2.18 | 0.34 | 8.26 | 5.49 | 10.54 |
| | NM_005890 | | | | | | | |
| | NM_201432 | | | | | | | |
| | NM_201433 | | | | | | | |
| 202923_s_at | NM_001498 | 63.33 | 145.68 | 2.30 | 0.36 | 4.04 | 4.23 | 9.96 |
| 204295_at | NM_003172 | 123.97 | 211.17 | 1.70 | 0.23 | 5.99 | 3.85 | 8.86 |
| 207629_s_at | NM_004723 | 103.61 | 177.50 | 1.71 | 0.23 | 5.56 | 2.82 | 8.82 |
| 217850_at | NM_014366 | 291.05 | 119.42 | 2.44 | 0.39 | 2.98 | 4.54 | 22.82 |
| | NM_206825 | | | | | | | |
| | NM_206826 | | | | | | | |
| 203315_at | NM_001004720 | 121.02 | 61.68 | 1.96 | 0.29 | 0.66 | 2.06 | 21.78 |
| | NM_001004722 | | | | | | | |
| | NM_003581 | | | | | | | |
| 218607_s_at | NM_018115 | 160.90 | 96.30 | 1.67 | 0.22 | 1.92 | 4.54 | 9.99 |
| 209511_at | NM_021974 | 127.46 | 83.32 | 1.53 | 0.18 | 2.55 | 1.92 | 9.87 |
| 221699_s_at | NM_024045 | 189.21 | 93.24 | 2.03 | 0.31 | 4.49 | 5.34 | 9.77 |

fold change 3-4.5

| | | | | | | | | |
|---|---|---|---|---|---|---|---|---|
| 202902_s_at | NM_004079 | 65.75 | 262.67 | 3.99 | 0.60 | 8.96 | 3.98 | 15.22 |
| 201413_at | NM_000414 | 77.30 | 335.21 | 4.34 | 0.64 | 10.18 | 8.52 | 13.79 |
| 212135_s_at | NM_001001396 | 92.80 | 332.51 | 3.58 | 0.55 | 2.52 | 14.99 | 13.69 |
| | NM_001684 | | | | | | | |
| 208485_x_at | NM_003879 | 60.99 | 214.30 | 3.51 | 0.55 | 7.62 | 5.12 | 12.04 |
| 201565_s_at | NM_002166 | 105.04 | 340.67 | 3.24 | 0.51 | 6.80 | 12.79 | 12.03 |
| 208581_x_at | NM_005952 | 305.95 | 93.48 | 3.27 | 0.51 | 10.39 | 2.12 | 16.98 |
| 201890_at | NM_001034 | 352.52 | 104.62 | 3.37 | 0.53 | 13.89 | 2.55 | 15.08 |
| 201516_at | NM_003132 | 428.63 | 113.75 | 3.77 | 0.58 | 19.76 | 2.03 | 14.45 |
| 221652_s_at | NM_018164 | 280.86 | 78.45 | 3.58 | 0.55 | 13.83 | 3.01 | 12.02 |
| 212282_at | NM_014573 | 300.99 | 96.70 | 3.11 | 0.49 | 11.04 | 8.12 | 10.66 |

fold change >5

| | | | | | | | | |
|---|---|---|---|---|---|---|---|---|
| 209030_s_at | NM_014333 | 114.63 | 3138.68 | 27.38 | 1.44 | 8.58 | 21.28 | 101.28 |
| 200701_at | NM_006432 | 101.26 | 992.64 | 9.80 | 0.99 | 5.45 | 8.88 | 62.17 |
| 209949_at | NM_000433 | 64.04 | 431.32 | 6.74 | 0.83 | 5.21 | 3.41 | 42.63 |
| 202838_at | NM_000147 | 98.39 | 1727.68 | 17.56 | 1.24 | 17.24 | 66.39 | 19.48 |
| 211506_s_at | NM_000584 | 91.45 | 598.35 | 6.54 | 0.82 | 4.81 | 24.33 | 17.40 |
| 201013_s_at | NM_006452 | 645.25 | 105.67 | 6.11 | 0.79 | 2.52 | 4.11 | 81.40 |
| 201930_at | NM_005915 | 633.11 | 107.33 | 5.90 | 0.77 | 4.02 | 10.80 | 35.48 |
| 204351_at | NM_005980 | 1257.67 | 72.27 | 17.40 | 1.24 | 36.81 | 20.07 | 20.84 |
| 200790_at | NM_002539 | 949.56 | 101.20 | 9.38 | 0.97 | 63.91 | 4.53 | 12.40 |
| 202887_s_at | NM_019058 | 508.55 | 89.10 | 5.71 | 0.76 | 31.95 | 14.40 | 9.05 |

basal expression level >125 units
fold change 1.5-2.5

| | | | | | | | | |
|---|---|---|---|---|---|---|---|---|
| 200077_s_at | NM_004152 | 2228.65 | 3478.72 | 1.56 | 0.19 | 36.65 | 7.31 | 28.43 |
| 207320_x_at | NM_004602 | 159.09 | 243.61 | 1.53 | 0.19 | 4.33 | 0.65 | 16.96 |
| | NM_017452 | | | | | | | |
| | NM_017453 | | | | | | | |
| | NM_017454 | | | | | | | |
| 208641_s_at | NM_006908 | 125.43 | 286.94 | 2.29 | 0.36 | 1.61 | 7.94 | 16.91 |
| | NM_018890 | | | | | | | |
| | NM_198829 | | | | | | | |
| 213867_x_at | NM_001101 | 6437.29 | 10848.75 | 1.69 | 0.23 | 107.58 | 169.49 | 15.92 |
| 204158_s_at | NM_006019 | 183.26 | 446.89 | 2.44 | 0.39 | 3.84 | 12.91 | 15.74 |
| | NM_006053 | | | | | | | |
| 200691_s_at | NM_004134 | 450.19 | 188.06 | 2.39 | 0.38 | 10.10 | 6.16 | 16.12 |
| 201077_s_at | NM_001003796 | 675.17 | 379.69 | 1.78 | 0.25 | 11.15 | 7.98 | 15.45 |
| | NM_005008 | | | | | | | |
| 217810_x_at | NM_020117 | 352.53 | 218.24 | 1.62 | 0.21 | 5.20 | 3.67 | 15.14 |
| 200792_at | NM_001469 | 940.53 | 580.29 | 1.62 | 0.21 | 23.54 | 5.17 | 12.55 |
| 218140_x_at | NM_021203 | 400.95 | 197.86 | 2.03 | 0.31 | 8.19 | 8.61 | 12.09 |

fold change 3-4.5

| | | | | | | | | |
|---|---|---|---|---|---|---|---|---|
| 210908_s_at | NM_002624 | 857.33 | 2675.14 | 3.12 | 0.49 | 20.67 | 51.57 | 25.16 |
| | NM_145896 | | | | | | | |
| | NM_145897 | | | | | | | |
| 201460_at | NM_004759 | 142.58 | 473.41 | 3.32 | 0.52 | 4.71 | 9.73 | 22.92 |
| | NM_032960 | | | | | | | |
| 203470_s_at | NM_002664 | 167.89 | 689.86 | 4.11 | 0.61 | 3.62 | 23.36 | 19.34 |
| 202803_s_at | NM_000211 | 558.85 | 2149.86 | 3.85 | 0.59 | 30.29 | 61.10 | 17.41 |
| 209124_at | NM_002468 | 168.56 | 687.89 | 4.08 | 0.61 | 7.63 | 22.94 | 16.99 |
| 201892_s_at | NM_000884 | 1690.72 | 556.27 | 3.04 | 0.48 | 43.73 | 15.45 | 19.17 |
| 200647_x_at | NM_003752 | 2203.38 | 717.78 | 3.07 | 0.49 | 84.31 | 29.06 | 13.10 |
| 218512_at | NM_018256 | 458.15 | 145.51 | 3.15 | 0.50 | 13.13 | 10.86 | 13.03 |
| 209932_s_at | NM_001948 | 783.00 | 248.26 | 3.15 | 0.50 | 15.57 | 29.24 | 11.93 |
| 200650_s_at | NM_005566 | 1944.97 | 593.69 | 3.28 | 0.52 | 90.23 | 31.23 | 11.13 |

fold change >5

| | | | | | | | | |
|---|---|---|---|---|---|---|---|---|
| 217733_s_at | NM_021103 | 637.96 | 3221.75 | 5.05 | 0.70 | 33.65 | 82.85 | 22.18 |
| 210592_s_at | NM_002970 | 157.29 | 1070.71 | 6.81 | 0.83 | 11.56 | 37.71 | 18.54 |
| 204122_at | NM_003332 | 456.11 | 3465.79 | 7.60 | 0.88 | 14.27 | 154.50 | 17.83 |
| | NM_198125 | | | | | | | |
| 204232_at | NM_004106 | 200.54 | 1713.24 | 8.54 | 0.93 | 14.01 | 80.44 | 16.02 |
| 216598_s_at | NM_002982 | 132.79 | 5147.99 | 38.77 | 1.59 | 27.61 | 322.89 | 14.31 |
| 204798_at | NM_005375 | 877.47 | 132.27 | 6.63 | 0.82 | 20.74 | 14.06 | 21.41 |
| 203949_at | NM_000250 | 2732.30 | 170.06 | 16.07 | 1.21 | 148.73 | 13.39 | 15.80 |
| 202107_s_at | NM_004526 | 696.44 | 137.07 | 5.08 | 0.71 | 48.08 | 4.62 | 10.61 |
| 211951_at | NM_004741 | 752.52 | 135.10 | 5.57 | 0.75 | 42.57 | 19.86 | 9.89 |
| 202431_s_at | NM_002467 | 2723.42 | 174.53 | 15.60 | 1.19 | 381.41 | 6.76 | 6.57 |

TABLE 3
Probe Sequences
signature genes:

| Affy- | | | Flex- | | | | |
|---|---|---|---|---|---|---|---|
| metrix | RefSeq | RefSet | MAP | | downstream probe | | |
| ID | ID | ID | ID | upstream probe sequence | sequence | | |
| 200721_s_at | NM_005736 | HG_010_01195 | LUA#1 | TAATACGACTCACTATAGGGCTTTA | seq | CCCAGTGTACTGAAATAAAGT | seq |
| | | | | ATCTCAATCAATACAAATCAACCAC | id | CCCTTTAGTGAGGGTTAAT | id |
| | | | | ATTGCCTGGTGGGG | no: | | no: |
| | | | | | 1 | | 91 |
| 210944_s_at | NM_000070 | HG_010_18277 | LUA#2 | TAATACGACTCACTATAGGGCTTTA | seq | GACGCAGGATTCCACCTCAAT | seq |
| | | | | TCAATACATACTACAATCAAGATGC | id | CCCTTTAGTGAGGGTTAAT | id |
| | | | | GAAATGCAGTCAAC | no: | | no: |
| | | | | | 2 | | 92 |
| 218282_at | NM_018217 | HG_010_21926 | LUA#3 | TAATACGACTCACTATAGGGTACAC | seq | CATTAGTGGGACAGGTTTTCT | seq |
| | | | | TTTATCAAATCTTACAATCGCCCTT | id | CCCTTTAGTGAGGGTTAAT | id |
| | | | | CACCTCCAAGTTGG | no: | | no: |
| | | | | | 3 | | 93 |
| 218327_s_at | NM_004782 | HG_010_06845 | LUA#4 | TAATACGACTCACTATAGGGTACAT | seq | GGTTCCACTTACTGTAATTGT | seq |
| | | | | TACCAATAATCTTCAAATCGCAGAG | id | CCCTTTAGTGAGGGTTAAT | id |
| | | | | CAGCTTTTGTGCAC | no: | | no: |
| | | | | | 4 | | 94 |
| 202946_s_at | NM_014962 | HG_010_21147 | LUA#5 | TAATACGACTGACTATAGGGCAATT | seq | GTTGTTCATTCTGGGGATAAT | seq |
| | | | | CAAATCACAATAATCAATCTCTGGC | id | CCCTTTAGTGAGGGTTAAT | id |
| | | | | TGGCAGTCTTTGTC | no: | | no: |
| | | | | | 5 | | 95 |
| 203064_s_at | NM_004514 | HG_010_18737 | LUA#46 | TAATACGACTCACTATAGGGTACAT | seq | CATGTGGCTCGCGTGGACAGT | seq |
| | | | | CAACAATTCATTCAATACATTTATC | id | CCCTTTAGTGAGGGTTAAT | id |
| | | | | CACCTCCATTTCAG | no: | | no: |
| | | | | | 6 | | 96 |
| 208896_at | NM_006773 | HG_010_01959 | LUA#47 | TAATACGACTCACTATAGGGCTTCT | seq | CTGTGCTCACTGCTGTAAAAT | seq |
| | | | | CATTAACTTACTTCATAATGATTTT id | CCCTTTAGTGAGGGTTAAT | id | |
| | | | | TGTGGCATGGATTG | no: | | no: |
| | | | | | 7 | | 97 |
| 205176_s_at | NM_014288 | HG_010_08052 | LUA#48 | TAATACGACTCACTATAGGGAAACA | seq | CACTCACCATGAGCACCAACT | seq |
| | | | | AACTTCACATCTCAATAATTGAGGC | id | CCCTTTAGTGAGGGTTAAT | id |
| | | | | ATTAAGAAGAAATG | no: | | no: |
| | | | | | 8 | | 98 |
| 213761_at | NM_017440 | HG_010_16616 | LUA#49 | TAATACGAGTCACTATAGGGTCATC | seq | CAGAACCAGAAGCCCCGGAAT | seq |
| | | | | AATCTTTCAATTTACTTACGAGCAA | id | CCCTTTAGTGAGGGTTAAT | id |
| | | | | TGTGGTTGCATCAG | no: | | no: |
| | | | | | 9 | | 99 |
| 209054_s_at | NM_007331 | HG_010_20167 | LUA#50 | TAATAGGACTCACTATAGGGCAATA | seq | GGCAGCATCTTCAGCTCTTGT | seq |
| | | | | TACCAATATCATCATTTACAAGCGA | id | CCCTTTAGTGAGGGTTAAT | id |
| | | | | AATCGGGCTTCCAC | no: | | no: |
| | | | | | 10 | | 100 |
| 212467_at | NM_173823 | * | LUA#6 | TAATACGACTCACTATAGGGTCAAC | seq | CTGCCACCTCCTGTAGACCAT | seq |
| | | | | AATCTTTTACAATCAAATCCTACAT | id | CCCTTTAGTGAGGGTTAAT | id |
| | | | | CAGTCATGTCTAAC | no: | | no: |
| | | | | | 11 | | 101 |
| 205128_x_at | NM_000962 | HG_010_04807 | LUA#7 | TAATACGACTCACTATAGGGCAATT | seq | CCTGCTAGTCTGCCCTATGGT | seq |
| | | | | CATTTACCAATTTACCAATACTGCT | id | CCCTTTAGTGAGGGTTAAT | id |
| | | | | GCCTGAGTTTCCAG | no: | | no: |
| | | | | | 12 | | 102 |
| 214544_s_at | NM_003825 | HG_010_06841 | LUA#8 | TAATACGACTCACTATAGGGAATCC | seq | CATAATCAAGTTGATGTGGAT | seq |
| | | | | TTTTACATTCATTACTTACCTTGTG | id | CCCTTTAGTGAGGGTTAAT | id |
| | | | | TATTGAACTATGTC | no: | | no: |
| | | | | | 13 | | 103 |
| 217783_s_at | NM_016061 | HG_010_21524 | LUA#9 | TAATACGACTCACTATAGGGTAATC | seq | CTATTTGCCACTGGGCTGTTT | seq |
| | | | | TTCTATATCAACATCTTACTGAGTA | id | CCCTTTAGTGAGGGTTAAT | id |
| | | | | CAGTTAAGTTCCTC | no: | | no: |
| | | | | | 14 | | 104 |
| 204417_at | NM_000153 | HG_010_18368 | LUA#10 | TAATACGACTCACTATAGGGATCAT | seq | CTCAGTCAGTTCCTTTCACTT | seq |
| | | | | ACATACATACAAATCTACAAAGGTT | id | CCCTTTAGTGAGGGTTAAT | id |
| | | | | CTCTTGTATACCTG | no: | | no: |
| | | | | | 15 | | 105 |
| 202557_at | NM_006948 | HG_010_16269 | LUA#51 | TAATACGACTCACTATAGGGTCATT | seq | CTCATCTCATGTCCTGAAGTT | seq |
| | | | | TCAATCAATCATCAACAATTGACAA | id | CCCTTTAGTGAGGGTTAAT | id |
| | | | | AATAGGGCAGGCAG | no: | | no: |
| | | | | | 16 | | 106 |
| 208433_s_at | NM_004631 | HG_010_03370 | LUA#52 | TAATACGACTCACTATAGGGTCAAT | seq | CTGGAGAACGAGGCCATTTTT | seq |
| | | | | CATCTTTATACTTCACAATACAAGG | id | CCCTTTAGTGAGGGTTAAT | id |
| | | | | TGTTCTGGACAGAC | no: | | no: |
| | | | | | 17 | | 107 |
| 203362_s_at | NM_002358 | HG_010_20134 | LUA#53 | TAATACGACTCACTATAGGGTAATT | seq | GTCAAGTAGTTTGACTCAGTT | seq |
| | | | | ATACATCTCATCTTCTACATTCCTA | id | CCCTTTAGTGAGGGTTAAT | id |
| | | | | AATCAGATGTTTTG | no: | | no: |
| | | | | | 18 | | 108 |
| 208962_s_at | NM_013402 | HG_010_02173 | LUA#54 | TAATACGACTCACTATAGGGCTTTT | seq | CCTTCTCAGCCTACAGCAGTT | seq |
| | | | | TCAATCACTTTCAATTCATAAGCAC | id | CCCTTTAGTGAGGGTTAAT | id |
| | | | | CTGAACCACTGTGG | no: | | no: |
| | | | | | 19 | | 109 |
| 203627_at | NM_000875 | HG_010_00403 | LUA#55 | TAATACGACTCACTATAGGGTATAT | seq | CTTCTGACTAGATTATTATTT | seq |
| | | | | ACACTTCTCAATAACTAACCAGGCA | id | CCCTTTAGTGAGGGTTAAT | id |
| | | | | CACAGGTCTCATTG | no: | | no: |
| | | | | | 20 | | 110 |
| 207111_at | NM_001974 | HG_010_17076 | LUA#11 | TAATACGAGTCACTATAGGGTACAA | seq | CACTGATGAGAAATCAGACGT | seq |
| | | | | ATCATCAATCACTTTAATCCGTCTT | id | CCCTTTAGTGAGGGTTAAT | id |
| | | | | CCTGTGGTTGTATG | no: | | no: |
| | | | | | 21 | | 111 |
| 205786_s_at | NM_000632 | HG_010_20041 | LUA#12 | TAATACGACTCACTATAGGGTACAC | seq | CAGGCGATGTGCAAGTGTATT | seq |
| | | | | TTTCTTTCTTTCTTTCTTTGGTTTC | id | CCCTTTAGTGAGGGTTAAT | id |
| | | | | CTTCAGACAGATTC | no: | | no: |
| | | | | | 22 | | 112 |
| 212412_at | NM_006457 | HG_010_19532 | LUA#13 | TAATACGACTCACTATAGGGCAATA | seq | GATCAGTGGCACCAGCCAACT | seq |
| | | | | AACTATACTTCTTCACTAAAAACAG | id | CCCTTTAGTGAGGGTTAAT | id |
| | | | | CGCTACTTACTCAG | no: | | no: |
| | | | | | 23 | | 113 |
| 204446_s_at | NM_000698 | HG_010_16744 | LUA#14 | TAATACGACTCACTATAGGGCTACT | seq | GAGCAACAGCAAATCACGACT | seq |
| | | | | ATACATCTTACTATACTTTCTCAGC | id | CCCTTTAGTGAGGGTTAAT | id |
| | | | | ATTTCCACACCAAG | no: | | no: |
| | | | | | 24 | | 114 |
| 210724_at | NM_032571 | HG_010_15648 | LUA#15 | TAATACGACTGACTATAGGGATACT | seq | CTGACTCAAAACCCAGTGAGT | seq |
| | | | | TCATTCATTCATCAATTCAACTTTC | id | CCCTTTAGTGAGGGTTAAT | id |
| | | | | CAGCAAGATGGGTC | no: | | no: |
| | | | | | 25 | | 115 |
| 210254_at | NM_006138 | HG_010_15460 | LUA#56 | TAATACGACTCACTATAGGGCAATT | seq | GAACTCACACATGCCCTGATT | seq |
| | | | | TACTCATATACATCACTTTTTTATT | id | CCCTTTAGTGAGGGTTAAT | id |
| | | | | TCAGTGAACTGCTG | no: | | no: |
| | | | | | 26 | | 116 |
| 212563_at | NM_015201 | HG_010_10972 | LUA#57 | TAATACGACTCACTATAGGGCAATA | seq | CTGGTGTGGTTTGACCTGGAT | seq |
| | | | | TCATCATCTTTATCATTACGTGGGA | id | CCCTTTAGTGAGGGTTAAT | id |
| | | | | GCTACGATAGCAAG | no: | | no: |
| | | | | | 27 | | 117 |
| 204538_x_at | NM_006985 | * | LUA#58 | TAATACGACTCACTATAGGGCTACT | seq | GGAGTGTCTGCTCTATCCCCT | seq |
| | | | | AATTCATTAACATTACTACGATAAT | id | CCCTTTAGTGAGGGTTAAT | id |
| | | | | CTCAAGACACCTGC | no: | | no: |
| | | | | | 28 | | 118 |
| 221539_at | NM_004095 | HG_010_07678 | LUA#59 | TAATACGACTCACTATAGGGTCATC | seq | GGAAAGCTCCCTCCCCCTCCT | seq |
| | | | | AATCAATCTTTTTCACTTTTCCTTA | id | CCCTTTAGTGAGGGTTAAT | id |
| | | | | GGTTGATGTGCTTG | no: | | no: |
| | | | | | 29 | | 119 |
| 222036_s_at | NM_005914 | * | LUA#60 | TAATACGACTCACTATAGGGAATCT | seq | GCTTAAACCCAGGCGGCAGAT | seq |
| | | | | ACAAATCCAATAATCTCATGAGGTT | id | CCCTTTAGTGAGGGTTAAT | id |
| | | | | GAGGCAGGAGAATC | no: | | no: |
| | | | | | 30 | | 120 |
| 201779_s_at | NM_007282 | HG_010_08042 | LUA#16 | TAATACGACTCACTATAGGGAATCA | seq | GAGAGGCAACAAGGTAATTCT | seq |
| | | | | ATCTTCATTCAAATCATCACTGACC | id | CCCTTTAGTGAGGGTTAAT | id |
| | | | | TGCCAATCATTAGG | no: | | no: |
| | | | | | 31 | | 121 |
| 211067_s_at | NM_003644 | HG_010_17163 | LUA#17 | TAATACGACTCACTATAGGGCTTTA | seq | GAGAATGAGACAGAGGGCAAT | seq |
| | | | | ATCCTTTATCACTTTATCACCATTG | id | CCCTTTAGTGAGGGTTAAT | id |
| | | | | CAGCAGGTTAGAGC | no: | | no: |
| | | | | | 32 | | 122 |
| 202923_s_at | NM_001498 | HG_010_18372 | LUA#18 | TAATACGACTCACTATAGGGTCAAA | seq | CCCCAAGCTTTCCCCTCTGAT | seq |
| | | | | ATCTCAAATACTCAAATCAATAATC | id | CCCTTTAGTGAGGGTTAAT | id |
| | | | | ACTTGGTCACCTTG | no: | | no: |
| | | | | | 33 | | 123 |
| 204295_at | NM_003172 | HG_010_06973 | LUA#19 | TAATACGACTCACTATAGGGTCAAT | seq | CATTATCGAGACCTGGAAGCT | seq |
| | | | | CAATTACTTACTCAAATACATCCAG | id | CCCTTTAGTGAGGGTTAAT | id |
| | | | | AAAGGAACCACTGG | no: | | no: |
| | | | | | 34 | | 124 |
| 207629_s_at | NM_004723 | HG_010_03179 | LUA#20 | TAATACGACTCACTATAGGGCTTTT | seq | CAACCATGACCTGAAACCTCT | seq |
| | | | | ACAATACTTCAATACAATCGACCTC | id | CCCTTTAGTGAGGGTTAAT | id |
| | | | | ATCTTCCACCTCAG | no: | | no: |
| | | | | | 35 | | 125 |
| 217850_at | NM_014366 | HG_010_20659 | LUA#61 | TAATACGACTCACTATAGGGAATCT | seq | CAGGTGAACAGTCTACAAGGT | seq |
| | | | | TACCAATTCATAATCTTCACACTTC | id | CCCTTTAGTGAGGGTTAAT | id |
| | | | | TGAGGAGACTACAG | no: | | no: |
| | | | | | 36 | | 126 |
| 203315_at | NM_003581 | HG_010_17522 | LUA#62 | TAATACGACTCACTATAGGGTCAAT | seq | GTCAGGGAAGAACAAACACTT | seq |
| | | | | CATAATCTCATAATCCAATTTCTCC | id | CCCTTTAGTGAGGGTTAAT | id |
| | | | | GTGTCCCTTAAAGC | no: | | no: |
| | | | | | 37 | | 127 |
| 218607_s_at | NM_018115 | HG_010_21859 | LUA#63 | TAATACGACTCACTATAGGGCTACT | seq | CCTGTAATATTTTCAGCCCAT | seq |
| | | | | TCATATACTTTATACTACATTTCCT | id | CCCTTTAGTGAGGGTTAAT | id |
| | | | | CAGCCTTCCTTCAG | no: | | no: |
| | | | | | 38 | | 128 |
| 209511_at | NM_021974 | HG_010_02843 | LUA#64 | TAATACGACTCACTATAGGGCTACA | seq | GAGTCATCTTCGTGCCCTTGT | seq |
| | | | | TATTCAAATTACTACTTACCATCAT | id | CCCTTTAGTGAGGGTTAAT | id |
| | | | | CACCGACTGAGCTG | no: | | no: |
| | | | | | 39 | | 129 |
| 221699_s_at | NM_024045 | HG_010_01029 | LUA#65 | TAATACGACTCACTATAGGGCTTTT | seq | CATCAAGCTTTGAACCACGAT | seq |
| | | | | CATCAATAATCTTACCTTTTTTAGC | id | CCCTTTAGTGAGGGTTAAT | id |
| | | | | CCACATTTCTGGTG | no: | | no: |
| | | | | | 40 | | 130 |
| 202902_s_at | NM_004079 | HG_010_15445 | LUA#21 | TAATACGACTCACTATAGGGAATCC | seq | GAATCTAAACAAACAGGCCTT | seq |
| | | | | TTTCTTTAATCTCAAATCAAAGCAC | id | CCCTTTAGTGAGGGTTAAT | id |
| | | | | AGGGACACAAAGAG | no: | | no: |
| | | | | | 41 | | 131 |
| 201413_at | NM_000414 | HG_010_17294 | LUA#22 | TAATACGACTCACTATAGGGAATCC | seq | CCAGAGGGAACATCATGCTGT | seq |
| | | | | TTTTTACTCAATTCAATCACTTTAG | id | CCCTTTAGTGAGGGTTAAT | id |
| | | | | TGGCAGGCTGAAGG | no: | | no: |
| | | | | | 42 | | 132 |
| 212135_s_at | NM_001684 | HG_010_16788 | LUA#23 | TAATACGACTCACTATAGGGTTCAA | seq | CATCACCCCACCCCACATTCT | seq |
| | | | | TCATTCAAATCTCAACTTTAATGAT | id | CCCTTTAGTGAGGGTTAAT | id |
| | | | | GACAATCCTGTTGG | no: | | no: |
| | | | | | 43 | | 133 |
| 208485 x_at | NM_003879 | * | LUA#24 | TAATACGACTCACTATAGGGTCAAT | seq CACACTCTGAGAAAGAAACTT | seq | |
| | | | | TACCTTTTCAATACAATACAATATT | id | CCCTTTAGTGAGGGTTAAT | id |
| | | | | ATGTCTGGCTGCAG | no: | | no: |
| | | | | | 44 | | 134 |
| 201565_s_at | NM_002166 | HG_010_17313 | LUA#25 | TAATACGACTCACTATAGGGCTTTT | seq | CCTTCTGAGTTAATGTCAAAT | seq |
| | | | | CAATTACTTCAAATCTTCACCTTGC | id | CCCTTTAGTGAGGGTTAAT | id |
| | | | | AGGCTTCTGAATTC | no: | | no: |
| | | | | | 45 | | 135 |
| 208581 x_at | NM_005952 | * | LUA#66 | TAATACGACTCACTATAGGGTAACA | seq | CAACCTATATAAACCTGGATT | seq |
| | | | | TTACAACTATACTATCTACGCTCTC | id | CCCTTTAGTGAGGGTTAAT | id |
| | | | | AGATGTAAATAGAG | no: | | no: |
| | | | | | 46 | | 136 |
| 201890_at | NM_001034 | HG_010_18467 | LUA#67 | TAATACGACTCACTATAGGGTCATT | seq | CCCCTCTGAGTAGAGTGTTGT | seq |
| | | | | TACTCAACAATTACAAATCAGTGTG | id | CCCTTTAGTGAGGGTTAAT | id |
| | | | | CTGGGATTCTCTGC | no: | | no: |
| | | | | | 47 | | 137 |
| 201516_at | NM_003132 | HG_010_17983 | LUA#68 | TAATACGACTCACTATAGGGTCATA | seq | CCTATACCAGCTGTGTACAGT | seq |
| | | | | ATCTCAACAATCTTTCTTTTCTGGC | id | CCCTTTAGTGAGGGTTAAT | id |
| | | | | GTTCCACCTCCAAG | no: | | no: |
| | | | | | 48 | | 138 |
| 221652_s_at | NM_018164 | HG_010_00331 | LUA#69 | TAATACGACTCACTATAGGGCTATA | seq | GGCAGTGAAGAGTGACTTGAT | seq |
| | | | | AACATATTACATTCACATCAGAAAA | id | CCCTTTAGTGAGGGTTAAT | id |
| | | | | TGGAAAAGCCAGCC | no: | | no: |
| | | | | | 49 | | 139 |
| 212282_at | NM_014573 | * | LUA#70 | TAATACGACTCACTATAGGGATACC | seq | CATCTCAAGGGTGATCTGGAT | seq |
| | | | | AATAATCCAATTCATATCATCCCTG | id | CCCTTTAGTGAGGGTTAAT | id |
| | | | | TATCTGAAGTCTAG | no: | | no: |
| | | | | | 50 | | 140 |
| 209030_s_at | NM_014333 | HG_010_14934 | LUA#26 | TAATACGACTCACTATAGGGTTACT | seq | GCACTTAACCAAGACAAAAAT | seq |
| | | | | CAAAATCTACACTTTTTCATACCCC | id | CCCTTTAGTGAGGGTTAAT | id |
| | | | | TCCCCTATCCCTAG | no: | | no: |
| | | | | | 51 | | 141 |
| 200701_at | NM_006432 | HG_010_08035 | LUA#27 | TAATACGACTCACTATAGGGCTTTT | seq | GCTGGTTCTCAGTGGTTGTCT | seq |
| | | | | CAAATCAATACTCAACTTTCAGAAA | id | CCCTTTAGTGAGGGTTAAT | id |
| | | | | CTGAGCTCCGGGTG | no: | | no: |
| | | | | | 52 | | 142 |
| 209949_at | NM_000433 | HG_010_18441 | LUA#28 | TAATACGACTCACTATAGGGCTACA | seq | CAGGTACTGATCCTGTTTCTT | seq |
| | | | | AACAAACAAACATTATCAAAAGGGC | id | CCCTTTAGTGAGGGTTAAT | id |
| | | | | ACGAGAGAGTCTTC | no: | | no: |
| | | | | | 53 | | 143 |
| 202838_at | NM_000147 | HG_010_16435 | LUA#29 | TAATACGACTCACTATAGGGAATCT | seq | CTATGGTCAACTCTTCAGAAT | seq |
| | | | | TACTACAAATCCTTTCTTTGGAAAA | id | CCCTTTAGTGAGGGTTAAT | id |
| | | | | GGCTTACCAGGCTG | no: | | no: |
| | | | | | 54 | | 144 |
| 211506_s_at | NM_000584 | HG_010_00131 | LUA#30 | TAATACGACTCACTATAGGGTTACC | seq | CAGTCTTGTCATTGCCAGCTT | seq |
| | | | | TTTATACCTTTCTTTTTACCAATCC | id | CCCTTTAGTGAGGGTTAAT | id |
| | | | | TAGTTTGATACTCC | no: | | no: |
| | | | | | 55 | | 145 |
| 201013_s_at | NM_006452 | HG_010_04110 | LUA#71 | TAATACGACTCACTATAGGGATCAT | seq | CTTTAGTTCTCTGAAGGCCCT | seq |
| | | | | TACAATCCAATCAATTCATGGACTG | id | CCCTTTAGTGAGGGTTAAT | id |
| | | | | CCACACATTGGTAC | no: | | no: |
| | | | | | 56 | | 146 |
| 201930_at | NM_005915 | HG_010_16268 | LUA#72 | TAATACGACTCACTATAGGGTCATT | seq | CCTTGATGTCTGAGCTTTCCT | seq |
| | | | | TACCTTTAATCCAATAATCACCCAT | id | CCCTTTAGTGAGGGTTAAT | id |
| | | | | GAGTACTCAACTTG | no: | | no: |
| | | | | | 57 | | 147 |
| 204351_at | NM_005980 | HG_010_19452 | LUA#73 | TAATACGACTCACTATAGGGATCAA | seq | CCGTGGATAAATTGCTCAAGT | seq |
| | | | | ATCTCATCAATTCAACAATGAGTGG | id | CCCTTTAGTGAGGGTTAAT | id |
| | | | | AAAAGACAAGGATG | no: | | no: |
| | | | | | 58 | | 148 |
| 200790_at | NM_002539 | HG_010_17575 | LUA#74 | TAATACGAGTCACTATAGGGTACAC | seq | CATTTGTAGCTTGTACAATGT | seq |
| | | | | ATCTTACAAACTAATTTCACCCCTC | id | CCCTTTAGTGAGGGTTAAT | id |
| | | | | AGCTGCTGAACAAG | no: | | no: |
| | | | | | 59 | | 149 |
| 202887_s_at | NM_019058 | * | LUA#75 | TAATACGACTCACTATAGGGAATCA | seq | CCTTCCCCCATCGTGTACTGT | seq |
| | | | | TACCTTTCAATCTTTTACAACCTGG | id | CCCTTTAGTGAGGGTTAAT | id |
| | | | | CAGCTGCGTTTAAG | no: | | no: |
| | | | | | 60 | | 150 |
| 200077_s_at | NM_004152 | HG_010_22476 | LUA#31 | TAATACGACTCACTATAGGGTTCAC | seq | GTGCAAATAAACGCTCACTCT | seq |
| | | | | TTTTCAATCAACTTTAATCTTTGTC | id | CCCTTTAGTGAGGGTTAAT | id |
| | | | | CGCATGTTGTAATC | no: | | no: |
| | | | | | 61 | | 151 |
| 207320_x_at | NM_004602 | HG_010_18893 | LUA#32 | TAATACGACTCACTATAGGGATTAT | seq | AGAACTAAATGCACTGTGCAT | seq |
| | | | | TCACTTCAAACTAATCTACGAAAGC | id | CCCTTTAGTGAGGGTTAAT | id |
| | | | | ATAACCCCTACTGT | no: | | no: |
| | | | | | 62 | | 152 |
| 208641_s_at | NM_018890 | HG_010_22573 | LUA#33 | TAATACGACTCACTATAGGGTCAAT | seq | GAGAAGAAGCTGACTCCCATT | seq |
| | | | | TACTTCACTTTAATCCTTTACACGA | id | CCCTTTAGTGAGGGTTAAT | id |
| | | | | TCGAGAAACTGAAG | no: | | no: |
| | | | | | 63 | | 153 |
| 213867_x_at | NM_001101 | HG_010_19208 | LUA#34 | TAATACGACTCACTATAGGGTCATT | seq | CACAGAGGGGAGGTGATAGCT | seq |
| | | | | CATATACATACCAATTCATGCCCAG | id | CCCTTTAGTGAGGGTTAAT | id |
| | | | | TCCTCTCCCAAGTC | no: | | no: |
| | | | | | 64 | | 154 |
| 204158_s_at | NM_006019 | HG_010_07626 | LUA#35 | TAATACGACTGACTATAGGGCAATT | seq | GCATCTGTGAATGGCTGGAGT | seq |
| | | | | TCATCATTCATTCATTTCAGGTTGC | id | CCCTTTAGTGAGGGTTAAT | id |
| | | | | TGGACCTGCCTGAC | no: | | no: |
| | | | | | 65 | | 155 |
| 200691_s_at | NM_004134 | HG_010_15879 | LUA#76 | TAATACGACTCACTATAGGGAATCT | seq | CTGTGTCTGGCACCTACATCT | seq |
| | | | | AACAAACTCATCTAAATACTTTTCT | id | CCCTTTAGTGAGGGTTAAT | id |
| | | | | AGCTACCTTCTGCC | no: | | no: |
| | | | | | 66 | | 156 |
| 201077_s_at | NM_005008 | HG_010_18994 | LUA#77 | TAATACGACTCACTATAGGGCAATT | seq | CTGGCATGAAGGATTCCAGGT | seq |
| | | | | AACTACATACAATACATACTCAGAG | id | CCCTTTAGTGAGGGTTAAT | id |
| | | | | AGCATGAACTGATG | no: | | no: |
| | | | | | 67 | | 157 |
| 217810_x_at | NM_020117 | HG_010_16506 | LUA#78 | TAATACGACTCACTATAGGGCTATC | seq | GCTATCAGAACCTTAGGCTGT | seq |
| | | | | TATCTAACTATCTATATCACTGATT | id | CCCTTTAGTGAGGGTTAAT | id |
| | | | | GTGTCTACTGATTG | no: | | no: |
| | | | | | 68 | | 158 |
| 200792_at | NM_001469 | HG_010_07661 | LUA#79 | TAATACGACTCACTATAGGGTTCAT | seq | GTGTAGCCCTGCCAGTTTGCT | seq |
| | | | | AACTACAATACATCATCATTTTCTG | id | CCCTTTAGTGAGGGTTAAT | id |
| | | | | TTGCCATGGTGATG | no: | | no: |
| | | | | | 69 | | 159 |
| 218140_x_at | NM_021203 | HG_010_03138 | LUA#80 | TAATACGACTCACTATAGGGCTAAC | seq | CTGCTCTGCTGCTCTGGATGT | seq |
| | | | | TAACAATAATCTAACTAACAGTGTG | id | CCCTTTAGTGAGGGTTAAT | id |
| | | | | TGGAGATTTAGGTG | no: | | no: |
| | | | | | 70 | | 160 |
| 210908_s_at | NM_002624 | HG_010_15000 | LUA#36 | TAATACGACTCACTATAGGGCAATT | seq | GAGAAGCACGCCATGAAACAT | seq |
| | | | | CATTTCATTCACAATCAATAAATCC | id | CCCTTTAGTGAGGGTTAAT | id |
| | | | | AACCAGCTCTTCAG | no: | | no: |
| | | | | | 71 | | 161 |
| 201460_at | NM_004759 | HG_010_02788 | LUA#37 | TAATACGACTCACTATAGGGCTTTT | seq | CAATAACTCTCTACAGGAATT | seq |
| | | | | CATCTTTTCATCTTTCAATCCTGCC | id | CCCTTTAGTGAGGGTTAAT | id |
| | | | | CACGGGAGGACAAG | no: | | no: |
| | | | | | 72 | | 162 |
| 203470 s_at | NM_002664 | HG_010_17685 | LUA#38 | TAATACGACTGACTATAGGGTCAAT | seq | CTGTTCCCACTCCCAGATGGT | seq |
| | | | | CATTACACTTTTCAACAATGCCCTG | id | CCCTTTAGTGAGGGTTAAT | id |
| | | | | TAACATTCCTGAAG | no: | | no: |
| | | | | | 73 | | 163 |
| 202803_s_at | NM_000211 | HG_010_18487 | LUA#39 | TAATACGACTCACTATAGGGTACAC | seq | GCCTCAAAATGACAGCCATGT | seq |
| | | | | AATCTTTTCATTACATCATAGAAAT | id | CCCTTTAGTGAGGGTTAAT | id |
| | | | | CCAGTTATTTTCCG | no: | | no: |
| | | | | | 74 | | 164 |
| 209124_at | NM_002468 | HG_010_07210 | LUA#40 | TAATACGACTCACTATAGGGCTTTC | seq | CCATGGACCTGTCCCCCTTTT | seq |
| | | | | TACATTATTCACAACATTACTTGTT | id | CCCTTTAGTGAGGGTTAAT | id |
| | | | | GAGGCATTTAGCTG | no: | | no: |
| | | | | | 75 | | 165 |
| 201892_s_at | NM_000884 | HG_010_17352 | LUA#81 | TAATACGACTCACTATAGGGCTTTA | seq | CTGGCATCCAACACTCATGCT | seq |
| | | | | ATCTACACTTTCTAACAATATTTGT | id | CCCTTTAGTGAGGGTTAAT | id |
| | | | | CCCTTACCTGATTG | no: | | no: |
| | | | | | 76 | | 166 |
| 200647_x_at | NM_003752 | HG_010_19669 | LUA#82 | TAATACGACTCACTATAGGGTACAT | seq | CTGCTACCACATGACAGACAT | seq |
| | | | | ACACTAATAACATACTCATTTGCTG | id | CCCTTTAGTGAGGGTTAAT | id |
| | | | | ATTATACTTCTGAG | no: | | no: |
| | | | | | 77 | | 167 |
| 218512_at | NM_018256 | HG_010_03754 | LUA#83 | TAATACGACTCACTATAGGGATACA | seq | GACAGACAGAGGGCTACTTCT | seq |
| | | | | ATCTAACTTCACTATTACAAAAGTT | id | CCCTTTAGTGAGGGTTAAT | id |
| | | | | CTGAGTGTAGACTG | no: | | no: |
| | | | | | 78 | | 168 |
| 209932_s_at | NM_001948 | HG_010_10582 | LUA#84 | TAATACGACTCACTATAGGGTCAAC | seq | CACAGGCAAGAGTGTTCTTTT | seq |
| | | | | TAACTAATCATCTATCAATGACCAC | id | CCCTTTAGTGAGGGTTAAT | id |
| | | | | CCAGTTTGTGGAAG | no: | | no: |
| | | | | | 79 | | 169 |
| 200650_s_at | NM_005566 | HG_010_19291 | LUA#85 | TAATACGACTCACTATAGGGATACT | seq | GCACCACTGCCAATGCTGTAT | seq |
| | | | | ACATCATAATCAAACATCAATAGTT | id | CCCTTTAGTGAGGGTTAAT | id |
| | | | | CTGCCACCTCTGAC | no: | | no: |
| | | | | | 80 | | 170 |
| 217733_s_at | NM_021103 | HG_010_00217 | LUA#41 | TAATACGACTCACTATAGGGTTACT | seq | GAGAAGCGGAGTGAAATTTCT | seq |
| | | | | ACACAATATACTCATCAATCCAAAG | id | CCCTTTAGTGAGGGTTAAT | id |
| | | | | AGACCATTGAGCAG | no: | | no: |
| | | | | | 81 | | 171 |
| 210592_s_at | NM_002970 | HG_010_17875 | LUA#42 | TAATACGACTCACTATAGGGCTATC | seq | GAGTGCTGCTGTAGATGACAT | seq |
| | | | | TTCATATTTCACTATAAACAATGGC | id | CCCTTTAGTGAGGGTTAAT | id |
| | | | | AACAGAGGAGTGAG | no: | | no: |
| | | | | | 82 | | 172 |
| 204122_at | NM_003332 | HG_010_18121 | LUA#43 | TAATACGACTCACTATAGGGCTTTC | seq | CAGACCGCTCCCCAATACTCT | seq |
| | | | | AATTACAATACTCATTACAGAGTGC | id | CCCTTTAGTGAGGGTTAAT | id |
| | | | | CATCCCTGAGAGAC | no: | | no: |
| | | | | | 83 | | 173 |
| 204232_at | NM_004106 | HG_010_18680 | LUA#44 | TAATACGACTCACTATAGGGTCATT | seq | GAGACTCTGAAGCATGAGAAT | seq |
| | | | | TACCAATCTTTCTTTATACCCAGGA | id | CCCTTTAGTGAGGGTTAAT | id |
| | | | | ACCAGGAGACTTAC | no: | | no: |
| | | | | | 84 | | 174 |
| 216598_s_at | NM_002982 | HG_010_15183 | LUA#45 | TAATACGACTCACTATAGGGTCATT | seq | CCTGGGATGTTTTGAGGGTCT | seq |
| | | | | TCACAATTCAATTACTCAATCTTGA | id | CCCTTTAGTGAGGGTTAAT | id |
| | | | | ACCACAGTTCTACC | no: | | no: |
| | | | | | 85 | | 175 |
| 204798_at | NM_005375 | HG_010_19159 | LUA#86 | TAATAGGACTCACTATAGGGCTAAT | seq | CATGGATCCTGTGTTTGCAAT | seq |
| | | | | TACTAACATCACTAACAATGTATGG | id | CCCTTTAGTGAGGGTTAAT | id |
| | | | | TCTCAGAACTGTTG | no: | | no: |
| | | | | | 86 | | 176 |
| 203949_at | NM_000250 | HG_010_18429 | LUA#87 | TAATACGACTCACTATAGGGAAACT | seq | CTTATTCACTGAAGTTCTCCT | seq |
| | | | | AACATCAATACTTACATCATTCCTC | id | CCCTTTAGTGAGGGTTAAT | id |
| | | | | ACCCTGATTTCTTG | no: | | no: |
| | | | | | 87 | | 177 |
| 202107_s_at | NM_004526 | HG_010_18766 | LUA#88 | TAATACGACTCACTATAGGGTTACT | seq | CTCCCTGTCTGTTTCCCCACT | seq |
| | | | | TCACTTTCTATTTACAATCACAGTT | id | CCCTTTAGTGAGGGTTAAT | id |
| | | | | ATCAGCTGCCATTG | no: | | no: |
| | | | | | 88 | | 178 |
| 211951_at | NM_004741 | HG_010_18809 | LUA#89 | TAATACGACTCACTATAGGGTATAC | seq | GGTCTTGATGAGGACAGAAGT | seq |
| | | | | TATCAACTCAACAACATATCCCTCA | id | CCCTTTAGTGAGGGTTAAT | id |
| | | | | GGTCTCTAGGTGAG | no: | | no: |
| | | | | | 89 | | 179 |
| 202431_s_at | NM_002467 | HG_010_00920 | LUA#90 | TAATACGACTCACTATAGGGCTAAA | seq | GTCCAAGCAGAGGAGCAAAAT | seq |
| | | | | TACTTCACAATTCATCTAACCACAG | id | CCCTTTAGTGAGGGTTAAT | id |
| | | | | CATACATCCTGTCC | no: | | no: |
| | | | | | 90 | | 180 |

control features:

| | | | | | |
|---|---|---|---|---|---|
| descrip- | RefSeq | RefSet | Flex- | | downstream probe |
| tion | ID | ID | MAP | upstream probe sequence | sequence |

| | | | | | | | |
|---|---|---|---|---|---|---|---|
| ACTB | NM_001101 | * | LUA#91 | TAATACGACTCACTATAGGGTTCAT | seq | CATTGTTACAGGAAGTCCCTT | seq |
| | | | | AACATCAATCATAACTTACGTCATT | id | CCCTTTAGTGAGGGTTAAT | id |
| | | | | CCAAATATGAGATG | no: | | no: |
| | | | | | 181 | | 186 |
| TFRC | NM_003234 | * | LUA#92 | TAATACGACTCACTATAGGGCTATT | seq | GTGATCAATTAAATGTAGGTT | seq |
| | | | | ACACTTTAAACATCAATACCGTCTG | id | CCCTTTAGTGAGGGTTAAT | id |
| | | | | CCTACCCATTCGTG | no: | | no: |
| | | | | | 182 | | 187 |
| GAPDH_5 | NM_002046 | * | LUA#93 | TAATACGACTCACTATAGGGCTTTC | seq | GTTTACATGTTCGAATATGAT | seq |
| | | | | TATTCATCTAAATACAAACTCATTG | id | CCCTTTAGTGAGGGTTAAT | id |
| | | | | AGCTCAACTACATG | no: | | no: |
| | | | | | 183 | | 188 |
| GAPDH_M | NM_002046 | * | LUA#94 | TAATACGACTCACTATAGGGCTTTC | seq | CCACCCAGAAGACTGTGGATT | seq |
| | | | | TATCTTTCTACTCAATAATCACAGT | id | CCCTTTAGTGAGGGTTAAT | id |
| | | | | CCATGCCATCACTG | no: | | no: |
| | | | | | 184 | | 189 |
| GAPDH_3 | NM_002046 | * | LUA#95 | TAATACGACTCACTATAGGGTACAC | seq | CAAGAGCACAAGAGGAAGAGT | seq |
| | | | | TTTAAACTTACTACACTAACCCTGG | id | CCCTTTAGTGAGGGTTAAT | id |
| | | | | ACCACCAGCCCCAG | no: | | no: |
| | | | | | 185 | | 190 |

*probes designed against RefSeq
FlexMAP sequence shown in red
gene specific sequences shown in blue
FlexMAP sequence of upstream primer bases 21-44
gene specific sequences of upstream probe bases 45-64
gene specific sequences of downstream probe bases 1-20
TABLE 4

Capture Probes

| | | | |
|---|---|---|---|
| +HL,1 | FlexMAP | +HL,15 | +HL,32 |
| bead ID | ID | capture probe sequence+HZ,1/32 | |

| | | | | |
|---|---|---|---|---|
| Bead #1 | LUA-1 | GATTTGTATTGATTGAGATTAAAG | +TL,32 | |
| | | | | seq id no:191 |
| Bead #2 | LUA-2 | TGATTGTAGTATGTATTGATAAAG | | |
| | | | | seq id no:192 |
| Bead #3 | LUA-3 | GATTGTAAGATTTGATAAAGTGTA | | |
| | | | | seq id no:193 |
| Bead #4 | LUA-4 | GATTTGAAGATTATTGGTAATGTA | | |
| | | | | seq id no:194 |
| Bead #5 | LUA-5 | GATTGATTATTGTGATTTGAATTG | | |
| | | | | seq id no:195 |
| Bead #46 | LUA-46 | TGTATTGAATGAATTGTTGATGTA | | |
| | | | | seq id no:196 |
| Bead #47 | LUA-47 | ATTATGAAGTAAGTTAATGAGAAG | | |
| | | | | seq id no:197 |
| Bead #48 | LUA-48 | ATTATTGAGATGTGAAGTTTGTTT | | |
| | | | | seq id no:198 |
| Bead #49 | LUA-49 | GTAAGTAAATTGAAAGATTGATGA | | |
| | | | | seq id no:199 |
| Bead #50 | LUA-50 | GTAAATGATGATATTGGTATATTG | | |
| | | | | seq id no:200 |
| Bead #6 | LUA-6 | GATTTGATTGTAAAAGATTGTTGA | | |
| | | | | seq id no:201 |
| Bead #7 | LUA-7 | ATTGGTAAATTGGTAAATGAATTG | | |
| | | | | seq id no:202 |
| Bead #8 | LUA-8 | GTAAGTAATGAATGTAAAAGGATT | | |
| | | | | seq id no:203 |
| Bead #9 | LUA-9 | GTAAGATGTTGATATAGAAGATTA | | |
| | | | | seq id no:204 |
| Bead #10 | LUA-10 | TGTAGATTTGTATGTATGTATGAT | | |
| | | | | seq id no:205 |
| Bead #51 | LUA-51 | ATTGTTGATGATTGATTGAAATGA | | |
| | | | | seq id no:206 |
| Bead #52 | LUA-52 | ATTGTGAAGTATAAAGATGATTGA | | |
| | | | | seq id no:207 |
| Bead #53 | LUA-53 | TGTAGAAGATGAGATGTATAATTA | | |
| | | | | seq id no:208 |
| Bead #54 | LUA-54 | ATGAATTGAAAGTGATTGAAAAAG | | |
| | | | | seq id no:209 |
| Bead #55 | LUA-55 | GTTAGTTATTGAGAAGTGTATATA | | |
| | | | | seq id no:210 |
| Bead #11 | LUA-11 | GATTAAAGTGATTGATGATTTGTA | | |
| | | | | seq id no:211 |
| Bead #12 | LUA-12 | AAAGAAAGAAAGAAAGAAAGTGTA | | |
| | | | | seq id no:212 |
| Bead #13 | LUA-13 | TTAGTGAAGAAGTATAGTTTATTG | | |
| | | | | seq id no:213 |
| Bead #14 | LUA-14 | AAAGTATAGTAAGATGTATAGTAG | | |
| | | | | seq id no:214 |
| Bead #15 | LUA-15 | TGAATTGATGAATGAATGAAGTAT | | |
| | | | | seq id no:215 |
| Bead #56 | LUA-56 | AAAGTGATGTATATGAGTAAATTG | | |
| | | | | seq id no:216 |
| Bead #57 | LUA-57 | GTAATGATAAAGATGATGATATTG | | |
| | | | | seq id no:217 |
| Bead #58 | LUA-58 | GTAGTAATGTTAATGAATTAGTAG | | |
| | | | | seq id no:218 |
| Bead #59 | LUA-59 | AAAGTGAAAAAGATTGATTGATGA | | |
| | | | | seq id no:219 |
| Bead #60 | LUA-60 | ATGAGATTATTGGATTTGTAGATT | | |
| | | | | seq id no:220 |
| Bead #16 | LUA-16 | TGATGATTTGAATGAAGATTGATT | | |
| | | | | seq id no:221 |
| Bead #17 | LUA-17 | TGATAAAGTGATAAAGGATTAAAG | | |
| | | | | seq id no:222 |
| Bead #18 | LUA-18 | TGATTTGAGTATTTGAGATTTTGA | | |
| | | | | seq id no:223 |
| Bead #19 | LUA-19 | GTATTTGAGTAAGTAATTGATTGA | | |
| | | | | seq id no:224 |
| Bead #20 | LUA-20 | GATTGTATTGAAGTATTGTAAAAG | | |
| | | | | seq id no:225 |
| Bead #61 | LUA-61 | TGAAGATTATGAATTGGTAAGATT | | |
| | | | | seq id no:226 |
| Bead #62 | LUA-62 | ATTGGATTATGAGATTATGATTGA | | |
| | | | | seq id no:227 |
| Bead #63 | LUA-63 | TGTAGTATAAAGTATATGAAGTAG | | |
| | | | | seq id no:228 |
| Bead #64 | LUA-64 | GTAAGTAGTAATTTGAATATGTAG | | |
| | | | | seq id no:229 |
| Bead #65 | LUA-65 | AAAGGTAAGATTATTGATGAAAAG | | |
| | | | | seq id no:230 |
| Bead #21 | LUA-21 | TGATTTGAGATTAAAGAAAGGATT | | |
| | | | | seq id no:231 |
| Bead #22 | LUA-22 | TGATTGAATTGAGTAAAAAGGATT | | |
| | | | | seq id no:232 |
| Bead #23 | LUA-23 | AAAGTTGAGATTTGAATGATTGAA | | |
| | | | | seq id no:233 |
| Bead #24 | LUA-24 | GTATTGTATTGAIAAGGTAATTGA | | |
| | | | | seq id no:234 |
| Bead #25 | LUA-25 | TGAAGATTTGAAGTAATTGAAAAG | | |
| | | | | seq id no:235 |
| Bead #66 | LUA-66 | GTAGATAGTATAGTTGTAATGTTA | | |
| | | | | seq id no:236 |
| Bead #67 | LUA-67 | GATTTGTAATTGTTGAGTAAATGA | | |
| | | | | seq id no:237 |
| Bead #68 | LUA-68 | AAAGAAAGATTGTTGAGATTATGA | | |
| | | | | seq id no:238 |
| Bead #69 | LUA-69 | GATGTGAATGTAATATGTTTATAG | | |
| | | | | seq id no:239 |
| Bead #70 | LUA-70 | TGATATGAATTGGATTATTGGTAT | | |
| | | | | seq id no:240 |
| Bead #26 | LUA-26 | TGAAAAAGTGTAGATTTTGAGTAA | | |
| | | | | seq id no:241 |
| Bead #27 | LUA-27 | AAAGTTGAGTATTGATTTGAAAAG | | |
| | | | | seq id no:242 |
| Bead #28 | LUA-28 | TTGATAATGTTTGTTTGTTTGTAG | | |
| | | | | seq id no:243 |
| Bead #29 | LUA-29 | AAAGAAAGGATTTGTAGTAAGATT | | |
| | | | | seq id no:244 |
| Bead #30 | LUA-30 | GTAAAAAGAAAGGTATAAAGGTAA | | |
| | | | | seq id no:245 |
| Bead #71 | LUA-71 | ATGAATTGATTGGATTGTAATGAT | | |
| | | | | seq id no:246 |
| Bead #72 | LUA-72 | GATTATTGGATTAAAGGTAAATGA | | |
| | | | | seq id no:247 |
| Bead #73 | LUA-73 | ATTGTTGAATTGATGAGATTTGAT | | |
| | | | | seq id no:248 |
| Bead #74 | LUA-74 | TGAAATTAGTTTGTAAGATGTGTA | | |
| | | | | seq id no:249 |
| Bead #75 | LUA-75 | TGTAAAAGATTGAAAGGTATGATT | | |
| | | | | seq id no:250 |
| Bead #31 | LUA-31 | GATTAAAGTTGATTGAAAAGTGAA | | |
| | | | | seq id no:251 |
| Bead #32 | LUA-32 | GTAGATTAGTTTGAAGTGAATAAT | | |
| | | | | seq id no:252 |
| Bead #33 | LUA-33 | AAAGGATTAAAGTGAAGTAATTGA | | |
| | | | | seq id no:253 |
| Bead #34 | LUA-34 | ATGAATTGGTATGTATATGAATGA | | |
| | | | | seq id no:254 |
| Bead #35 | LUA-35 | TGAAATGAATGAATGATGAAATTG | | |
| | | | | seq id no:255 |
| Bead #76 | LUA-76 | GTATTTAGATCAGTTTGTTAGATT | | |
| | | | | seq id no:256 |
| Bead #77 | LUA-77 | GTATGTATTGTATGTAGTTAATTG | | |
| | | | | seq id no:257 |
| Bead #78 | LUA-78 | TGATATAGATAGTTAGATAGATAG | | |
| | | | | seq id no:258 |
| Bead #79 | LUA-79 | ATGATGATGTATTGTAGTTATGAA | | |
| | | | | seq id no:259 |
| Bead #80 | LUA-80 | GTTAGTTAGATTATTGTTAGTTAG | | |
| | | | | seq id no:260 |
| Bead #36 | LUA-36 | ATTGATTGTGAATGAAATGAATTG | | |
| | | | | seq id no:261 |
| Bead #37 | LUA-37 | ATTGAAAGATGAAAAGATGAAAAG | | |
| | | | | seq id no:262 |
| Bead #38 | LUA-38 | ATTGTTGAAAAGTGTAATGATTGA | | |
| | | | | seq id no:263 |
| Bead #39 | LUA-39 | ATGATGTAATGAAAAGATTGTGTA | | |
| | | | | seq id no:264 |
| Bead #40 | LUA-40 | TAATGTTGTGAATAATGTAGAAAG | | |
| | | | | seq id no:265 |
| Bead #81 | LUA-81 | ATTGTTAGAAAGTGTAGATTAAAG | | |
| | | | | seq id no:266 |
| Bead #82 | LUA-82 | ATGAGTATGTTATTAGTGTATGTA | | |
| | | | | seq id no:267 |
| Bead #83 | LUA-83 | TGTAATAGTGAAGTTAGATTGTAT | | |
| | | | | seq id no:268 |
| Bead #84 | LUA-84 | ATTGATAGATGATTAGTTAGTTGA | | |
| | | | | seq id no:269 |
| Bead #85 | LUA-85 | TGATGTTTGATTATGATGTAGTAT | | |
| | | | | seq id no:270 |
| Bead #41 | LUA-41 | ATTGATGAGTATATTGTGTAGTAA | | |
| | | | | seq id no:271 |
| Bead #42 | LUA-42 | GTTTATAGTGAAATATGAAGATAG | | |
| | | | | seq id no:272 |
| Bead #43 | LUA-43 | TGTAATGAGTATTGTAATTGAAAG | | |
| | | | | seq id no:273 |
| Bead #44 | LUA-44 | GTATAAAGAAAGATTGGTAAATGA | | |
| | | | | seq id no:274 |
| Bead #45 | LUA-45 | TTGAGTAATTGAATTGTGAAATGA | | |
| | | | | seq id no:275 |
| Bead #86 | LUA-86 | ATTGTTAGTGATGTTAGTAATTAG | | |
| | | | | seq id no:276 |
| Bead #87 | LUA-87 | TGATGTAAGTATTGATGTTAGTTT | | |
| | | | | seq id no:277 |
| Bead #88 | LUA-88 | GATTGTAAATAGAAAGTGAAGTAA | | |
| | | | | seq id no:278 |
| Bead #89 | LUA-89 | ATATGTTGTTGAGTTGATAGTATA | | |
| | | | | seq id no:279 |
| Bead #90 | LUA-90 | TTAGATGAATTGTGAAGTATTTAG | | |
| | | | | seq id no:280 |
| Bead #91 | LUA-91 | GTAAGTTATGATTGATGTTATGAA | | |
| | | | | seq id no:281 |
| Bead #92 | LUA-92 | GTATTGATGTTTAAAGTGTAATAG | | |
| | | | | seq id no:282 |
| Bead #93 | LUA-93 | GTTTGTATTTAGATGAATAGAAAG | | |
| | | | | seq id no:283 |
| Bead #94 | LUA-94 | ATTATTGAGTAGAAAGATAGAAAG | | |
| | | | | seq id no:284 |
| Bead #95 | LUA-95 | TTAGTGTAGTAAGTTTAAAGTGTA | | |
| | | | | seq id no:285+TZ,1/32 |

TABLE 5
Table 5A. Microtiter plates.

| description | FlexMap ID | blank | blank | dmso1 | dmso2 | dmso3 | dmso4 | dmso5 | dmso6 | dmso7 | dmso8 | dmso9 | dmso10 |
|---|---|---|---|---|---|---|---|---|---|---|---|---|---|
| NM_005736 | LUA#1 | 40 | 33.5 | 902 | 774 | 850.5 | 914 | 836.5 | 900 | 888 | 563 | 803.5 | 692.5 |
| NM_000070 | LUA#2 | 39 | 36 | 653.5 | 434 | 571 | 624 | 650 | 609 | 575.5 | 265 | 499.5 | 499.5 |
| NM_018217 | LUA#3 | 42 | 30 | 1547 | 1243 | 1382 | 1463 | 1448 | 1444.5 | 1416 | 713 | 1276.5 | 1180 |
| NM_004782 | LUA#4 | 45 | 39 | 1402 | 1082 | 1284 | 1397 | 1324 | 1234 | 1389.5 | 724.5 | 1105 | 1140.5 |
| NM_014962 | LUA#5 | 49 | 39 | 1724 | 1597 | 1549 | 1670 | 1554 | 1467 | 1437 | 732 | 1251 | 1222 |
| NM_004514 | LUA#46 | 39 | 30.5 | 1490.5 | 1130 | 1389 | 1498 | 1455 | 1394 | 1420.5 | 804.5 | 1235 | 1160.5 |
| NM_006773 | LUA#47 | 34.5 | 40 | 682 | 571 | 683 | 734 | 698 | 672.5 | 664 | 409 | 683 | 635 |
| NM_014288 | LUA#48 | 41 | 37 | 713 | 527 | 655 | 721 | 710 | 761 | 657 | 364 | 672 | 643 |
| NM_017440 | LUA#49 | 28 | 32 | 621 | 443 | 568 | 629 | 599 | 613 | 562 | 303 | 499 | 481 |
| NM_007331 | LUA#50 | 38.5 | 29 | 1011 | 821.5 | 931.5 | 956 | 988 | 981.5 | 839 | 359 | 755 | 736 |
| NM_173823 | LUA#6 | 38 | 27 | 1411 | 1222.5 | 1272.5 | 1413 | 1326 | 1203.5 | 1333 | 475 | 850 | 861 |
| NM_000962 | LUA#7 | 33 | 37 | 472 | 401 | 416.5 | 435 | 406 | 368.5 | 387 | 138 | 306 | 287 |
| NM_003825 | LUA#8 | 42 | 34.5 | 574.5 | 483 | 474.5 | 575 | 482 | 430.5 | 434 | 188 | 336 | 324 |
| NM_016061 | LUA#9 | 46 | 37 | 1208 | 1137 | 1050.5 | 1049 | 962 | 909.5 | 905 | 365 | 714 | 683 |
| NM_000153 | LUA#10 | 35 | 43 | 63 | 57.5 | 59 | 62.5 | 48 | 44.5 | 46 | 38 | 46 | 48 |
| NM_006948 | LUA#51 | 36.5 | 32.5 | 71 | 55 | 75 | 68 | 74 | 79.5 | 60.5 | 46 | 50 | 41 |
| NM_004631 | LUA#52 | 41 | 26 | 1544.5 | 1163 | 1288 | 1230 | 1170.5 | 1060 | 1047 | 364 | 731.5 | 729 |
| NM_002358 | LUA#53 | 33 | 32.5 | 564 | 409 | 570 | 611.5 | 616 | 671 | 583 | 275 | 547 | 464 |
| NM_013402 | LUA#54 | 34.5 | 31 | 1273.5 | 943.5 | 1181 | 1190 | 1216.5 | 1153.5 | 1108 | 456 | 976 | 945 |
| NM_000875 | LUA#55 | 42 | 30 | 1243.5 | 1137.5 | 1219.5 | 1507 | 1425 | 1383 | 1250 | 854.5 | 1158 | 1168 |
| NM_001974 | LUA#11 | 33 | 34 | 147 | 137 | 170 | 221.5 | 273 | 213.5 | 183 | 58 | 139 | 130 |
| NM_000632 | LUA#12 | 41 | 35 | 500.5 | 399 | 483 | 509 | 499.5 | 519.5 | 492 | 282 | 378 | 338.5 |
| NM_006457 | LUA#13 | 33.5 | 30 | 94 | 75 | 82.5 | 91 | 82 | 68 | 75 | 38 | 60 | 59 |
| NM_000698 | LUA#14 | 38.5 | 28 | 188 | 153 | 163.5 | 209 | 215.5 | 184 | 149 | 99 | 134 | 133 |
| NM_032571 | LUA#15 | 34.5 | 49.5 | 209 | 146 | 172 | 223 | 198 | 173.5 | 187 | 87 | 152 | 150 |
| NM_006138 | LUA#56 | 44 | 38.5 | 145.5 | 150 | 157 | 229 | 199 | 209 | 158 | 133 | 140 | 130 |
| NM_015201 | LUA#57 | 42 | 33 | 878 | 689 | 822 | 965 | 877.5 | 927 | 932 | 381 | 635 | 570 |
| NM_006985 | LUA#58 | 38 | 34 | 919 | 775 | 826 | 897 | 857 | 925 | 751 | 292.5 | 727 | 619 |
| NM_004095 | LUA#59 | 41 | 32 | 695 | 536.5 | 595 | 574 | 562 | 655 | 565.5 | 183.5 | 345 | 337.5 |
| NM_005914 | LUA#60 | 46 | 37 | 2195.5 | 1744 | 2157 | 2234 | 2262 | 2579 | 2082 | 1102 | 2212 | 2079 |
| NM_007282 | LUA#16 | 34 | 20 | 4387 | 3871 | 4222 | 4458 | 4248 | 4005 | 4536 | 3049.5 | 3935 | 3689 |
| NM_003644 | LUA#17 | 36 | 33 | 526 | 406 | 480.5 | 528 | 498 | 450 | 494 | 246.5 | 411.5 | 391.5 |
| NM_001498 | LUA#18 | 42 | 36 | 1913 | 1585 | 1809.5 | 2005 | 1957 | 1776.5 | 1849 | 805 | 1607 | 1538 |
| NM_003172 | LUA#19 | 39 | 33 | 3589 | 2978.5 | 3400 | 3500 | 3410.5 | 3151 | 3536 | 3020 | 3531 | 3474 |
| NM_004723 | LUA#20 | 60 | 48 | 832 | 591.5 | 736.5 | 873 | 807.5 | 813 | 798 | 329.5 | 716.5 | 652 |
| NM_014366 | LUA#61 | 38 | 28 | 1995 | 1551 | 1903.5 | 2057 | 1962 | 1912.5 | 1996 | 1294.5 | 1720 | 1635 |
| NM_003581 | LUA#62 | 38 | 39 | 360 | 341.5 | 317.5 | 455 | 640.5 | 540 | 412 | 151.5 | 429 | 402 |
| NM_018115 | LUA#63 | 38 | 31.5 | 3024 | 2378 | 2960 | 3112 | 2963 | 2980 | 2866 | 1873 | 2710 | 2595 |
| NM_021974 | LUA#64 | 36 | 35 | 2077.5 | 1654.5 | 2019 | 2122 | 2051 | 2001 | 1859.5 | 973.5 | 1770.5 | 1771 |
| NM_024045 | LUA#65 | 42 | 40 | 734 | 526.5 | 675 | 775 | 713 | 729 | 683 | 264 | 520 | 494 |
| NM_004079 | LUA#21 | 42 | 31 | 4089 | 3862.5 | 3968 | 3977 | 3945.5 | 3731 | 3760 | 2211 | 3375 | 3283.5 |
| NM_000414 | LUA#22 | 30.5 | 38.5 | 604 | 446 | 533 | 594 | 583 | 764 | 580 | 203 | 475 | 440.5 |
| NM_001684 | LUA#23 | 36 | 38 | 2409.5 | 1974 | 2345 | 2586 | 2361 | 2644 | 2639 | 1719.5 | 2063 | 2080 |
| NM_003879 | LUA#24 | 31 | 29.5 | 960 | 709.5 | 920 | 1061 | 1060.5 | 1079.5 | 920.5 | 446 | 891 | 871 |
| NM_002166 | LUA#25 | 41 | 29 | 1321.5 | 1026 | 1432 | 1466 | 1409 | 1475.5 | 1220 | 663.5 | 1490.5 | 1453 |
| NM_005952 | LUA#66 | 40 | 36 | 1423 | 1277.5 | 1395.5 | 1459.5 | 1482 | 1431 | 1332 | 675.5 | 1259 | 1185 |
| NM_001034 | LUA#67 | 40 | 36 | 607 | 491.5 | 520.5 | 777 | 713 | 635.5 | 580 | 255 | 614 | 609 |
| NM_003132 | LUA#68 | 36 | 42 | 789 | 626 | 706 | 671 | 617 | 563.5 | 583 | 198.5 | 518.5 | 524 |
| NM_018164 | LUA#69 | 41 | 34 | 205.5 | 149 | 182 | 235 | 274 | 250 | 198 | 100.5 | 189 | 142 |
| NM_014573 | LUA#70 | 41 | 39 | 292 | 225.5 | 240 | 328.5 | 314.5 | 272 | 244.5 | 114 | 257.5 | 232 |
| NM_014333 | LUA#26 | 28.5 | 27 | 1505 | 1147 | 1369.5 | 1467 | 1427 | 1484 | 1415 | 774.5 | 1217.5 | 1236 |
| NM_006432 | LUA#27 | 38.5 | 33 | 699 | 534 | 646 | 713.5 | 703 | 718 | 636 | 315 | 562 | 550 |
| NM_000433 | LUA#28 | 45 | 44 | 878 | 576 | 830.5 | 896 | 906 | 796 | 844 | 351 | 893 | 824 |
| NM_000147 | LUA#29 | 42 | 24 | 639 | 466 | 629 | 651 | 659 | 597.5 | 645 | 256 | 532.5 | 499 |
| NM_000584 | LUA#30 | 41.5 | 36 | 394 | 346 | 379 | 483.5 | 407 | 340.5 | 306.5 | 120 | 268.5 | 289 |
| NM_006452 | LUA#71 | 35 | 36 | 2704.5 | 2307.5 | 2678 | 2654.5 | 2673 | 2689 | 2707 | 1357.5 | 2109 | 1953 |
| NM_005915 | LUA#72 | 45.5 | 39 | 1061.5 | 874 | 1025 | 1120 | 1087 | 921 | 1013 | 478 | 1105 | 1020 |
| NM_005980 | LUA#73 | 40.5 | 44.5 | 159 | 108 | 139 | 145.5 | 144.5 | 144 | 145 | 92.5 | 138.5 | 130 |
| NM_002539 | LUA#74 | 47 | 43 | 2035.5 | 1756 | 2051.5 | 2189.5 | 2318 | 1930 | 1994 | 1204.5 | 2047.5 | 2038 |
| NM_019058 | LUA#75 | 48 | 37 | 2504 | 2473 | 2482 | 2914 | 3027.5 | 2942.5 | 2642 | 1576 | 2562.5 | 2616.5 |
| NM_004152 | LUA#31 | 44 | 42 | 1205 | 983 | 1218 | 1317 | 1344 | 1212 | 1299 | 547.5 | 1317.5 | 1129.5 |
| NM_004602 | LUA#32 | 38 | 30 | 182 | 293 | 205 | 255 | 222 | 159 | 170 | 770 | 223.5 | 139 |
| NM_018890 | LUA#33 | 51 | 44 | 2917 | 2521.5 | 2741.5 | 2699 | 3109 | 2785 | 3028.5 | 2194 | 2814 | 2125 |
| NM_001101 | LUA#34 | 47 | 41 | 3269.5 | 2707 | 3122.5 | 3280 | 3254.5 | 2939 | 3057 | 2117 | 3070 | 2979 |
| NM_006019 | LUA#35 | 40 | 32.5 | 732 | 617.5 | 657.5 | 710.5 | 678 | 550.5 | 633 | 242 | 493 | 479.5 |
| NM_004134 | LUA#76 | 53 | 49 | 1773 | 1613 | 1923 | 1777.5 | 1756.5 | 1565 | 1674 | 812.5 | 1734 | 1752 |
| NM_005008 | LUA#77 | 38 | 37 | 1466 | 1175 | 1420 | 1489 | 1546 | 1279 | 1331 | 613.5 | 1198 | 1154 |
| NM_020117 | LUA#78 | 37 | 32.5 | 3623 | 3228 | 3691 | 3649 | 3820 | 3251 | 3418 | 2516 | 3693.5 | 3553 |
| NM_001469 | LUA#79 | 35 | 30.5 | 609.5 | 490 | 632.5 | 745 | 811 | 727 | 615.5 | 295 | 646 | 600 |
| NM_021203 | LUA#80 | 43 | 48 | 854.5 | 657 | 825 | 824 | 830.5 | 702 | 752 | 289.5 | 812 | 729 |
| NM_002624 | LUA#36 | 54 | 45.5 | 483 | 414 | 462 | 482.5 | 490 | 414.5 | 426 | 178 | 314 | 300.5 |
| NM_004759 | LUA#37 | 45 | 40 | 210 | 160 | 207 | 214.5 | 192 | 162 | 157 | 97 | 190 | 175 |
| NM_002664 | LUA#38 | 42.5 | 44 | 758.5 | 572 | 687 | 715.5 | 717 | 676 | 717 | 272 | 683 | 690 |
| NM_000211 | LUA#39 | 43 | 47 | 2399 | 2085 | 2457.5 | 2480 | 2328 | 1741 | 2234 | 1125 | 2855 | 2765 |
| NM_002468 | LUA#40 | 36 | 41.5 | 434 | 421 | 408.5 | 461 | 466 | 408 | 403 | 238 | 396.5 | 335 |
| NM_000884 | LUA#81 | 48 | 53 | 1425.5 | 1158 | 1403 | 1476 | 1501.5 | 1293 | 1396 | 661 | 1224.5 | 1201.5 |
| NM_003752 | LUA#82 | 51 | 46 | 2178 | 1591 | 1908 | 2000 | 2057 | 1847 | 2035 | 1041.5 | 1743 | 1589 |
| NM_018256 | LUA#83 | 38 | 42 | 1960 | 1487 | 1947.5 | 1945.5 | 1933 | 1831 | 1798 | 1027 | 1858.5 | 1781 |
| NM_001948 | LUA#84 | 51 | 44 | 3639 | 3037 | 3513 | 3628 | 3641 | 3222 | 3639 | 1898.5 | 3089 | 3064 |
| NM_005566 | LUA#85 | 50 | 46.5 | 2849 | 2508 | 2754 | 2860 | 2845 | 2649 | 2739.5 | 1334 | 2560 | 2497 |
| NM_021103 | LUA#41 | 51 | 45 | 3369.5 | 2796 | 3116 | 3286.5 | 3175 | 2888 | 3034 | 2155 | 2887 | 2663.5 |
| NM_002970 | LUA#42 | 50 | 53 | 1390 | 1330 | 1252 | 1169.5 | 1144.5 | 922.5 | 1002 | 381 | 800.5 | 798 |
| NM_003332 | LUA#43 | 37 | 42 | 3442 | 3303 | 2960 | 2860 | 2644 | 2238 | 2494 | 1006 | 1976 | 2066.5 |
| NM_004106 | LUA#44 | 43 | 40 | 756 | 623 | 688 | 662 | 601 | 546 | 562 | 203 | 416.5 | 430.5 |
| NM_002982 | LUA#45 | 48 | 38.5 | 4465 | 4583 | 4733 | 4626 | 4576 | 4067.5 | 4536 | 2998 | 4098 | 3942.5 |
| NM_005375 | LUA#86 | 53.5 | 53 | 3445 | 2883 | 3140 | 3429 | 3216 | 3079 | 3213.5 | 1598.5 | 2714 | 2510 |
| NM_000250 | LUA#87 | 50 | 40.5 | 3990 | 3233.5 | 3862 | 3996 | 3850 | 3694.5 | 3993 | 2672 | 3456 | 3368 |
| NM_004526 | LUA#88 | 42 | 31 | 2129 | 1933 | 2176 | 2149 | 2161 | 1926 | 1970.5 | 1115 | 1947 | 1890.5 |
| NM_004741 | LUA#89 | 50.5 | 39 | 1970 | 1864 | 1808.5 | 1645 | 1661 | 1432.5 | 1528 | 561.5 | 1340 | 1146.5 |
| NM_002467 | LUA#90 | 67 | 56.5 | 3253 | 2824 | 3142 | 3156.5 | 3104 | 2666 | 2784 | 1819.5 | 2700.5 | 2541 |
| ACTB | LUA#91 | 54 | 51 | 3126 | 2638 | 3086 | 3191 | 3160 | 3024 | 3100 | 1853.5 | 3149 | 3002.5 |
| TFRC | LUA#92 | 76 | 79.5 | 1348 | 983.5 | 1283.5 | 1329.5 | 1267 | 1098 | 1256 | 467.5 | 946 | 967 |
| GAPDH_5 | LUA#93 | 59 | 46 | 2708 | 1911 | 2385.5 | 2693 | 2523 | 2374.5 | 2539.5 | 1475 | 2364 | 2243.5 |
| GAPDH_M | LUA#94 | 48 | 49.5 | 4772 | 3907 | 4477 | 5031.5 | 4540 | 4282 | 4848 | 3529 | 4163 | 4180 |
| GAPDH_3 | LUA#95 | 74.5 | 69 | 4277 | 3837 | 4461.5 | 4434 | 4414 | 4444 | 4482 | 3794.5 | 4211 | 4058 |

Table 5B. Microtiter plates

| description | FlexMap ID | dmso11 | dmso12 | dmso13 | dmso14 | dmso15 | dmso16 | dmso17 | dmso18 | dmso19 | dmso20 | dmso21 | dmso22 |
|---|---|---|---|---|---|---|---|---|---|---|---|---|---|
| NM_005736 | LUA#1 | 863 | 780.5 | 645 | 792.5 | 662 | 690 | 686.5 | 690 | 744 | 752 | 821 | 824.5 |
| NM_000070 | LUA#2 | 602 | 551 | 497.5 | 605 | 489 | 519 | 524.5 | 532 | 532.5 | 541 | 574 | 575 |
| NM_018217 | LUA#3 | 1301 | 1291 | 1131 | 1309.5 | 1049 | 1136 | 1159 | 1144 | 1216.5 | 1295 | 1334 | 1278 |
| NM_004782 | LUA#4 | 1261.5 | 1219 | 1206 | 1280 | 936.5 | 1113 | 1077 | 1085 | 1223 | 1228 | 1291.5 | 1200 |
| NM_014962 | LUA#5 | 1351 | 1339 | 1064 | 1149.5 | 1037 | 1121 | 1101 | 1135 | 1245 | 1246.5 | 1325 | 1246.5 |
| NM_004514 | LUA#46 | 1269 | 1286.5 | 1143 | 1367 | 1083 | 1216 | 1144 | 1196 | 1271 | 1302 | 1276 | 1284.5 |
| NM_006773 | LUA#47 | 742.5 | 671 | 677.5 | 757 | 598 | 690.5 | 691.5 | 689 | 687 | 707 | 730 | 706 |
| NM_014288 | LUA#48 | 754 | 671 | 683 | 764 | 579 | 735.5 | 701 | 704 | 708 | 718 | 733 | 708 |
| NM_017440 | LUA#49 | 533 | 498 | 481.5 | 569 | 436 | 529 | 490 | 506 | 499 | 527 | 533 | 544.5 |
| NM_007331 | LUA#50 | 756 | 792 | 605 | 745 | 636 | 718 | 726 | 692 | 711 | 767 | 785 | 786 |
| NM_173823 | LUA#6 | 876 | 1030 | 673.5 | 802 | 672 | 763 | 735 | 738 | 861.5 | 954 | 913.5 | 959 |
| NM_000962 | LUA#7 | 293 | 363 | 281 | 328.5 | 281.5 | 275 | 278 | 278 | 291 | 340 | 348 | 342 |
| NM_003825 | LUA#8 | 350 | 335 | 293 | 267.5 | 254 | 222 | 245.5 | 265 | 313 | 315 | 347 | 310 |
| NM_016061 | LUA#9 | 737 | 740 | 530.5 | 653.5 | 623 | 649 | 597 | 618 | 659 | 648 | 707 | 681 |
| NM_000153 | LUA#10 | 44.5 | 46 | 46 | 44 | 39 | 41 | 44 | 42 | 43 | 50 | 53 | 51 |
| NM_006948 | LUA#51 | 51 | 55.5 | 56 | 65 | 56 | 62 | 55 | 60 | 55.5 | 64 | 57 | 60.5 |
| NM_004631 | LUA#52 | 792.5 | 864 | 593.5 | 702.5 | 575 | 698.5 | 641 | 698.5 | 709.5 | 756 | 744.5 | 779 |
| NM_002358 | LUA#53 | 614 | 582.5 | 560 | 676 | 542.5 | 606 | 503 | 526 | 553 | 560.5 | 578.5 | 574.5 |
| NM_013402 | LUA#54 | 974 | 1061 | 870 | 999 | 812 | 940.5 | 906.5 | 918 | 941 | 970.5 | 977 | 1031 |
| NM_000875 | LUA#55 | 1337 | 1263 | 1215 | 1372.5 | 1168 | 1101 | 1141.5 | 1096 | 1191 | 1173.5 | 1280 | 1223 |
| NM_001974 | LUA#11 | 194 | 214 | 175 | 216 | 163.5 | 117 | 114 | 119 | 164 | 178 | 222.5 | 205.5 |
| NM_000632 | LUA#12 | 360 | 389.5 | 361 | 404 | 333 | 383 | 372 | 307 | 361.5 | 376 | 396 | 397 |
| NM_006457 | LUA#13 | 71 | 62.5 | 56 | 64.5 | 55 | 56.5 | 50 | 60 | 67 | 65 | 67 | 64 |
| NM_000698 | LUA#14 | 132.5 | 136 | 123.5 | 166.5 | 146 | 130 | 114 | 129 | 115.5 | 135 | 142 | 145 |
| NM_032571 | LUA#15 | 132.5 | 173 | 141 | 190 | 108 | 160.5 | 135 | 142.5 | 164.5 | 170 | 178 | 157 |
| NM_006138 | LUA#56 | 128 | 133.5 | 142 | 134 | 140 | 138 | 137 | 117 | 146.5 | 150 | 154 | 153 |
| NM_015201 | LUA#57 | 688 | 692 | 583 | 736.5 | 650 | 684.5 | 588.5 | 557 | 637 | 652 | 691.5 | 698 |
| NM_006985 | LUA#58 | 630 | 701 | 543.5 | 707.5 | 550 | 672 | 684 | 635 | 653.5 | 678 | 720.5 | 692 |
| NM_004095 | LUA#59 | 334 | 407 | 294.5 | 363 | 352.5 | 440 | 347.5 | 319 | 372 | 340 | 398.5 | 391 |
| NM_005914 | LUA#60 | 1967.5 | 2255 | 1967 | 2196 | 1708 | 2021 | 2120 | 1877 | 2054 | 2334 | 2477 | 2222 |
| NM_007282 | LUA#16 | 4208 | 4000.5 | 3735 | 4128 | 3643 | 3554 | 3724 | 3707 | 4109 | 3898.5 | 4083 | 3866 |
| NM_003644 | LUA#17 | 461 | 445.5 | 422.5 | 467 | 331 | 409 | 394 | 418 | 430 | 437.5 | 462.5 | 465.5 |
| NM_001498 | LUA#18 | 1627.5 | 1631 | 1477 | 1773 | 1383 | 1618.5 | 1582 | 1614 | 1701 | 1727 | 1700 | 1716 |
| NM_003172 | LUA#19 | 3838 | 3647 | 3528 | 3823.5 | 3374 | 3493 | 3499.5 | 3566 | 3683 | 3672 | 3821 | 3575 |
| NM_004723 | LUA#20 | 848.5 | 770 | 717 | 823.5 | 607 | 677 | 702.5 | 717 | 709 | 705 | 759 | 789.5 |
| NM_014366 | LUA#61 | 2015 | 1794.5 | 1782 | 2122.5 | 1726.5 | 1787 | 1758 | 1753 | 1799 | 1782 | 1903 | 1815 |
| NM_003581 | LUA#62 | 561 | 312 | 364 | 462 | 507 | 198.5 | 275 | 245 | 268 | 472.5 | 540 | 562.5 |
| NM_018115 | LUA#63 | 2942 | 2980 | 2750 | 3020 | 2659.5 | 2912 | 2714 | 2598 | 2775 | 2741 | 2898 | 2815 |
| NM_021974 | LUA#64 | 1949 | 1868 | 1777 | 2001 | 1535.5 | 1806 | 1739.5 | 1752 | 1837 | 1744 | 1888 | 1869.5 |
| NM_024045 | LUA#65 | 520 | 585 | 448 | 599 | 494 | 545.5 | 509 | 475 | 489 | 513.5 | 539.5 | 530 |
| NM_004079 | LUA#21 | 3630 | 3578.5 | 3061 | 3459 | 3268.5 | 3368 | 3393.5 | 3216 | 3473.5 | 3414 | 3469.5 | 3382 |
| NM_000414 | LUA#22 | 554 | 514 | 473 | 566 | 440 | 552 | 539 | 518 | 523 | 534 | 546.5 | 539 |
| NM_001684 | LUA#23 | 2436 | 2350 | 2186 | 2429 | 2206 | 2209 | 2068 | 2000 | 2280 | 2214.5 | 2479 | 2192.5 |
| NM_003879 | LUA#24 | 897 | 985 | 843.5 | 1017 | 839 | 918.5 | 909 | 959 | 942.5 | 961 | 1003 | 983 |
| NM_002166 | LUA#25 | 1616.5 | 1692 | 1460 | 1463 | 1050.5 | 1282 | 1493 | 1420 | 1532 | 1618 | 1690 | 1601 |
| NM_005952 | LUA#66 | 1343.5 | 1432.5 | 1069 | 1249 | 1130.5 | 1206.5 | 1195.5 | 1107.5 | 1166.5 | 1214 | 1263.5 | 1259 |
| NM_001034 | LUA#67 | 537 | 642 | 588 | 647 | 495.5 | 491 | 523.5 | 545 | 572 | 667 | 680 | 675.5 |
| NM_003132 | LUA#68 | 457 | 536 | 413 | 517 | 403 | 507 | 516 | 462 | 529 | 548 | 538 | 522 |
| NM_018164 | LUA#69 | 195.5 | 184 | 178 | 231 | 184 | 122 | 129 | 142.5 | 186 | 209.5 | 214 | 203 |
| NM_014573 | LUA#70 | 230 | 271 | 230 | 293 | 193.5 | 212 | 214 | 230 | 212 | 256 | 280 | 259 |
| NM_014333 | LUA#26 | 1335 | 1361 | 1221.5 | 1387 | 1155 | 1214.5 | 1230 | 1281 | 1305 | 1393 | 1462 | 1429 |
| NM_006432 | LUA#27 | 585 | 632 | 533 | 689 | 499 | 575 | 534 | 545 | 594 | 662 | 702.5 | 671.5 |
| NM_000433 | LUA#28 | 920 | 893 | 911 | 1009 | 655 | 928.5 | 927 | 928.5 | 950.5 | 969 | 1001 | 979 |
| NM_000147 | LUA#29 | 500 | 521 | 468 | 541 | 462 | 505 | 484.5 | 459.5 | 511.5 | 506 | 555 | 539 |
| NM_000584 | LUA#30 | 256 | 366 | 256 | 269 | 251 | 183 | 169.5 | 207 | 243.5 | 316 | 301.5 | 315 |
| NM_006452 | LUA#71 | 2099 | 2084 | 1892 | 2120 | 2006 | 2266 | 2093 | 1950 | 2197 | 2076.5 | 2209 | 2167 |
| NM_005915 | LUA#72 | 1226 | 1099 | 1053 | 1205.5 | 860 | 943 | 1063 | 1093.5 | 1138 | 1053 | 1123.5 | 1079 |
| NM_005980 | LUA#73 | 142 | 132 | 131 | 147 | 131 | 150.5 | 147.5 | 140 | 142.5 | 157 | 140 | 144 |
| NM_002539 | LUA#74 | 2425 | 2316 | 2087 | 2311 | 1877 | 1927.5 | 2018 | 1928 | 2145 | 2151 | 2222 | 2192 |
| NM_019058 | LUA#75 | 3031 | 2880 | 2490.5 | 2668 | 2516.5 | 2095 | 2271 | 2515 | 2626 | 2535 | 2676 | 2717 |
| NM_004152 | LUA#31 | 1242.5 | 1194 | 1192 | 1395.5 | 1060 | 1213 | 1238 | 1194.5 | 1259 | 1273 | 1272.5 | 1273 |
| NM_004602 | LUA#32 | 160 | 171 | 118 | 146 | 139 | 185.5 | 224 | 144 | 136 | 148 | 144 | 175 |
| NM_018890 | LUA#33 | 3178 | 2820 | 2759 | 3652 | 2563.5 | 2013 | 2134 | 1994.5 | 2953 | 3108 | 3381 | 3102.5 |
| NM_001101 | LUA#34 | 3390 | 3286 | 3055 | 3351 | 3058 | 2997 | 3223 | 3069 | 3164 | 3182.5 | 3260 | 3244.5 |
| NM_006019 | LUA#35 | 429 | 517 | 421 | 502 | 432 | 439 | 465 | 472 | 469 | 520 | 559 | 517.5 |
| NM_004134 | LUA#76 | 1839 | 1854.5 | 1599 | 1770 | 1402 | 1666 | 1823 | 1718 | 1844 | 1891.5 | 1836 | 1747.5 |
| NM_005008 | LUA#77 | 1088 | 1303 | 1122.5 | 1276.5 | 1020 | 1110 | 1139.5 | 1151.5 | 1213 | 1281 | 1304 | 1340 |
| NM_020117 | LUA#78 | 4107 | 3817 | 3879 | 4057 | 3458.5 | 3506 | 3738 | 3565.5 | 3943 | 3851.5 | 4059.5 | 3820.5 |
| NM_001469 | LUA#79 | 710.5 | 619 | 678 | 842 | 686 | 630 | 612 | 622.5 | 670.5 | 688 | 825 | 835 |
| NM_021203 | LUA#80 | 780 | 794 | 768 | 808.5 | 590 | 757 | 807 | 745.5 | 808 | 803 | 818 | 784.5 |
| NM_002624 | LUA#36 | 336 | 353 | 250 | 305 | 275 | 297 | 283 | 299 | 334.5 | 335 | 381 | 331 |
| NM_004759 | LUA#37 | 186 | 205.5 | 183 | 212 | 157 | 194 | 186 | 173 | 184 | 194 | 197 | 206 |
| NM_002664 | LUA#38 | 797 | 730.5 | 691 | 732 | 548 | 671 | 688 | 703 | 750 | 757 | 769 | 762 |
| NM_000211 | LUA#39 | 3211 | 2924 | 2886 | 2921 | 1857 | 2278 | 2657 | 2797 | 3053 | 2908 | 3039.5 | 2797.5 |
| NM_002468 | LUA#40 | 429 | 379.5 | 347 | 429 | 297 | 306.5 | 343 | 339 | 349 | 375 | 391 | 371.5 |
| NM_000884 | LUA#81 | 1428 | 1318 | 1197 | 1324 | 1090 | 1199 | 1217 | 1234 | 1315 | 1314.5 | 1350 | 1293 |
| NM_003752 | LUA#82 | 1846 | 1808.5 | 1603 | 1888 | 1612 | 1740.5 | 1728.5 | 1633 | 1761 | 1702 | 1825 | 1762 |
| NM_018256 | LUA#83 | 2062 | 1861 | 1845.5 | 2074.5 | 1645 | 1792 | 1851 | 1876 | 1910 | 1907 | 1960 | 1898.5 |
| NM_001948 | LUA#84 | 3418 | 3494 | 3142.5 | 3336 | 2664 | 2977 | 3066 | 3045 | 3170 | 3332 | 3464 | 3272.5 |
| NM_005566 | LUA#85 | 2977 | 2714 | 2584 | 2752 | 2214 | 2342 | 2574 | 2571 | 2654.5 | 2695 | 2715 | 2669.5 |
| NM_021103 | LUA#41 | 3189.5 | 3018 | 2718 | 3105 | 2604.5 | 2742 | 2818 | 2882 | 2966 | 2956 | 3130 | 2913.5 |
| NM_002970 | LUA#42 | 879 | 899 | 596 | 638 | 621 | 698 | 698 | 707 | 813 | 778 | 840 | 748 |
| NM_003332 | LUA#43 | 2000 | 2210 | 1489 | 1631.5 | 1664.5 | 1829 | 1865 | 1854 | 2095.5 | 2120.5 | 2375 | 2163 |
| NM_004106 | LUA#44 | 416.5 | 450.5 | 371 | 410 | 366 | 407 | 398 | 375 | 392 | 398 | 463 | 418 |
| NM_002982 | LUA#45 | 4022 | 4124 | 3811.5 | 4093 | 3650 | 3735 | 3781.5 | 3873 | 4035 | 4073 | 4216 | 3981.5 |
| NM_005375 | LUA#86 | 3004.5 | 2906 | 2558 | 2880 | 2360 | 2568 | 2646 | 2638.5 | 2846 | 2892 | 3039.5 | 2842 |
| NM_000250 | LUA#87 | 3741.5 | 3571 | 3474 | 3656 | 3421.5 | 3371 | 3432 | 3378 | 3509 | 3505 | 3695 | 3495 |
| NM_004526 | LUA#88 | 2058 | 2055 | 1808 | 1911 | 1680 | 1726.5 | 1825.5 | 1736 | 1909.5 | 1896.5 | 1978 | 1990 |
| NM_004741 | LUA#89 | 1108 | 1321.5 | 947.5 | 1238 | 1024 | 1083 | 1073 | 1051.5 | 1149 | 1280 | 1378.5 | 1306 |
| NM_002467 | LUA#90 | 2459.5 | 2556 | 2463.5 | 2716 | 2442.5 | 2612 | 2700 | 2639 | 2735 | 2770 | 2847 | 2864.5 |
| ACTB | LUA#91 | 3366 | 3226 | 2978 | 3292 | 2667 | 3186 | 3158 | 3128 | 3408.5 | 3183 | 3323 | 3238.5 |
| TFRC | LUA#92 | 948 | 1112 | 883 | 1059 | 758 | 1009 | 944.5 | 929 | 1063.5 | 1069 | 1197 | 1157 |
| GAPDH_5 | LUA#93 | 2063 | 2310 | 2363 | 2598 | 2157 | 2324 | 2337 | 2442 | 2468 | 2425.5 | 2655 | 2417 |
| GAPDH_M | LUA#94 | 4206 | 4269 | 4371 | 4733.5 | 4179.5 | 4071 | 4252.5 | 4207 | 4413 | 4315 | 4737 | 4324.5 |
| GAPDH_3 | LUA#95 | 4477 | 4343.5 | 4445 | 4632 | 3923 | 4014 | 4259.5 | 4169 | 4620.5 | 4371 | 4726 | 4365 |

Table 5C. Microtiter plates

| description | FlexMap ID | dmso23 | dmso24 | dmso25 | dmso26 | dmso27 | dmso28 | dmso29 | dmso30 | dmso31 | dmso32 | dmso33 | dmso34 |
|---|---|---|---|---|---|---|---|---|---|---|---|---|---|
| NM_005736 | LUA#1 | 821.5 | 761.5 | 188 | 697 | 774.5 | 787.5 | 819 | 983.5 | 981.5 | 798 | 306.5 | 708 |
| NM_000070 | LUA#2 | 594.5 | 430.5 | 145.5 | 562 | 569.5 | 544.5 | 596.5 | 671 | 648 | 486 | 165 | 548.5 |
| NM_018217 | LUA#3 | 1272 | 1058 | 376 | 1157 | 1280 | 1212 | 1311 | 1475 | 1368 | 1128 | 433 | 1123 |
| NM_004782 | LUA#4 | 1254 | 1072 | 442 | 1106 | 1257 | 1209.5 | 1279 | 1435 | 1295 | 1042 | 496.5 | 1128.5 |
| NM_014962 | LUA#5 | 1284.5 | 950 | 381 | 1032 | 1210 | 1259.5 | 1287 | 1466 | 1347 | 1046 | 405 | 1094 |
| NM_004514 | LUA#46 | 1275 | 1119 | 432 | 1216 | 1259 | 1206 | 1358 | 1503.5 | 1391.5 | 1179 | 512 | 1155.5 |
| NM_006773 | LUA#47 | 691 | 616 | 242 | 666 | 731 | 726 | 757 | 756 | 731 | 663 | 283 | 684 |
| NM_014288 | LUA#48 | 701 | 566 | 240 | 703 | 741 | 758 | 751 | 738 | 705 | 616 | 285 | 687 |
| NM_017440 | LUA#49 | 568.5 | 487 | 184.5 | 503.5 | 534 | 536.5 | 553 | 615 | 619 | 504 | 214 | 489.5 |
| NM_007331 | LUA#50 | 842 | 569.5 | 172 | 659 | 721 | 712.5 | 770.5 | 918 | 866 | 625 | 207 | 673 |
| NM_173823 | LUA#6 | 1025 | 607 | 154 | 705 | 814 | 832.5 | 938 | 1231 | 1222 | 732 | 173.5 | 845 |
| NM_000962 | LUA#7 | 352 | 211 | 64 | 253 | 284 | 279 | 369 | 400.5 | 441.5 | 264 | 78 | 293 |
| NM_003825 | LUA#8 | 381 | 251.5 | 111 | 235 | 283 | 306 | 350 | 401 | 379 | 249 | 117 | 325.5 |
| NM_016061 | LUA#9 | 745 | 481 | 166 | 546 | 662 | 645 | 728 | 788 | 830 | 574.5 | 181 | 539 |
| NM_000153 | LUA#10 | 55 | 45 | 32 | 43 | 43.5 | 43 | 54.5 | 62 | 65 | 51 | 37 | 44.5 |
| NM_006948 | LUA#51 | 81 | 46.5 | 45 | 62.5 | 59 | 53 | 70 | 75 | 73 | 70.5 | 34 | 62.5 |
| NM_004631 | LUA#52 | 856 | 460 | 151 | 651.5 | 676 | 654 | 697 | 822.5 | 826.5 | 502.5 | 148 | 646 |
| NM_002358 | LUA#53 | 656 | 440 | 130 | 575 | 518 | 562 | 675 | 706 | 732 | 536 | 162 | 547 |
| NM_013402 | LUA#54 | 1023.5 | 761 | 223 | 871 | 927 | 926.5 | 975 | 1112 | 1116 | 832 | 250 | 883 |
| NM_000875 | LUA#55 | 1365 | 1238 | 416 | 1061 | 1243 | 1287 | 1314 | 1444 | 1351 | 1231 | 477.5 | 1182.5 |
| NM_001974 | LUA#11 | 210 | 101.5 | 49.5 | 148 | 138 | 150 | 217 | 378.5 | 312.5 | 119 | 51.5 | 131.5 |
| NM_000632 | LUA#12 | 422 | 322.5 | 70 | 324.5 | 355 | 343.5 | 371 | 420 | 466 | 365 | 101.5 | 346 |
| NM_006457 | LUA#13 | 82 | 45 | 39 | 55 | 64 | 67 | 67.5 | 89 | 100 | 51 | 37 | 62 |
| NM_000698 | LUA#14 | 196 | 141 | 53 | 133 | 119 | 131 | 158 | 214 | 204 | 156 | 49.5 | 135 |
| NM_032571 | LUA#15 | 171 | 126 | 43 | 146 | 184 | 147 | 184 | 200 | 220 | 135.5 | 54 | 161 |
| NM_006138 | LUA#56 | 187.5 | 154.5 | 53.5 | 125.5 | 140 | 118.5 | 156.5 | 179 | 181 | 152 | 66 | 140.5 |
| NM_015201 | LUA#57 | 785 | 654 | 157 | 602 | 652 | 676.5 | 745 | 864 | 989.5 | 753 | 187 | 597 |
| NM_006985 | LUA#58 | 748 | 480 | 115.5 | 632.5 | 634 | 596 | 693 | 721 | 753.5 | 549.5 | 136 | 577 |
| NM_004095 | LUA#59 | 449 | 277 | 74 | 298 | 339 | 355.5 | 377.5 | 423 | 534 | 335 | 97.5 | 282 |
| NM_005914 | LUA#60 | 2065 | 1570 | 585.5 | 1976 | 2363 | 2060 | 2352 | 2412.5 | 2143 | 1828 | 640 | 1900 |
| NM_007282 | LUA#16 | 3898 | 3815 | 2119 | 3519 | 4076.5 | 4109 | 4055 | 4442 | 4132 | 3867 | 2338 | 3559 |
| NM_003644 | LUA#17 | 463 | 361 | 151 | 404.5 | 468 | 438.5 | 476 | 510 | 481 | 365 | 173.5 | 435 |
| NM_001498 | LUA#18 | 1764 | 1346.5 | 396 | 1556.5 | 1713 | 1626 | 1775 | 1908 | 1881 | 1507 | 461 | 1600.5 |
| NM_003172 | LUA#19 | 3530.5 | 3727 | 2201 | 3535 | 3813 | 3727 | 3844 | 3804 | 3566 | 3747 | 2476 | 3638.5 |
| NM_004723 | LUA#20 | 733.5 | 581 | 164.5 | 714 | 744 | 778 | 808 | 839.5 | 848 | 691.5 | 205 | 742 |
| NM_014366 | LUA#61 | 1790 | 1932 | 755 | 1697 | 1841 | 1830 | 1930 | 1971 | 1885.5 | 2015 | 854 | 1815.5 |
| NM_003581 | LUA#62 | 508 | 264 | 59 | 336.5 | 263 | 336 | 362 | 671 | 472 | 392 | 100 | 295 |
| NM_018115 | LUA#63 | 2891 | 2754 | 1010 | 2590 | 2914.5 | 2749 | 3009 | 3130 | 3163 | 2849 | 1137 | 2663.5 |
| NM_021974 | LUA#64 | 1800 | 1540 | 533 | 1673 | 1868 | 1801 | 1844 | 1955 | 1839.5 | 1621 | 613 | 1682.5 |
| NM_024045 | LUA#65 | 580.5 | 456 | 127 | 458 | 493 | 477 | 564.5 | 602 | 684.5 | 541 | 149 | 490 |
| NM_004079 | LUA#21 | 3373 | 2792 | 1173 | 3108.5 | 3361 | 3403 | 3373 | 3610.5 | 3401 | 2919 | 1179 | 3060 |
| NM_000414 | LUA#22 | 573 | 384 | 101 | 508 | 570.5 | 556 | 556 | 574.5 | 625 | 456 | 113 | 509.5 |
| NM_001684 | LUA#23 | 2316 | 2260 | 966 | 2120.5 | 2395 | 2329.5 | 2457 | 2669.5 | 2647.5 | 2428 | 1083 | 2158 |
| NM_003879 | LUA#24 | 919 | 761 | 233 | 892.5 | 963 | 916.5 | 985 | 1092 | 1063 | 893 | 283.5 | 903 |
| NM_002166 | LUA#25 | 1348 | 993 | 408 | 1513 | 1746 | 1565 | 1930 | 1844 | 1355 | 1134 | 467 | 1441.5 |
| NM_005952 | LUA#66 | 1283 | 1016.5 | 336 | 1102 | 1159 | 1200 | 1283 | 1387 | 1325 | 1101 | 353 | 1138 |
| NM_001034 | LUA#67 | 689.5 | 450 | 112 | 505 | 540 | 557 | 672 | 811.5 | 809 | 423 | 132 | 611 |
| NM_003132 | LUA#68 | 535 | 319 | 94 | 458.5 | 473 | 475 | 503 | 592 | 559 | 372 | 105 | 444 |
| NM_018164 | LUA#69 | 226 | 130 | 59 | 149 | 147 | 157 | 221.5 | 281.5 | 252 | 165 | 63 | 160.5 |
| NM_014573 | LUA#70 | 277 | 201.5 | 61.5 | 219 | 207 | 238 | 288.5 | 333 | 436 | 224 | 73 | 237 |
| NM_014333 | LUA#26 | 1363.5 | 1109 | 448 | 1216 | 1325 | 1285.5 | 1464.5 | 1547 | 1503.5 | 1192 | 521 | 1233 |
| NM_006432 | LUA#27 | 716.5 | 478 | 170.5 | 548.5 | 613 | 585 | 701 | 828 | 774.5 | 545.5 | 207.5 | 565.5 |
| NM_000433 | LUA#28 | 900 | 625 | 185 | 906 | 976.5 | 938 | 971.5 | 1056 | 926 | 710 | 226.5 | 875 |
| NM_000147 | LUA#29 | 565 | 447 | 136 | 456 | 509 | 496 | 566 | 633.5 | 674 | 499 | 160 | 509 |
| NM_000584 | LUA#30 | 332 | 167.5 | 51.5 | 194 | 216 | 250 | 440.5 | 577.5 | 679 | 232 | 64 | 266.5 |
| NM_006452 | LUA#71 | 2382 | 1862 | 572 | 1894.5 | 2078 | 2134 | 2062 | 2363 | 2464.5 | 1959 | 642 | 1927 |
| NM_005915 | LUA#72 | 1040.5 | 812 | 258 | 1015 | 1145 | 1113 | 1142 | 1191 | 1078 | 905.5 | 298 | 1096.5 |
| NM_005980 | LUA#73 | 162 | 113.5 | 46 | 145.5 | 150 | 140 | 143.5 | 142 | 143.5 | 133.5 | 65 | 134 |
| NM_002539 | LUA#74 | 2219.5 | 1712 | 716.5 | 1940 | 2176 | 2201 | 2248 | 2379 | 2236 | 1902 | 756 | 1957.5 |
| NM_019058 | LUA#75 | 2565.5 | 2239 | 883 | 2338.5 | 2445 | 2617 | 2767.5 | 2997 | 2585 | 2218.5 | 937.5 | 2571 |
| NM_004152 | LUA#31 | 1238.5 | 885 | 254.5 | 1116 | 1248 | 1222 | 1313 | 1419 | 1325.5 | 1031 | 326 | 1154.5 |
| NM_004602 | LUA#32 | 207.5 | 581 | 86 | 127.5 | 144 | 138 | 172 | 214 | 245 | 385 | 210 | 165.5 |
| NM_018890 | LUA#33 | 3131.5 | 2053.5 | 683 | 2273 | 2061 | 2264 | 3223 | 3293 | 2669 | 2625 | 1280 | 1831 |
| NM_001101 | LUA#34 | 3138 | 2898 | 1256 | 2946 | 3193.5 | 3261 | 3363 | 3638 | 3259 | 3136 | 1401 | 3093.5 |
| NM_006019 | LUA#35 | 552 | 373 | 118 | 421 | 443 | 456 | 541 | 679 | 653 | 420.5 | 131 | 419 |
| NM_004134 | LUA#76 | 1621 | 1208 | 429 | 1734 | 1827 | 1817 | 1814.5 | 1856 | 1730 | 1437 | 510.5 | 1704.5 |
| NM_005008 | LUA#77 | 1325 | 876 | 284 | 1091 | 1131.5 | 1088 | 1258 | 1464 | 1405.5 | 945 | 321 | 1145 |
| NM_020117 | LUA#78 | 3812 | 3366 | 1892.5 | 3512 | 3795 | 3850 | 3953 | 4053.5 | 3597.5 | 3343.5 | 2066 | 3664 |
| NM_001469 | LUA#79 | 816 | 489 | 131.5 | 644 | 627 | 637 | 698 | 1000 | 783 | 548 | 176 | 613.5 |
| NM_021203 | LUA#80 | 802 | 445 | 136 | 682 | 771.5 | 789.5 | 811 | 929 | 728 | 523 | 163.5 | 740.5 |
| NM_002624 | LUA#36 | 396 | 259.5 | 81 | 285 | 310 | 308.5 | 361 | 445 | 448 | 296 | 101 | 332.5 |
| NM_004759 | LUA#37 | 212 | 146 | 56 | 191.5 | 195 | 200 | 218 | 230 | 229 | 174 | 73 | 180 |
| NM_002664 | LUA#38 | 713 | 463 | 147 | 679 | 769.5 | 768 | 798 | 850 | 820 | 551 | 158 | 712 |
| NM_000211 | LUA#39 | 2300 | 1665 | 817 | 2789 | 3080.5 | 3084 | 3060 | 3115 | 2385.5 | 1682 | 880 | 2773 |
| NM_002468 | LUA#40 | 427 | 289 | 84 | 328.5 | 354 | 368 | 391.5 | 437 | 500 | 341 | 105 | 374.5 |
| NM_000884 | LUA#81 | 1285 | 948 | 318 | 1168 | 1347 | 1357 | 1357.5 | 1526.5 | 1366 | 1084 | 349 | 1198 |
| NM_003752 | LUA#82 | 1763 | 1642 | 538.5 | 1556 | 1773 | 1728.5 | 1820 | 2059 | 2038 | 1723 | 603.5 | 1651 |
| NM_018256 | LUA#83 | 1856 | 1542 | 566.5 | 1765 | 1956.5 | 2005 | 1978 | 2084.5 | 1880 | 1678 | 628 | 1815.5 |
| NM_001948 | LUA#84 | 3267 | 2654 | 1083 | 2928 | 3321 | 3331 | 3460 | 3698 | 3453.5 | 2621.5 | 1165.5 | 3034 |
| NM_005566 | LUA#85 | 2590 | 1917.5 | 682 | 2426 | 2711 | 2775 | 2726 | 2956 | 2650.5 | 2079 | 728 | 2471 |
| NM_021103 | LUA#41 | 2956 | 2604.5 | 1342 | 2649 | 2997 | 3023.5 | 3125.5 | 3151 | 2850 | 2606 | 1390 | 2874 |
| NM_002970 | LUA#42 | 879 | 470.5 | 177 | 617 | 645 | 720.5 | 854 | 935 | 819 | 484 | 175 | 626 |
| NM_003332 | LUA#43 | 2270 | 1228 | 527 | 1706 | 2044 | 2248.5 | 2272.5 | 2640 | 2476.5 | 1319.5 | 496.5 | 1835.5 |
| NM_004106 | LUA#44 | 486 | 268 | 100 | 347 | 378.5 | 379 | 441.5 | 519 | 499.5 | 309 | 101.5 | 338 |
| NM_002982 | LUA#45 | 4008 | 3520 | 1385.5 | 3498 | 3857.5 | 3867 | 3911.5 | 4376.5 | 4090 | 3402 | 1612 | 3652 |
| NM_005375 | LUA#86 | 2929 | 2138 | 780 | 2591 | 3000 | 3007 | 2930 | 3132 | 3068 | 2187 | 846.5 | 2508 |
| NM_000250 | LUA#87 | 3651 | 3489 | 1765 | 3299 | 3612 | 3693 | 3847 | 4025 | 3686.5 | 3647 | 1803 | 3408 |
| NM_004526 | LUA#88 | 1880 | 1497 | 585 | 1628 | 1882 | 1926 | 2035.5 | 2119 | 2056 | 1644 | 650 | 1709 |
| NM_004741 | LUA#89 | 1381 | 700 | 242 | 992.5 | 1014 | 1103 | 1378 | 1416 | 1429 | 794 | 253 | 998.5 |
| NM_002467 | LUA#90 | 2628 | 2183.5 | 955 | 2420 | 2720 | 2741 | 2784 | 2979 | 2694 | 2237.5 | 1032 | 2422 |
| ACTB | LUA#91 | 3135 | 2568 | 1118.5 | 3053.5 | 3423.5 | 3204 | 3422 | 3556 | 3070 | 2801.5 | 1285 | 3053 |
| TFRC | LUA#92 | 1174 | 664 | 207 | 912 | 1113 | 1032 | 1189 | 1370 | 1388 | 813 | 235 | 1034 |
| GAPDH_5 | LUA#93 | 2447 | 2014.5 | 859 | 2239.5 | 2438 | 2261 | 2400 | 2572 | 2390.5 | 2139 | 1023 | 2433 |
| GAPDH_M | LUA#94 | 4314 | 4528 | 2358.5 | 4048 | 4414 | 4150 | 4464 | 4643 | 4474 | 4483.5 | 2639.5 | 4111 |
| GAPDH_3 | LUA#95 | 4468 | 4283 | 3479 | 3998 | 4500 | 4518 | 4621 | 4645.5 | 4414 | 4249 | 3411 | 4026 |

Table 5D. Microtiter plates

| description | FlexMap ID | dmso35 | dmso36 | dmso37 | dmso38 | dmso39 | dmso40 | dmso41 | dmso42 | dmso43 | dmso44 | dmso45 | dmso46 |
|---|---|---|---|---|---|---|---|---|---|---|---|---|---|
| NM_005736 | LUA#1 | 800 | 833 | 740.5 | 838.5 | 652 | 751.5 | 746 | 714.5 | 87 | 136 | 806 | 835 |
| NM_000070 | LUA#2 | 605.5 | 588.5 | 578.5 | 652.5 | 538 | 377.5 | 350 | 518 | 67 | 62.5 | 534.5 | 556 |
| NM_018217 | LUA#3 | 1308.5 | 1279.5 | 1242 | 1243.5 | 1030 | 901 | 915 | 1109.5 | 89 | 167 | 1158.5 | 1114 |
| NM_004782 | LUA#4 | 1238 | 1228 | 1232 | 1133 | 974 | 882.5 | 885.5 | 1085 | 96 | 187.5 | 1077.5 | 1018 |
| NM_014962 | LUA#5 | 1158 | 1208 | 1175 | 1187 | 1015 | 823 | 819 | 1036 | 87 | 161 | 1104 | 1084 |
| NM_004514 | LUA#46 | 1237 | 1258 | 1186 | 1216 | 1067.5 | 909 | 946 | 1072 | 88 | 178 | 1161 | 1144 |
| NM_006773 | LUA#47 | 769 | 741 | 699.5 | 701.5 | 619 | 544 | 528 | 685.5 | 88 | 128 | 653 | 647 |
| NM_014288 | LUA#48 | 733 | 721.5 | 667 | 692 | 609.5 | 478 | 510 | 676 | 132 | 140.5 | 643.5 | 702 |
| NM_017440 | LUA#49 | 582 | 547 | 529 | 527 | 462 | 423 | 387 | 490 | 73 | 97.5 | 501 | 562 |
| NM_007331 | LUA#50 | 744 | 743 | 749.5 | 756 | 670.5 | 465 | 464 | 657 | 66 | 92 | 679 | 711.5 |
| NM_173823 | LUA#6 | 761 | 855 | 839 | 836 | 801 | 520.5 | 491.5 | 695 | 47 | 64 | 894 | 880 |
| NM_000962 | LUA#7 | 309 | 343.5 | 293 | 332 | 297 | 178 | 186 | 245 | 44 | 30 | 295 | 316.5 |
| NM_003825 | LUA#8 | 286 | 240 | 257 | 299 | 278.5 | 182 | 215.5 | 256 | 62 | 72 | 306 | 331 |
| NM_016061 | LUA#9 | 586 | 658.5 | 613 | 659 | 536 | 369 | 398.5 | 507 | 66 | 83.5 | 557.5 | 606 |
| NM_000153 | LUA#10 | 47.5 | 56 | 50 | 57 | 56 | 46 | 40.5 | 41 | 29 | 28 | 54 | 64 |
| NM_006948 | LUA#51 | 62 | 61.5 | 69.5 | 67 | 67 | 56 | 51 | 51 | 29 | 15 | 57.5 | 71 |
| NM_004631 | LUA#52 | 643.5 | 646 | 686.5 | 663 | 591 | 328 | 336 | 545 | 80 | 92.5 | 615 | 650 |
| NM_002358 | LUA#53 | 573.5 | 540 | 564 | 592 | 580 | 419 | 385 | 538 | 36.5 | 49 | 559 | 606.5 |
| NM_013402 | LUA#54 | 966 | 978 | 921 | 940.5 | 856.5 | 563.5 | 599 | 823 | 46 | 95 | 875 | 880 |
| NM_000875 | LUA#55 | 1285.5 | 1133 | 1138 | 1263 | 1075 | 1092 | 1048 | 1124 | 106 | 188.5 | 1221 | 1110 |
| NM_001974 | LUA#11 | 138 | 141 | 155.5 | 212 | 134 | 83 | 93 | 119 | 36 | 35 | 137 | 207 |
| NM_000632 | LUA#12 | 387 | 363 | 342 | 400 | 356 | 348 | 296 | 342 | 47.5 | 53 | 353 | 359.5 |
| NM_006457 | LUA#13 | 62 | 71.5 | 61 | 81 | 78.5 | 55 | 49 | 48.5 | 28 | 23 | 75 | 63 |
| NM_000698 | LUA#14 | 146 | 141 | 122 | 167 | 160.5 | 135 | 125.5 | 121.5 | 40 | 33.5 | 148 | 200 |
| NM_032571 | LUA#15 | 164 | 176.5 | 167.5 | 169 | 138 | 110 | 109 | 129.5 | 28 | 33 | 160 | 172 |
| NM_006138 | LUA#56 | 166.5 | 146 | 121 | 158.5 | 152 | 141.5 | 125 | 125 | 39 | 46 | 135 | 151 |
| NM_015201 | LUA#57 | 686.5 | 706 | 631.5 | 729 | 656 | 569 | 485 | 618 | 42 | 74 | 666 | 624 |
| NM_006985 | LUA#58 | 638 | 615 | 623 | 582 | 538 | 345 | 345 | 555 | 37 | 54 | 584 | 588 |
| NM_004095 | LUA#59 | 304 | 350 | 338.5 | 374 | 354 | 235 | 211 | 281 | 38 | 46 | 316 | 357 |
| NM_005914 | LUA#60 | 2448.5 | 2103 | 2338 | 1967.5 | 1571 | 1343 | 1339 | 2015 | 118 | 230 | 1893 | 1677 |
| NM_007282 | LUA#16 | 3893.5 | 3874.5 | 3637.5 | 3391 | 3071 | 3437 | 3494 | 3535 | 359 | 966 | 3373 | 3307 |
| NM_003644 | LUA#17 | 446 | 449.5 | 439 | 424 | 365.5 | 311 | 308.5 | 406 | 53 | 79 | 411 | 413 |
| NM_001498 | LUA#18 | 1703 | 1725 | 1714 | 1637 | 1450 | 1059 | 1094 | 1550 | 69 | 141 | 1618 | 1541 |
| NM_003172 | LUA#19 | 3764 | 3726.5 | 3602 | 3543 | 2943 | 3368 | 3715.5 | 3362 | 481 | 1067 | 3343 | 3330 |
| NM_004723 | LUA#20 | 813.5 | 783 | 707 | 724 | 636 | 453 | 457 | 671.5 | 41 | 77 | 685 | 708 |
| NM_014366 | LUA#61 | 1911 | 1754 | 1710 | 1737.5 | 1493 | 1770 | 1731 | 1733 | 126 | 331.5 | 1643 | 1713 |
| NM_003581 | LUA#62 | 257.5 | 265 | 351 | 494.5 | 436 | 221.5 | 299 | 340 | 41 | 42 | 405 | 615.5 |
| NM_018115 | LUA#63 | 2928.5 | 2916 | 2747 | 2814 | 2454.5 | 2212 | 2288.5 | 2701 | 162.5 | 384 | 2539.5 | 2803 |
| NM_021974 | LUA#64 | 1901 | 1937 | 1813 | 1847 | 1596 | 1284.5 | 1344 | 1669 | 113 | 212.5 | 1647.5 | 1703 |
| NM_024045 | LUA#65 | 479 | 524 | 444 | 568 | 465 | 367 | 340.5 | 409.5 | 40.5 | 56 | 475 | 450 |
| NM_004079 | LUA#21 | 3238 | 3361 | 3198 | 3133 | 2881 | 2391 | 2398 | 2889 | 181 | 455 | 3041.5 | 2926 |
| NM_000414 | LUA#22 | 568 | 553 | 535 | 523 | 514 | 336 | 297 | 495 | 40.5 | 46 | 491.5 | 489 |
| NM_001684 | LUA#23 | 2275 | 2323.5 | 2187 | 2171 | 1887.5 | 1959.5 | 1968 | 2061 | 177.5 | 450.5 | 2037.5 | 2024 |
| NM_003879 | LUA#24 | 986.5 | 968.5 | 943.5 | 939 | 784 | 585 | 663 | 892 | 49 | 93 | 827 | 895.5 |
| NM_002166 | LUA#25 | 1657 | 1602 | 1549 | 1381 | 965.5 | 726 | 980.5 | 1589 | 75 | 166 | 1280 | 1227.5 |
| NM_005952 | LUA#66 | 1260 | 1233.5 | 1097 | 1147 | 991 | 801.5 | 831 | 1068 | 58 | 116.5 | 1122.5 | 1144 |
| NM_001034 | LUA#67 | 626 | 529 | 574 | 618 | 505 | 295 | 404 | 608.5 | 45 | 58 | 619 | 704 |
| NM_003132 | LUA#68 | 482 | 469 | 474.5 | 473 | 389 | 253.5 | 259.5 | 423 | 46.5 | 46 | 408 | 387 |
| NM_018164 | LUA#69 | 170 | 161 | 167 | 261 | 164 | 111.5 | 136 | 151 | 45 | 39 | 201 | 256 |
| NM_014573 | LUA#70 | 267.5 | 271 | 267 | 275.5 | 244 | 160 | 170 | 247 | 32 | 43.5 | 243 | 207 |
| NM_014333 | LUA#26 | 1364.5 | 1335.5 | 1312 | 1377.5 | 1155 | 983 | 962.5 | 1199.5 | 104 | 191 | 1229.5 | 1285 |
| NM_006432 | LUA#27 | 642 | 639 | 657 | 680.5 | 551 | 408 | 416 | 547 | 59 | 84 | 598 | 584.5 |
| NM_000433 | LUA#28 | 1066 | 942 | 920 | 924 | 750 | 515 | 567.5 | 860 | 47 | 80 | 841 | 797 |
| NM_000147 | LUA#29 | 529 | 555.5 | 529 | 517 | 462.5 | 387 | 380 | 487.5 | 45 | 65 | 533 | 539 |
| NM_000584 | LUA#30 | 242 | 278 | 258 | 423 | 221 | 124 | 161 | 199.5 | 40 | 40 | 257.5 | 294 |
| NM_006452 | LUA#71 | 2013 | 2089 | 2061 | 1989 | 1848 | 1611.5 | 1467.5 | 1781 | 101 | 221.5 | 1882 | 1980.5 |
| NM_005915 | LUA#72 | 1188 | 1179 | 1051.5 | 1098 | 845 | 584.5 | 691 | 948 | 59 | 101 | 1030.5 | 1063 |
| NM_005980 | LUA#73 | 153 | 160 | 142 | 150.5 | 126.5 | 112 | 104 | 137 | 38 | 32 | 140.5 | 128 |
| NM_002539 | LUA#74 | 2191 | 2195 | 2121 | 2170 | 1808.5 | 1433 | 1564.5 | 1898 | 118 | 287.5 | 2001.5 | 1947 |
| NM_019058 | LUA#75 | 2782 | 2271.5 | 2318 | 2538.5 | 2171 | 1860 | 2008 | 2257 | 134 | 341 | 2417 | 2269 |
| NM_004152 | LUA#31 | 1261 | 1241 | 1153 | 1334 | 991 | 696 | 753 | 1089 | 54 | 100 | 1113 | 1186 |
| NM_004602 | LUA#32 | 265.5 | 213 | 162 | 220 | 199.5 | 625 | 660 | 131 | 93 | 90 | 275 | 257.5 |
| NM_018890 | LUA#33 | 2075 | 2485 | 2508.5 | 3390.5 | 1910 | 2162 | 2101 | 2009.5 | 165.5 | 458 | 2526 | 2784.5 |
| NM_001101 | LUA#34 | 3429.5 | 3266.5 | 3048 | 3076 | 2572 | 2528 | 2638 | 3090 | 204 | 538.5 | 2953 | 2968 |
| NM_006019 | LUA#35 | 466 | 541 | 465 | 530.5 | 460 | 286 | 331 | 389.5 | 40.5 | 51 | 471 | 498 |
| NM_004134 | LUA#76 | 1792.5 | 1804.5 | 1765 | 1703.5 | 1324 | 1003 | 1120 | 1633.5 | 80 | 164 | 1503.5 | 1611.5 |
| NM_005008 | LUA#77 | 1191 | 1314 | 1203 | 1286 | 965.5 | 702 | 700 | 1069 | 68 | 121 | 1117 | 1163 |
| NM_020117 | LUA#78 | 3834 | 3886 | 3716.5 | 3752.5 | 3058 | 2885 | 3210 | 3558 | 317 | 821.5 | 3341 | 3770 |
| NM_001469 | LUA#79 | 681 | 598.5 | 718.5 | 792 | 616 | 403 | 431 | 600 | 49 | 70 | 579.5 | 749 |
| NM_021203 | LUA#80 | 807 | 744 | 750 | 772 | 661 | 371 | 410 | 686 | 49 | 69 | 730.5 | 642.5 |
| NM_002624 | LUA#36 | 278 | 328.5 | 338 | 388 | 292 | 234 | 213.5 | 274.5 | 34 | 48.5 | 323.5 | 411 |
| NM_004759 | LUA#37 | 193 | 202 | 188 | 206 | 158 | 134 | 138 | 184.5 | 38 | 42 | 180 | 185 |
| NM_002664 | LUA#38 | 714 | 737 | 750 | 734 | 590 | 385.5 | 418 | 645.5 | 40 | 64.5 | 656 | 700.5 |
| NM_000211 | LUA#39 | 3006 | 2869 | 2683 | 2721 | 1875 | 1271 | 1745 | 2569 | 132 | 322.5 | 2468.5 | 2468 |
| NM_002468 | LUA#40 | 379 | 415 | 338.5 | 380 | 305 | 294 | 281.5 | 294 | 45 | 51.5 | 378 | 353.5 |
| NM_000884 | LUA#81 | 1287 | 1282 | 1226 | 1286.5 | 1040 | 779.5 | 865 | 1131.5 | 70 | 124 | 1225 | 1136 |
| NM_003752 | LUA#82 | 1821.5 | 1763 | 1615.5 | 1734 | 1487 | 1332.5 | 1338 | 1538 | 91.5 | 208.5 | 1630 | 1496 |
| NM_018256 | LUA#83 | 2020.5 | 1982 | 1850 | 1812.5 | 1542.5 | 1282 | 1341.5 | 1768 | 89 | 218.5 | 1730.5 | 1731 |
| NM_001948 | LUA#84 | 3271 | 3345 | 3206 | 3253 | 2653 | 2216.5 | 2299 | 2996 | 214 | 499 | 3014.5 | 2966 |
| NM_005566 | LUA#85 | 2684 | 2614.5 | 2520 | 2485 | 2077 | 1560 | 1596 | 2313 | 109 | 268 | 2317 | 2122.5 |
| NM_021103 | LUA#41 | 2997 | 2869 | 2675 | 2722 | 2340 | 2323.5 | 2451 | 2529 | 321 | 666 | 2658 | 2720.5 |
| NM_002970 | LUA#42 | 648 | 665.5 | 668 | 715 | 587.5 | 354 | 371 | 529 | 72 | 91 | 654.5 | 656 |
| NM_003332 | LUA#43 | 1942.5 | 2132 | 2127 | 2213 | 1879 | 953.5 | 987 | 1653.5 | 218.5 | 335 | 1969 | 1813 |
| NM_004106 | LUA#44 | 375 | 377.5 | 366 | 397.5 | 359 | 217.5 | 210 | 330 | 47 | 55 | 348 | 355 |
| NM_002982 | LUA#45 | 3896.5 | 3808 | 3711 | 3649 | 3206 | 3020 | 3081 | 3635 | 273 | 694 | 3579 | 3162 |
| NM_005375 | LUA#86 | 2763 | 2813 | 2692 | 2661.5 | 2436 | 1824 | 1784.5 | 2537 | 157 | 354 | 2526.5 | 2672 |
| NM_000250 | LUA#87 | 3517 | 3557 | 3405 | 3467 | 3013 | 3199.5 | 3142 | 3251 | 299 | 711 | 3253 | 3188.5 |
| NM_004526 | LUA#88 | 1885 | 1915 | 1804.5 | 1852 | 1579.5 | 1229 | 1326.5 | 1636 | 115 | 248 | 1701 | 1706.5 |
| NM_004741 | LUA#89 | 1002 | 1136 | 1118 | 1351 | 929.5 | 501 | 537.5 | 825 | 90 | 128 | 977.5 | 1228.5 |
| NM_002467 | LUA#90 | 2713 | 2738 | 2634 | 2516 | 2218 | 1932 | 1877 | 2262 | 270 | 462 | 2574 | 2413 |
| ACTB | LUA#91 | 3240 | 3312 | 3154 | 3122.5 | 2542.5 | 2334 | 2382.5 | 2809.5 | 185 | 452 | 2830 | 2846.5 |
| TFRC | LUA#92 | 1052 | 1166 | 1040 | 1153 | 979.5 | 598 | 566.5 | 952 | 71 | 108.5 | 1087 | 990 |
| GAPDH_5 | LUA#93 | 2458 | 2471 | 2312 | 2286 | 1881 | 1785.5 | 1872 | 2132 | 141.5 | 332 | 2197 | 1991.5 |
| GAPDH_M | LUA#94 | 4477.5 | 4376 | 3992.5 | 4130 | 3535.5 | 4220 | 4298 | 3887.5 | 405 | 961.5 | 3835 | 3521 |
| GAPDH_3 | LUA#95 | 4410 | 4411 | 4111.5 | 4179 | 3477.5 | 4067 | 4018.5 | 3937 | 1107 | 2164 | 3853 | 3345 |

Table 5E. Microtiter plates

| description | FlexMap ID | dmso47 | tretinoin1 | tretinoin2 | tretinoin3 | tretinoin4 | tretinoin5 | tretinoin6 | tretinoin7 | tretinoin8 | tretinoin9 |
|---|---|---|---|---|---|---|---|---|---|---|---|
| NM_005736 | LUA#1 | 712.5 | 1007 | 600 | 745 | 120 | 784.5 | 969 | 868 | 403 | 1056 |
| NM_000070 | LUA#2 | 542 | 645 | 609.5 | 617.5 | 257 | 804.5 | 748 | 679 | 244 | 752 |
| NM_018217 | LUA#3 | 972 | 1449 | 1280.5 | 1420 | 201 | 1539.5 | 1583 | 1510 | 682.5 | 1494.5 |
| NM_004782 | LUA#4 | 880.5 | 1159.5 | 1019.5 | 1093 | 191.5 | 1254 | 1263 | 1211.5 | 610.5 | 1219.5 |
| NM_014962 | LUA#5 | 1037 | 1464 | 1254 | 1316.5 | 176 | 1544 | 1556 | 1381 | 600 | 1344 |
| NM_004514 | LUA#46 | 941 | 1137 | 1091 | 1095 | 124 | 1305 | 1280 | 1218 | 659 | 1230.5 |
| NM_006773 | LUA#47 | 518 | 891 | 958 | 980.5 | 376 | 1067 | 994 | 1039 | 537 | 1062.5 |
| NM_014288 | LUA#48 | 524 | 640 | 712 | 763.5 | 370 | 801 | 816 | 737 | 395.5 | 809 |
| NM_017440 | LUA#49 | 461 | 544 | 516 | 515 | 226.5 | 586 | 612.5 | 615 | 298 | 622 |
| NM_007331 | LUA#50 | 638.5 | 912 | 911 | 865 | 183 | 1163.5 | 1068 | 987 | 369 | 958 |
| NM_173823 | LUA#6 | 960 | 1186.5 | 1029 | 1067 | 66 | 1345 | 1453 | 1179 | 381.5 | 1145.5 |
| NM_000962 | LUA#7 | 353 | 753 | 749 | 829.5 | 56 | 863 | 827.5 | 775 | 259 | 910.5 |
| NM_003825 | LUA#8 | 399 | 472 | 311 | 338 | 90 | 452 | 463 | 392 | 149 | 374 |
| NM_016061 | LUA#9 | 615 | 1280 | 1287 | 1337 | 110 | 1411 | 1519 | 1429 | 611 | 1267 |
| NM_000153 | LUA#10 | 119 | 141 | 148 | 144 | 44 | 160 | 184 | 146 | 57 | 152 |
| NM_006948 | LUA#51 | 75 | 75.5 | 64.5 | 65.5 | 37.5 | 75 | 66 | 94 | 47 | 67.5 |
| NM_004631 | LUA#52 | 651 | 893.5 | 845 | 865 | 133 | 1055 | 1218 | 998.5 | 283 | 808 |
| NM_002358 | LUA#53 | 498 | 418.5 | 426 | 405 | 34 | 477 | 491 | 522 | 210.5 | 523 |
| NM_013402 | LUA#54 | 789 | 1188.5 | 1164 | 1216 | 51 | 1393.5 | 1428 | 1345 | 506 | 1246 |
| NM_000875 | LUA#55 | 958 | 1248 | 1018.5 | 1094.5 | 75 | 1151.5 | 1201 | 1151.5 | 672 | 1198 |
| NM_001974 | LUA#11 | 198 | 826 | 132 | 221 | 30 | 240 | 313 | 382 | 72 | 590 |
| NM_000632 | LUA#12 | 363 | 485.5 | 406 | 446.5 | 45.5 | 519 | 580 | 537 | 172 | 496.5 |
| NM_006457 | LUA#13 | 135 | 83 | 79 | 67 | 36 | 91 | 109.5 | 88.5 | 38 | 81 |
| NM_000698 | LUA#14 | 220 | 252 | 202 | 222 | 47 | 259 | 284.5 | 292 | 92 | 236 |
| NM_032571 | LUA#15 | 191 | 193 | 192 | 197 | 53 | 236 | 253 | 217 | 71 | 239 |
| NM_006138 | LUA#56 | 210 | 557 | 420 | 445 | 45 | 467.5 | 500 | 464 | 203 | 494 |
| NM_015201 | LUA#57 | 705.5 | 1456 | 1263 | 1605 | 73 | 1699.5 | 1741 | 1620 | 797 | 1647.5 |
| NM_006985 | LUA#58 | 486.5 | 1364 | 1663 | 1539 | 48 | 1704.5 | 1609 | 1657 | 540 | 1438.5 |
| NM_004095 | LUA#59 | 376 | 714 | 733 | 799.5 | 43 | 891 | 900 | 902 | 277 | 764 |
| NM_005914 | LUA#60 | 1384 | 1942 | 1765.5 | 1972 | 209 | 2367 | 2086.5 | 2213 | 1002 | 1994 |
| NM_007282 | LUA#16 | 2507 | 3727.5 | 3308 | 3659.5 | 148 | 4025.5 | 3945 | 3663.5 | 2556 | 3643 |
| NM_003644 | LUA#17 | 374.5 | 374 | 336 | 376 | 136.5 | 402.5 | 436 | 387.5 | 203 | 400 |
| NM_001498 | LUA#18 | 1440.5 | 1427 | 1476.5 | 1522.5 | 89 | 1721 | 1766 | 1670 | 620 | 1578 |
| NM_003172 | LUA#19 | 2385.5 | 3240 | 3377 | 3457 | 142 | 3452 | 3345.5 | 3194.5 | 2743 | 3711 |
| NM_004723 | LUA#20 | 588 | 977 | 863 | 1030.5 | 44 | 1074 | 1047 | 982 | 435 | 1148 |
| NM_014366 | LUA#61 | 1280.5 | 1716 | 1736 | 1892 | 51.5 | 1915 | 1973 | 1899 | 1422.5 | 1937.5 |
| NM_003581 | LUA#62 | 345 | 742 | 360 | 455 | 48 | 551 | 988 | 918.5 | 186 | 626.5 |
| NM_018115 | LUA#63 | 2140 | 3715 | 3778.5 | 3863 | 104 | 3963 | 3999.5 | 3870.5 | 2808.5 | 3954 |
| NM_021974 | LUA#64 | 1382 | 2119 | 2344.5 | 2289 | 107.5 | 2544 | 2617.5 | 2309 | 1258 | 2411.5 |
| NM_024045 | LUA#65 | 484 | 771 | 761 | 793 | 47 | 917 | 904 | 960.5 | 346 | 825 |
| NM_004079 | LUA#21 | 2374.5 | 3579.5 | 3604 | 3848 | 137.5 | 4150 | 4022 | 3854 | 1810 | 3690.5 |
| NM_000414 | LUA#22 | 504.5 | 669 | 806.5 | 897 | 37 | 930.5 | 889.5 | 848 | 319 | 954 |
| NM_001684 | LUA#23 | 1613 | 3259 | 2761.5 | 3205 | 115 | 3451 | 3522 | 3269 | 2585 | 3440 |
| NM_003879 | LUA#24 | 707 | 1579.5 | 1854 | 1864 | 56 | 2010 | 2086 | 1929 | 996 | 1963 |
| NM_002166 | LUA#25 | 838 | 2678.5 | 2699 | 3180 | 82 | 2976 | 2983 | 2559 | 1905 | 3511.5 |
| NM_005952 | LUA#66 | 943 | 957 | 924 | 940 | 58 | 976 | 1108 | 1027.5 | 375.5 | 941 |
| NM_001034 | LUA#67 | 554 | 891 | 421 | 558 | 64.5 | 662 | 644 | 688 | 186.5 | 824 |
| NM_003132 | LUA#68 | 411.5 | 374 | 402.5 | 388 | 53.5 | 506 | 493 | 404 | 97.5 | 371 |
| NM_018164 | LUA#69 | 207 | 258 | 161 | 205 | 45 | 239 | 301 | 343 | 88.5 | 244 |
| NM_014573 | LUA#70 | 283 | 446.5 | 172 | 196 | 52 | 244 | 306 | 260 | 86 | 369 |
| NM_014333 | LUA#26 | 1010.5 | 1288 | 1167 | 1274.5 | 249 | 1456.5 | 1539 | 1513 | 672.5 | 1394.5 |
| NM_006432 | LUA#27 | 528 | 663 | 465 | 539 | 116.5 | 748.5 | 749 | 805 | 261 | 687 |
| NM_000433 | LUA#28 | 594 | 353 | 431.5 | 443.5 | 37 | 540 | 453.5 | 429 | 158 | 476.5 |
| NM_000147 | LUA#29 | 472 | 271 | 214 | 261 | 49 | 313 | 299 | 265 | 94 | 297.5 |
| NM_000584 | LUA#30 | 380 | 1027 | 270 | 453 | 50 | 411 | 600 | 505.5 | 116 | 723 |
| NM_006452 | LUA#71 | 1689 | 1715 | 1680 | 1742.5 | 83.5 | 2089 | 2031 | 1986 | 764 | 1717 |
| NM_005915 | LUA#72 | 768 | 481 | 443 | 497.5 | 50 | 517 | 560 | 541 | 173 | 543 |
| NM_005980 | LUA#73 | 147 | 106 | 90 | 96 | 46 | 92.5 | 99 | 112 | 47 | 90 |
| NM_002539 | LUA#74 | 1486 | 794.5 | 825 | 806.5 | 62.5 | 906 | 907 | 906 | 341 | 879 |
| NM_019058 | LUA#75 | 1861 | 2700.5 | 2316 | 2808 | 62.5 | 2607 | 2827 | 2598 | 1106 | 2635 |
| NM_004152 | LUA#31 | 844 | 613.5 | 664 | 635.5 | 50 | 804.5 | 811 | 920 | 225 | 661 |
| NM_004602 | LUA#32 | 439 | 967.5 | 114 | 307 | 49 | 178.5 | 275.5 | 231 | 315 | 364 |
| NM_018890 | LUA#33 | 1456 | 3289.5 | 2445.5 | 3142 | 144 | 3827.5 | 3781 | 4048.5 | 1671 | 3276.5 |
| NM_001101 | LUA#34 | 2148 | 2044 | 2141 | 2169 | 72 | 2194 | 2152 | 2208 | 1143.5 | 2249.5 |
| NM_006019 | LUA#35 | 475.5 | 431 | 378 | 402 | 61 | 452 | 533 | 447 | 108 | 403 |
| NM_004134 | LUA#76 | 1078 | 1012 | 1176 | 1010.5 | 67 | 1224.5 | 1261.5 | 1071.5 | 361 | 1060 |
| NM_005008 | LUA#77 | 895 | 1088 | 865 | 951 | 60 | 1096.5 | 1252.5 | 1117.5 | 281 | 1095 |
| NM_020117 | LUA#78 | 2483 | 2041 | 2196.5 | 2274 | 75.5 | 2432 | 2308 | 2248 | 1261 | 2246 |
| NM_001469 | LUA#79 | 496.5 | 850 | 405 | 467.5 | 47 | 592.5 | 796 | 833 | 164 | 637 |
| NM_021203 | LUA#80 | 559 | 396.5 | 401 | 428 | 50 | 487 | 504 | 437.5 | 92 | 406 |
| NM_002624 | LUA#36 | 354.5 | 378 | 268 | 348 | 52 | 451.5 | 542.5 | 417 | 124 | 342.5 |
| NM_004759 | LUA#37 | 183.5 | 130 | 145 | 140 | 49 | 150.5 | 165 | 169 | 45.5 | 133 |
| NM_002664 | LUA#38 | 573 | 797 | 806 | 872 | 56.5 | 987 | 938.5 | 870 | 293 | 950.5 |
| NM_000211 | LUA#39 | 1417 | 1438.5 | 1493 | 1537 | 64 | 1639 | 1647 | 1273.5 | 446 | 1558 |
| NM_002468 | LUA#40 | 370 | 463 | 273 | 333 | 55 | 355 | 441 | 352.5 | 117.5 | 387 |
| NM_000884 | LUA#81 | 967 | 907 | 802.5 | 882 | 79 | 1048.5 | 1027.5 | 931 | 331.5 | 962 |
| NM_003752 | LUA#82 | 1327 | 1015 | 949 | 1033 | 56 | 1119 | 1129.5 | 1088 | 467 | 1103 |
| NM_018256 | LUA#83 | 1250.5 | 951 | 1138 | 1055 | 66.5 | 1193 | 1204.5 | 1181 | 447.5 | 1205 |
| NM_001948 | LUA#84 | 2244.5 | 2685 | 2401.5 | 2620 | 110.5 | 2687 | 2842.5 | 2584 | 1071 | 2714 |
| NM_005566 | LUA#85 | 1709 | 1526.5 | 1628 | 1860.5 | 67.5 | 1881 | 1746.5 | 1895 | 630 | 1630 |
| NM_021103 | LUA#41 | 1926 | 2244.5 | 2051 | 2229 | 111.5 | 2407 | 2540 | 2141 | 1271 | 2148 |
| NM_002970 | LUA#42 | 624 | 821 | 659.5 | 791 | 125 | 970.5 | 975 | 816 | 233 | 695 |
| NM_003332 | LUA#43 | 1965 | 1940.5 | 1658 | 1865 | 312 | 2451 | 2442.5 | 2074 | 594 | 1769 |
| NM_004106 | LUA#44 | 347.5 | 348.5 | 281 | 335 | 53 | 351 | 392 | 399 | 96 | 302 |
| NM_002982 | LUA#45 | 2713 | 3642 | 2895 | 3096 | 129.5 | 3463.5 | 3752 | 3173 | 1511 | 3062 |
| NM_005375 | LUA#86 | 2159 | 2531 | 2256 | 2421 | 167.5 | 2822 | 2752 | 2748 | 1102 | 2492.5 |
| NM_000250 | LUA#87 | 2546.5 | 2107 | 2130.5 | 2120 | 88 | 2263 | 2364 | 2168 | 1006 | 2120 |
| NM_004526 | LUA#88 | 1386 | 1245 | 1195 | 1263 | 84 | 1418 | 1400.5 | 1287 | 493.5 | 1316.5 |
| NM_004741 | LUA#89 | 933 | 1599 | 1127.5 | 1113.5 | 153 | 1546 | 1679 | 1620 | 315.5 | 1160 |
| NM_002467 | LUA#90 | 1956 | 1673 | 1851 | 1710.5 | 295 | 2200 | 2298 | 1831 | 739.5 | 1677 |
| ACTB | LUA#91 | 2149.5 | 2840.5 | 3108 | 3160.5 | 93 | 3297 | 3543 | 3123 | 1706 | 3268 |
| TFRC | LUA#92 | 1049 | 775 | 707.5 | 768 | 73 | 1002.5 | 1062 | 878.5 | 259 | 904 |
| GAPDH_5 | LUA#93 | 1561.5 | 2061 | 2169.5 | 2073 | 80 | 2401 | 2387 | 2222 | 1175 | 2449 |
| GAPDH_M | LUA#94 | 2911.5 | 3948 | 3761 | 3945 | 135 | 4111 | 4218 | 3809 | 2710.5 | 4026 |
| GAPDH_3 | LUA#95 | 2910 | 4091 | 3621 | 4239.5 | 277 | 4336 | 4378.5 | 3889 | 3607 | 4420.5 |

Table 5F. Microtiter plates

| description | FlexMap ID | tretinoin10 | tretinoin11 | tretinoin12 | tretinoin13 | tretinoin14 | tretinoin15 | tretinoin16 | tretinoin17 | tretinoin18 | tretinoin19 |
|---|---|---|---|---|---|---|---|---|---|---|---|
| NM_005736 | LUA#1 | 645 | 651.5 | 674.5 | 735.5 | 698 | 796 | 882 | 791 | 689 | 699 |
| NM_000070 | LUA#2 | 664 | 625 | 565 | 704 | 699 | 728.5 | 711.5 | 723.5 | 700 | 635 |
| NM_018217 | LUA#3 | 1364 | 1259 | 1292.5 | 1313 | 1384.5 | 1423 | 1521.5 | 1476 | 1348 | 1316 |
| NM_004782 | LUA#4 | 1107 | 1102 | 1041 | 1177.5 | 1148 | 1130 | 1235 | 1243 | 1214 | 1168 |
| NM_014962 | LUA#5 | 1169 | 1120 | 1197 | 1283 | 1243 | 1247.5 | 1277.5 | 1244 | 1215.5 | 1154 |
| NM_004514 | LUA#46 | 1104.5 | 1065.5 | 1095 | 1188.5 | 1228 | 1147 | 1236 | 1267 | 1212 | 1126.5 |
| NM_006773 | LUA#47 | 1012 | 1004.5 | 946 | 1062 | 1037 | 1097 | 1217.5 | 1141 | 1139.5 | 1101.5 |
| NM_014288 | LUA#48 | 777.5 | 770 | 765 | 771 | 805.5 | 793 | 896.5 | 895 | 861 | 801.5 |
| NM_017440 | LUA#49 | 557 | 520 | 523 | 557 | 591 | 626.5 | 692 | 633 | 588 | 568 |
| NM_007331 | LUA#50 | 881 | 812 | 753 | 849 | 919 | 879 | 897 | 978 | 854.5 | 837.5 |
| NM_173823 | LUA#6 | 963 | 952 | 995.5 | 1024.5 | 1050 | 1081 | 1034 | 1040 | 1026 | 946 |
| NM_000962 | LUA#7 | 762 | 738.5 | 738.5 | 864 | 902 | 752.5 | 845 | 860 | 784 | 779 |
| NM_003825 | LUA#8 | 299 | 334 | 341.5 | 358 | 252 | 310 | 327.5 | 324 | 309 | 274.5 |
| NM_016061 | LUA#9 | 1213 | 1145.5 | 1169.5 | 1280 | 1352 | 1241 | 1351 | 1380 | 1258 | 1197.5 |
| NM_000153 | LUA#10 | 156.5 | 142 | 135 | 150 | 148.5 | 138 | 166 | 175 | 157 | 144.5 |
| NM_006948 | LUA#51 | 69 | 62 | 63.5 | 72.5 | 65.5 | 66 | 72 | 80 | 64 | 62.5 |
| NM_004631 | LUA#52 | 768 | 722.5 | 723 | 823 | 790 | 782 | 743 | 734 | 715 | 668.5 |
| NM_002358 | LUA#53 | 472 | 428 | 395 | 462 | 455 | 552 | 548 | 527 | 445.5 | 414 |
| NM_013402 | LUA#54 | 1081.5 | 1089.5 | 1098 | 1196 | 1271 | 1266.5 | 1222 | 1215 | 1143 | 1087 |
| NM_000875 | LUA#55 | 1088.5 | 1079.5 | 1051 | 1151 | 1112 | 1167 | 1284 | 1241 | 1177 | 1060 |
| NM_001974 | LUA#11 | 169.5 | 194 | 254 | 355 | 223.5 | 263 | 231 | 205 | 211 | 175 |
| NM_000632 | LUA#12 | 393.5 | 405 | 394 | 451.5 | 442 | 478 | 551 | 484.5 | 434.5 | 403 |
| NM_006457 | LUA#13 | 74 | 71 | 80 | 75 | 77 | 84 | 82 | 75.5 | 77 | 67 |
| NM_000698 | LUA#14 | 190 | 205 | 169 | 233 | 215 | 234 | 237.5 | 218.5 | 214 | 184 |
| NM_032571 | LUA#15 | 194 | 177 | 178 | 217 | 217 | 219 | 212 | 217.5 | 198 | 208 |
| NM_006138 | LUA#56 | 412.5 | 383 | 396 | 459.5 | 498 | 441 | 511.5 | 528.5 | 429 | 436 |
| NM_015201 | LUA#57 | 1394 | 1432 | 1363 | 1500 | 1520 | 1702 | 1654.5 | 1623 | 1554 | 1485 |
| NM_006985 | LUA#58 | 1445 | 1332 | 1321.5 | 1558.5 | 1539.5 | 1511 | 1579 | 1614 | 1465 | 1349 |
| NM_004095 | LUA#59 | 677.5 | 673 | 714 | 723 | 761 | 813 | 888 | 849 | 713 | 743 |
| NM_005914 | LUA#60 | 2195 | 1712.5 | 1855 | 1767.5 | 1964 | 2001.5 | 2183 | 2217.5 | 1849 | 1801 |
| NM_007282 | LUA#16 | 3404 | 3317 | 3420 | 3720 | 3640 | 3628 | 3771 | 3742 | 3829 | 3740 |
| NM_003644 | LUA#17 | 382 | 379 | 360 | 396 | 403 | 384 | 420 | 424 | 420.5 | 395.5 |
| NM_001498 | LUA#18 | 1428 | 1422 | 1451 | 1542.5 | 1650 | 1646.5 | 1659 | 1643 | 1534 | 1547 |
| NM_003172 | LUA#19 | 3489.5 | 3393 | 3442.5 | 3630 | 3640 | 3407 | 3561.5 | 3713.5 | 3684 | 3729 |
| NM_004723 | LUA#20 | 1006 | 1055 | 941.5 | 1072 | 1070 | 1033.5 | 1103.5 | 1121 | 1101 | 1058 |
| NM_014366 | LUA#61 | 1858 | 1818.5 | 1815 | 1958 | 1893 | 1955 | 2124 | 2045 | 1954.5 | 1939 |
| NM_003581 | LUA#62 | 461 | 459 | 490 | 1104 | 560.5 | 575.5 | 784 | 492 | 442 | 292.5 |
| NM_018115 | LUA#63 | 3868 | 3688 | 3621.5 | 3997.5 | 4012 | 4038 | 4183 | 4186 | 3995.5 | 3973 |
| NM_021974 | LUA#64 | 2317 | 2285 | 2229 | 2359.5 | 2501 | 2395 | 2375 | 2577 | 2473 | 2448 |
| NM_024045 | LUA#65 | 751 | 715.5 | 652 | 803 | 823 | 922 | 958 | 891.5 | 766 | 704 |
| NM_004079 | LUA#21 | 3401 | 3346 | 3386 | 3649 | 3578.5 | 3621 | 3487 | 3722 | 3500 | 3466 |
| NM_000414 | LUA#22 | 849 | 886 | 876 | 922 | 959 | 923 | 991 | 997 | 1006 | 975.5 |
| NM_001684 | LUA#23 | 3100 | 3084 | 3141 | 3356.5 | 3190 | 3092 | 3330 | 3482 | 3482 | 3375 |
| NM_003879 | LUA#24 | 1887.5 | 1750.5 | 1837 | 1884.5 | 1982 | 2019 | 1981.5 | 2000 | 1908 | 1739 |
| NM_002166 | LUA#25 | 3185 | 2943.5 | 2941 | 3269 | 3091 | 2616 | 2919 | 3433 | 3462 | 2977 |
| NM_005952 | LUA#66 | 876 | 786 | 799 | 803.5 | 902 | 990 | 1022.5 | 960 | 878 | 811 |
| NM_001034 | LUA#67 | 493 | 529 | 561 | 721 | 515.5 | 682 | 767.5 | 716 | 677 | 449.5 |
| NM_003132 | LUA#68 | 347.5 | 309 | 330 | 344 | 404 | 351.5 | 355 | 349.5 | 350.5 | 323 |
| NM_018164 | LUA#69 | 193 | 163 | 225 | 324.5 | 196 | 284 | 359 | 269 | 277.5 | 175 |
| NM_014573 | LUA#70 | 193 | 198 | 204 | 268 | 232.5 | 272 | 262 | 277.5 | 256 | 187 |
| NM_014333 | LUA#26 | 1261 | 1234 | 1285 | 1432 | 1339 | 1336.5 | 1431.5 | 1414 | 1322.5 | 1274 |
| NM_006432 | LUA#27 | 589 | 537 | 543 | 644.5 | 587 | 652 | 654 | 625 | 592.5 | 501 |
| NM_000433 | LUA#28 | 519.5 | 465 | 439 | 478 | 544 | 475 | 576 | 543 | 515.5 | 491.5 |
| NM_000147 | LUA#29 | 231 | 253 | 261 | 242 | 286 | 280 | 269 | 256.5 | 266 | 242 |
| NM_000584 | LUA#30 | 268 | 331.5 | 415.5 | 491 | 316 | 397 | 371.5 | 438 | 375.5 | 271 |
| NM_006452 | LUA#71 | 1525.5 | 1457 | 1651 | 1599 | 1661 | 1895 | 1960 | 1641.5 | 1669 | 1592 |
| NM_005915 | LUA#72 | 442 | 446 | 457 | 449 | 507.5 | 501 | 489 | 502 | 463 | 469 |
| NM_005980 | LUA#73 | 94 | 88 | 90.5 | 85 | 94.5 | 97 | 114 | 91 | 95 | 93.5 |
| NM_002539 | LUA#74 | 793.5 | 759 | 750.5 | 783 | 845 | 953 | 975 | 909 | 783 | 795 |
| NM_019058 | LUA#75 | 2292.5 | 2282.5 | 2565 | 2388 | 2535.5 | 2484 | 2654.5 | 2545 | 2583 | 2289 |
| NM_004152 | LUA#31 | 630 | 607 | 706.5 | 700 | 697 | 751 | 782 | 717 | 646 | 677 |
| NM_004602 | LUA#32 | 102 | 102 | 113 | 124 | 98 | 205 | 540 | 400 | 104 | 138 |
| NM_018890 | LUA#33 | 2566.5 | 2827.5 | 3299 | 3828 | 3031.5 | 3314.5 | 3385 | 2517 | 3080.5 | 2211 |
| NM_001101 | LUA#34 | 2072 | 1968.5 | 2040.5 | 2060.5 | 2190 | 2259 | 2320 | 2389.5 | 2222 | 2133.5 |
| NM_006019 | LUA#35 | 336.5 | 316.5 | 346 | 403 | 354 | 404 | 367 | 363 | 348 | 346 |
| NM_004134 | LUA#76 | 952 | 989 | 1087 | 1135 | 1163 | 1017.5 | 1092 | 1083 | 1053.5 | 1056 |
| NM_005008 | LUA#77 | 826 | 834.5 | 886 | 963.5 | 996 | 949 | 884 | 937.5 | 914 | 857 |
| NM_020117 | LUA#78 | 2051 | 2083 | 2189 | 2086.5 | 2301 | 2289.5 | 2334 | 2460 | 2260 | 2288 |
| NM_001469 | LUA#79 | 433 | 407 | 554 | 697 | 555 | 594.5 | 461 | 448.5 | 502.5 | 369 |
| NM_021203 | LUA#80 | 345 | 387 | 409 | 387 | 426 | 427 | 412 | 427 | 416 | 381.5 |
| NM_002624 | LUA#36 | 273 | 266 | 280 | 324 | 295 | 328 | 304 | 291 | 286 | 271 |
| NM_004759 | LUA#37 | 122 | 120 | 127 | 128 | 144.5 | 135.5 | 147 | 132 | 124 | 134 |
| NM_002664 | LUA#38 | 847.5 | 834 | 832 | 873 | 937.5 | 902 | 875 | 929 | 902.5 | 944 |
| NM_000211 | LUA#39 | 1491 | 1458.5 | 1552 | 1507 | 1624 | 1206 | 1321 | 1614 | 1564.5 | 1554 |
| NM_002468 | LUA#40 | 259.5 | 253.5 | 269 | 284 | 279 | 315 | 376 | 349.5 | 262 | 279 |
| NM_000884 | LUA#81 | 784 | 800 | 844 | 868 | 921 | 887 | 940 | 932 | 895 | 849 |
| NM_003752 | LUA#82 | 952 | 992 | 998 | 943 | 993 | 1078 | 1145 | 1140 | 982.5 | 993 |
| NM_018256 | LUA#83 | 1089 | 1074.5 | 1091 | 1043 | 1123 | 1201.5 | 1267 | 1209.5 | 1127 | 1218 |
| NM_001948 | LUA#84 | 2343 | 2452 | 2481 | 2585.5 | 2510 | 2541 | 2508 | 2564.5 | 2473 | 2549 |
| NM_005566 | LUA#85 | 1548.5 | 1448 | 1550.5 | 1600 | 1689 | 1693.5 | 1735 | 1768 | 1561 | 1536.5 |
| NM_021103 | LUA#41 | 2065 | 1955.5 | 2142 | 2153 | 2196 | 2101 | 2263.5 | 2283 | 2152 | 2177 |
| NM_002970 | LUA#42 | 513.5 | 548.5 | 668 | 657 | 594 | 594.5 | 606 | 530 | 611 | 610.5 |
| NM_003332 | LUA#43 | 1333 | 1474 | 1892 | 1924 | 1823 | 1789 | 1557 | 1583.5 | 1724 | 1751.5 |
| NM_004106 | LUA#44 | 243.5 | 224 | 272 | 284 | 273 | 267 | 288 | 241 | 272.5 | 253 |
| NM_002982 | LUA#45 | 2470 | 2373.5 | 2700 | 2650 | 2725 | 2864 | 2951.5 | 2762 | 2734.5 | 2708 |
| NM_005375 | LUA#86 | 2294.5 | 2336.5 | 2443 | 2444.5 | 2426 | 2521 | 2719 | 2526.5 | 2478 | 2523.5 |
| NM_000250 | LUA#87 | 2043 | 1985 | 1993 | 2045 | 2198.5 | 2355.5 | 2463 | 2159 | 2135 | 2254 |
| NM_004526 | LUA#88 | 1153 | 1095.5 | 1178 | 1222.5 | 1322.5 | 1285 | 1299 | 1245 | 1257.5 | 1168.5 |
| NM_004741 | LUA#89 | 755.5 | 845 | 1009 | 1203 | 1030 | 1084 | 1146 | 1021 | 897 | 788 |
| NM_002467 | LUA#90 | 1510 | 1469 | 1648 | 1680 | 1767.5 | 1684 | 1670 | 1715.5 | 1649.5 | 1681 |
| ACTB | LUA#91 | 3243 | 3090 | 3181 | 3245 | 3443.5 | 3098.5 | 3174.5 | 3348 | 3385 | 3370 |
| TFRC | LUA#92 | 692 | 743 | 812 | 830 | 855 | 839 | 816 | 843.5 | 762.5 | 801 |
| GAPDH_5 | LUA#93 | 2242 | 1971.5 | 2105 | 2295 | 2386.5 | 2392 | 2183 | 2284 | 2124 | 2235 |
| GAPDH_M | LUA#94 | 3858 | 3566.5 | 3872 | 3913 | 3933 | 3983 | 3872 | 4090 | 3926 | 3949.5 |
| GAPDH_3 | LUA#95 | 3915 | 3968 | 4259 | 4355 | 4446 | 4043 | 4225.5 | 4362 | 4541 | 4571 |

Table 5G. Microtiter plates

| description | FlexMap ID | tretinoin20 | tretinoin21 | tretinoin22 | tretinoin23 | tretinoin24 | tretinoin25 | tretinoin26 | tretinoin27 | tretinoin28 | tretinoin29 |
|---|---|---|---|---|---|---|---|---|---|---|---|
| NM_005736 | LUA#1 | 640 | 730 | 788 | 766 | 718.5 | 145.5 | 751 | 792.5 | 778.5 | 741.5 |
| NM_000070 | LUA#2 | 685 | 687.5 | 755 | 686 | 478 | 115 | 700 | 690 | 707.5 | 750 |
| NM_018217 | LUA#3 | 1359 | 1442 | 1484 | 1465 | 1196 | 235 | 1307 | 1438 | 1438 | 1492 |
| NM_004782 | LUA#4 | 1134 | 1250 | 1263 | 1217 | 962 | 226 | 1119 | 1230 | 1371 | 1286.5 |
| NM_014962 | LUA#5 | 1192 | 1211 | 1294 | 1182 | 872 | 218 | 1154 | 1277.5 | 1326.5 | 1336 |
| NM_004514 | LUA#46 | 1159 | 1194 | 1197.5 | 1151 | 971.5 | 243 | 1114 | 1193.5 | 1243 | 1193 |
| NM_006773 | LUA#47 | 1043 | 1086 | 1044.5 | 1145 | 874 | 229.5 | 1091 | 1167 | 1177.5 | 1171 |
| NM_014288 | LUA#48 | 867 | 887 | 849 | 859 | 606 | 198 | 905 | 883.5 | 896 | 853 |
| NM_017440 | LUA#49 | 563.5 | 606.5 | 635 | 668 | 504 | 126.5 | 572 | 648.5 | 629 | 633.5 |
| NM_007331 | LUA#50 | 804 | 879 | 877 | 868 | 608 | 128 | 875 | 901 | 875 | 857 |
| NM_173823 | LUA#6 | 1031.5 | 1028 | 1074.5 | 1021 | 710 | 89 | 1015.5 | 996 | 1138 | 1126 |
| NM_000962 | LUA#7 | 786 | 838 | 835 | 742.5 | 502 | 92 | 805 | 820 | 873.5 | 847 |
| NM_003825 | LUA#8 | 293.5 | 304 | 303.5 | 303 | 208 | 84.5 | 314 | 265.5 | 328 | 358.5 |
| NM_016061 | LUA#9 | 1161 | 1212 | 1243.5 | 1255 | 942 | 195.5 | 1273 | 1292 | 1309 | 1366 |
| NM_000153 | LUA#10 | 142.5 | 166 | 147 | 124.5 | 108 | 42 | 176 | 157 | 173.5 | 164 |
| NM_006948 | LUA#51 | 63.5 | 72 | 79.5 | 82 | 59 | 30.5 | 88 | 79 | 73 | 75.5 |
| NM_004631 | LUA#52 | 675 | 735.5 | 722 | 673 | 420 | 123 | 680.5 | 683 | 730 | 736 |
| NM_002358 | LUA#53 | 404 | 452 | 468 | 552 | 396 | 65 | 522 | 505 | 462 | 479.5 |
| NM_013402 | LUA#54 | 1170 | 1190.5 | 1234.5 | 1175 | 847 | 159 | 1146 | 1184.5 | 1207 | 1299 |
| NM_000875 | LUA#55 | 1106 | 1147 | 1164 | 1109.5 | 1133 | 244 | 1050 | 1207.5 | 1186.5 | 1240 |
| NM_001974 | LUA#11 | 192.5 | 244 | 328 | 229 | 131 | 41 | 187 | 206 | 326 | 280.5 |
| NM_000632 | LUA#12 | 416 | 462 | 441 | 466 | 399 | 62 | 417 | 427.5 | 472 | 485.5 |
| NM_006457 | LUA#13 | 71 | 77 | 88 | 76.5 | 62 | 29 | 91 | 70 | 77 | 88 |
| NM_000698 | LUA#14 | 184 | 221 | 218.5 | 240 | 183 | 51 | 234 | 217 | 231 | 250 |
| NM_032571 | LUA#15 | 197 | 206 | 211 | 210 | 146.5 | 39 | 207.5 | 199.5 | 237 | 225 |
| NM_006138 | LUA#56 | 439 | 465 | 463 | 492 | 400 | 76 | 505.5 | 508 | 481 | 455 |
| NM_015201 | LUA#57 | 1474 | 1697 | 1545.5 | 1613 | 1288 | 239 | 1501.5 | 1566.5 | 1693 | 1688 |
| NM_006985 | LUA#58 | 1404 | 1426.5 | 1391 | 1466 | 1025 | 152.5 | 1520 | 1559.5 | 1588.5 | 1516.5 |
| NM_004095 | LUA#59 | 781 | 826.5 | 724 | 833.5 | 508 | 80.5 | 750 | 786 | 836.5 | 825 |
| NM_005914 | LUA#60 | 1889 | 2185 | 2217 | 2583 | 1861 | 314 | 1944 | 2405 | 2103 | 2084.5 |
| NM_007282 | LUA#16 | 3819 | 3830 | 3905 | 3599 | 3355 | 1195 | 3365 | 3717 | 3937 | 3873 |
| NM_003644 | LUA#17 | 418 | 413 | 423 | 392.5 | 312.5 | 90.5 | 407 | 394 | 423 | 439.5 |
| NM_001498 | LUA#18 | 1596 | 1583 | 1644.5 | 1592.5 | 1126 | 194.5 | 1507 | 1592 | 1675 | 1729 |
| NM_003172 | LUA#19 | 3734 | 3740 | 3765 | 3485.5 | 3527 | 1393.5 | 3551 | 3869 | 3875.5 | 3546 |
| NM_004723 | LUA#20 | 1025 | 1135.5 | 1076 | 967 | 712.5 | 142 | 1098 | 1078 | 1129 | 1205.5 |
| NM_014366 | LUA#61 | 1866 | 1983.5 | 1990 | 1945 | 2046 | 505.5 | 1985 | 2063 | 2011 | 2041 |
| NM_003581 | LUA#62 | 349 | 459 | 725 | 486 | 433.5 | 59 | 330 | 467 | 542 | 538 |
| NM_018115 | LUA#63 | 4087.5 | 4059 | 3982 | 3939 | 3665 | 1204 | 4071 | 4113 | 4167 | 4218 |
| NM_021974 | LUA#64 | 2484 | 2467.5 | 2483 | 2350 | 1757 | 441 | 2372.5 | 2637 | 2567 | 2591 |
| NM_024045 | LUA#65 | 729.5 | 798 | 779.5 | 770 | 587 | 99 | 747 | 790 | 826 | 797 |
| NM_004079 | LUA#21 | 3552 | 3605.5 | 3495 | 3377 | 2652.5 | 688.5 | 3443.5 | 3592 | 3531 | 3582 |
| NM_000414 | LUA#22 | 941 | 1003 | 979 | 933 | 633 | 96.5 | 1010 | 1015 | 1053 | 1005 |
| NM_001684 | LUA#23 | 3316.5 | 3530 | 3511 | 3136.5 | 3092 | 1247 | 3338 | 3430.5 | 3590 | 3642.5 |
| NM_003879 | LUA#24 | 1848 | 2031 | 1977 | 1896.5 | 1645 | 342 | 1984 | 2060 | 2126.5 | 2034 |
| NM_002166 | LUA#25 | 3071 | 3382.5 | 3762.5 | 2832 | 2572 | 617 | 3016 | 3708 | 3766.5 | 3602.5 |
| NM_005952 | LUA#66 | 814.5 | 845 | 850.5 | 894.5 | 737 | 127 | 851.5 | 850.5 | 872 | 913 |
| NM_001034 | LUA#67 | 484 | 710 | 707.5 | 608 | 337 | 64 | 394.5 | 517 | 729.5 | 805 |
| NM_003132 | LUA#68 | 333.5 | 304 | 353 | 334 | 199 | 50.5 | 314 | 304.5 | 342 | 355.5 |
| NM_018164 | LUA#69 | 170 | 247 | 293 | 223 | 225.5 | 51 | 168 | 196 | 284 | 297 |
| NM_014573 | LUA#70 | 189 | 231 | 251 | 273.5 | 154 | 52 | 216.5 | 199 | 237 | 267.5 |
| NM_014333 | LUA#26 | 1265 | 1399.5 | 1470 | 1456.5 | 1091 | 254 | 1237 | 1396 | 1489.5 | 1499 |
| NM_006432 | LUA#27 | 554.5 | 605.5 | 690 | 686 | 480 | 101.5 | 574 | 628.5 | 679 | 694.5 |
| NM_000433 | LUA#28 | 541 | 499 | 552 | 488.5 | 308 | 69 | 478 | 551.5 | 545 | 548.5 |
| NM_000147 | LUA#29 | 273 | 266 | 299 | 278 | 198.5 | 49 | 207 | 257.5 | 286 | 277.5 |
| NM_000584 | LUA#30 | 231 | 358 | 433 | 352 | 199 | 57 | 314 | 280 | 411 | 431 |
| NM_006452 | LUA#71 | 1830 | 1544.5 | 1789 | 1876.5 | 1259.5 | 252 | 1497 | 1660 | 1647 | 1637 |
| NM_005915 | LUA#72 | 460.5 | 493.5 | 510 | 433.5 | 306 | 76 | 502.5 | 519 | 491 | 498 |
| NM_005980 | LUA#73 | 97 | 94 | 96 | 96 | 83.5 | 40 | 101.5 | 104 | 95 | 90 |
| NM_002539 | LUA#74 | 902 | 784.5 | 850 | 893 | 623 | 129 | 754 | 831 | 786 | 828 |
| NM_019058 | LUA#75 | 2347 | 2353 | 2439.5 | 2270 | 1909 | 415 | 2069.5 | 2436 | 2667 | 2983 |
| NM_004152 | LUA#31 | 679 | 718 | 764 | 752 | 450 | 88 | 610 | 631 | 712 | 768 |
| NM_004602 | LUA#32 | 95 | 108 | 161 | 115 | 685 | 100 | 321.5 | 251 | 106 | 111 |
| NM_018890 | LUA#33 | 2323 | 3545 | 3734 | 3090 | 3474.5 | 868 | 2600 | 2734.5 | 3577 | 3830 |
| NM_001101 | LUA#34 | 2216 | 2108.5 | 2276.5 | 2231.5 | 1848.5 | 439 | 2166.5 | 2230 | 2157.5 | 2312 |
| NM_006019 | LUA#35 | 388 | 349 | 414 | 328.5 | 242 | 56 | 344 | 357.5 | 392 | 417 |
| NM_004134 | LUA#76 | 1041 | 1069 | 1060.5 | 987 | 651 | 132 | 1092 | 1095 | 1138.5 | 1200 |
| NM_005008 | LUA#77 | 853 | 895 | 1077 | 907 | 520 | 118 | 791 | 852.5 | 942 | 939.5 |
| NM_020117 | LUA#78 | 2468 | 2232 | 2341 | 2280.5 | 1844.5 | 467 | 2069 | 2395 | 2201 | 2287 |
| NM_001469 | LUA#79 | 425 | 580 | 740.5 | 599 | 359 | 81.5 | 467 | 500.5 | 586 | 636 |
| NM_021203 | LUA#80 | 415 | 396 | 437 | 372 | 217.5 | 57 | 363 | 405 | 409.5 | 445 |
| NM_002624 | LUA#36 | 274 | 294 | 336 | 309.5 | 241.5 | 48 | 285 | 292 | 355 | 346 |
| NM_004759 | LUA#37 | 149 | 116 | 151 | 130 | 114 | 42 | 178 | 122 | 140 | 139 |
| NM_002664 | LUA#38 | 977.5 | 914 | 985 | 863 | 577.5 | 111.5 | 817 | 950 | 905 | 914 |
| NM_000211 | LUA#39 | 1765 | 1572.5 | 1714.5 | 1155 | 740 | 226 | 1502 | 1608 | 1601.5 | 1621 |
| NM_002468 | LUA#40 | 284 | 300 | 313 | 272 | 277 | 55 | 266.5 | 327 | 329 | 307.5 |
| NM_000884 | LUA#81 | 903 | 886.5 | 945 | 903 | 570 | 118 | 819.5 | 863 | 870 | 906 |
| NM_003752 | LUA#82 | 1041 | 1060 | 1104 | 1061 | 837.5 | 170 | 938.5 | 1068 | 1038 | 1035 |
| NM_018256 | LUA#83 | 1108 | 1137 | 1196 | 1134 | 827.5 | 167 | 1206.5 | 1275 | 1135 | 1244 |
| NM_001948 | LUA#84 | 2580 | 2584 | 2660 | 2423.5 | 1685.5 | 408 | 2252.5 | 2493 | 2630 | 2638 |
| NM_005566 | LUA#85 | 1561 | 1647 | 1700 | 1554 | 1127.5 | 209 | 1471 | 1537 | 1646 | 1690.5 |
| NM_021103 | LUA#41 | 2307 | 2172 | 2274 | 2057 | 1814 | 618 | 2016 | 2179 | 2230 | 2237.5 |
| NM_002970 | LUA#42 | 606 | 572 | 551 | 556 | 317 | 112 | 622 | 574 | 616.5 | 627 |
| NM_003332 | LUA#43 | 1942.5 | 1967.5 | 1914 | 1850 | 794.5 | 344 | 1313 | 1673 | 2015 | 2047 |
| NM_004106 | LUA#44 | 272 | 246 | 291 | 273 | 172 | 64.5 | 313 | 251 | 305 | 293 |
| NM_002982 | LUA#45 | 2717.5 | 2736 | 2784.5 | 2777 | 2331 | 489 | 2387.5 | 2700.5 | 2681.5 | 2746.5 |
| NM_005375 | LUA#86 | 2474 | 2594 | 2548 | 2496 | 1566.5 | 360 | 2233 | 2415 | 2634 | 2525 |
| NM_000250 | LUA#87 | 2214.5 | 2002.5 | 2248 | 2313 | 1710 | 399.5 | 1844 | 2084 | 2015 | 2121 |
| NM_004526 | LUA#88 | 1164 | 1159 | 1256 | 1110 | 796 | 197 | 1091 | 1190 | 1226 | 1219 |
| NM_004741 | LUA#89 | 883 | 948.5 | 1013.5 | 920 | 617 | 168 | 738 | 768 | 924 | 987 |
| NM_002467 | LUA#90 | 1628.5 | 1730 | 1731 | 1783 | 1174 | 317 | 1467 | 1738 | 1729.5 | 1861 |
| ACTB | LUA#91 | 3368 | 3386 | 3350.5 | 3125 | 2605 | 672 | 3275 | 3524.5 | 3469 | 3388 |
| TFRC | LUA#92 | 860.5 | 835 | 930.5 | 845 | 477 | 109 | 774 | 838 | 892 | 890 |
| GAPDH_5 | LUA#93 | 2317.5 | 2229 | 2328 | 2155 | 1768 | 404.5 | 2142 | 2223 | 2241 | 2203 |
| GAPDH_M | LUA#94 | 3957 | 3991 | 4132 | 3551.5 | 3745 | 1082 | 3577 | 4067 | 3950.5 | 3995 |
| GAPDH_3 | LUA#95 | 4351 | 4364 | 4325 | 4183.5 | 3891.5 | 2279 | 3800.5 | 4394 | 4434 | 4483 |

Table 5H. Microtiter plates

| description | FlexMap ID | tretinoin30 | tretinoin31 | tretinoin32 | tretinoin33 | tretinoin34 | tretinoin35 | tretinoin36 | tretinoin37 | tretinoin38 | tretinoin39 |
|---|---|---|---|---|---|---|---|---|---|---|---|
| NM_005736 | LUA#1 | 769 | 747 | 1238.5 | 1115 | 813.5 | 721 | 962.5 | 1272 | 847.5 | 790 |
| NM_000070 | LUA#2 | 689.5 | 657 | 758.5 | 754 | 803 | 540.5 | 741 | 761 | 740.5 | 589.5 |
| NM_018217 | LUA#3 | 1322 | 1374 | 1573 | 1436.5 | 1463 | 1150 | 1445 | 1403 | 1383 | 1174.5 |
| NM_004782 | LUA#4 | 1185.5 | 1099 | 1239 | 1216.5 | 1215 | 921 | 1141.5 | 1079 | 1213.5 | 956 |
| NM_014962 | LUA#5 | 1150.5 | 1180 | 1240.5 | 1191 | 1253 | 853 | 1132 | 1133 | 1258 | 1011 |
| NM_004514 | LUA#46 | 1094.5 | 1087 | 1186 | 1169.5 | 1242 | 941 | 1167 | 1131 | 1193 | 977.5 |
| NM_006773 | LUA#47 | 1033 | 990 | 1208 | 1122 | 1156 | 847 | 1103.5 | 1060.5 | 1077 | 922 |
| NM_014288 | LUA#48 | 810 | 728 | 911.5 | 842 | 881.5 | 617 | 825.5 | 791 | 787 | 644 |
| NM_017440 | LUA#49 | 589 | 605.5 | 756 | 676 | 636.5 | 465 | 606 | 652 | 646 | 518.5 |
| NM_007331 | LUA#50 | 817 | 843 | 963 | 889 | 941 | 646.5 | 873 | 889.5 | 926 | 727 |
| NM_173823 | LUA#6 | 1059 | 1019.5 | 1053 | 1013.5 | 1122 | 648 | 994 | 985.5 | 1175.5 | 938 |
| NM_000962 | LUA#7 | 852 | 700.5 | 819.5 | 858 | 900 | 571.5 | 816 | 798 | 844.5 | 643 |
| NM_003825 | LUA#8 | 306 | 343 | 267 | 332 | 340 | 243 | 288 | 287 | 360 | 352 |
| NM_016061 | LUA#9 | 1193 | 1073.5 | 1220.5 | 1219 | 1260 | 928.5 | 1243.5 | 1193 | 1168 | 1021 |
| NM_000153 | LUA#10 | 142 | 139 | 155 | 171 | 143.5 | 110.5 | 152 | 145 | 173 | 140 |
| NM_006948 | LUA#51 | 76.5 | 77.5 | 91 | 84 | 92 | 68.5 | 75.5 | 79 | 78 | 74 |
| NM_004631 | LUA#52 | 698 | 714 | 696 | 645 | 717 | 415 | 656 | 645 | 773.5 | 621 |
| NM_002358 | LUA#53 | 461 | 581 | 624 | 570 | 530.5 | 354 | 470.5 | 458 | 553 | 484 |
| NM_013402 | LUA#54 | 1164 | 1118 | 1162 | 1205 | 1236 | 814 | 1171 | 1055 | 1330 | 987 |
| NM_000875 | LUA#55 | 1102 | 1155 | 1372 | 1375 | 1322 | 1146 | 1294 | 1286 | 1195 | 1045.5 |
| NM_001974 | LUA#11 | 305.5 | 276 | 191 | 246 | 259 | 162.5 | 192 | 206 | 295 | 226 |
| NM_000632 | LUA#12 | 441 | 482 | 621 | 688 | 451 | 309 | 426.5 | 453 | 450 | 447 |
| NM_006457 | LUA#13 | 73 | 106 | 88 | 88 | 92.5 | 59 | 79.5 | 84 | 96 | 98 |
| NM_000698 | LUA#14 | 261 | 264 | 271.5 | 260 | 282 | 176 | 237 | 269 | 279.5 | 260 |
| NM_032571 | LUA#15 | 201 | 224.5 | 213.5 | 200 | 216 | 141 | 223 | 213 | 231 | 214 |
| NM_006138 | LUA#56 | 460 | 420.5 | 539 | 534 | 484 | 367 | 443 | 470 | 470.5 | 449 |
| NM_015201 | LUA#57 | 1524 | 1740 | 1601 | 1563 | 1686 | 1294 | 1589.5 | 1575 | 1698 | 1441 |
| NM_006985 | LUA#58 | 1336 | 1304 | 1469 | 1513.5 | 1622.5 | 982 | 1517.5 | 1475 | 1453 | 1167.5 |
| NM_004095 | LUA#59 | 733 | 717 | 786 | 697 | 764.5 | 433 | 732 | 708.5 | 793.5 | 612 |
| NM_005914 | LUA#60 | 1856 | 2038.5 | 2571 | 2012.5 | 1880 | 1606 | 1870 | 1839 | 1896 | 1530 |
| NM_007282 | LUA#16 | 3508 | 3192.5 | 3565 | 3677 | 3822.5 | 3381 | 3679 | 3410 | 3550.5 | 2848 |
| NM_003644 | LUA#17 | 386.5 | 383 | 417.5 | 396 | 423 | 301 | 374 | 379 | 432 | 336 |
| NM_001498 | LUA#18 | 1574 | 1494 | 1634 | 1631 | 1704 | 1106 | 1567 | 1552 | 1756 | 1327 |
| NM_003172 | LUA#19 | 3400.5 | 3075 | 3448 | 3697 | 3747 | 3740 | 3828 | 3719 | 3455.5 | 2881 |
| NM_004723 | LUA#20 | 979 | 935.5 | 1064 | 1087 | 1182.5 | 753 | 1069 | 975.5 | 1032.5 | 798 |
| NM_014366 | LUA#61 | 1912 | 1786 | 2169.5 | 2125 | 2094 | 1938 | 2057 | 2071 | 1931 | 1729.5 |
| NM_003581 | LUA#62 | 580 | 836 | 497.5 | 667 | 676 | 406 | 453 | 544 | 633 | 625.5 |
| NM_018115 | LUA#63 | 3832.5 | 3397 | 4093 | 4088 | 4279.5 | 3877 | 4112.5 | 3898.5 | 3945 | 3394 |
| NM_021974 | LUA#64 | 2311 | 2023 | 2396 | 2418 | 2572 | 2001 | 2468 | 2388 | 2492 | 1925 |
| NM_024045 | LUA#65 | 778 | 821.5 | 869 | 774.5 | 847.5 | 557.5 | 752 | 697 | 801 | 700.5 |
| NM_004079 | LUA#21 | 3270 | 2953 | 3391 | 3470 | 3448 | 2730.5 | 3407.5 | 3299 | 3366 | 2635 |
| NM_000414 | LUA#22 | 997 | 865.5 | 1028 | 974 | 1061 | 630.5 | 996 | 885.5 | 948 | 778.5 |
| NM_001684 | LUA#23 | 3231 | 3000 | 3194.5 | 3326 | 3417 | 3351 | 3506.5 | 3213.5 | 3205 | 2689 |
| NM_003879 | LUA#24 | 1900 | 1735 | 2096 | 2056 | 2072 | 1621.5 | 2080 | 1945 | 1980.5 | 1569.5 |
| NM_002166 | LUA#25 | 2926 | 2752 | 2950 | 2945 | 2932 | 2688.5 | 2896 | 2989 | 2573 | 2189.5 |
| NM_005952 | LUA#66 | 843 | 867.5 | 978 | 977.5 | 953 | 645 | 842 | 796 | 927 | 766.5 |
| NM_001034 | LUA#67 | 718 | 878 | 591.5 | 781 | 904 | 488 | 618.5 | 586.5 | 694 | 572 |
| NM_003132 | LUA#68 | 342 | 274 | 322 | 361.5 | 318.5 | 210 | 307 | 316 | 370.5 | 250 |
| NM_018164 | LUA#69 | 315 | 320 | 241.5 | 360.5 | 357 | 178.5 | 201 | 241 | 354 | 292 |
| NM_014573 | LUA#70 | 252.5 | 283 | 251 | 305 | 286 | 220 | 233 | 257 | 280 | 232 |
| NM_014333 | LUA#26 | 1319 | 1406 | 1465 | 1494 | 1419 | 1092.5 | 1371.5 | 1338 | 1429.5 | 1161 |
| NM_006432 | LUA#27 | 661 | 705.5 | 664 | 722 | 683 | 461.5 | 592 | 634 | 716 | 572 |
| NM_000433 | LUA#28 | 523 | 441 | 538 | 528 | 568 | 326 | 499 | 457 | 537 | 346 |
| NM_000147 | LUA#29 | 258 | 300 | 313 | 275 | 294 | 195 | 304 | 253.5 | 326 | 246 |
| NM_000584 | LUA#30 | 391.5 | 493 | 276 | 416 | 451 | 259 | 288 | 399.5 | 432 | 343.5 |
| NM_006452 | LUA#71 | 1577.5 | 1658 | 1888 | 1709 | 1735 | 1113 | 1497 | 1516.5 | 1840 | 1397.5 |
| NM_005915 | LUA#72 | 481.5 | 510.5 | 523 | 583.5 | 576 | 356 | 504.5 | 415 | 564 | 392.5 |
| NM_005980 | LUA#73 | 90 | 99 | 124 | 123 | 112 | 81 | 100 | 99 | 105.5 | 108 |
| NM_002539 | LUA#74 | 830 | 864 | 906.5 | 944.5 | 939 | 599 | 833 | 759 | 913 | 662 |
| NM_019058 | LUA#75 | 2325 | 2239 | 2435 | 2754 | 2662 | 1983 | 2392.5 | 2181 | 2399.5 | 1863 |
| NM_004152 | LUA#31 | 707 | 677 | 734 | 995 | 718 | 405 | 615.5 | 675 | 744 | 547 |
| NM_004602 | LUA#32 | 153.5 | 214 | 909 | 1332 | 203 | 575 | 362.5 | 661 | 173 | 545 |
| NM_018890 | LUA#33 | 3389 | 3144 | 3168 | 4165 | 3486 | 2889 | 2506 | 3509 | 3510 | 2841.5 |
| NM_001101 | LUA#34 | 2091 | 2067 | 2357 | 2333 | 2374 | 1832 | 2294 | 2114.5 | 2236 | 1732 |
| NM_006019 | LUA#35 | 361.5 | 380 | 336.5 | 375 | 398 | 238 | 335 | 355 | 418 | 362 |
| NM_004134 | LUA#76 | 1049 | 803.5 | 933.5 | 1017 | 1043.5 | 699.5 | 1017 | 1031 | 1077.5 | 763 |
| NM_005008 | LUA#77 | 878 | 900 | 828 | 936.5 | 1033 | 581 | 886.5 | 860.5 | 949.5 | 736 |
| NM_020117 | LUA#78 | 2230 | 2093 | 2431 | 2387.5 | 2540.5 | 1912 | 2311 | 2187 | 2354 | 1724 |
| NM_001469 | LUA#79 | 691.5 | 645 | 471 | 654 | 680.5 | 506.5 | 464.5 | 577 | 767 | 627 |
| NM_021203 | LUA#80 | 400 | 353 | 369 | 437 | 476 | 210 | 385.5 | 351.5 | 442 | 327 |
| NM_002624 | LUA#36 | 312.5 | 357 | 327 | 291 | 357 | 234 | 326 | 320 | 376 | 332.5 |
| NM_004759 | LUA#37 | 147 | 125.5 | 134 | 177 | 156 | 107 | 133 | 132 | 158 | 121.5 |
| NM_002664 | LUA#38 | 867 | 808 | 927 | 916.5 | 1041 | 612 | 854.5 | 843 | 960.5 | 655 |
| NM_000211 | LUA#39 | 1459.5 | 1090 | 1164 | 1588 | 1684 | 1053 | 1460.5 | 1363 | 1622.5 | 766.5 |
| NM_002468 | LUA#40 | 270 | 364 | 428 | 462.5 | 356 | 285 | 336 | 540 | 393 | 353.5 |
| NM_000884 | LUA#81 | 847 | 824 | 971 | 938 | 986.5 | 575 | 838 | 813 | 932 | 759.5 |
| NM_003752 | LUA#82 | 968 | 1037 | 1171.5 | 1058 | 1106 | 797 | 1027.5 | 973 | 1082 | 863 |
| NM_018256 | LUA#83 | 1156.5 | 1089 | 1223 | 1153 | 1309 | 858 | 1084.5 | 996 | 1123 | 870 |
| NM_001948 | LUA#84 | 2417 | 2293.5 | 2372 | 2431 | 2615 | 1881.5 | 2387 | 2286 | 2542 | 1863 |
| NM_005566 | LUA#85 | 1603.5 | 1464.5 | 1570 | 1600.5 | 1792 | 1039 | 1520.5 | 1348 | 1667.5 | 1186.5 |
| NM_021103 | LUA#41 | 2062 | 1819 | 2376 | 2712 | 2331.5 | 1867.5 | 2178 | 2117 | 2183 | 1664 |
| NM_002970 | LUA#42 | 557 | 632 | 554 | 689 | 557 | 324.5 | 443 | 542 | 579 | 454 |
| NM_003332 | LUA#43 | 2024 | 1928 | 1382 | 1319.5 | 1562 | 875.5 | 1550.5 | 1649 | 2060 | 1620 |
| NM_004106 | LUA#44 | 259 | 257.5 | 287.5 | 297 | 295 | 172 | 238 | 298 | 282 | 235 |
| NM_002982 | LUA#45 | 2586 | 2449 | 3029.5 | 3071 | 2895 | 2314 | 2978 | 3409 | 2749.5 | 2503.5 |
| NM_005375 | LUA#86 | 2374.5 | 2331 | 2476 | 2278.5 | 2453.5 | 1650 | 2201 | 2224 | 2486 | 2014 |
| NM_000250 | LUA#87 | 2007 | 1994 | 2529 | 2190 | 2281 | 1760 | 2035 | 2053.5 | 2251 | 1773 |
| NM_004526 | LUA#88 | 1199.5 | 1072 | 1200 | 1291 | 1302 | 863 | 1242 | 1132.5 | 1240 | 874 |
| NM_004741 | LUA#89 | 948 | 938 | 694 | 1441 | 960 | 494 | 724 | 909 | 941 | 817 |
| NM_002467 | LUA#90 | 1735 | 1597 | 1675.5 | 1910.5 | 1668 | 1121 | 1574.5 | 1764.5 | 1806 | 1401 |
| ACTB | LUA#91 | 3074 | 2741 | 3236 | 3178 | 3290 | 2654.5 | 3268 | 3235 | 3265 | 2408.5 |
| TFRC | LUA#92 | 899 | 882 | 882 | 800.5 | 940 | 534 | 833 | 845 | 1049.5 | 774.5 |
| GAPDH_5 | LUA#93 | 2041.5 | 1918 | 2170.5 | 2325 | 2409.5 | 1721 | 2170.5 | 2079 | 2197 | 1628 |
| GAPDH_M | LUA#94 | 3615 | 3383 | 3924 | 4094 | 4111 | 3702 | 4060 | 3901 | 3849 | 3216 |
| GAPDH_3 | LUA#95 | 4065 | 3741 | 4295 | 4166 | 4220 | 4140 | 4339.5 | 4356 | 4121 | 3415 |

Table 5I. Microtiter plates

| description | FlexMap ID | tretinoin40 | tretinoin41 | tretinoin42 | tretinoin43 | tretinoin44 | tretinoin45 | tretinoin46 | tretinoin47 |
|---|---|---|---|---|---|---|---|---|---|
| NM_005736 | LUA#1 | 92 | 522 | 909 | 1432 | 1896 | 847 | 1205.5 | 695 |
| NM_000070 | LUA#2 | 84.5 | 400 | 756 | 769.5 | 717.5 | 638.5 | 741 | 490.5 |
| NM_018217 | LUA#3 | 189 | 990 | 1514 | 1581 | 1499 | 1286 | 1386 | 854.5 |
| NM_004782 | LUA#4 | 163 | 811 | 1306 | 1264 | 1063.5 | 1077 | 1094 | 639 |
| NM_014962 | LUA#5 | 152.5 | 726 | 1269 | 1290 | 1213 | 1076 | 1134 | 717 |
| NM_004514 | LUA#46 | 176 | 834 | 1159.5 | 1224 | 964 | 992 | 1017 | 626 |
| NM_006773 | LUA#47 | 164 | 717 | 1137 | 1120.5 | 783 | 937.5 | 907 | 576.5 |
| NM_014288 | LUA#48 | 154 | 477.5 | 801 | 816 | 527.5 | 706 | 650 | 453 |
| NM_017440 | LUA#49 | 101 | 405.5 | 707 | 774 | 716 | 607 | 693 | 477 |
| NM_007331 | LUA#50 | 88 | 474 | 915.5 | 927.5 | 770 | 739 | 835 | 583 |
| NM_173823 | LUA#6 | 84 | 594.5 | 1106.5 | 1168.5 | 1130.5 | 1080 | 1163 | 807 |
| NM_000962 | LUA#7 | 63.5 | 391 | 761 | 776 | 470 | 646.5 | 671.5 | 452 |
| NM_003825 | LUA#8 | 81.5 | 192.5 | 338 | 383.5 | 307.5 | 331 | 502 | 476.5 |
| NM_016061 | LUA#9 | 134 | 674 | 1086 | 1267.5 | 863 | 975 | 1073.5 | 741 |
| NM_000153 | LUA#10 | 35 | 90 | 138 | 150 | 120 | 138 | 171.5 | 223 |
| NM_006948 | LUA#51 | 34 | 49 | 82 | 95 | 83 | 79.5 | 85.5 | 106.5 |
| NM_004631 | LUA#52 | 86.5 | 322 | 705 | 629 | 524 | 633 | 667 | 532 |
| NM_002358 | LUA#53 | 64 | 339 | 572 | 611 | 366 | 481 | 531 | 433 |
| NM_013402 | LUA#54 | 114 | 705 | 1150 | 1150 | 806 | 1051.5 | 1087 | 711 |
| NM_000875 | LUA#55 | 184 | 1002 | 1389.5 | 1772 | 1303 | 1192 | 1271 | 810 |
| NM_001974 | LUA#11 | 38.5 | 98 | 351 | 256 | 258 | 253.5 | 286.5 | 224 |
| NM_000632 | LUA#12 | 60 | 298 | 544 | 994 | 932 | 486 | 500 | 525.5 |
| NM_006457 | LUA#13 | 36 | 51 | 83 | 94 | 84 | 95 | 132 | 200 |
| NM_000698 | LUA#14 | 45.5 | 156 | 269 | 394 | 342 | 297.5 | 363 | 352 |
| NM_032571 | LUA#15 | 31 | 109 | 203 | 222.5 | 165.5 | 191 | 270.5 | 253.5 |
| NM_006138 | LUA#56 | 70 | 325.5 | 443 | 659.5 | 488 | 437 | 429 | 341 |
| NM_015201 | LUA#57 | 182 | 1154 | 1768 | 1714 | 1251 | 1398.5 | 1507 | 930.5 |
| NM_006985 | LUA#58 | 90 | 720 | 1223 | 1310 | 813 | 1136.5 | 983 | 635 |
| NM_004095 | LUA#59 | 72 | 383 | 714 | 665 | 496.5 | 650.5 | 593.5 | 442 |
| NM_005914 | LUA#60 | 303 | 1363 | 2538.5 | 2273 | 1739 | 1694 | 1933.5 | 1154.5 |
| NM_007282 | LUA#16 | 778.5 | 3067.5 | 3626 | 3678 | 3097 | 3055 | 2958.5 | 1505 |
| NM_003644 | LUA#17 | 69 | 255 | 415 | 428 | 359 | 374 | 395 | 302 |
| NM_001498 | LUA#18 | 126.5 | 890.5 | 1632 | 1563.5 | 1134 | 1467 | 1510 | 888 |
| NM_003172 | LUA#19 | 818 | 3200.5 | 3348 | 3577.5 | 2983 | 2898 | 2747 | 1471 |
| NM_004723 | LUA#20 | 89 | 620 | 1056 | 982 | 761 | 886 | 853 | 494.5 |
| NM_014366 | LUA#61 | 383 | 1773 | 2236 | 2380 | 1850 | 1787.5 | 1681.5 | 1009.5 |
| NM_003581 | LUA#62 | 62.5 | 372 | 962 | 808 | 751 | 735.5 | 801 | 515 |
| NM_018115 | LUA#63 | 746.5 | 3193 | 3722 | 4141 | 3225.5 | 3292.5 | 3054 | 1569 |
| NM_021974 | LUA#64 | 268 | 1606 | 2307 | 2301 | 1472 | 1996 | 1890 | 1117.5 |
| NM_024045 | LUA#65 | 85 | 495 | 831 | 833.5 | 527 | 681 | 728.5 | 471 |
| NM_004079 | LUA#21 | 350 | 2202 | 3008 | 3030.5 | 2326 | 2816 | 2701 | 1488 |
| NM_000414 | LUA#22 | 79.5 | 477 | 902 | 920 | 503 | 800 | 838 | 646 |
| NM_001684 | LUA#23 | 884 | 3012 | 3316.5 | 3512 | 3036 | 2755.5 | 2662 | 1478 |
| NM_003879 | LUA#24 | 225 | 1355.5 | 1937.5 | 1983.5 | 1275 | 1677 | 1564 | 923 |
| NM_002166 | LUA#25 | 411.5 | 1693.5 | 2999 | 2964 | 2068 | 1985 | 2518 | 1239 |
| NM_005952 | LUA#66 | 104 | 573 | 891 | 961 | 623.5 | 847.5 | 775 | 571 |
| NM_001034 | LUA#67 | 65 | 447 | 989 | 779 | 733.5 | 645 | 1063 | 476 |
| NM_003132 | LUA#68 | 41 | 126 | 264.5 | 264 | 195.5 | 257 | 296 | 327.5 |
| NM_018164 | LUA#69 | 52 | 170 | 429 | 380 | 304.5 | 304 | 358.5 | 234 |
| NM_014573 | LUA#70 | 43 | 143 | 320 | 325 | 274 | 243 | 407.5 | 300 |
| NM_014333 | LUA#26 | 180.5 | 949.5 | 1577 | 1549 | 1309 | 1337 | 1390 | 811 |
| NM_006432 | LUA#27 | 90 | 394 | 854.5 | 800 | 695 | 670 | 719 | 450 |
| NM_000433 | LUA#28 | 53 | 234 | 451.5 | 466 | 318 | 421 | 370.5 | 244.5 |
| NM_000147 | LUA#29 | 48 | 184 | 276.5 | 330.5 | 274 | 275 | 318 | 288 |
| NM_000584 | LUA#30 | 45 | 189 | 470 | 461 | 435 | 379 | 597 | 352 |
| NM_006452 | LUA#71 | 207.5 | 1176 | 1736 | 1656.5 | 1374 | 1531 | 1587 | 911.5 |
| NM_005915 | LUA#72 | 48 | 300 | 495 | 474 | 315.5 | 496 | 401.5 | 256.5 |
| NM_005980 | LUA#73 | 35.5 | 83 | 102 | 162 | 149 | 95 | 114 | 168 |
| NM_002539 | LUA#74 | 94 | 559 | 886 | 952 | 715 | 861.5 | 822 | 535 |
| NM_019058 | LUA#75 | 258 | 1804 | 2807 | 2882 | 2288 | 2430 | 2201 | 1184 |
| NM_004152 | LUA#31 | 58 | 337 | 710 | 811 | 628.5 | 782 | 652 | 408.5 |
| NM_004602 | LUA#32 | 458.5 | 678 | 736.5 | 1957 | 1765.5 | 664 | 571.5 | 773.5 |
| NM_018890 | LUA#33 | 562 | 2557 | 3812.5 | 4178 | 4310 | 3724.5 | 2984 | 1462.5 |
| NM_001101 | LUA#34 | 260.5 | 1606 | 2206 | 2257 | 1647 | 1943 | 1881 | 904 |
| NM_006019 | LUA#35 | 41 | 192.5 | 363 | 400.5 | 353 | 361 | 402 | 332 |
| NM_004134 | LUA#76 | 78 | 432.5 | 873 | 866 | 531 | 759 | 744 | 493 |
| NM_005008 | LUA#77 | 73 | 425 | 885 | 923 | 779.5 | 801 | 828 | 525 |
| NM_020117 | LUA#78 | 236.5 | 1585 | 2193 | 2242.5 | 1690 | 1988 | 1734 | 848.5 |
| NM_001469 | LUA#79 | 65 | 320.5 | 885 | 739 | 765 | 734 | 561 | 370 |
| NM_021203 | LUA#80 | 53 | 171 | 386 | 377.5 | 283.5 | 327 | 348 | 279 |
| NM_002624 | LUA#36 | 53 | 180.5 | 397 | 381 | 330.5 | 336 | 400.5 | 394 |
| NM_004759 | LUA#37 | 40.5 | 67.5 | 186.5 | 169 | 134.5 | 142 | 145 | 190 |
| NM_002664 | LUA#38 | 73 | 478 | 906 | 798 | 599 | 773 | 748 | 512 |
| NM_000211 | LUA#39 | 81 | 598.5 | 1229 | 1162.5 | 868 | 1061 | 973 | 440.5 |
| NM_002468 | LUA#40 | 48 | 285.5 | 324 | 728 | 896.5 | 343 | 632 | 464 |
| NM_000884 | LUA#81 | 82 | 506 | 875 | 897 | 732.5 | 789 | 805 | 513.5 |
| NM_003752 | LUA#82 | 115.5 | 536.5 | 1061 | 1015.5 | 768.5 | 1020 | 912.5 | 596 |
| NM_018256 | LUA#83 | 102 | 717 | 1252 | 1104 | 692.5 | 1077 | 929 | 470 |
| NM_001948 | LUA#84 | 255 | 1430 | 2470.5 | 2273.5 | 1891 | 2131 | 2132.5 | 1104 |
| NM_005566 | LUA#85 | 129.5 | 862.5 | 1842.5 | 1550 | 1000 | 1282 | 1196 | 592 |
| NM_021103 | LUA#41 | 591.5 | 1730 | 2238 | 2731 | 2283 | 1975 | 1828.5 | 1027 |
| NM_002970 | LUA#42 | 92 | 298 | 598 | 589 | 622.5 | 577.5 | 586 | 478 |
| NM_003332 | LUA#43 | 274 | 646 | 1341.5 | 1324 | 1984.5 | 1651 | 2226 | 1241.5 |
| NM_004106 | LUA#44 | 47 | 142 | 253.5 | 302 | 321 | 271 | 273.5 | 267 |
| NM_002982 | LUA#45 | 286 | 2331 | 2514 | 4037 | 4231 | 2722 | 2898 | 1696.5 |
| NM_005375 | LUA#86 | 273 | 1380 | 2455 | 2357.5 | 2068 | 2193 | 2264 | 1273.5 |
| NM_000250 | LUA#87 | 261.5 | 1504 | 2135 | 2243 | 1820 | 2094 | 1863 | 1124 |
| NM_004526 | LUA#88 | 108 | 642 | 1081.5 | 1122 | 840 | 1033 | 1012 | 641.5 |
| NM_004741 | LUA#89 | 131 | 383 | 972 | 1003 | 933 | 920 | 1126 | 571 |
| NM_002467 | LUA#90 | 383 | 983.5 | 1643 | 1800 | 1920 | 1518 | 1725 | 1034 |
| ACTB | LUA#91 | 344 | 2113 | 2976 | 3020 | 2200 | 2601 | 2521 | 1195 |
| TFRC | LUA#92 | 89 | 400 | 794 | 910.5 | 811 | 827.5 | 1042 | 592 |
| GAPDH_5 | LUA#93 | 238 | 1501 | 2205.5 | 2064 | 1482 | 1768.5 | 1737 | 843 |
| GAPDH_M | LUA#94 | 642 | 3318 | 3783.5 | 3886 | 3205 | 3303 | 3248 | 1513 |
| GAPDH_3 | LUA#95 | 1659 | 3754 | 4065 | 4240 | 3880 | 3379 | 3353.5 | 1707.5 |

Table 6A-6B Experiment 1

TABLE 6
Table 6A Experiment 1- Blank and DMSO

| description | FlexMap ID | BLANK | BLANK | DMSO | DMSO | DMSO | DMSO | DMSO | DMSO | DMSO | DMSO | DMSO | DMSO |
|---|---|---|---|---|---|---|---|---|---|---|---|---|---|
| NM_005736 | LUA#1 | 15 | 30 | 232 | 237 | 270.5 | 227.5 | 243 | 224 | 230 | 261 | 275.5 | 258 |
| NM_000070 | LUA#2 | 23.5 | 30 | 234.5 | 198 | 193 | 219.5 | 197.5 | 187 | 203 | 225.5 | 242.5 | 234 |
| NM_018217 | LUA#3 | 16 | 21 | 510 | 513 | 510 | 505 | 507.5 | 458 | 490 | 523 | 534 | 530 |
| NM_004782 | LUA#4 | 34.5 | 31 | 449.5 | 592.5 | 581 | 603 | 605 | 552 | 606 | 605 | 615.5 | 608.5 |
| NM_014962 | LUA#5 | 26.5 | 28 | 318.5 | 457 | 473 | 482.5 | 486 | 438 | 467 | 456 | 500 | 460 |
| NM_004514 | LUA#46 | 29 | 35.5 | 553 | 424 | 419 | 452.5 | 436 | 394 | 449 | 509 | 477 | 470.5 |
| NM_006773 | LUA#47 | 30 | 38 | 252 | 308 | 313 | 339 | 338 | 285 | 325 | 323.5 | 326 | 335 |
| NM_014288 | LUA#48 | 31 | 37.5 | 203.5 | 138 | 132.5 | 137 | 136 | 125.5 | 138 | 141 | 143 | 137 |
| NM_017440 | LUA#49 | 34 | 30 | 106 | 98.5 | 105.5 | 110 | 118 | 94 | 107 | 121 | 116 | 117 |
| NM_007331 | LUA#50 | 19 | 24 | 187 | 130 | 120 | 134 | 128.5 | 121 | 140 | 150 | 149.5 | 138 |
| NM_173823 | LUA#6 | 33 | 28.5 | 428 | 500 | 495 | 500.5 | 533 | 460 | 522 | 544 | 505 | 517.5 |
| NM_000962 | LUA#7 | 29 | 39.5 | 425 | 368.5 | 370 | 383.5 | 376 | 339 | 395 | 423 | 419 | 404 |
| NM_003825 | LUA#8 | 32 | 26 | 500.5 | 352 | 327 | 357 | 355 | 311 | 369 | 381 | 376 | 370.5 |
| NM_016061 | LUA#9 | 28 | 27 | 261 | 224 | 217 | 222 | 223 | 203 | 234 | 250 | 237 | 237.5 |
| NM_000153 | LUA#10 | 20 | 32.5 | 287 | 213.5 | 203.5 | 213 | 213 | 183 | 221.5 | 244 | 231 | 226 |
| NM_006948 | LUA#51 | 31 | 34 | 588 | 600 | 609.5 | 609.5 | 623 | 565 | 621.5 | 647 | 629 | 659.5 |
| NM_004631 | LUA#52 | 22 | 12 | 291 | 268 | 269 | 284.5 | 285.5 | 261 | 274 | 297 | 287 | 281.5 |
| NM_002358 | LUA#53 | 29 | 33 | 343 | 355 | 354.5 | 386 | 378 | 328.5 | 361 | 387 | 397 | 374 |
| NM_013402 | LUA#54 | 24 | 33 | 291.5 | 283 | 276 | 301 | 284.5 | 248.5 | 281 | 282 | 301 | 298 |
| NM_000875 | LUA#55 | 25 | 24 | 51 | 60 | 56 | 65 | 64 | 52.5 | 58.5 | 60.5 | 57 | 66 |
| NM_001974 | LUA#11 | 28 | 37 | 98 | 105 | 101 | 104 | 113 | 96 | 109.5 | 104 | 108 | 109.5 |
| NM_000632 | LUA#12 | 24 | 29 | 84 | 55.5 | 63.5 | 56 | 66 | 55 | 55 | 59 | 53 | 66 |
| NM_006457 | LUA#13 | 32.5 | 36 | 110 | 117 | 124 | 126 | 145 | 118 | 133 | 115 | 134 | 143 |
| NM_000698 | LUA#14 | 28 | 30 | 375.5 | 380.5 | 398 | 392.5 | 379.5 | 357 | 401 | 372 | 411 | 385 |
| NM_032571 | LUA#15 | 23 | 32 | 25 | 28 | 35 | 34 | 27 | 30 | 33 | 37 | 36 | 31.5 |
| NM_006138 | LUA#56 | 25 | 33 | 986.5 | 1084 | 1076 | 1125 | 1116.5 | 986 | 1104 | 1154 | 1109 | 1139 |
| NM_015201 | LUA#57 | 28 | 29 | 772 | 752 | 787 | 792 | 735 | 698 | 745 | 806 | 840 | 793 |
| NM_006985 | LUA#58 | 37 | 37 | 171 | 130 | 135.5 | 134 | 134 | 116 | 127 | 129 | 131.5 | 139 |
| NM_004095 | LUA#59 | 46 | 35 | 1656 | 1443.5 | 1428 | 1459 | 1379 | 1264 | 1389 | 1369.5 | 1487.5 | 1530 |
| NM_005914 | LUA#60 | 39 | 28 | 1214 | 1110 | 1128 | 1193 | 1211.5 | 1044.5 | 1117 | 1091 | 1211 | 1243 |
| NM_007282 | LUA#16 | 22 | 26.5 | 50 | 49 | 45 | 53 | 54 | 42 | 53 | 47.5 | 53.5 | 60 |
| NM_003644 | LUA#17 | 36.5 | 35 | 226 | 231.5 | 232 | 255 | 246.5 | 238.5 | 260 | 243 | 240 | 233.5 |
| NM_001498 | LUA#18 | 26.5 | 24 | 401.5 | 209 | 205.5 | 211 | 210 | 173.5 | 207 | 236 | 229.5 | 214 |
| NM_003172 | LUA#19 | 20 | 31 | 259 | 231 | 229 | 249 | 245 | 200 | 246 | 242 | 253 | 260 |
| NM_004723 | LUA#20 | 29 | 28 | 598 | 410 | 414 | 404.5 | 420.5 | 329.5 | 372.5 | 382 | 421 | 452 |
| NM_014366 | LUA#61 | 41 | 34 | 705 | 625.5 | 632 | 653 | 617 | 582 | 643 | 655.5 | 677 | 661 |
| NM_003581 | LUA#62 | 21 | 32.5 | 278 | 50 | 115 | 61 | 64 | 48 | 58.5 | 60 | 56.5 | 53.5 |
| NM_018115 | LUA#63 | 32 | 27 | 601.5 | 675 | 694 | 689 | 724 | 574.5 | 665 | 645 | 735.5 | 787 |
| NM_021974 | LUA#64 | 34.5 | 33 | 1652 | 1660 | 1680.5 | 1724 | 1666 | 1479 | 1664 | 1617 | 1804 | 1849 |
| NM_024045 | LUA#65 | 34 | 28 | 262.5 | 235.5 | 241 | 247 | 242 | 208 | 231 | 242 | 253 | 252 |
| NM_004079 | LUA#21 | 33.5 | 28 | 73 | 65 | 73 | 71 | 67 | 54.5 | 71 | 62 | 63 | 73 |
| NM_000414 | LUA#22 | 37.5 | 24.5 | 222 | 134 | 144.5 | 152 | 143.5 | 124 | 136.5 | 142 | 147 | 138 |
| NM_001684 | LUA#23 | 20 | 32 | 39 | 38 | 34 | 49 | 43 | 37 | 44 | 46 | 45 | 45 |
| NM_003879 | LUA#24 | 39 | 28 | 51 | 46 | 56 | 53 | 58 | 45.5 | 54.5 | 56 | 54 | 56 |
| NM_002166 | LUA#25 | 29.5 | 32 | 60 | 66.5 | 82 | 81 | 76 | 70 | 74 | 75.5 | 75.5 | 79.5 |
| NM_005952 | LUA#66 | 29 | 40.5 | 534.5 | 534 | 573 | 602.5 | 553 | 529 | 592.5 | 619 | 556 | 570 |
| NM_001034 | LUA#67 | 24 | 21 | 552 | 584 | 586 | 586 | 599 | 530 | 603 | 644 | 604 | 601 |
| NM_003132 | LUA#68 | 32.5 | 29 | 1555 | 1730 | 1763 | 1807 | 1782.5 | 1645 | 1830 | 1833 | 1824.5 | 1844.5 |
| NM_018164 | LUA#69 | 29 | 28 | 428.5 | 431 | 425 | 418 | 411.5 | 361 | 433 | 438 | 462 | 499 |
| NM_014573 | LUA#70 | 41 | 44 | 589 | 360 | 361.5 | 387 | 383 | 338 | 399.5 | 419 | 400.5 | 397 |
| NM_014333 | LUA#26 | 23 | 29 | 69 | 69 | 85.5 | 84 | 85 | 65.5 | 77 | 82 | 83 | 82 |
| NM_006432 | LUA#27 | 25 | 31 | 312 | 272 | 276 | 294 | 275 | 247 | 270 | 306 | 302 | 290 |
| NM_000433 | LUA#28 | 30 | 22.5 | 252 | 142 | 135 | 135 | 138 | 120 | 141 | 153 | 146 | 160 |
| NM_000147 | LUA#29 | 34 | 25 | 102 | 101 | 102 | 106.5 | 106 | 84 | 97 | 102.5 | 106 | 100 |
| NM_000584 | LUA#30 | 30.5 | 31 | 1070 | 726 | 741 | 743 | 750 | 661 | 757 | 785 | 777.5 | 788 |
| NM_006452 | LUA#71 | 41.5 | 42.5 | 147.5 | 108.5 | 115 | 115 | 114.5 | 106 | 120.5 | 125 | 111 | 110 |
| NM_005915 | LUA#72 | 27 | 30.5 | 159.5 | 116 | 112 | 117 | 118 | 102 | 113 | 121 | 125 | 123.5 |
| NM_005980 | LUA#73 | 29.5 | 39 | 1277 | 1452 | 1399.5 | 1493 | 1439 | 1372 | 1425 | 1473 | 1473 | 1479 |
| NM_002539 | LUA#74 | 34 | 32 | 1594.5 | 1793 | 1769 | 1801 | 1828 | 1620 | 1725 | 1827 | 1992 | 1916 |
| NM_019058 | LUA#75 | 38 | 39.5 | 1044.5 | 886.5 | 872 | 897 | 876.5 | 792 | 830 | 814.5 | 946 | 930 |
| NM_004152 | LUA#31 | 26 | 28.5 | 1525 | 1952.5 | 2027 | 1926 | 2057 | 1856 | 1823 | 1940 | 1987 | 2025 |
| NM_004602 | LUA#32 | 34 | 28 | 195.5 | 192 | 193 | 200 | 203 | 178 | 200 | 203.5 | 204.5 | 198 |
| NM_018890 | LUA#33 | 40 | 39.5 | 771.5 | 596.5 | 617 | 647 | 633 | 592.5 | 692.5 | 700 | 684 | 645 |
| NM_001101 | LUA#34 | 31 | 27 | 1771.5 | 1972.5 | 1931 | 2061 | 1922 | 1789 | 1912 | 2051 | 2122.5 | 2118.5 |
| NM_006019 | LUA#35 | 38 | 22 | 514 | 534 | 509 | 553 | 526 | 486 | 567 | 589 | 577 | 552 |
| NM_004134 | LUA#76 | 33 | 32 | 955 | 610.5 | 597 | 619 | 626 | 576 | 611 | 646 | 607 | 605.5 |
| NM_005008 | LUA#77 | 36 | 51 | 962 | 911 | 889 | 908.5 | 906 | 806 | 874 | 855.5 | 958.5 | 916 |
| NM_020117 | LUA#78 | 31 | 35 | 1235.5 | 1359 | 1327 | 1435.5 | 1350.5 | 1243 | 1362 | 1424 | 1399.5 | 1404.5 |
| NM_001469 | LUA#79 | 39.5 | 40 | 1511 | 1917 | 1890 | 1972.5 | 1994.5 | 1780.5 | 1858 | 1848 | 1988 | 2024 |
| NM_021203 | LUA#80 | 41 | 42 | 1421.5 | 1578 | 1531.5 | 1552 | 1535.5 | 1367 | 1535 | 1558 | 1637 | 1653 |
| NM_002624 | LUA#36 | 33 | 26.5 | 1100 | 1042 | 1019.5 | 1048 | 1005 | 957.5 | 1035 | 1063 | 1020 | 1055 |
| NM_004759 | LUA#37 | 35 | 39 | 70.5 | 84 | 70.5 | 73 | 75 | 58 | 71 | 72.5 | 62 | 131 |
| NM_002664 | LUA#38 | 29 | 25 | 1467 | 1319 | 1313.5 | 1370 | 1303.5 | 1184 | 1326.5 | 1428 | 1394 | 1398 |
| NM_000211 | LUA#39 | 36 | 33.5 | 932 | 663 | 621.5 | 675 | 660 | 612.5 | 679 | 699 | 702 | 686 |
| NM_002468 | LUA#40 | 23 | 25 | 134.5 | 130 | 139 | 152 | 143.5 | 131.5 | 143.5 | 144 | 147 | 152 |
| NM_000884 | LUA#81 | 40 | 46 | 1284 | 1582 | 1611 | 1668 | 1647 | 1514 | 1652 | 1669 | 1670 | 1688 |
| NM_003752 | LUA#82 | 41 | 46 | 216 | 245 | 255 | 244 | 249 | 219 | 230.5 | 266.5 | 244 | 254.5 |
| NM_018256 | LUA#83 | 31.5 | 28.5 | 665.5 | 1012 | 1090 | 1120 | 1141.5 | 1160 | 1115 | 953.5 | 1044 | 1014 |
| NM_001948 | LUA#84 | 34 | 27 | 180.5 | 155.5 | 156 | 157 | 154 | 137 | 150 | 168.5 | 161 | 159 |
| NM_005566 | LUA#85 | 41 | 34 | 2231 | 2060 | 2128.5 | 2169 | 2106.5 | 1939.5 | 2064.5 | 2145 | 2116.5 | 2100 |
| NM_021103 | LUA#41 | 34.5 | 30 | 1272 | 1437 | 1473 | 1503 | 1443 | 1414 | 1506 | 1456 | 1501 | 1461 |
| NM_002970 | LUA#42 | 41 | 24.5 | 396 | 450.5 | 462 | 478 | 479 | 430.5 | 496 | 471.5 | 480 | 481 |
| NM_003332 | LUA#43 | 34.5 | 35 | 838.5 | 1008 | 1029.5 | 982.5 | 978 | 931 | 1004 | 1061 | 1030 | 1037 |
| NM_004106 | LUA#44 | 27.5 | 30 | 296 | 278 | 282.5 | 302.5 | 282.5 | 276 | 306 | 324 | 291 | 291 |
| NM_002982 | LUA#45 | 25.5 | 32 | 504 | 487 | 513 | 542 | 502 | 467 | 524 | 578.5 | 529 | 521.5 |
| NM_005375 | LUA#86 | 46 | 38 | 1101.5 | 1745 | 1753 | 1785 | 1811 | 1641 | 1712.5 | 1731 | 1726 | 1662.5 |
| NM_000250 | LUA#87 | 39 | 37 | 2256 | 2007.5 | 2043 | 2043 | 2031.5 | 1774 | 1918.5 | 1971 | 2128 | 2032.5 |
| NM_004526 | LUA#88 | 37 | 29 | 853 | 854 | 851 | 889 | 854 | 770 | 833 | 834.5 | 872 | 873 |
| NM_004741 | LUA#89 | 40 | 36.5 | 484.5 | 567 | 584 | 610 | 603 | 564 | 622 | 652 | 598 | 554.5 |
| NM_002467 | LUA#90 | 44 | 52 | 1411.5 | 2347 | 2409 | 2476 | 2397.5 | 2416 | 2415.5 | 2431 | 2435 | 2296 |
| ACTB | LUA#91 | 40 | 39 | 1480 | 1420 | 1437 | 1536.5 | 1514 | 1336 | 1470 | 1606 | 1527 | 1524.5 |
| TFRC | LUA#92 | 49 | 55 | 508 | 556 | 585 | 587.5 | 578.5 | 519 | 572 | 623 | 603 | 591 |
| GAPDH_5 | LUA#93 | 55 | 57.5 | 1707 | 2319.5 | 2460 | 2510 | 2602 | 2496 | 2654 | 2758.5 | 2441.5 | 2356 |
| GAPDH_M | LUA#94 | 51 | 29 | 2351 | 2607 | 2800 | 2937 | 2802 | 2679 | 2809 | 2793 | 2767 | 2698 |
| GAPDH_3 | LUA#95 | 53 | 47 | 2550 | 3645 | 3798 | 3870 | 3894 | 3590.5 | 3663.5 | 3824 | 3859 | 3782 |

Table 6B Experiment 1- Tretinoin

| description | FlexMap ID | Tretinoin | Tretinoin | Tretinoin | Tretinoin | Tretinoin | Tretinoin | Tretinoin | Tretinoin | Tretinoin | Tretinoin |
|---|---|---|---|---|---|---|---|---|---|---|---|
| NM_005736 | LUA#1 | 319 | 351 | 89 | 329 | 319.5 | 138.5 | 309 | 279 | 308 | 336 |
| NM_000070 | LUA#2 | 269.5 | 370 | 104.5 | 354 | 372 | 159 | 336 | 318 | 329.5 | 386 |
| NM_018217 | LUA#3 | 659.5 | 824 | 225.5 | 823 | 800 | 343 | 785 | 727 | 747 | 886 |
| NM_004782 | LUA#4 | 635 | 855 | 232 | 870 | 812.5 | 341 | 855 | 750 | 790.5 | 907 |
| NM_014962 | LUA#5 | 414.5 | 556 | 171.5 | 552.5 | 567 | 232 | 576.5 | 516.5 | 537 | 575.5 |
| NM_004514 | LUA#46 | 262 | 320 | 96 | 306 | 313 | 144.5 | 302 | 286 | 312.5 | 356 |
| NM_006773 | LUA#47 | 139 | 213 | 56.5 | 216 | 219 | 79.5 | 235 | 174 | 213 | 257 |
| NM_014288 | LUA#48 | 55 | 61 | 45 | 60 | 56 | 41.5 | 60 | 59 | 64 | 62 |
| NM_017440 | LUA#49 | 55 | 68 | 40 | 67 | 68 | 48.5 | 66.5 | 62 | 59 | 66 |
| NM_007331 | LUA#50 | 70 | 81 | 42 | 96 | 87 | 53 | 91 | 80 | 85.5 | 97 |
| NM_173823 | LUA#6 | 482.5 | 654 | 186.5 | 675 | 646 | 291 | 604.5 | 573 | 591.5 | 718 |
| NM_000962 | LUA#7 | 663 | 823 | 294 | 793.5 | 807 | 398 | 772 | 764 | 754 | 894 |
| NM_003825 | LUA#8 | 476.5 | 630.5 | 256 | 580 | 609 | 318 | 584 | 550 | 557 | 658 |
| NM_016061 | LUA#9 | 297 | 401 | 128 | 385 | 382 | 173 | 357.5 | 357 | 362.5 | 422 |
| NM_000153 | LUA#10 | 324 | 419.5 | 120 | 384 | 408 | 170 | 374 | 377.5 | 357.5 | 432 |
| NM_006948 | LUA#51 | 592.5 | 791.5 | 224 | 773.5 | 795 | 325 | 762 | 716.5 | 742 | 840.5 |
| NM_004631 | LUA#52 | 238 | 334 | 103 | 331 | 310.5 | 135.5 | 326 | 289.5 | 304 | 352 |
| NM_002358 | LUA#53 | 72 | 98 | 53 | 96 | 99 | 58 | 98 | 85 | 99 | 113 |
| NM_013402 | LUA#54 | 62 | 75 | 43 | 72 | 75.5 | 48.5 | 75 | 71.5 | 72 | 71 |
| NM_000875 | LUA#55 | 53 | 62 | 36 | 62 | 65 | 48 | 74 | 54 | 63 | 68 |
| NM_001974 | LUA#11 | 288 | 435.5 | 95 | 414 | 430 | 141 | 420.5 | 396 | 403 | 466 |
| NM_000632 | LUA#12 | 222 | 292 | 78 | 307 | 277 | 114 | 275.5 | 251 | 254 | 323.5 |
| NM_006457 | LUA#13 | 113 | 135 | 78 | 123 | 136 | 90 | 151.5 | 132 | 144 | 135 |
| NM_000698 | LUA#14 | 1332.5 | 1743 | 503 | 1688 | 1686.5 | 787 | 1606 | 1552.5 | 1562 | 1779 |
| NM_032571 | LUA#15 | 125 | 161 | 60 | 169 | 164.5 | 81 | 172.5 | 146 | 144 | 188 |
| NM_006138 | LUA#56 | 93 | 124 | 51.5 | 113.5 | 115.5 | 66 | 122 | 114.5 | 114 | 130 |
| NM_015201 | LUA#57 | 139 | 222 | 64 | 208 | 203 | 87 | 194.5 | 183 | 181 | 226.5 |
| NM_006985 | LUA#58 | 47 | 59 | 37 | 55 | 56 | 40 | 57 | 51 | 52 | 54 |
| NM_004095 | LUA#59 | 148 | 227 | 78 | 212 | 212 | 101 | 214.5 | 194.5 | 203 | 247 |
| NM_005914 | LUA#60 | 566.5 | 843 | 209 | 866 | 848.5 | 351 | 885 | 795 | 881.5 | 948 |
| NM_007282 | LUA#16 | 39 | 64.5 | 42.5 | 64 | 62 | 56 | 61 | 64.5 | 60.5 | 60 |
| NM_003644 | LUA#17 | 195.5 | 252 | 105 | 266.5 | 259 | 142 | 271.5 | 260 | 282 | 272 |
| NM_001498 | LUA#18 | 279 | 402 | 96 | 374 | 412 | 140 | 360 | 335.5 | 361 | 424 |
| NM_003172 | LUA#19 | 208 | 286 | 86 | 264 | 257 | 121 | 260 | 235 | 243 | 276 |
| NM_004723 | LUA#20 | 318 | 394 | 127 | 418.5 | 388.5 | 184 | 400 | 363.5 | 380 | 446 |
| NM_014366 | LUA#61 | 163 | 235 | 66 | 231 | 235 | 97 | 231 | 209.5 | 213 | 263 |
| NM_003581 | LUA#62 | 147 | 80 | 50 | 65 | 52 | 43 | 50 | 42 | 53 | 57 |
| NM_018115 | LUA#63 | 552.5 | 735.5 | 143 | 690 | 601 | 227.5 | 582 | 418 | 506.5 | 644 |
| NM_021974 | LUA#64 | 872 | 1105 | 293.5 | 1102.5 | 1052.5 | 477.5 | 1068 | 955 | 1003 | 1158 |
| NM_024045 | LUA#65 | 100 | 145 | 52 | 141 | 142 | 69 | 133 | 119 | 124 | 146 |
| NM_004079 | LUA#21 | 93 | 124.5 | 55 | 127 | 122 | 70 | 125.5 | 99.5 | 113 | 129 |
| NM_000414 | LUA#22 | 270 | 442 | 100 | 408.5 | 415 | 145 | 402.5 | 373 | 372.5 | 470.5 |
| NM_001684 | LUA#23 | 54 | 66.5 | 41 | 65 | 65 | 43 | 61 | 63 | 69 | 68 |
| NM_003879 | LUA#24 | 57 | 80 | 41 | 71 | 76 | 53 | 80 | 67 | 72.5 | 77.5 |
| NM_002166 | LUA#25 | 124.5 | 159.5 | 61 | 168 | 159 | 79.5 | 156.5 | 152 | 154 | 180 |
| NM_005952 | LUA#66 | 149 | 198.5 | 72.5 | 189 | 203 | 95.5 | 188.5 | 182 | 172 | 212 |
| NM_001034 | LUA#67 | 157 | 225 | 73 | 209 | 212.5 | 92 | 219 | 185 | 191 | 243 |
| NM_003132 | LUA#68 | 410 | 540 | 148 | 523 | 517 | 212 | 488 | 467.5 | 488 | 596 |
| NM_018164 | LUA#69 | 131 | 165 | 57 | 152.5 | 155 | 75 | 143 | 142 | 140.5 | 177 |
| NM_014573 | LUA#70 | 99 | 153.5 | 61 | 138 | 155 | 79 | 138.5 | 144.5 | 135 | 155 |
| NM_014333 | LUA#26 | 366.5 | 531 | 134 | 492 | 530 | 197.5 | 497 | 459.5 | 472 | 584 |
| NM_006432 | LUA#27 | 1081.5 | 1409.5 | 397 | 1446 | 1405 | 625 | 1345 | 1203 | 1294.5 | 1495 |
| NM_000433 | LUA#28 | 442.5 | 640 | 144 | 605 | 622 | 228.5 | 564 | 532 | 536.5 | 647 |
| NM_000147 | LUA#29 | 573 | 861 | 195.5 | 822.5 | 850 | 302 | 783 | 763 | 764 | 894 |
| NM_000584 | LUA#30 | 1464 | 1938.5 | 476 | 1981.5 | 1945 | 799.5 | 1938 | 1717 | 1765 | 2115 |
| NM_006452 | LUA#71 | 67.5 | 79.5 | 41 | 75 | 68 | 53.5 | 82 | 74 | 78.5 | 76 |
| NM_005915 | LUA#72 | 39 | 54 | 34.5 | 44 | 56 | 41 | 51 | 47 | 44 | 59 |
| NM_005980 | LUA#73 | 106 | 163 | 57 | 142 | 163 | 74 | 149.5 | 151 | 145 | 173 |
| NM_002539 | LUA#74 | 231 | 326 | 85 | 313 | 314.5 | 131 | 300 | 281.5 | 276 | 362 |
| NM_019058 | LUA#75 | 143 | 164 | 59 | 158 | 152 | 79.5 | 148.5 | 129 | 143.5 | 158 |
| NM_004152 | LUA#31 | 1662 | 2073 | 775 | 2127.5 | 2117 | 1110 | 2045 | 1823 | 1944 | 2194 |
| NM_004602 | LUA#32 | 182 | 239 | 88 | 232.5 | 227.5 | 113.5 | 224 | 212 | 215.5 | 258 |
| NM_018890 | LUA#33 | 537.5 | 758 | 187.5 | 788 | 743.5 | 293.5 | 719 | 712 | 705 | 824 |
| NM_001101 | LUA#34 | 2773 | 2969.5 | 1490 | 2968.5 | 2890 | 1977 | 2893.5 | 2694 | 2722 | 3119.5 |
| NM_006019 | LUA#35 | 569 | 818 | 186 | 828 | 762 | 287 | 767 | 734.5 | 746 | 867 |
| NM_004134 | LUA#76 | 207 | 277 | 83.5 | 292 | 306 | 111 | 280 | 266.5 | 278 | 318 |
| NM_005008 | LUA#77 | 307 | 392 | 123 | 401 | 382.5 | 167 | 372 | 338.5 | 343 | 416 |
| NM_020117 | LUA#78 | 408 | 584.5 | 145 | 554 | 564 | 230 | 529.5 | 519 | 527 | 607 |
| NM_001469 | LUA#79 | 809 | 1179 | 284 | 1191 | 1179 | 463 | 1140.5 | 1077 | 1076 | 1221 |
| NM_021203 | LUA#80 | 442.5 | 642.5 | 151 | 578.5 | 611 | 228.5 | 563 | 546 | 547 | 654 |
| NM_002624 | LUA#36 | 1267 | 1418 | 576 | 1447 | 1402 | 820 | 1369 | 1224.5 | 1288 | 1492.5 |
| NM_004759 | LUA#37 | 148 | 139.5 | 53 | 128 | 141 | 67 | 134 | 103 | 116 | 149.5 |
| NM_002664 | LUA#38 | 2157 | 2552 | 892 | 2527 | 2504 | 1337 | 2394 | 2330 | 2325 | 2761 |
| NM_000211 | LUA#39 | 1125 | 1420 | 454.5 | 1349 | 1366.5 | 682 | 1361 | 1315 | 1294.5 | 1488 |
| NM_002468 | LUA#40 | 325 | 448.5 | 121 | 496.5 | 473 | 174.5 | 426.5 | 399.5 | 418 | 492 |
| NM_000884 | LUA#81 | 676 | 871.5 | 250 | 871 | 865 | 389 | 830.5 | 799 | 799 | 946 |
| NM_003752 | LUA#82 | 114 | 144.5 | 71 | 128.5 | 142 | 94 | 137.5 | 124.5 | 130.5 | 145.5 |
| NM_018256 | LUA#83 | 897 | 726 | 388 | 903 | 998 | 589 | 1256.5 | 1208 | 1557 | 747 |
| NM_001948 | LUA#84 | 61 | 73 | 47 | 63 | 71 | 51 | 76 | 66 | 58 | 75 |
| NM_005566 | LUA#85 | 583.5 | 642 | 150 | 607 | 596 | 219 | 577 | 523.5 | 540 | 632.5 |
| NM_021103 | LUA#41 | 2257.5 | 2689 | 925 | 2668.5 | 2611 | 1370 | 2590 | 2454 | 2412 | 2719 |
| NM_002970 | LUA#42 | 1181 | 1595 | 400 | 1478 | 1584 | 651 | 1503 | 1459 | 1455 | 1683.5 |
| NM_003332 | LUA#43 | 2219 | 2571.5 | 1100 | 2688.5 | 2573.5 | 1470 | 2528 | 2325 | 2387.5 | 2641 |
| NM_004106 | LUA#44 | 994 | 1303 | 373 | 1308 | 1315 | 576.5 | 1274.5 | 1218 | 1187 | 1452 |
| NM_002982 | LUA#45 | 3231 | 3797 | 1738 | 3852 | 3752 | 2466 | 3667 | 3451 | 3488 | 3786 |
| NM_005375 | LUA#86 | 523 | 594.5 | 238 | 631.5 | 617 | 351 | 734 | 717 | 780.5 | 638 |
| NM_000250 | LUA#87 | 137 | 194 | 74 | 177 | 187 | 95.5 | 173 | 166 | 164 | 198 |
| NM_004526 | LUA#88 | 150 | 213 | 77 | 208 | 192.5 | 101 | 206.5 | 182.5 | 192 | 223 |
| NM_004741 | LUA#89 | 168 | 215 | 100 | 204 | 198 | 116 | 214 | 204.5 | 205 | 208 |
| NM_002467 | LUA#90 | 702 | 736 | 256 | 792.5 | 842.5 | 399 | 957 | 976 | 1030 | 801 |
| ACTB | LUA#91 | 1929 | 2483 | 818 | 2425.5 | 2563 | 1173 | 2347 | 2296.5 | 2313 | 2609 |
| TFRC | LUA#92 | 191.5 | 285 | 81.5 | 280.5 | 289 | 109 | 272 | 251.5 | 250 | 305 |
| GAPDH_5 | LUA#93 | 998.5 | 1419 | 378.5 | 1433 | 1505.5 | 632 | 1469 | 1347 | 1299 | 1629 |
| GAPDH_M | LUA#94 | 1134 | 1511.5 | 460 | 1520 | 1502 | 698.5 | 1462 | 1334 | 1360.5 | 1575 |
| GAPDH_3 | LUA#95 | 2428 | 2979 | 1102.5 | 2911 | 2912 | 1645 | 2823 | 2559 | 2580 | 2982 |

TABLE 7
Table 7A Experiment 2- Blank and DMSO

| description | FlexMap ID | BLANK | BLANK | DMSO | DMSO | DMSO | DMSO | DMSO | DMSO | DMSO | DMSO | DMSO | DMSO |
|---|---|---|---|---|---|---|---|---|---|---|---|---|---|
| NM_00573 | LUA#1 | 30 | 38 | 48 | 53 | 245 | 256 | 258.5 | 259 | 226 | 275 | 219 | 208 |
| NM_00007 | LUA#2 | 31 | 26.5 | 39 | 39 | 198 | 207 | 202 | 235 | 180.5 | 201 | 193 | 202 |
| NM_01821 | LUA#3 | 34 | 26 | 50.5 | 94 | 550 | 605 | 604 | 639.5 | 531 | 629.5 | 544 | 531 |
| NM_00478 | LUA#4 | 36.5 | 37 | 46 | 85.5 | 600.5 | 569 | 593 | 654.5 | 556 | 689 | 629.5 | 538 |
| NM_01496 | LUA#5 | 39 | 36.5 | 50 | 74 | 486 | 492 | 469 | 496.5 | 415 | 590.5 | 469 | 411 |
| NM_00451 | LUA#46 | 29 | 29.5 | 39 | 90 | 562.5 | 607 | 641 | 633 | 497.5 | 539 | 597.5 | 605 |
| NM_00677 | LUA#47 | 26 | 27.5 | 29 | 60 | 489 | 496 | 528 | 572 | 475 | 479 | 499 | 491 |
| NM_01428 | LUA#48 | 23 | 26 | 27 | 35 | 171 | 170 | 154 | 163.5 | 138.5 | 158 | 161 | 145 |
| NM_01744 | LUA#49 | 35 | 32 | 26 | 36 | 135 | 144 | 129 | 159 | 128 | 134 | 146 | 141 |
| NM_00733 | LUA#50 | 31 | 23 | 25 | 31 | 148 | 161.5 | 182.5 | 182 | 119 | 149.5 | 150 | 150 |
| NM_17382 | LUA#6 | 18 | 20 | 42 | 71 | 502 | 447 | 463 | 504 | 432 | 573.5 | 501 | 462 |
| NM_00096 | LUA#7 | 25 | 25 | 59 | 91 | 401 | 406 | 398 | 403 | 330 | 411 | 397 | 371.5 |
| NM_00382 | LUA#8 | 26.5 | 34 | 101 | 114 | 433 | 423.5 | 419 | 420.5 | 352.5 | 418.5 | 400 | 405 |
| NM_01606 | LUA#9 | 21.5 | 23 | 41 | 60 | 256.5 | 250.5 | 257 | 268 | 222 | 275 | 243.5 | 233 |
| NM_00015 | LUA#10 | 29 | 30 | 38 | 47.5 | 250 | 268 | 260 | 264 | 226 | 264 | 243.5 | 229 |
| NM_00694 | LUA#51 | 28.5 | 41 | 51 | 100 | 708 | 696 | 668 | 684 | 579 | 724 | 667 | 631 |
| NM_00463 | LUA#52 | 32.5 | 35 | 37 | 49 | 308 | 320 | 323 | 328 | 280 | 335 | 309 | 307 |
| NM_00235 | LUA#53 | 30 | 33 | 35 | 42 | 431 | 395 | 435 | 419 | 362 | 429 | 418 | 401 |
| NM_01340 | LUA#54 | 23 | 28 | 32 | 56.5 | 349 | 342 | 360 | 380 | 313.5 | 353 | 340 | 349 |
| NM_00087 | LUA#55 | 20 | 28.5 | 27 | 27 | 85 | 90 | 92 | 110 | 105 | 90.5 | 93 | 79 |
| NM_00197 | LUA#11 | 19 | 30 | 24 | 27 | 129 | 146 | 121.5 | 125 | 94 | 139 | 102 | 102 |
| NM_00063 | LUA#12 | 20 | 33.5 | 24 | 26 | 72 | 80 | 82 | 82 | 76 | 72 | 67 | 81 |
| NM_00645 | LUA#13 | 33 | 35 | 49 | 51 | 140 | 153 | 132 | 152 | 126 | 143 | 118.5 | 92 |
| NM_00069 | LUA#14 | 30 | 27.5 | 47 | 80 | 467 | 500 | 484 | 483 | 398 | 475 | 451 | 418 |
| NM_03257 | LUA#15 | 25 | 23 | 22 | 21 | 21 | 30 | 26 | 22 | 32.5 | 29 | 29 | 23 |
| NM_00613 | LUA#56 | 36 | 29 | 71 | 200 | 1270 | 1262 | 1328.5 | 1397 | 1193 | 1225 | 1253 | 1285 |
| NM_01520 | LUA#57 | 26 | 30 | 45 | 117.5 | 849.5 | 896 | 938.5 | 929 | 725 | 845 | 846 | 878 |
| NM_00698 | LUA#58 | 26 | 33 | 33 | 34 | 146 | 144 | 144.5 | 133 | 111 | 145 | 147 | 111 |
| NM_00409 | LUA#59 | 31 | 38 | 115 | 311 | 1642 | 1798 | 1731 | 1809 | 1469 | 1644 | 1613 | 1462.5 |
| NM_00591 | LUA#60 | 32 | 24 | 71.5 | 218 | 1471 | 1443 | 1509 | 1635.5 | 1124 | 1420.5 | 1406.5 | 1263 |
| NM_00728 | LUA#16 | 27 | 35 | 24.5 | 20 | 42.5 | 46 | 43 | 46 | 45 | 44 | 46 | 40 |
| NM_00364 | LUA#17 | 30 | 34 | 49 | 53.5 | 252 | 221 | 229 | 232 | 192 | 223 | 198 | 217 |
| NM_00149 | LUA#18 | 22 | 35 | 27 | 37 | 236 | 268 | 276.5 | 300 | 252 | 263 | 258 | 266 |
| NM_00317 | LUA#19 | 33 | 27 | 30 | 43 | 257 | 266 | 270 | 268 | 218 | 276.5 | 245 | 240 |
| NM_00472 | LUA#20 | 30 | 17 | 45 | 90 | 536 | 581 | 535 | 621 | 482.5 | 569 | 496 | 439 |
| NM_01436 | LUA#61 | 26.5 | 23.5 | 39 | 93 | 765 | 795 | 829 | 876 | 725 | 785 | 785 | 741.5 |
| NM_00358 | LUA#62 | 12.5 | 28.5 | 72.5 | 52 | 69 | 62 | 68 | 55 | 56 | 51 | 62 | 58 |
| NM_01811 | LUA#63 | 32 | 44.5 | 66 | 163 | 1006 | 1121 | 1018 | 1181 | 1223 | 1257 | 1010 | 902 |
| NM_02197 | LUA#64 | 27.5 | 32 | 125 | 353 | 1802.5 | 1974.5 | 2019.5 | 2034 | 1663 | 1901.5 | 1782 | 1687 |
| NM_02404 | LUA#65 | 27.5 | 27.5 | 31 | 47 | 313.5 | 294 | 302 | 313 | 258 | 298 | 293.5 | 261 |
| NM_00407 | LUA#21 | 22.5 | 33 | 23 | 29 | 83 | 81.5 | 66 | 77 | 76 | 84 | 77 | 71 |
| NM_00041 | LUA#22 | 29 | 26 | 35 | 31 | 178 | 175 | 188 | 202 | 163 | 186 | 186 | 167 |
| NM_00168 | LUA#23 | 39 | 32 | 22 | 20.5 | 43 | 41 | 41.5 | 42 | 34 | 27 | 40 | 37 |
| NM_00387 | LUA#24 | 27 | 34 | 27.5 | 23 | 54 | 52.5 | 56 | 58 | 52 | 60 | 50 | 47 |
| NM_00216 | LUA#25 | 29 | 25 | 23 | 27 | 87 | 97 | 96 | 108 | 82 | 86 | 94 | 93 |
| NM_00595 | LUA#66 | 34 | 43.5 | 44.5 | 106 | 752 | 774 | 816 | 850 | 743.5 | 716.5 | 746 | 723 |
| NM_00103 | LUA#67 | 36 | 30 | 45 | 88 | 685 | 724 | 735 | 752 | 501 | 688 | 679 | 713 |
| NM_00313 | LUA#68 | 28 | 28 | 132 | 426 | 2030 | 2020 | 2077.5 | 2026.5 | 1779 | 1959 | 1928.5 | 1955 |
| NM_01816 | LUA#69 | 42.5 | 33 | 40 | 63 | 475 | 477 | 510 | 533 | 439 | 517 | 504 | 497 |
| NM_01457 | LUA#70 | 36 | 41.5 | 43 | 51 | 450 | 419 | 422 | 409 | 334 | 451 | 428 | 397 |
| NM_01433 | LUA#26 | 46 | 39 | 34 | 31 | 98 | 89 | 86 | 100 | 87 | 94 | 84 | 66 |
| NM_00643 | LUA#27 | 37 | 35 | 29 | 53.5 | 339 | 356 | 364 | 386 | 345 | 340 | 360 | 381 |
| NM_00043 | LUA#28 | 33 | 34 | 59 | 29 | 170 | 171 | 158 | 153 | 123 | 171 | 148 | 125 |
| NM_00014 | LUA#29 | 35.5 | 31 | 26 | 26 | 121 | 118 | 137 | 133 | 117 | 117 | 123 | 122.5 |
| NM_00058 | LUA#30 | 23 | 32 | 63 | 127 | 993 | 1017.5 | 1080.5 | 1142.5 | 950 | 993 | 1000 | 1062 |
| NM_00645 | LUA#71 | 49 | 36 | 33.5 | 34 | 140 | 135 | 121 | 128 | 105.5 | 135 | 117 | 112 |
| NM_00591 | LUA#72 | 34 | 30 | 35 | 29 | 114 | 124 | 126 | 128 | 116 | 135 | 122 | 110 |
| NM_00598 | LUA#73 | 39 | 32 | 76 | 270.5 | 1527 | 1547 | 1567 | 1651 | 1425 | 1597 | 1547 | 1462 |
| NM_00253 | LUA#74 | 43 | 35.5 | 117 | 366 | 2091 | 2193.5 | 2209 | 2175 | 1830 | 2082 | 2100 | 1912.5 |
| NM_01905 | LUA#75 | 35 | 31 | 62 | 157 | 1015 | 1152 | 1188 | 1217 | 952 | 1044 | 1044 | 1030 |
| NM_00415 | LUA#31 | 31 | 29.5 | 89.5 | 327 | 1999 | 1862 | 1854 | 1955 | 1630 | 2131 | 1980 | 1691 |
| NM_00460 | LUA#32 | 18 | 39.5 | 45 | 77.5 | 233.5 | 267 | 235 | 228.5 | 174 | 269.5 | 219 | 221 |
| NM_01889 | LUA#33 | 39 | 39 | 36 | 89 | 796.5 | 742 | 744 | 789.5 | 607 | 728.5 | 728 | 731.5 |
| NM_00110 | LUA#34 | 32.5 | 36.5 | 162.5 | 461 | 2101 | 2075 | 2065 | 2052.5 | 1748.5 | 2089.5 | 2074 | 1945 |
| NM_00601 | LUA#35 | 34 | 35 | 39 | 87 | 598 | 650 | 705 | 709 | 566 | 616 | 646 | 700 |
| NM_00413 | LUA#76 | 47.5 | 33 | 54 | 116 | 808 | 818 | 818 | 830 | 705 | 810 | 760.5 | 725 |
| NM_00500 | LUA#77 | 43 | 35 | 57 | 151 | 975.5 | 1026 | 1002.5 | 1038 | 842.5 | 1024 | 953 | 860 |
| NM_02011 | LUA#78 | 35 | 31.5 | 83 | 292 | 1653 | 1701 | 1746.5 | 1779 | 1445 | 1605.5 | 1611 | 1656 |
| NM_00146 | LUA#79 | 47 | 33.5 | 114 | 331 | 2049 | 2042 | 2027 | 2124 | 1772 | 2105.5 | 1958.5 | 1868 |
| NM_02120 | LUA#80 | 44 | 32 | 88 | 252 | 1671 | 1685 | 1722 | 1739 | 1458 | 1583.5 | 1673.5 | 1542 |
| NM_00262 | LUA#36 | 25 | 30 | 73 | 245.5 | 1176.5 | 1202.5 | 1226 | 1248 | 1132 | 1204 | 1139.5 | 1123 |
| NM_00475 | LUA#37 | 36 | 33 | 26 | 31 | 124 | 121 | 109 | 136 | 135 | 136 | 115 | 117 |
| NM_00266 | LUA#38 | 41 | 37 | 82.5 | 266 | 1521 | 1584 | 1621 | 1668 | 1378 | 1474 | 1502.5 | 1492 |
| NM_00021 | LUA#39 | 33 | 27 | 67 | 123.5 | 769.5 | 707 | 672 | 674 | 541 | 741.5 | 646 | 629.5 |
| NM_00246 | LUA#40 | 20 | 32.5 | 28 | 39 | 153 | 199 | 205 | 208.5 | 161 | 183 | 168 | 171.5 |
| NM_00088 | LUA#81 | 42 | 45 | 166 | 373 | 1693.5 | 1578 | 1629 | 1658 | 1421 | 1696 | 1631 | 1512 |
| NM_00375 | LUA#82 | 49 | 44 | 56 | 67 | 323 | 322 | 329 | 342 | 266 | 307.5 | 293.5 | 274 |
| NM_01825 | LUA#83 | 41 | 40 | 250 | 291 | 1045 | 1031 | 1078 | 1037.5 | 826 | 1007 | 961 | 985 |
| NM_00194 | LUA#84 | 40 | 40 | 35 | 42 | 213.5 | 203 | 219 | 225 | 180 | 203 | 201 | 195.5 |
| NM_00556 | LUA#85 | 39.5 | 44 | 199 | 520 | 2411.5 | 2445 | 2535.5 | 2462.5 | 2077 | 2326 | 2375 | 2334 |
| NM_02110 | LUA#41 | 30.5 | 38 | 97 | 247 | 1549 | 1351 | 1575 | 1693 | 1430.5 | 1500 | 1527.5 | 1296.5 |
| NM_00297 | LUA#42 | 36 | 45 | 24.5 | 52 | 522 | 484 | 507 | 532.5 | 440 | 542 | 529 | 519 |
| NM_00333 | LUA#43 | 35 | 35 | 60 | 178 | 1034.5 | 1140 | 1065 | 1157 | 988 | 1085 | 1058 | 1004 |
| NM_00410 | LUA#44 | 24 | 27 | 30.5 | 55 | 393 | 378 | 404 | 433.5 | 367 | 407 | 389 | 403 |
| NM_00298 | LUA#45 | 20 | 34 | 32 | 94 | 652 | 675 | 707 | 713.5 | 646 | 638 | 670 | 685 |
| NM_00537 | LUA#86 | 34.5 | 37 | 149.5 | 354 | 1811 | 1867 | 2001.5 | 1991.5 | 1718 | 1807 | 1813 | 1899 |
| NM_00025 | LUA#87 | 33 | 40 | 147.5 | 510 | 2353 | 2404 | 2415.5 | 2430 | 2078 | 2358 | 2296 | 2225 |
| NM_00452 | LUA#88 | 40 | 36 | 75 | 182.5 | 1064 | 1120 | 1093 | 1099 | 896 | 1035 | 1034 | 954.5 |
| NM_00474 | LUA#89 | 33 | 33 | 74 | 119.5 | 810 | 852 | 879.5 | 840 | 689.5 | 792 | 797.5 | 738 |
| NM_00246 | LUA#90 | 52.5 | 56 | 369 | 760 | 2507.5 | 2577 | 2640 | 2642 | 2322 | 2586 | 2604.5 | 2599 |
| ACTB | LUA#91 | 55.5 | 44 | 100 | 318 | 1796 | 1791 | 1930 | 1940.5 | 1558 | 1747.5 | 1799 | 1831 |
| TFRC | LUA#92 | 53 | 46.5 | 56 | 94 | 737 | 788 | 844 | 868.5 | 630 | 733 | 791 | 797 |
| GAPDH_5 | LUA#93 | 50 | 39 | 192 | 807 | 2708.5 | 2707 | 2729 | 2828 | 2320 | 2618 | 2741 | 2716 |
| GAPDH_M | LUA#94 | 43 | 42 | 201 | 737 | 3051 | 3052 | 3041 | 3075.5 | 2623 | 3060 | 2962 | 2834.5 |
| GAPDH_3 | LUA#95 | 45.5 | 41 | 616.5 | 1663 | 3524 | 3712 | 3728 | 3841 | 3284 | 3651.5 | 3806 | 3593 |

Table 7B Experiment 2- Tretinoin

| description | FlexMap ID | Tretinoin | Tretinoin | Tretinoin | Tretinoin | Tretinoin | Tretinoin | Tretinoin | Tretinoin | Tretinoin | Tretinoin |
|---|---|---|---|---|---|---|---|---|---|---|---|
| NM_005736 | LUA#1 | 36.5 | 390 | 90.5 | 408 | 411 | 385 | 392 | 414.5 | 384.5 | 298.5 |
| NM_000070 | LUA#2 | 34 | 444 | 120 | 393 | 393 | 419 | 422 | 444 | 437.5 | 358 |
| NM_018217 | LUA#3 | 48 | 935 | 258 | 992 | 1053.5 | 947 | 966 | 1022.5 | 980 | 922 |
| NM_004782 | LUA#4 | 45 | 979.5 | 253 | 1005 | 1030 | 914 | 929 | 1044 | 1036.5 | 932 |
| NM_014962 | LUA#5 | 39 | 595.5 | 160 | 670.5 | 705 | 677.5 | 678 | 710 | 691 | 618 |
| NM_004514 | LUA#46 | 25 | 469 | 99 | 428 | 445 | 422 | 420.5 | 460 | 399 | 424.5 |
| NM_006773 | LUA#47 | 28 | 270 | 68 | 273 | 283 | 272 | 279 | 305 | 284 | 354.5 |
| NM_014288 | LUA#48 | 22 | 52 | 33 | 60 | 57 | 57 | 57 | 65 | 63.5 | 55 |
| NM_017440 | LUA#49 | 29 | 88 | 42 | 78 | 97 | 87.5 | 84 | 86.5 | 91 | 108 |
| NM_007331 | LUA#50 | 24 | 115 | 47 | 95.5 | 96 | 97.5 | 94.5 | 111.5 | 102 | 115 |
| NM_173823 | LUA#6 | 36 | 736 | 182.5 | 758 | 734.5 | 658.5 | 681 | 816 | 760 | 592 |
| NM_000962 | LUA#7 | 65 | 900 | 253 | 845 | 904.5 | 846 | 846 | 930 | 873 | 822.5 |
| NM_003825 | LUA#8 | 102 | 772 | 220 | 800 | 733 | 738 | 727 | 764 | 721 | 689.5 |
| NM_016061 | LUA#9 | 48 | 458 | 121 | 470.5 | 464 | 468 | 459 | 503.5 | 450 | 440 |
| NM_000153 | LUA#10 | 45 | 539 | 124 | 511 | 505 | 473 | 501 | 542 | 487 | 484 |
| NM_006948 | LUA#51 | 51 | 974 | 248 | 1014 | 1006 | 963.5 | 958.5 | 1024.5 | 980 | 887 |
| NM_004631 | LUA#52 | 38.5 | 391 | 97 | 396 | 399.5 | 399 | 401.5 | 424 | 399 | 397 |
| NM_002358 | LUA#53 | 33.5 | 103 | 39 | 109 | 103 | 96 | 97 | 119 | 109 | 94 |
| NM_013402 | LUA#54 | 30 | 82 | 46 | 90.5 | 95.5 | 85 | 84.5 | 97 | 89 | 83 |
| NM_000875 | LUA#55 | 28.5 | 81 | 36 | 79 | 80 | 85 | 90 | 87.5 | 89 | 104 |
| NM_001974 | LUA#11 | 30 | 516.5 | 92.5 | 533 | 515 | 493 | 470 | 539 | 494 | 340.5 |
| NM_000632 | LUA#12 | 26 | 491.5 | 96 | 406 | 400 | 360.5 | 374 | 395 | 369.5 | 476 |
| NM_006457 | LUA#13 | 43 | 115.5 | 65 | 115.5 | 118 | 120 | 131 | 117 | 131 | 121 |
| NM_000698 | LUA#14 | 64.5 | 1902 | 539 | 1934.5 | 1914.5 | 1773 | 1769 | 1871.5 | 1787 | 1685 |
| NM_032571 | LUA#15 | 22.5 | 244 | 58.5 | 228 | 234 | 208 | 205 | 239 | 228 | 244 |
| NM_006138 | LUA#56 | 32.5 | 115.5 | 48 | 111 | 127 | 118 | 119.5 | 120 | 124 | 117 |
| NM_015201 | LUA#57 | 27 | 266 | 63 | 252 | 243 | 230 | 244 | 268.5 | 241 | 245.5 |
| NM_006985 | LUA#58 | 33 | 55 | 33 | 50 | 52 | 54 | 60 | 63 | 56 | 50 |
| NM_004095 | LUA#59 | 31 | 287 | 77 | 286.5 | 293 | 270 | 294 | 331 | 293 | 227 |
| NM_005914 | LUA#60 | 42 | 871 | 213.5 | 1091 | 1131 | 1081.5 | 1125 | 1172.5 | 1161.5 | 1104 |
| NM_007282 | LUA#16 | 22 | 61 | 26 | 69 | 64 | 59 | 62 | 61 | 58 | 53 |
| NM_003644 | LUA#17 | 41 | 319 | 69 | 269 | 274 | 192 | 231 | 300 | 274 | 259 |
| NM_001498 | LUA#18 | 32 | 521 | 110.5 | 507.5 | 513 | 441.5 | 467 | 531.5 | 494.5 | 511 |
| NM_003172 | LUA#19 | 36 | 333 | 82 | 370 | 365 | 330 | 352 | 380 | 333 | 312.5 |
| NM_004723 | LUA#20 | 34 | 472.5 | 134.5 | 498 | 506 | 470 | 487.5 | 538 | 501 | 420 |
| NM_014366 | LUA#61 | 30 | 304.5 | 66 | 297 | 291 | 286.5 | 300 | 310.5 | 298.5 | 321 |
| NM_003581 | LUA#62 | 39 | 114 | 50 | 78 | 85 | 59.5 | 55 | 53 | 55 | 54 |
| NM_018115 | LUA#63 | 54 | 1440 | 356 | 1028 | 1210 | 935.5 | 1175 | 1004 | 1238 | 1270 |
| NM_021974 | LUA#64 | 49.5 | 1272 | 295 | 1368.5 | 1315 | 1193.5 | 1269 | 1365 | 1286.5 | 1160 |
| NM_024045 | LUA#65 | 29 | 163.5 | 48 | 164.5 | 175.5 | 158 | 157 | 182 | 178 | 149 |
| NM_004079 | LUA#21 | 28 | 144 | 49 | 133 | 133.5 | 142 | 143 | 152 | 150 | 147 |
| NM_000414 | LUA#22 | 34 | 547 | 115.5 | 596 | 561.5 | 550.5 | 543 | 598.5 | 564.5 | 544 |
| NM_001684 | LUA#23 | 30 | 98 | 38.5 | 66 | 79 | 68 | 77.5 | 83 | 71 | 75 |
| NM_003879 | LUA#24 | 22 | 93 | 39 | 91.5 | 86 | 84 | 82 | 97 | 95 | 96.5 |
| NM_002166 | LUA#25 | 30 | 237 | 60 | 230 | 240 | 221 | 218 | 242 | 227 | 254.5 |
| NM_005952 | LUA#66 | 40 | 265 | 65 | 274 | 260 | 263 | 224 | 268 | 243 | 261.5 |
| NM_001034 | LUA#67 | 32.5 | 280 | 65 | 281 | 253 | 271 | 252 | 276 | 260 | 246 |
| NM_003132 | LUA#68 | 37.5 | 721 | 157 | 690.5 | 680 | 633 | 648 | 716 | 635 | 618 |
| NM_018164 | LUA#69 | 34 | 205.5 | 61 | 182 | 188.5 | 182 | 186 | 211 | 198 | 200 |
| NM_014573 | LUA#70 | 32 | 187 | 49 | 179 | 161.5 | 172 | 157.5 | 198 | 178 | 154 |
| NM_014333 | LUA#26 | 41 | 706 | 166 | 712 | 720 | 662 | 633.5 | 726.5 | 720 | 704 |
| NM_006432 | LUA#27 | 52.5 | 1767 | 522 | 1718 | 1798 | 1725 | 1678 | 1814 | 1693 | 1842 |
| NM_000433 | LUA#28 | 34 | 810 | 158 | 860 | 842 | 753 | 724 | 823 | 786.5 | 574 |
| NM_000147 | LUA#29 | 36 | 1106 | 236 | 1132 | 1122.5 | 1051 | 1086 | 1171 | 1102 | 1135 |
| NM_000584 | LUA#30 | 72 | 2315 | 665 | 2389 | 2341.5 | 2247 | 2269 | 2450 | 2339.5 | 2517.5 |
| NM_006452 | LUA#71 | 27 | 83.5 | 39 | 93 | 90 | 88 | 96 | 90 | 86 | 86 |
| NM_005915 | LUA#72 | 30 | 47 | 33 | 47 | 43 | 44 | 49 | 54 | 47 | 47 |
| NM_005980 | LUA#73 | 33 | 197 | 52 | 212 | 204 | 187 | 188 | 209.5 | 202 | 190 |
| NM_002539 | LUA#74 | 33 | 437 | 89 | 423 | 409 | 392 | 368 | 441 | 416 | 397 |
| NM_019058 | LUA#75 | 35 | 192 | 55.5 | 181.5 | 179 | 176 | 171 | 194 | 177.5 | 194 |
| NM_004152 | LUA#31 | 74 | 2313 | 811 | 2471.5 | 2477.5 | 2269 | 2335.5 | 2470 | 2347 | 1882 |
| NM_004602 | LUA#32 | 42 | 320 | 116 | 337 | 334 | 363 | 355.5 | 296 | 303 | 274 |
| NM_018890 | LUA#33 | 38 | 1071 | 200 | 983 | 992 | 923.5 | 879 | 988 | 934 | 873 |
| NM_001101 | LUA#34 | 211 | 3046 | 1578 | 3029 | 3195 | 2934 | 2997 | 3139.5 | 3017 | 2733 |
| NM_006019 | LUA#35 | 36 | 1059 | 209.5 | 938.5 | 911.5 | 862 | 871 | 956 | 907 | 1033 |
| NM_004134 | LUA#76 | 36.5 | 423.5 | 90 | 417 | 415 | 415 | 383 | 426 | 406 | 372 |
| NM_005008 | LUA#77 | 34 | 452.5 | 108 | 501 | 482 | 432 | 439.5 | 502.5 | 452.5 | 393 |
| NM_020117 | LUA#78 | 35 | 750 | 149 | 739 | 734 | 606 | 654 | 748 | 697 | 734 |
| NM_001469 | LUA#79 | 73 | 1230 | 333.5 | 1437 | 1331.5 | 1308 | 1281.5 | 1360 | 1315 | 1107 |
| NM_021203 | LUA#80 | 37 | 757.5 | 152 | 703 | 701.5 | 650 | 660 | 741 | 657.5 | 657 |
| NM_002624 | LUA#36 | 71 | 1714 | 670 | 1664 | 1757 | 1580.5 | 1616 | 1772.5 | 1647.5 | 1643 |
| NM_004759 | LUA#37 | 26 | 284 | 60.5 | 179 | 207.5 | 192 | 195 | 201 | 206.5 | 235 |
| NM_002664 | LUA#38 | 91 | 2815 | 1002 | 2840.5 | 2805 | 2642 | 2652 | 2875 | 2701.5 | 2723 |
| NM_000211 | LUA#39 | 89 | 1828 | 470 | 1717 | 1734.5 | 1593.5 | 1570 | 1763 | 1639.5 | 1219 |
| NM_002468 | LUA#40 | 35 | 511 | 135 | 556.5 | 549 | 498 | 494 | 589.5 | 531.5 | 584 |
| NM_000884 | LUA#81 | 61 | 951 | 259.5 | 1004 | 988 | 925 | 916 | 1010 | 963.5 | 841 |
| NM_003752 | LUA#82 | 44 | 168 | 66 | 149.5 | 150 | 153 | 162 | 164 | 139 | 142 |
| NM_018256 | LUA#83 | 158 | 455 | 229 | 571 | 556 | 643 | 655 | 483.5 | 568 | 635.5 |
| NM_001948 | LUA#84 | 33 | 79.5 | 40 | 68 | 70.5 | 62 | 68 | 81 | 72 | 70 |
| NM_005566 | LUA#85 | 49 | 851 | 181.5 | 821 | 830 | 748 | 752 | 776.5 | 744 | 745 |
| NM_021103 | LUA#41 | 99 | 2331.5 | 939 | 2558 | 2559 | 793 | 1990 | 2996.5 | 2724 | 2581.5 |
| NM_002970 | LUA#42 | 42 | 1624 | 435.5 | 1759.5 | 1743 | 1655.5 | 1575 | 1823 | 1716.5 | 1563 |
| NM_003332 | LUA#43 | 120 | 2589.5 | 1244 | 2832 | 2821 | 2704 | 2692 | 2772 | 2733 | 2691.5 |
| NM_004106 | LUA#44 | 55 | 1762 | 508 | 1756 | 1799 | 1678 | 1587.5 | 1827 | 1697 | 1748 |
| NM_002982 | LUA#45 | 294 | 3328 | 2094 | 3522 | 3632 | 3562 | 3485 | 3768 | 3697 | 3859 |
| NM_005375 | LUA#86 | 78 | 552 | 158 | 568 | 593 | 585 | 589 | 587 | 565 | 642 |
| NM_000250 | LUA#87 | 39 | 249 | 70 | 246 | 243 | 232 | 239.5 | 253 | 236 | 228 |
| NM_004526 | LUA#88 | 31 | 244 | 69 | 270 | 260 | 240.5 | 243 | 277 | 256 | 233 |
| NM_004741 | LUA#89 | 57 | 370.5 | 99 | 329 | 324 | 317 | 325 | 328 | 327 | 402.5 |
| NM_002467 | LUA#90 | 108 | 762.5 | 205 | 792 | 798 | 823 | 776 | 759 | 741 | 823 |
| ACTB | LUA#91 | 107 | 2939 | 1020 | 2820 | 2870 | 2791 | 2727 | 2873.5 | 2807 | 2986 |
| TFRC | LUA#92 | 48 | 413 | 83 | 375 | 381 | 358 | 345.5 | 382 | 346 | 400.5 |
| GAPDH_5 | LUA#93 | 72 | 1965.5 | 509 | 1847 | 2001 | 1834.5 | 1691 | 1994 | 1888 | 1977.5 |
| GAPDH_M | LUA#94 | 73 | 1871 | 514 | 1911 | 2010.5 | 1693.5 | 1762.5 | 1932.5 | 1814 | 1595.5 |
| GAPDH_3 | LUA#95 | 139.5 | 2850 | 1137 | 3025 | 3066 | 2936 | 2973 | 3162.5 | 3075 | 2896 |

TABLE 8
Table 8A Experiment 3- Blank and DMSO

| description | FlexMap ID | BLANK | BLANK | DMSO | DMSO | DMSO | DMSO | DMSO | DMSO | DMSO | DMSO | DMSO | DMSO |
|---|---|---|---|---|---|---|---|---|---|---|---|---|---|
| NM_005736 | LUA#1 | 28 | 33.5 | 247 | 240.5 | 214.5 | 233 | 240 | 250 | 272 | 276 | 278.5 | 286.5 |
| NM_000070 | LUA#2 | 26 | 29.5 | 179 | 187.5 | 181 | 162.5 | 162 | 182.5 | 226 | 229 | 239.5 | 231 |
| NM_018217 | LUA#3 | 25 | 32 | 484 | 551.5 | 483 | 494.5 | 485 | 543 | 601 | 647.5 | 630 | 584 |
| NM_004782 | LUA#4 | 27.5 | 38.5 | 467 | 617 | 560.5 | 616 | 627.5 | 630 | 688 | 723.5 | 787.5 | 652.5 |
| NM_014962 | LUA#5 | 26.5 | 32 | 364.5 | 495 | 443 | 455 | 463 | 497 | 492 | 511 | 543 | 485.5 |
| NM_004514 | LUA#46 | 26 | 28 | 585 | 474.5 | 436 | 454 | 440 | 463 | 547 | 554.5 | 530 | 493 |
| NM_006773 | LUA#47 | 32 | 19 | 328 | 444 | 453 | 412 | 408 | 448.5 | 475.5 | 487 | 477 | 443 |
| NM_014288 | LUA#48 | 29 | 29 | 169 | 131.5 | 122.5 | 122 | 129 | 139.5 | 161 | 155.5 | 151.5 | 138 |
| NM_017440 | LUA#49 | 33.5 | 28 | 150 | 137 | 127.5 | 129 | 128 | 151 | 168 | 160.5 | 156 | 146 |
| NM_007331 | LUA#50 | 28 | 27.5 | 188 | 151 | 128 | 143 | 134.5 | 151 | 161 | 178 | 167 | 157 |
| NM_173823 | LUA#6 | 33.5 | 28 | 393 | 516 | 444 | 460.5 | 492 | 529 | 559.5 | 560 | 558 | 553.5 |
| NM_000962 | LUA#7 | 28 | 25 | 386.5 | 348 | 336 | 360 | 334 | 380 | 421.5 | 451 | 403.5 | 409 |
| NM_003825 | LUA#8 | 26 | 24.5 | 436 | 356 | 336.5 | 354.5 | 340 | 398 | 423 | 431 | 429.5 | 414 |
| NM_016061 | LUA#9 | 32 | 29 | 268 | 250 | 217 | 241.5 | 233.5 | 264 | 306 | 301 | 301 | 279 |
| NM_000153 | LUA#10 | 35 | 33 | 252 | 211.5 | 203 | 205 | 204 | 222 | 261 | 265 | 258 | 237 |
| NM_006948 | LUA#51 | 25 | 36 | 593 | 643 | 593 | 617 | 609 | 681 | 746 | 742 | 731.5 | 703.5 |
| NM_004631 | LUA#52 | 25 | 25 | 261 | 263 | 246.5 | 264 | 268.5 | 294 | 322 | 331.5 | 319 | 308 |
| NM_002358 | LUA#53 | 26 | 26 | 349 | 419 | 380 | 408 | 394.5 | 444 | 502 | 514 | 485 | 466 |
| NM_013402 | LUA#54 | 21 | 26.5 | 306 | 378 | 334.5 | 336.5 | 340 | 357 | 397.5 | 395 | 388 | 373 |
| NM_000875 | LUA#55 | 23.5 | 30 | 61 | 82 | 87 | 74 | 70 | 88 | 90 | 91 | 94 | 87 |
| NM_001974 | LUA#11 | 28 | 11 | 85 | 84 | 69 | 61 | 67 | 77 | 90 | 87 | 95.5 | 80 |
| NM_000632 | LUA#12 | 33 | 27 | 76 | 59 | 57 | 51 | 50 | 53 | 55.5 | 59 | 58 | 54 |
| NM_006457 | LUA#13 | 31.5 | 24 | 94 | 124 | 145 | 114 | 106 | 89 | 111 | 123 | 110 | 107 |
| NM_000698 | LUA#14 | 17 | 27.5 | 353 | 360.5 | 325 | 320 | 319 | 316.5 | 368 | 400 | 368 | 368 |
| NM_032571 | LUA#15 | 27 | 25 | 25.5 | 19 | 24 | 25.5 | 25 | 23 | 25 | 24 | 25 | 24 |
| NM_006138 | LUA#56 | 32 | 31 | 1076 | 1274 | 1213.5 | 1224 | 1176 | 1226 | 1316 | 1329 | 1312 | 1257 |
| NM_015201 | LUA#57 | 34 | 35 | 805.5 | 834 | 765.5 | 799 | 791 | 852.5 | 958 | 958 | 907 | 885.5 |
| NM_006985 | LUA#58 | 40 | 29.5 | 200 | 157 | 137 | 154 | 161 | 165 | 188 | 200 | 190 | 187 |
| NM_004095 | LUA#59 | 43 | 27 | 1904 | 1757.5 | 1707 | 1798 | 1644 | 1666 | 1925 | 2035 | 1825.5 | 1758.5 |
| NM_005914 | LUA#60 | 34 | 37 | 1376.5 | 1508 | 1561.5 | 1339 | 1246 | 1355 | 1448 | 1595 | 1420 | 1337 |
| NM_007282 | LUA#16 | 21 | 28 | 47 | 36 | 38 | 35.5 | 36 | 42 | 39 | 53 | 44 | 42 |
| NM_003644 | LUA#17 | 34 | 37 | 177.5 | 213 | 201 | 169.5 | 163 | 171 | 177 | 190 | 180.5 | 184 |
| NM_001498 | LUA#18 | 27 | 31 | 342 | 205 | 211 | 226 | 213.5 | 232 | 266 | 284 | 264 | 257 |
| NM_003172 | LUA#19 | 27 | 30 | 252 | 234 | 213 | 226 | 230.5 | 241 | 276 | 286.5 | 272.5 | 265.5 |
| NM_004723 | LUA#20 | 16.5 | 25.5 | 677 | 476 | 477.5 | 461 | 416 | 432.5 | 523 | 560.5 | 511 | 461 |
| NM_014366 | LUA#61 | 32.5 | 36.5 | 853 | 901 | 904 | 928 | 883 | 943.5 | 1045.5 | 1025.5 | 1014 | 934 |
| NM_003581 | LUA#62 | 26 | 24 | 280 | 66 | 48.5 | 39 | 41 | 50 | 42 | 46 | 50 | 40 |
| NM_018115 | LUA#63 | 31 | 39.5 | 1274 | 1143 | 1113 | 1353 | 1430 | 1382 | 1281 | 1275 | 1475.5 | 1295 |
| NM_021974 | LUA#64 | 38 | 40.5 | 1787 | 1842 | 1743 | 1855 | 1727 | 1723 | 2087 | 2161 | 1976 | 1987 |
| NM_024045 | LUA#65 | 28.5 | 30 | 255 | 265 | 267 | 262.5 | 251 | 268.5 | 303 | 311 | 296 | 298 |
| NM_004079 | LUA#21 | 36 | 33 | 73 | 69 | 64 | 60 | 67 | 74 | 87 | 68 | 99 | 71.5 |
| NM_000414 | LUA#22 | 25 | 42 | 240 | 180 | 157 | 170 | 162 | 184.5 | 198 | 198 | 200 | 184 |
| NM_001684 | LUA#23 | 25 | 24.5 | 26 | 29 | 28 | 27.5 | 37 | 36.5 | 35 | 38 | 35 | 39 |
| NM_003879 | LUA#24 | 21.5 | 18 | 56 | 44 | 50 | 50 | 49 | 47 | 54 | 59.5 | 54.5 | 54 |
| NM_002166 | LUA#25 | 28 | 29 | 75.5 | 91 | 97 | 103 | 89 | 94.5 | 108 | 107 | 104 | 98 |
| NM_005952 | LUA#66 | 38 | 27 | 561.5 | 627.5 | 648 | 673 | 600 | 640 | 672.5 | 738 | 687.5 | 650 |
| NM_001034 | LUA#67 | 26 | 30 | 575.5 | 674 | 631 | 619 | 595 | 641 | 764 | 768 | 739 | 692 |
| NM_003132 | LUA#68 | 27 | 21.5 | 1705 | 1840 | 1717 | 1797.5 | 1693.5 | 1803 | 2003 | 2030 | 1902.5 | 1861.5 |
| NM_018164 | LUA#69 | 37 | 27.5 | 511 | 495.5 | 484 | 532 | 507 | 569 | 633 | 645 | 655 | 594 |
| NM_014573 | LUA#70 | 36.5 | 40 | 463 | 485 | 425.5 | 418.5 | 431.5 | 478 | 534 | 552 | 501 | 510 |
| NM_014333 | LUA#26 | 33 | 28 | 89 | 79 | 94 | 81 | 75 | 89 | 86 | 89 | 83.5 | 79 |
| NM_006432 | LUA#27 | 26 | 26 | 302 | 287 | 273.5 | 302 | 292 | 317.5 | 349 | 360.5 | 335 | 338.5 |
| NM_000433 | LUA#28 | 23 | 36 | 187 | 137 | 111 | 115 | 117 | 133 | 153 | 150 | 136 | 136.5 |
| NM_000147 | LUA#29 | 32 | 34 | 110 | 120 | 117 | 129 | 126 | 125.5 | 142 | 148 | 140 | 137 |
| NM_000584 | LUA#30 | 29 | 29 | 1147 | 905 | 868 | 922 | 872.5 | 926 | 1019 | 1058 | 990 | 959.5 |
| NM_006452 | LUA#71 | 33 | 32 | 178 | 107 | 102 | 91 | 108 | 110.5 | 124 | 130 | 126 | 130.5 |
| NM_005915 | LUA#72 | 37 | 24 | 141.5 | 108 | 96 | 112 | 102 | 114 | 132 | 125 | 126.5 | 117 |
| NM_005980 | LUA#73 | 41 | 28 | 1314 | 1559 | 1544 | 1534 | 1467 | 1517 | 1660 | 1634.5 | 1617 | 1561 |
| NM_002539 | LUA#74 | 43 | 50 | 1863 | 1961.5 | 1903 | 2012.5 | 1865 | 1987 | 2241 | 2169.5 | 2041 | 2033 |
| NM_019058 | LUA#75 | 28.5 | 37.5 | 1168 | 1015 | 974 | 1004 | 959 | 957.5 | 1134 | 1130.5 | 1077 | 1035 |
| NM_004152 | LUA#31 | 34 | 32 | 1698 | 1990 | 1909 | 1973 | 1935.5 | 2211.5 | 2125 | 2252 | 2198 | 2153.5 |
| NM_004602 | LUA#32 | 25 | 22 | 206 | 222 | 198 | 216.5 | 213 | 229 | 275 | 261 | 279 | 255 |
| NM_018890 | LUA#33 | 30 | 44.5 | 703 | 648 | 591.5 | 627 | 607.5 | 669 | 715 | 737 | 695.5 | 690 |
| NM_001101 | LUA#34 | 22 | 23.5 | 2023.5 | 2026 | 1824 | 1841 | 1885 | 2013 | 2164 | 2148 | 2108 | 2048.5 |
| NM_006019 | LUA#35 | 38 | 34 | 489 | 556 | 511 | 540.5 | 528 | 535 | 631.5 | 620 | 610 | 583 |
| NM_004134 | LUA#76 | 26.5 | 26 | 953.5 | 677 | 576 | 622 | 589 | 620.5 | 715.5 | 744 | 688.5 | 681 |
| NM_005008 | LUA#77 | 33 | 37.5 | 882 | 839 | 752 | 791 | 785 | 832.5 | 942 | 961 | 951 | 884 |
| NM_020117 | LUA#78 | 38 | 43 | 1342 | 1519 | 1444.5 | 1498 | 1342 | 1459 | 1657 | 1641 | 1534 | 1457 |
| NM_001469 | LUA#79 | 40 | 43 | 1531 | 2065 | 1894.5 | 1953 | 1964.5 | 1969 | 2199 | 2216 | 2182 | 2115 |
| NM_021203 | LUA#80 | 39 | 45.5 | 1398 | 1482 | 1416 | 1418 | 1394 | 1424.5 | 1659 | 1692 | 1607 | 1533 |
| NM_002624 | LUA#36 | 27 | 24 | 1157.5 | 1111 | 1048.5 | 1073.5 | 1031 | 1095 | 1171.5 | 1194 | 1158 | 1135.5 |
| NM_004759 | LUA#37 | 34 | 24.5 | 115.5 | 84 | 84 | 87.5 | 102.5 | 140 | 130 | 108 | 150 | 114 |
| NM_002664 | LUA#38 | 35 | 29 | 1451 | 1230.5 | 1161 | 1253 | 1186 | 1241 | 1415 | 1470.5 | 1375 | 1311 |
| NM_000211 | LUA#39 | 34 | 35.5 | 778 | 580 | 496 | 516 | 551.5 | 624 | 688.5 | 724.5 | 690.5 | 671 |
| NM_002468 | LUA#40 | 27.5 | 20.5 | 145 | 144 | 156 | 164.5 | 168 | 161 | 168 | 206.5 | 177.5 | 181.5 |
| NM_000884 | LUA#81 | 39 | 43 | 1374 | 1662 | 1457.5 | 1477.5 | 1517.5 | 1579.5 | 1786 | 1770 | 1660 | 1608.5 |
| NM_003752 | LUA#82 | 40 | 44.5 | 206 | 265 | 245 | 260 | 232 | 223 | 257 | 273 | 246 | 241 |
| NM_018256 | LUA#83 | 35 | 32.5 | 583 | 948 | 927 | 859.5 | 840 | 833.5 | 885.5 | 923 | 915.5 | 934 |
| NM_001948 | LUA#84 | 30 | 31.5 | 171.5 | 166 | 151 | 152 | 142 | 158 | 172 | 175 | 169 | 167 |
| NM_005566 | LUA#85 | 39 | 23 | 2576 | 2426 | 2343.5 | 2313 | 2211.5 | 2208.5 | 2364 | 2414 | 2310 | 2268 |
| NM_021103 | LUA#41 | 29.5 | 34 | 1235.5 | 1639 | 1600 | 1483 | 1501 | 1430.5 | 1490 | 1618.5 | 1478 | 1415 |
| NM_002970 | LUA#42 | 25.5 | 28 | 353.5 | 489 | 460 | 492.5 | 480 | 537 | 579.5 | 614 | 565 | 566.5 |
| NM_003332 | LUA#43 | 35 | 38 | 954.5 | 987 | 937 | 1025 | 995 | 1066 | 1181 | 1206 | 1179 | 1122 |
| NM_004106 | LUA#44 | 21 | 26 | 373.5 | 394 | 372.5 | 394 | 375.5 | 419.5 | 457 | 461 | 420 | 418.5 |
| NM_002982 | LUA#45 | 32 | 31 | 603 | 673.5 | 609 | 665 | 596 | 652 | 735.5 | 760 | 709.5 | 655 |
| NM_005375 | LUA#86 | 39.5 | 33 | 1128 | 1776 | 1693 | 1718.5 | 1608 | 1662 | 1785.5 | 1801 | 1732.5 | 1749 |
| NM_000250 | LUA#87 | 45 | 38 | 2308 | 1891 | 1773 | 1821 | 1758 | 1852 | 2090 | 2148.5 | 2015 | 1937 |
| NM_004526 | LUA#88 | 42 | 30 | 1019 | 1079 | 955 | 1021 | 955 | 1007 | 1114 | 1164 | 1092 | 1040 |
| NM_004741 | LUA#89 | 33 | 43.5 | 623 | 778 | 763 | 713 | 731 | 813 | 816 | 821 | 808 | 773 |
| NM_002467 | LUA#90 | 40 | 44 | 1658 | 2414 | 2353 | 2281 | 2242.5 | 2287.5 | 2464 | 2519.5 | 2469.5 | 2358 |
| ACTB | LUA#91 | 37 | 42.5 | 1668 | 1753 | 1743 | 1832 | 1683 | 1785 | 1924 | 1902 | 1857 | 1793 |
| TFRC | LUA#92 | 59.5 | 51 | 543 | 595 | 578 | 659 | 620 | 658.5 | 750 | 715 | 718 | 678 |
| GAPDH_5 | LUA#93 | 42 | 51 | 1954 | 3132.5 | 2965 | 2946 | 2848 | 2897 | 2953 | 2930 | 2799 | 2733.5 |
| GAPDH_M | LUA#94 | 41 | 45.5 | 2721 | 3317 | 3109 | 3039 | 2963 | 3139 | 3320 | 3320 | 3195 | 3068.5 |
| GAPDH_3 | LUA#95 | 47.5 | 45 | 2788.5 | 3887 | 3821 | 3905 | 3912.5 | 3908.5 | 4244.5 | 4050.5 | 4090 | 4030 |

Table 8B Experiment 3- Tretinoin

| description | FlexMap ID | Tretinoin | Tretinoin | Tretinoin | Tretinoin | Tretinoin | Tretinoin | Tretinoin | Tretinoin | Tretinoin | Tretinoin |
|---|---|---|---|---|---|---|---|---|---|---|---|
| NM_005736 | LUA#1 | 55 | 84 | 113 | 205.5 | 298 | 336 | 235 | 38 | 236.5 | 280 |
| NM_000070 | LUA#2 | 54 | 82.5 | 118 | 224 | 328.5 | 330 | 254 | 42 | 274 | 303 |
| NM_018217 | LUA#3 | 109 | 187.5 | 275.5 | 571 | 779 | 801 | 582.5 | 61 | 690.5 | 742 |
| NM_004782 | LUA#4 | 105 | 184.5 | 279 | 539 | 825.5 | 866 | 689 | 62 | 764 | 803.5 |
| NM_014962 | LUA#5 | 82 | 120 | 188.5 | 369 | 544.5 | 551.5 | 435 | 52.5 | 474.5 | 507 |
| NM_004514 | LUA#46 | 47 | 73 | 120 | 233.5 | 284.5 | 300.5 | 206 | 33 | 287 | 281 |
| NM_006773 | LUA#47 | 37 | 51 | 74.5 | 133 | 213 | 245 | 189.5 | 34 | 243 | 218 |
| NM_014288 | LUA#48 | 30 | 25 | 30.5 | 39 | 48 | 48 | 43 | 33 | 45 | 48 |
| NM_017440 | LUA#49 | 34 | 34.5 | 41 | 74 | 72 | 98 | 77 | 31 | 102 | 86.5 |
| NM_007331 | LUA#50 | 29 | 40.5 | 43 | 65 | 87 | 88 | 81 | 28 | 81.5 | 79 |
| NM_173823 | LUA#6 | 93 | 147.5 | 213 | 415 | 597 | 627.5 | 479 | 51.5 | 517 | 573 |
| NM_000962 | LUA#7 | 142 | 230 | 296 | 559 | 717.5 | 722.5 | 545.5 | 98.5 | 665 | 712 |
| NM_003825 | LUA#8 | 172 | 244 | 276 | 446 | 630 | 617 | 500 | 162.5 | 575 | 613 |
| NM_016061 | LUA#9 | 78 | 117 | 154 | 268 | 406 | 421 | 311 | 60 | 359 | 392 |
| NM_000153 | LUA#10 | 69.5 | 108 | 141.5 | 277 | 400 | 396 | 292 | 61 | 336 | 378 |
| NM_006948 | LUA#51 | 111 | 189 | 284 | 558.5 | 772 | 781 | 604 | 63 | 709.5 | 736 |
| NM_004631 | LUA#52 | 56 | 78 | 109 | 213.5 | 315.5 | 325 | 269 | 45 | 306 | 303 |
| NM_002358 | LUA#53 | 31.5 | 39 | 42 | 68 | 100 | 100 | 76 | 33 | 87 | 93 |
| NM_013402 | LUA#54 | 29 | 29.5 | 44.5 | 56.5 | 73 | 76 | 63 | 30 | 68 | 75 |
| NM_000875 | LUA#55 | 27.5 | 33 | 41 | 59 | 60 | 70 | 70 | 26 | 75 | 70 |
| NM_001974 | LUA#11 | 41 | 68 | 94 | 202 | 344 | 371 | 253 | 32 | 276 | 318.5 |
| NM_000632 | LUA#12 | 40 | 56 | 89 | 189 | 272 | 293 | 236 | 33 | 277.5 | 300 |
| NM_006457 | LUA#13 | 50 | 53.5 | 63 | 82 | 102 | 111.5 | 91 | 42 | 89 | 95 |
| NM_000698 | LUA#14 | 188 | 330 | 526 | 1026 | 1241 | 1325 | 997 | 65 | 1148 | 1215.5 |
| NM_032571 | LUA#15 | 29 | 39 | 56 | 98 | 152 | 157 | 121 | 30.5 | 132 | 146 |
| NM_006138 | LUA#56 | 37 | 40 | 49 | 67 | 97 | 102.5 | 86 | 38 | 87 | 95 |
| NM_015201 | LUA#57 | 40 | 49 | 64 | 139 | 197 | 228 | 156 | 31 | 208 | 198 |
| NM_006985 | LUA#58 | 31 | 32 | 37.5 | 45.5 | 52 | 59 | 46.5 | 30 | 48 | 59 |
| NM_004095 | LUA#59 | 46 | 66 | 86.5 | 166.5 | 221 | 208.5 | 147 | 36 | 169 | 205 |
| NM_005914 | LUA#60 | 102 | 218.5 | 314 | 594 | 914 | 889 | 716.5 | 44 | 525.5 | 804.5 |
| NM_007282 | LUA#16 | 27 | 29 | 30 | 42 | 53 | 61.5 | 58 | 28 | 46 | 62.5 |
| NM_003644 | LUA#17 | 53 | 72 | 75.5 | 137 | 220 | 238.5 | 179 | 39 | 181.5 | 218 |
| NM_001498 | LUA#18 | 40 | 71 | 121 | 262 | 386 | 382 | 279 | 30 | 397 | 391.5 |
| NM_003172 | LUA#19 | 45 | 68 | 97 | 199 | 234 | 250 | 179 | 33 | 208 | 227 |
| NM_004723 | LUA#20 | 61 | 105 | 151 | 271.5 | 350 | 352 | 258 | 37 | 281 | 329 |
| NM_014366 | LUA#61 | 38 | 61 | 89.5 | 191 | 291.5 | 299.5 | 235 | 33.5 | 217 | 294 |
| NM_003581 | LUA#62 | 40 | 36 | 42 | 47 | 46.5 | 46 | 40 | 33 | 40.5 | 39.5 |
| NM_018115 | LUA#63 | 109.5 | 234 | 409 | 710.5 | 896 | 1206.5 | 927 | 61 | 1217 | 860.5 |
| NM_021974 | LUA#64 | 136.5 | 255.5 | 403 | 781.5 | 929 | 940 | 667.5 | 55 | 776 | 891 |
| NM_024045 | LUA#65 | 35 | 40 | 57 | 92.5 | 135.5 | 137 | 110 | 29 | 112 | 127.5 |
| NM_004079 | LUA#21 | 34 | 41 | 53 | 88 | 122 | 124 | 121 | 30 | 113 | 124 |
| NM_000414 | LUA#22 | 48 | 74 | 114 | 272.5 | 479 | 477.5 | 378 | 35 | 384 | 448 |
| NM_001684 | LUA#23 | 30 | 31.5 | 31 | 52 | 66 | 60 | 49 | 25 | 52.5 | 60 |
| NM_003879 | LUA#24 | 23 | 29 | 36 | 51 | 77 | 76 | 59 | 34 | 65 | 69 |
| NM_002166 | LUA#25 | 37 | 51 | 74 | 132 | 188.5 | 211 | 170 | 29 | 169.5 | 207 |
| NM_005952 | LUA#66 | 43 | 56 | 74 | 143 | 200 | 197.5 | 156 | 34 | 204 | 199 |
| NM_001034 | LUA#67 | 42 | 47 | 73 | 122 | 178 | 184 | 155 | 33 | 170 | 171 |
| NM_003132 | LUA#68 | 74 | 117.5 | 194 | 367 | 461 | 482 | 344 | 38 | 431 | 428 |
| NM_018164 | LUA#69 | 37 | 46 | 67 | 139 | 150 | 156.5 | 133 | 36 | 148.5 | 149 |
| NM_014573 | LUA#70 | 40 | 45 | 56 | 93 | 156 | 161.5 | 123 | 38.5 | 138.5 | 144.5 |
| NM_014333 | LUA#26 | 74 | 128 | 211 | 427 | 674 | 705 | 572 | 41 | 613 | 656.5 |
| NM_006432 | LUA#27 | 190 | 358.5 | 574 | 1118 | 1397.5 | 1403 | 1160 | 68 | 1364 | 1394 |
| NM_000433 | LUA#28 | 62 | 105 | 169 | 371.5 | 580.5 | 600.5 | 406 | 32 | 454 | 548 |
| NM_000147 | LUA#29 | 95 | 188.5 | 323 | 713 | 1109 | 1156 | 905 | 43 | 987 | 1072 |
| NM_000584 | LUA#30 | 240 | 426 | 663 | 1369.5 | 1886 | 1908.5 | 1555 | 96.5 | 1878.5 | 1840 |
| NM_006452 | LUA#71 | 25 | 37 | 37.5 | 43 | 57 | 71 | 44 | 23.5 | 52 | 60 |
| NM_005915 | LUA#72 | 25 | 27 | 30 | 35 | 40 | 37 | 38 | 29 | 46 | 42 |
| NM_005980 | LUA#73 | 32 | 43 | 52 | 102.5 | 157 | 166 | 132 | 34 | 160 | 157 |
| NM_002539 | LUA#74 | 46 | 74 | 106 | 209 | 271 | 323.5 | 225 | 36 | 276 | 278.5 |
| NM_019058 | LUA#75 | 37 | 48 | 56 | 90 | 126 | 137 | 97 | 34 | 102 | 129 |
| NM_004152 | LUA#31 | 319 | 601 | 837 | 1645 | 2020 | 2157.5 | 1685 | 90.5 | 1845 | 1993 |
| NM_004602 | LUA#32 | 61 | 87.5 | 116 | 185 | 270 | 293 | 288 | 46 | 255 | 274.5 |
| NM_018890 | LUA#33 | 82 | 151.5 | 240 | 534.5 | 760 | 762 | 652 | 41 | 737 | 769 |
| NM_001101 | LUA#34 | 799 | 1279 | 1711 | 2759.5 | 2895 | 2880 | 2335 | 235 | 2622 | 2644 |
| NM_006019 | LUA#35 | 73 | 140 | 227 | 497 | 747 | 787.5 | 655 | 34 | 810 | 792 |
| NM_004134 | LUA#76 | 47.5 | 67 | 93 | 182 | 284 | 297 | 240 | 36 | 268 | 286 |
| NM_005008 | LUA#77 | 53.5 | 87 | 114 | 239.5 | 315 | 316 | 232 | 41 | 242 | 301.5 |
| NM_020117 | LUA#78 | 65 | 114 | 184 | 371 | 519 | 543 | 415 | 45 | 487 | 502 |
| NM_001469 | LUA#79 | 141.5 | 246.5 | 395 | 731.5 | 1124 | 1134 | 921 | 75.5 | 988.5 | 1104 |
| NM_021203 | LUA#80 | 65 | 109 | 165 | 355 | 480 | 514 | 369 | 46 | 445.5 | 471 |
| NM_002624 | LUA#36 | 253.5 | 500 | 700.5 | 1234 | 1402 | 1395 | 1167.5 | 79 | 1359 | 1343 |
| NM_004759 | LUA#37 | 32 | 41 | 60 | 119 | 132.5 | 213 | 159 | 28 | 242 | 139.5 |
| NM_002664 | LUA#38 | 383 | 702 | 1051.5 | 1832 | 2052 | 2064 | 1635.5 | 101 | 1922 | 1934 |
| NM_000211 | LUA#39 | 204.5 | 356 | 501 | 939 | 1215 | 1215 | 924.5 | 109 | 1072 | 1160 |
| NM_002468 | LUA#40 | 57.5 | 93 | 132.5 | 303 | 450 | 473.5 | 357 | 36.5 | 367 | 421 |
| NM_000884 | LUA#81 | 126 | 209 | 304 | 612 | 794.5 | 804 | 624 | 58 | 670 | 692 |
| NM_003752 | LUA#82 | 54 | 54 | 66 | 93 | 105 | 123 | 96 | 41 | 116 | 113 |
| NM_018256 | LUA#83 | 178.5 | 177 | 268 | 371 | 633 | 804.5 | 779 | 151 | 638.5 | 688.5 |
| NM_001948 | LUA#84 | 34 | 38 | 38 | 50 | 61 | 59 | 54 | 33 | 63 | 62 |
| NM_005566 | LUA#85 | 89 | 117 | 183 | 197.5 | 597 | 632 | 489 | 45 | 539.5 | 610.5 |
| NM_021103 | LUA#41 | 467.5 | 781.5 | 992 | 1953 | 2465.5 | 2408 | 495 | 142 | 629 | 2096 |
| NM_002970 | LUA#42 | 164 | 329 | 528 | 1106 | 1508 | 1570.5 | 1247 | 57 | 1316 | 1443 |
| NM_003332 | LUA#43 | 591 | 1003 | 1446 | 2239 | 2492 | 2467 | 2041 | 177.5 | 2312 | 2346.5 |
| NM_004106 | LUA#44 | 235 | 457 | 672 | 1276 | 1500 | 1506 | 1234 | 68 | 1458 | 1423 |
| NM_002982 | LUA#45 | 1175 | 1759 | 2328 | 3359 | 3548 | 3612.5 | 3101 | 373 | 3449 | 3440 |
| NM_005375 | LUA#86 | 106 | 131 | 196 | 334 | 547.5 | 602 | 531 | 86 | 548 | 553 |
| NM_000250 | LUA#87 | 46 | 52 | 67 | 115 | 159 | 166 | 131 | 38 | 141 | 157 |
| NM_004526 | LUA#88 | 43 | 61 | 76.5 | 137 | 187.5 | 191 | 138 | 37 | 153.5 | 172 |
| NM_004741 | LUA#89 | 70 | 77 | 131 | 204.5 | 315 | 415.5 | 300.5 | 58 | 397 | 326 |
| NM_002467 | LUA#90 | 136 | 162 | 239 | 409.5 | 576 | 644 | 527 | 86 | 571 | 552.5 |
| ACTB | LUA#91 | 452 | 812 | 1191 | 2112.5 | 2760 | 2845 | 2391 | 144.5 | 2538 | 2604.5 |
| TFRC | LUA#92 | 54 | 66 | 90 | 168 | 256 | 272 | 213 | 45 | 252 | 255 |
| GAPDH_5 | LUA#93 | 261.5 | 439.5 | 741 | 1388 | 1787 | 1865 | 1714 | 97 | 1699 | 1739.5 |
| GAPDH_M | LUA#94 | 221.5 | 396 | 590.5 | 1179.5 | 1602 | 1586 | 1267.5 | 79 | 1342.5 | 1462 |
| GAPDH_3 | LUA#95 | 579 | 1017 | 1438 | 2495.5 | 2680 | 2718 | 2148 | 172 | 2330.5 | 2534 |

TABLE 9
Sample Information

| | Data | | | | | | | | | | | | | | N-T | MultiC | |
|---|---|---|---|---|---|---|---|---|---|---|---|---|---|---|---|---|---|
| Name | Set | SR Name | HuFL Scan | Hu35KsubA Scan | BV | SSC | MAL | TT | CLT | PDT | AS | EP | GI | Culture | CLS | CLS | RNA |
| N_STOM_1 | 1 | | | | 1 | 1 | 1 | 1 | 1 | 0 | 0 | 1 | 1 | NA | 0 | 0 | 10 |
| N_STOM_2 | 1 | | | | 1 | 1 | 1 | 1 | 1 | 0 | 0 | 1 | 1 | NA | 0 | 0 | 10 |
| N_STOM_3 | 1 | | | | 1 | 1 | 1 | 1 | 1 | 0 | 0 | 1 | 1 | NA | 0 | 0 | 10 |
| N_STOM_4 | 1 | | | | 1 | 1 | 1 | 1 | 1 | 0 | 0 | 1 | 1 | NA | 0 | 0 | 10 |
| N_STOM_5 | 1 | | | | 1 | 1 | 1 | 1 | 1 | 0 | 0 | 1 | 1 | NA | 0 | 0 | 10 |
| N_STOM_6 | 1 | | | | 1 | 1 | 1 | 1 | 1 | 0 | 0 | 1 | 1 | NA | 0 | 0 | 10 |
| N_COLON_1 | 1 | | CL2000090529AA | CL2000090729AA | 1 | 1 | 1 | 2 | 1 | 0 | 0 | 1 | 1 | NA | 1 | 0 | 10 |
| N_COLON_2 | 1 | | | | 1 | 1 | 1 | 2 | 1 | 0 | 0 | 1 | 1 | NA | 1 | 0 | 10 |
| N_COLON_3 | 1 | | CL2000091210AA | CL2000091510AA | 1 | 1 | 1 | 2 | 1 | 0 | 0 | 1 | 1 | NA | 1 | 0 | 10 |
| N_COLON_4 | 1 | | CL2000090527AA | CL2000090727AA | 1 | 1 | 1 | 2 | 1 | 0 | 0 | 1 | 1 | NA | 1 | 0 | 10 |
| N_COLON_5 | 1 | | CL2000090523AA | CL2000090723AA | 1 | 1 | 1 | 2 | 1 | 0 | 0 | 1 | 1 | NA | 1 | 0 | 10 |
| T_COLON_1 | 1 | | | | 1 | 1 | 2 | 2 | 1 | 0 | 0 | 1 | 1 | NA | 1 | 0 | 10 |
| T_COLON_2 | 1 | Colorectal_Adeno_mCRT2_(9752) | CH2000030408AA | SR2000042821AA | 1 | 1 | 2 | 2 | 1 | 0 | 0 | 1 | 1 | NA | 1 | 1 | 10 |
| T_COLON_3 | 1 | Colorectal_Adeno_9912c055_CC | CH2000031308AA | SR2000042828AA | 1 | 1 | 2 | 2 | 1 | 0 | 0 | 1 | 1 | NA | 1 | 1 | 10 |
| T_COLON_4 | 1 | Colorectal_Adeno_95_I_175 | CH2000030516AA | SR2000042819AA | 1 | 1 | 2 | 2 | 1 | 0 | 0 | 1 | 1 | NA | 1 | 1 | 10 |
| T_COLON_5 | 1 | Colorectal_Adeno_0001c038_CC | CH2000031317AA | SR2000042826AA | 1 | 1 | 2 | 2 | 1 | 0 | 0 | 1 | 1 | NA | 1 | 1 | 10 |
| T_COLON_6 | 1 | | | | 1 | 1 | 2 | 2 | 1 | 0 | 0 | 1 | 1 | NA | 1 | 0 | 10 |
| T_COLON_7 | 1 | Colorectal_Adeno_95_I_057 | CH2000030507AA | SR2000042824AA | 1 | 1 | 2 | 2 | 1 | 0 | 0 | 1 | 1 | NA | 1 | 1 | 10 |
| T_COLON_8 | 1 | | | SR2000051017AA | 1 | 1 | 2 | 2 | 1 | 0 | 0 | 1 | 1 | NA | 1 | 0 | 10 |
| T_COLON_9 | 1 | Colorectal_Adeno_0001c040_CC | CH2000031309AA | CL2000091537AA | 1 | 1 | 2 | 2 | 1 | 0 | 0 | 1 | 1 | NA | 1 | 1 | 10 |
| T_COLON_10 | 1 | Colorectal_Adeno_HCTN_CRT1_(18851_A1B) | SR1999121605AA | SR2000042825AA | 1 | 1 | 2 | 2 | 1 | 0 | 0 | 1 | 1 | NA | 1 | 1 | 10 |
| N_PAN_1 | 1 | | CL2000090543AA | CL2000090743AA | 1 | 1 | 1 | 3 | 1 | 0 | 0 | 1 | 1 | NA | 0 | 0 | 10 |
| T_PAN_1 | 1 | Pancreas_Adeno_Pan_3T | CH2000031008AA | SR2000042222AA | 1 | 1 | 2 | 3 | 1 | 0 | 0 | 1 | 1 | NA | 0 | 1 | 10 |
| T_PAN_2 | 1 | Pancreas_Adeno_Pan_6T | CH2000031312AA | SR2000042224AA | 1 | 1 | 2 | 3 | 1 | 0 | 0 | 1 | 1 | NA | 0 | 1 | 10 |
| T_PAN_3 | 1 | Pancreas_Adeno_97_I_077 | CH2000031020AA | | 1 | 1 | 2 | 3 | 1 | 0 | 0 | 1 | 1 | NA | 0 | 0 | 10 |
| T_PAN_4 | 1 | Pancreas_Adeno_Pan_2T | CH2000031318AA | SR2000042221AA | 1 | 1 | 2 | 3 | 1 | 0 | 0 | 1 | 1 | NA | 0 | 1 | 10 |
| T_PAN_5 | 1 | Pancreas_Adeno_Pan_7T | CH2000031311AA | SR2000042225AA | 1 | 1 | 2 | 3 | 1 | 0 | 0 | 1 | 1 | NA | 0 | 1 | 10 |
| T_PAN_6 | 1 | Pancreas_Adeno_Pan_17T | CL2000071414AA | CL2000071840AA | 1 | 1 | 2 | 3 | 1 | 0 | 0 | 1 | 1 | NA | 0 | 1 | 10 |
| T_PAN_7 | 1 | Pancreas_Adeno_Pan_4T | CH2000031024AA | SR2000042223AA | 1 | 1 | 2 | 3 | 1 | 0 | 0 | 1 | 1 | NA | 0 | 1 | 10 |
| T_PAN_8 | 1 | Pancreas_Adeno_Pan_1T | CH2000031306AA | SR2000042220AA | 1 | 1 | 2 | 3 | 1 | 0 | 0 | 1 | 1 | NA | 0 | 1 | 10 |
| T_PAN_9 | 1 | Pancreas_Adeno_Pan_29T | CL2000071409AA | CL2000081524AA | 1 | 1 | 2 | 3 | 1 | 0 | 0 | 1 | 1 | NA | 0 | 1 | 10 |
| N_LVR_1 | 1 | | | | 1 | 1 | 1 | 4 | 1 | 0 | 0 | 1 | 1 | NA | 0 | 0 | 10 |
| N_LVR_2 | 1 | | | | 1 | 1 | 1 | 4 | 1 | 0 | 0 | 1 | 1 | NA | 0 | 0 | 10 |
| N_LVR_3 | 1 | | | | 1 | 1 | 1 | 4 | 1 | 0 | 0 | 1 | 1 | NA | 0 | 0 | 10 |
| N_KID_1 | 1 | | CL2000091226AA | CL2000091526AA | 1 | 1 | 1 | 5 | 1 | 0 | 0 | 1 | 0 | NA | 1 | 0 | 10 |
| N_KID_2 | 1 | | CL2000090539AA | CL2000090739AA | 1 | 1 | 1 | 5 | 1 | 0 | 0 | 1 | 0 | NA | 1 | 0 | 10 |
| N_KID_3 | 1 | | CL2000091214AA | CL2000091514AA | 1 | 1 | 1 | 5 | 1 | 0 | 0 | 1 | 0 | NA | 1 | 0 | 10 |
| T_KID_1 | 1 | Renal_Carcinoma_Carc_628TG_{—} | MG1999030902AA | SR2000060917AA | 1 | 1 | 2 | 5 | 1 | 0 | 0 | 1 | 0 | NA | 1 | 1 | 10 |
| T_KID_2 | 1 | | | SR2000060913AA | 1 | 1 | 2 | 5 | 1 | 0 | 0 | 1 | 0 | NA | 1 | 0 | 10 |
| T_KID_3 | 1 | Renal_Carcinoma_Carc_614TO_{—} | MG1999030904AA | SR2000060914AA | 1 | 1 | 2 | 5 | 1 | 0 | 0 | 1 | 0 | NA | 1 | 1 | 10 |
| T_KID_4 | 1 | Renal_Carcinoma_Carc_609TO_{—} | MG1999030901AA | SR2000060916AA | 1 | 1 | 2 | 5 | 1 | 0 | 0 | 1 | 0 | NA | 1 | 1 | 10 |
| T_KID_5 | 1 | Renal_Carcinoma_92_I_126 | CH2000030508AA | SR2000050421AA | 1 | 1 | 2 | 5 | 1 | 0 | 0 | 1 | 0 | NA | 1 | 1 | 10 |
| TCL_293_1 | 1 | | | | 1 | 4 | 3 | 5 | 8 | 0 | 0 | 1 | 0 | NA | 0 | 0 | 10 |
| TCL_293_2 | 1 | | | | 1 | 4 | 3 | 5 | 8 | 0 | 0 | 1 | 0 | NA | 0 | 0 | 10 |
| TCL_293_3 | 1 | | | | 1 | 4 | 3 | 5 | 8 | 0 | 0 | 1 | 0 | NA | 0 | 0 | 10 |
| N_BLDR_1 | 1 | | CL2000090532AA | CL2000090732AA | 1 | 1 | 1 | 6 | 1 | 0 | 0 | 1 | 0 | NA | 0 | 0 | 10 |
| N_BLDR_2 | 1 | | | | 1 | 1 | 1 | 6 | 1 | 0 | 0 | 1 | 0 | NA | 0 | 0 | 10 |
| T_BLDR_1 | 1 | Bladder_TCC_9858 | SR2000042208AA | SR2000051014AA | 1 | 1 | 2 | 6 | 1 | 0 | 0 | 1 | 0 | NA | 0 | 1 | 10 |
| T_BLDR_2 | 1 | | | | 1 | 1 | 2 | 6 | 1 | 0 | 0 | 1 | 0 | NA | 0 | 0 | 10 |
| T_BLDR_3 | 1 | Bladder_TCC_11520 | SR2000042201AA | SR2000051005AA | 1 | 1 | 2 | 6 | 1 | 0 | 0 | 1 | 0 | NA | 0 | 1 | 10 |
| T_BLDR_4 | 1 | Bladder_TCC_B_0004 | CL2000080113AA | CL2000080314AA | 1 | 1 | 2 | 6 | 1 | 0 | 0 | 1 | 0 | NA | 0 | 1 | 10 |
| T_BLDR_5 | 1 | Bladder_TCC_B_0008 | CL2000080115AA | CL2000080803AA | 1 | 1 | 2 | 6 | 1 | 0 | 0 | 1 | 0 | NA | 0 | 1 | 10 |
| T_BLDR_6 | 1 | Bladder_TCC_B_0001 | CL2000080110AA | CL2000080311AA | 1 | 1 | 2 | 6 | 1 | 0 | 0 | 1 | 0 | NA | 0 | 1 | 10 |
| T_BLDR_7 | 1 | Bladder_TCC_07- | CL2000080109AA | CL2000080310AA | 1 | 1 | 2 | 6 | 1 | 0 | 0 | 1 | 0 | NA | 0 | 1 | 10 |
| | | B_003E | | | | | | | | | | | | | | | |
| N_PROST_1 | 1 | | CL2000090515AA | CL2000090715AA | 1 | 1 | 1 | 7 | 1 | 0 | 0 | 1 | 0 | NA | 1 | 0 | 10 |
| N_PROST_2 | 1 | | CL2000090518AA | CL2000090718AA | 1 | 1 | 1 | 7 | 1 | 0 | 0 | 1 | 0 | NA | 1 | 0 | 10 |
| N_PROST_3 | 1 | | | | 1 | 1 | 1 | 7 | 1 | 0 | 0 | 1 | 0 | NA | 1 | 0 | 10 |
| N_PROST_4 | 1 | | CL2000090514AA | CL2000090714AA | 1 | 1 | 1 | 7 | 1 | 0 | 0 | 1 | 0 | NA | 1 | 0 | 10 |
| N_PROST_5 | 1 | | | | 1 | 1 | 1 | 7 | 1 | 0 | 0 | 1 | 0 | NA | 1 | 0 | 10 |
| N_PROST_6 | 1 | | CL2000090517AA | CL2000090717AA | 1 | 1 | 1 | 7 | 1 | 0 | 0 | 1 | 0 | NA | 1 | 0 | 10 |
| N_PROST_7 | 1 | | CL2000090519AA | CL2000090719AA | 1 | 1 | 1 | 7 | 1 | 0 | 0 | 1 | 0 | NA | 1 | 0 | 10 |
| N_PROST_8 | 1 | | CL2000090516AA | CL2000090716AA | 1 | 1 | 1 | 7 | 1 | 0 | 0 | 1 | 0 | NA | 1 | 0 | 10 |
| T_PROST_1 | 1 | Prostate_Adeno_P_0025 | CL2000090506AA | CL2000090706AA | 1 | 1 | 2 | 7 | 1 | 0 | 0 | 1 | 0 | NA | 1 | 1 | 10 |
| T_PROST_2 | 1 | Prostate_Adeno_P_0030 | CL2000090507AA | CL2000090707AA | 1 | 1 | 2 | 7 | 1 | 0 | 0 | 1 | 0 | NA | 1 | 1 | 10 |
| T_PROST_3 | 1 | Prostate_Adeno_P_0036 | CL2000090509AA | CL2000090709AA | 1 | 1 | 2 | 7 | 1 | 0 | 0 | 1 | 0 | NA | 1 | 1 | 10 |
| T_PROST_4 | 1 | Prostate_Adeno_P_0033 | CL2000090508AA | CL2000090708AA | 1 | 1 | 2 | 7 | 1 | 0 | 0 | 1 | 0 | NA | 1 | 1 | 10 |
| T_PROST_5 | 1 | Prostate_Adeno_95_I_256 | CL2000071413AA | CL2000071839AA | 1 | 1 | 2 | 7 | 1 | 0 | 0 | 1 | 0 | NA | 1 | 1 | 10 |
| T_PROST_6 | 1 | Prostate_Adeno_94_I_052 | CH2000030405AA | SR2000050409AA | 1 | 1 | 2 | 7 | 1 | 0 | 0 | 1 | 0 | NA | 1 | 1 | 10 |
| TCL_PC- | 1 | | | | 1 | 4 | 3 | 7 | 4 | 0 | 0 | 1 | 0 | NA | 0 | 0 | 10 |
| 3_1 | | | | | | | | | | | | | | | | | |
| TCL_PC- | 1 | | | | 1 | 4 | 3 | 7 | 4 | 0 | 0 | 1 | 0 | NA | 0 | 0 | 10 |
| 3_2 | | | | | | | | | | | | | | | | | |
| TCL_PC- | 1 | | | | 1 | 4 | 3 | 7 | 4 | 0 | 0 | 1 | 0 | NA | 0 | 0 | 10 |
| 3_3 | | | | | | | | | | | | | | | | | |
| TCL_PC- | 1 | | | | 1 | 4 | 3 | 7 | 4 | 0 | 0 | 1 | 0 | NA | 0 | 0 | 10 |
| 3_4 | | | | | | | | | | | | | | | | | |
| T_OVARY_1 | 1 | Ovary_Adeno_mOVT1_(8691) | CH2000030411AA | SR2000050412AA | 1 | 1 | 2 | 8 | 1 | 0 | 0 | 1 | 0 | NA | 0 | 1 | 10 |
| T_OVARY_2 | 1 | | | | 1 | 1 | 2 | 8 | 1 | 0 | 0 | 1 | 0 | NA | 0 | 0 | 10 |
| T_OVARY_3 | 1 | Ovary_Adeno_H_6206 | CL2000080107AA | CL2000080308AA | 1 | 1 | 2 | 8 | 1 | 0 | 0 | 1 | 0 | NA | 0 | 1 | 10 |
| T_OVARY_4 | 1 | Ovary_Adeno_07- | CL2000080103AA | CL2000080304AA | 1 | 1 | 2 | 8 | 1 | 0 | 0 | 1 | 0 | NA | 0 | 1 | 10 |
| | | B_001B | | | | | | | | | | | | | | | |
| T_OVARY_5 | 1 | Ovary_Adeno_07- | CL2000080104AA | CL2000080305AA | 1 | 1 | 2 | 8 | 1 | 0 | 0 | 1 | 0 | NA | 0 | 1 | 10 |
| | | B_014G | | | | | | | | | | | | | | | |
| T_OVARY_6 | 1 | Ovary_Adeno_93_I_081 | CH2000030415AA | SR2000050411AA | 1 | 1 | 2 | 8 | 1 | 0 | 0 | 1 | 0 | NA | 0 | 1 | 10 |
| T_OVARY_7 | 1 | | | | 1 | 1 | 2 | 8 | 1 | 0 | 0 | 1 | 0 | NA | 0 | 0 | 10 |
| N_UT_1 | 1 | | | | 1 | 1 | 1 | 9 | 1 | 0 | 0 | 1 | 0 | NA | 1 | 0 | 10 |
| N_UT_2 | 1 | | | | 1 | 1 | 1 | 9 | 1 | 0 | 0 | 1 | 0 | NA | 1 | 0 | 10 |
| N_UT_3 | 1 | | | | 1 | 1 | 1 | 9 | 1 | 0 | 0 | 1 | 0 | NA | 1 | 0 | 10 |
| N_UT_4 | 1 | | | | 1 | 1 | 1 | 9 | 1 | 0 | 0 | 1 | 0 | NA | 1 | 0 | 10 |
| N_UT_5 | 1 | | | | 1 | 1 | 1 | 9 | 1 | 0 | 0 | 1 | 0 | NA | 1 | 0 | 10 |
| N_UT_6 | 1 | | | | 1 | 1 | 1 | 9 | 1 | 0 | 0 | 1 | 0 | NA | 1 | 0 | 10 |
| N_UT_7 | 1 | | | | 1 | 1 | 1 | 9 | 1 | 0 | 0 | 1 | 0 | NA | 1 | 0 | 10 |
| N_UT_8 | 1 | | CL2000091225AA | CL2000091525AA | 1 | 1 | 1 | 9 | 1 | 0 | 0 | 1 | 0 | NA | 1 | 0 | 10 |
| N_UT_9 | 1 | | | | 1 | 1 | 1 | 9 | 1 | 0 | 0 | 1 | 0 | NA | 1 | 0 | 10 |
| T_UT_1 | 1 | Uterus_Adeno_2967 | SR2000042205AA | SR2000051008AA | 1 | 1 | 2 | 9 | 1 | 0 | 0 | 1 | 0 | NA | 1 | 1 | 10 |
| T_UT_2 | 1 | Uterus_Adeno_3663 | SR2000042203AA | SR2000051003AA | 1 | 1 | 2 | 9 | 1 | 0 | 0 | 1 | 0 | NA | 1 | 1 | 10 |
| T_UT_3 | 1 | Uterus_Adeno_3226 | SR2000042207AA | SR2000051931AA | 1 | 1 | 2 | 9 | 1 | 0 | 0 | 1 | 0 | NA | 1 | 1 | 10 |
| T_UT_4 | 1 | Uterus_Adeno_4915 | SR2000042209AA | SR2000051001AA | 1 | 1 | 2 | 9 | 1 | 0 | 0 | 1 | 0 | NA | 1 | 1 | 10 |
| T_UT_5 | 1 | Uterus_Adeno_92_I_073 | CH2000030413AA | SR2000050424AA | 1 | 1 | 2 | 9 | 1 | 0 | 0 | 1 | 0 | NA | 1 | 1 | 10 |
| T_UT_6 | 1 | Uterus_Adeno_5116 | SR2000042206AA | SR2000051016AA | 1 | 1 | 2 | 9 | 1 | 0 | 0 | 1 | 0 | NA | 1 | 1 | 10 |
| T_UT_7 | 1 | Uterus_Adeno_4075 | SR2000042212AA | SR2000051010AA | 1 | 1 | 2 | 9 | 1 | 0 | 0 | 1 | 0 | NA | 1 | 1 | 10 |
| T_UT_8 | 1 | Uterus_Adeno_2552 | SR2000042210AA | SR2000051004AA | 1 | 1 | 2 | 9 | 1 | 0 | 0 | 1 | 0 | NA | 1 | 1 | 10 |
| T_UT_9 | 1 | Uterus_Adeno_4203 | SR2000042202AA | SR2000051009AA | 1 | 1 | 2 | 9 | 1 | 0 | 0 | 1 | 0 | NA | 1 | 1 | 10 |
| T_UT_10 | 1 | Uterus_Adeno_4840 | SR2000042214AA | SR2000051011AA | 1 | 1 | 2 | 9 | 1 | 0 | 0 | 1 | 0 | NA | 1 | 1 | 10 |
| N_LUNG_1 | 1 | | CL2000090521AA | CL2000090721AA | 1 | 1 | 1 | 10 | 1 | 0 | 0 | 1 | 0 | NA | 1 | 0 | 10 |
| N_LUNG_2 | 1 | | | | 1 | 1 | 1 | 10 | 1 | 0 | 0 | 1 | 0 | NA | 1 | 0 | 10 |
| N_LUNG_3 | 1 | | CL2000091223AA | CL2000091523AA | 1 | 1 | 1 | 10 | 1 | 0 | 0 | 1 | 0 | NA | 1 | 0 | 10 |
| N_LUNG_4 | 1 | | | | 1 | 1 | 1 | 10 | 1 | 0 | 0 | 1 | 0 | NA | 1 | 0 | 10 |
| T_LUNG_1 | 1 | Lung_Adeno_004_B | CL2000090501AA | CL2000090701AA | 1 | 1 | 2 | 10 | 1 | 0 | 0 | 1 | 0 | NA | 1 | 1 | 10 |
| T_LUNG_2 | 1 | Lung_Adeno_H_20154 | CL2000090504AA | CL2000090704AA | 1 | 1 | 2 | 10 | 1 | 0 | 0 | 1 | 0 | NA | 1 | 1 | 10 |
| T_LUNG_3 | 1 | Met_Lung_H_20300 | CL2000090505AA | CL2000090705AA | 1 | 1 | 2 | 10 | 1 | 0 | 0 | 1 | 0 | NA | 1 | 1 | 10 |
| T_LUNG_4 | 1 | Lung_Adeno_009_C | CL2000090502AA | CL2000090702AA | 1 | 1 | 2 | 10 | 1 | 0 | 0 | 1 | 0 | NA | 1 | 1 | 10 |
| T_LUNG_5 | 1 | | | | 1 | 1 | 2 | 10 | 1 | 0 | 0 | 1 | 0 | NA | 1 | 0 | 10 |
| T_LUNG_6 | 1 | Lung_Adeno_H_20387 | CL2000090503AA | CL2000090703AA | 1 | 1 | 2 | 10 | 1 | 0 | 0 | 1 | 0 | NA | 1 | 1 | 10 |
| T_MESO_1 | 1 | Mesothelioma_300_T | CH2000031101AA | SR2000050516AA | 1 | 1 | 2 | 11 | 1 | 0 | 0 | 1 | 0 | NA | 0 | 1 | 10 |
| T_MESO_2 | 1 | Mesothelioma_224_T5 | CH2000031015AA | SR2000050509AA | 1 | 1 | 2 | 11 | 1 | 0 | 0 | 1 | 0 | NA | 0 | 1 | 10 |
| T_MESO_3 | 1 | Mesothelioma_235_T6 | CH2000031018AA | SR2000050507AA | 1 | 1 | 2 | 11 | 1 | 0 | 0 | 1 | 0 | NA | 0 | 1 | 10 |
| T_MESO_4 | 1 | Mesothelioma_169_T7 | CH2000031004AA | SR2000050501AA | 1 | 1 | 2 | 11 | 1 | 0 | 0 | 1 | 0 | NA | 0 | 1 | 10 |
| T_MESO_5 | 1 | Mesothelioma_31_T10 | CH2000031014AA | SR2000050513AA | 1 | 1 | 2 | 11 | 1 | 0 | 0 | 1 | 0 | NA | 0 | 1 | 10 |
| T_MESO_6 | 1 | Mesothelioma_165_T5 | CH2000031019AA | SR2000050510AA | 1 | 1 | 2 | 11 | 1 | 0 | 0 | 1 | 0 | NA | 0 | 1 | 10 |
| T_MESO_7 | 1 | Mesothelioma_74_T6 | CH2000031021AA | SR2000050514AA | 1 | 1 | 2 | 11 | 1 | 0 | 0 | 1 | 0 | NA | 0 | 1 | 10 |
| T_MESO_8 | 1 | Mesothelioma_215_T5 | CH2000031017AA | SR2000050511AA | 1 | 1 | 2 | 11 | 1 | 0 | 0 | 1 | 0 | NA | 0 | 1 | 10 |
| T_MELA_1 | 1 | Melanoma_96_I_166 | CH2000031316AA | SR2000050518AA | 1 | 1 | 2 | 12 | 1 | 0 | 0 | 1 | 0 | NA | 0 | 1 | 10 |
| T_MELA_2 | 1 | Melanoma_94_I_149 | CH2000031011AA | SR2000050504AA | 1 | 1 | 2 | 12 | 1 | 0 | 0 | 1 | 0 | NA | 0 | 1 | 10 |
| T_MELA_3 | 1 | Melanoma_93_I_262 | CH2000031305AA | SR2000050519AA | 1 | 1 | 2 | 12 | 1 | 0 | 0 | 1 | 0 | NA | 0 | 1 | 10 |
| TCL_SKMEL- | 1 | | | | 1 | 4 | 3 | 12 | 3 | 0 | 0 | 1 | 0 | NA | 0 | 0 | 10 |
| 5_1 | | | | | | | | | | | | | | | | | |
| TCL_SKMEL- | 1 | | | | 1 | 4 | 3 | 12 | 3 | 0 | 0 | 1 | 0 | NA | 0 | 0 | 10 |
| 5_2 | | | | | | | | | | | | | | | | | |
| N_BRST_1 | 1 | | CL2000090513AA | CL2000090713AA | 1 | 1 | 1 | 13 | 1 | 0 | 0 | 1 | 0 | NA | 1 | 0 | 10 |
| N_BRST_2 | 1 | | CL2000090511AA | CL2000090711AA | 1 | 1 | 1 | 13 | 1 | 0 | 0 | 1 | 0 | NA | 1 | 0 | 10 |
| N_BRST_3 | 1 | | CL2000090512AA | CL2000090712AA | 1 | 1 | 1 | 13 | 1 | 0 | 0 | 1 | 0 | NA | 1 | 0 | 10 |
| T_BRST_1 | 1 | Breast_Adeno_9912c068_CC | CH2000031302AA | SR2000042806AA | 1 | 1 | 2 | 13 | 1 | 0 | 0 | 1 | 0 | NA | 1 | 1 | 10 |
| T_BRST_2 | 1 | Breast_Adeno_94_I_155 | CH2000030407AA | SR2000042804AA | 1 | 1 | 2 | 13 | 1 | 0 | 0 | 1 | 0 | NA | 1 | 1 | 10 |
| T_BRST_3 | 1 | Breast_Adeno_mBRT1_(8697) | CH2000030509AA | SR2000051018AA | 1 | 1 | 2 | 13 | 1 | 0 | 0 | 1 | 0 | NA | 1 | 1 | 10 |
| T_BRST_4 | 1 | Breast_Adeno_95_I_029 | CH2000030511AA | SR2000042803AA | 1 | 1 | 2 | 13 | 1 | 0 | 0 | 1 | 0 | NA | 1 | 1 | 10 |
| T_BRST_5 | 1 | Breast_Adeno_93_I_250 | CH2000031102AA | SR2000042807AA | 1 | 1 | 2 | 13 | 1 | 0 | 0 | 1 | 0 | NA | 1 | 1 | 10 |
| T_BRST_6 | 1 | Breast_Adeno_09- | CL2000080301AA | CL2000091505AA | 1 | 1 | 2 | 13 | 1 | 0 | 0 | 1 | 0 | NA | 1 | 1 | 10 |
| | | B_003A | | | | | | | | | | | | | | | |
| TCL_MCF- | 1 | | | | 1 | 4 | 3 | 13 | 2 | 0 | 0 | 1 | 0 | NA | 0 | 0 | 10 |
| 7_1 | | | | | | | | | | | | | | | | | |
| TCL_MCF- | 1 | | | | 1 | 4 | 3 | 13 | 2 | 0 | 0 | 1 | 0 | NA | 0 | 0 | 10 |
| 7_2 | | | | | | | | | | | | | | | | | |
| TCL_MCF- | 1 | | | | 1 | 4 | 3 | 13 | 2 | 0 | 0 | 1 | 0 | NA | 0 | 0 | 10 |
| 7_3 | | | | | | | | | | | | | | | | | |
| TCL_MCF- | 1 | | | | 1 | 4 | 3 | 13 | 2 | 0 | 0 | 1 | 0 | NA | 0 | 0 | 10 |
| 7_4 | | | | | | | | | | | | | | | | | |
| TCL_MCF- | 1 | | | | 1 | 4 | 3 | 13 | 2 | 0 | 0 | 1 | 0 | NA | 0 | 0 | 10 |
| 7_5 | | | | | | | | | | | | | | | | | |
| N_BRAIN_1 | 1 | | CL2000091228AA | CL2000091528AA | 1 | 1 | 1 | 14 | 1 | 0 | 0 | 0 | 0 | NA | 0 | 0 | 10 |
| N_BRAIN_2 | 1 | | CL2000090547AA | CL2000090747AA | 1 | 1 | 1 | 14 | 1 | 0 | 0 | 0 | 0 | NA | 0 | 0 | 10 |
| T_BALL_1 | 1 | | | | 1 | 2 | 2 | 19 | 1 | 0 | 0 | 0 | 0 | NA | 0 | 0 | 5 |
| T_BALL_2 | 1 | | | | 1 | 2 | 2 | 19 | 1 | 0 | 0 | 0 | 0 | NA | 0 | 0 | 5 |
| T_BALL_3 | 1 | | | | 1 | 2 | 2 | 19 | 1 | 0 | 0 | 0 | 0 | NA | 0 | 0 | 5 |
| T_BALL_4 | 1 | | | | 1 | 2 | 2 | 19 | 1 | 0 | 0 | 0 | 0 | NA | 0 | 0 | 5 |
| T_BALL_5 | 1 | | | | 1 | 2 | 2 | 19 | 1 | 0 | 0 | 0 | 0 | NA | 0 | 0 | 5 |
| T_BALL_6 | 1 | | | | 1 | 2 | 2 | 19 | 1 | 0 | 0 | 0 | 0 | NA | 0 | 0 | 5 |
| T_BALL_7 | 1 | | | | 1 | 2 | 2 | 19 | 1 | 0 | 0 | 0 | 0 | NA | 0 | 0 | 5 |
| T_BALL_8 | 1 | | | | 1 | 2 | 2 | 19 | 1 | 0 | 0 | 0 | 0 | NA | 0 | 0 | 5 |
| T_BALL_9 | 1 | | | | 1 | 2 | 2 | 19 | 1 | 0 | 0 | 0 | 0 | NA | 0 | 0 | 5 |
| T_BALL_10 | 1 | | | | 1 | 2 | 2 | 19 | 1 | 0 | 0 | 0 | 0 | NA | 0 | 0 | 5 |
| T_BALL_11 | 1 | | | | 1 | 2 | 2 | 19 | 1 | 0 | 0 | 0 | 0 | NA | 0 | 0 | 5 |
| T_BALL_12 | 1 | | | | 1 | 2 | 2 | 19 | 1 | 0 | 0 | 0 | 0 | NA | 0 | 0 | 5 |
| T_BALL_13 | 1 | | | | 1 | 2 | 2 | 19 | 1 | 0 | 0 | 0 | 0 | NA | 0 | 0 | 5 |
| T_BALL_14 | 1 | | | | 1 | 2 | 2 | 19 | 1 | 0 | 0 | 0 | 0 | NA | 0 | 0 | 5 |
| T_BALL_15 | 1 | | | | 1 | 2 | 2 | 19 | 1 | 0 | 0 | 0 | 0 | NA | 0 | 0 | 5 |
| T_BALL_16 | 1 | | | | 1 | 2 | 2 | 19 | 1 | 0 | 0 | 0 | 0 | NA | 0 | 0 | 5 |
| T_BALL_17 | 1 | | | | 1 | 2 | 2 | 19 | 1 | 0 | 0 | 0 | 0 | NA | 0 | 0 | 5 |
| T_BALL_18 | 1 | | | | 1 | 2 | 2 | 19 | 1 | 0 | 0 | 0 | 0 | NA | 0 | 0 | 5 |
| T_BALL_19 | 1 | | | | 1 | 2 | 2 | 19 | 1 | 0 | 0 | 0 | 0 | NA | 0 | 0 | 5 |
| T_BALL_20 | 1 | | | | 1 | 2 | 2 | 19 | 1 | 0 | 0 | 0 | 0 | NA | 0 | 0 | 5 |
| T_BALL_21 | 1 | | | | 1 | 2 | 2 | 19 | 1 | 0 | 0 | 0 | 0 | NA | 0 | 0 | 5 |
| T_BALL_22 | 1 | | | | 1 | 2 | 2 | 19 | 1 | 0 | 0 | 0 | 0 | NA | 0 | 0 | 5 |
| T_BALL_23 | 1 | | | | 1 | 2 | 2 | 19 | 1 | 0 | 0 | 0 | 0 | NA | 0 | 0 | 5 |
| T_BALL_24 | 1 | | | | 1 | 2 | 2 | 19 | 1 | 0 | 0 | 0 | 0 | NA | 0 | 0 | 5 |
| T_BALL_25 | 1 | | | | 1 | 2 | 2 | 19 | 1 | 0 | 0 | 0 | 0 | NA | 0 | 0 | 5 |
| T_BALL_26 | 1 | | | | 1 | 2 | 2 | 19 | 1 | 0 | 0 | 0 | 0 | NA | 0 | 0 | 5 |
| T_TALL_1 | 1 | | | | 1 | 2 | 2 | 20 | 1 | 0 | 0 | 0 | 0 | NA | 0 | 0 | 5 |
| T_TALL_2 | 1 | | | | 1 | 2 | 2 | 20 | 1 | 0 | 0 | 0 | 0 | NA | 0 | 0 | 5 |
| T_TALL_3 | 1 | | | | 1 | 2 | 2 | 20 | 1 | 0 | 0 | 0 | 0 | NA | 0 | 0 | 5 |
| T_TALL_4 | 1 | | | | 1 | 2 | 2 | 20 | 1 | 0 | 0 | 0 | 0 | NA | 0 | 0 | 5 |
| T_TALL_5 | 1 | | | | 1 | 2 | 2 | 20 | 1 | 0 | 0 | 0 | 0 | NA | 0 | 0 | 5 |
| T_TALL_6 | 1 | | | | 1 | 3 | 2 | 20 | 1 | 0 | 0 | 0 | 0 | NA | 0 | 0 | 2 |
| T_TALL_7 | 1 | | | | 1 | 3 | 2 | 20 | 1 | 0 | 0 | 0 | 0 | NA | 0 | 0 | 2 |
| T_TALL_8 | 1 | | | | 1 | 3 | 2 | 20 | 1 | 0 | 0 | 0 | 0 | NA | 0 | 0 | 10 |
| T_TALL_9 | 1 | | | | 1 | 3 | 2 | 20 | 1 | 0 | 0 | 0 | 0 | NA | 0 | 0 | 10 |
| T_TALL_10 | 1 | | | | 1 | 3 | 2 | 20 | 1 | 0 | 0 | 0 | 0 | NA | 0 | 0 | 10 |
| T_TALL_11 | 1 | | | | 1 | 3 | 2 | 20 | 1 | 0 | 0 | 0 | 0 | NA | 0 | 0 | 10 |
| T_TALL_12 | 1 | | | | 1 | 3 | 2 | 20 | 1 | 0 | 0 | 0 | 0 | NA | 0 | 0 | 2 |
| T_TALL_13 | 1 | | | | 1 | 3 | 2 | 20 | 1 | 0 | 0 | 0 | 0 | NA | 0 | 0 | 2 |
| T_TALL_14 | 1 | | | | 1 | 3 | 2 | 20 | 1 | 0 | 0 | 0 | 0 | NA | 0 | 0 | 2 |
| T_TALL_15 | 1 | | | | 1 | 3 | 2 | 20 | 1 | 0 | 0 | 0 | 0 | NA | 0 | 0 | 2 |
| T_TALL_16 | 1 | | | | 1 | 3 | 2 | 20 | 1 | 0 | 0 | 0 | 0 | NA | 0 | 0 | 1 |
| T_TALL_17 | 1 | | | | 1 | 3 | 2 | 20 | 1 | 0 | 0 | 0 | 0 | NA | 0 | 0 | 1 |
| T_TALL_18 | 1 | | | | 1 | 3 | 2 | 20 | 1 | 0 | 0 | 0 | 0 | NA | 0 | 0 | 1 |
| TCL_ALLCL_1 | 1 | | | | 1 | 3 | 3 | 20 | 10 | 0 | 0 | 0 | 0 | NA | 0 | 0 | 10 |
| TCL_ALLCL_2 | 1 | | | | 1 | 3 | 3 | 20 | 10 | 0 | 0 | 0 | 0 | NA | 0 | 0 | 10 |
| TCL_ALLCL_3 | 1 | | | | 1 | 3 | 3 | 20 | 10 | 0 | 0 | 0 | 0 | NA | 0 | 0 | 10 |
| TCL_ALLCL_4 | 1 | | | | 1 | 3 | 3 | 20 | 10 | 0 | 0 | 0 | 0 | NA | 0 | 0 | 10 |
| TCL_ALLCL_5 | 1 | | | | 1 | 3 | 3 | 20 | 10 | 0 | 0 | 0 | 0 | NA | 0 | 0 | 10 |
| TCL_ALLCL_6 | 1 | | | | 1 | 3 | 3 | 20 | 10 | 0 | 0 | 0 | 0 | NA | 0 | 0 | 10 |
| TCL_ALLCL_7 | 1 | | | | 1 | 3 | 3 | 20 | 10 | 0 | 0 | 0 | 0 | NA | 0 | 0 | 10 |
| TCL_ALLCL_8 | 1 | | | | 1 | 3 | 3 | 20 | 10 | 0 | 0 | 0 | 0 | NA | 0 | 0 | 10 |
| TCL_ALLCL_9 | 1 | | | | 1 | 3 | 3 | 20 | 10 | 0 | 0 | 0 | 0 | NA | 0 | 0 | 10 |
| TCL_ALLCL_10 | 1 | | | | 1 | 3 | 3 | 20 | 10 | 0 | 0 | 0 | 0 | NA | 0 | 0 | 10 |
| T_FCC_1 | 1 | | | | 1 | 3 | 2 | 21 | 1 | 0 | 0 | 0 | 0 | NA | 0 | 0 | 10 |
| T_FCC_2 | 1 | | | | 1 | 3 | 2 | 21 | 1 | 0 | 0 | 0 | 0 | NA | 0 | 0 | 10 |
| T_FCC_3 | 1 | | | | 1 | 3 | 2 | 21 | 1 | 0 | 0 | 0 | 0 | NA | 0 | 0 | 10 |
| T_FCC_4 | 1 | | | | 1 | 3 | 2 | 21 | 1 | 0 | 0 | 0 | 0 | NA | 0 | 0 | 10 |
| T_FCC_5 | 1 | FSCC_S98_14359_{—} | MG1999052110AA | SR2000060816AA | 1 | 3 | 2 | 21 | 1 | 0 | 0 | 0 | 0 | NA | 0 | 0 | 10 |
| T_FCC_6 | 1 | | | | 1 | 3 | 2 | 21 | 1 | 0 | 0 | 0 | 0 | NA | 0 | 0 | 10 |
| T_FCC_7 | 1 | | | | 1 | 3 | 2 | 21 | 1 | 0 | 0 | 0 | 0 | NA | 0 | 0 | 10 |
| T_FCC_8 | 1 | | | | 1 | 3 | 2 | 21 | 1 | 0 | 0 | 0 | 0 | NA | 0 | 0 | 10 |
| T_LBL_1 | 1 | | | | 1 | 3 | 2 | 22 | 1 | 0 | 0 | 0 | 0 | NA | 0 | 0 | 10 |
| T_LBL_2 | 1 | | | | 1 | 3 | 2 | 22 | 1 | 0 | 0 | 0 | 0 | NA | 0 | 0 | 10 |
| T_LBL_3 | 1 | | MG19991001015AA | | 1 | 3 | 2 | 22 | 1 | 0 | 0 | 0 | 0 | NA | 0 | 0 | 10 |
| T_LBL_4 | 1 | | | | 1 | 3 | 2 | 22 | 1 | 0 | 0 | 0 | 0 | NA | 0 | 0 | 10 |
| T_LBL_5 | 1 | | | | 1 | 3 | 2 | 22 | 1 | 0 | 0 | 0 | 0 | NA | 0 | 0 | 10 |
| T_LBL_6 | 1 | L_B_CELL_S97_27534_G_{—} | MG1999101304AA | SR2000060801AA | 1 | 3 | 2 | 22 | 1 | 0 | 0 | 0 | 0 | NA | 0 | 0 | 10 |
| T_LBL_7 | 1 | | | | 1 | 3 | 2 | 22 | 1 | 0 | 0 | 0 | 0 | NA | 0 | 0 | 10 |
| T_LBL_8 | 1 | | MG1999100110AA | | 1 | 3 | 2 | 22 | 1 | 0 | 0 | 0 | 0 | NA | 0 | 0 | 10 |
| T_MF_1 | 1 | | | | 1 | 4 | 2 | 23 | 1 | 0 | 0 | 0 | 0 | NA | 0 | 0 | 10 |
| T_MF_2 | 1 | | | | 1 | 4 | 2 | 23 | 1 | 0 | 0 | 0 | 0 | NA | 0 | 0 | 10 |
| T_MF_3 | 1 | | | | 1 | 4 | 2 | 23 | 1 | 0 | 0 | 0 | 0 | NA | 0 | 0 | 10 |
| TCL_K562_1 | 1 | | | | 1 | 4 | 3 | 24 | 5 | 0 | 0 | 0 | 0 | NA | 0 | 0 | 10 |
| TCL_K562_2 | 1 | | | | 1 | 4 | 3 | 24 | 5 | 0 | 0 | 0 | 0 | NA | 0 | 0 | 10 |
| TCL_HEL_1 | 1 | | | | 1 | 4 | 3 | 24 | 6 | 0 | 0 | 0 | 0 | NA | 0 | 0 | 10 |
| TCL_HEL_2 | 1 | | | | 1 | 4 | 3 | 24 | 6 | 0 | 0 | 0 | 0 | NA | 0 | 0 | 10 |
| TCL_HEL_3 | 1 | | | | 1 | 4 | 3 | 24 | 6 | 0 | 0 | 0 | 0 | NA | 0 | 0 | 10 |
| TCL_TF- | 1 | | | | 1 | 4 | 3 | 24 | 7 | 0 | 0 | 0 | 0 | NA | 0 | 0 | 10 |
| 1_1 | | | | | | | | | | | | | | | | | |
| TCL_TF- | 1 | | | | 1 | 4 | 3 | 24 | 7 | 0 | 0 | 0 | 0 | NA | 0 | 0 | 10 |
| 1_2 | | | | | | | | | | | | | | | | | |
| TCL_TF- | 1 | | | | 1 | 4 | 3 | 24 | 7 | 0 | 0 | 0 | 0 | NA | 0 | 0 | 10 |
| 1_3 | | | | | | | | | | | | | | | | | |
| PDT_BRST_1 | 2 | CUP_5 | CL2000080121AA | CL2000080818AA | 1 | 1 | 2 | 13 | 1 | 1 | 0 | 1 | 0 | NA | 0 | 0 | 10 |
| PDT_BRST_2 | 2 | CUP_2 | CL2000080117AA | CL2000080815AA | 1 | 1 | 2 | 13 | 1 | 1 | 0 | 1 | 0 | NA | 0 | 0 | 10 |
| PDT_BRST_3 | 2 | CUP_11 | CL2000080127AA | CL2000080824AA | 1 | 1 | 2 | 13 | 1 | 1 | 0 | 1 | 0 | NA | 0 | 0 | 10 |
| PDT_BRST_4 | 2 | CUP_3 | CL2000080119AA | CL2000080816AA | 1 | 1 | 2 | 13 | 1 | 1 | 0 | 1 | 0 | NA | 0 | 0 | 10 |
| PDT_BRST_5 | 2 | CUP_1 | CL2000080118AA | CL2000080814AA | 1 | 1 | 2 | 13 | 1 | 1 | 0 | 1 | 0 | NA | 0 | 0 | 10 |
| PDT_COLON_1 | 2 | CUP_15 | CL2000081105AA | CL2000081505AA | 1 | 1 | 2 | 2 | 1 | 1 | 0 | 1 | 1 | NA | 0 | 0 | 10 |
| PDT_LBL_1 | 2 | | | | 1 | 1 | 2 | 22 | 1 | 2 | 0 | 0 | 0 | NA | 0 | 0 | 10 |
| PDT_LUNG_1 | 2 | CUP_12 | CL2000081102AA | CL2000081502AA | 1 | 1 | 2 | 10 | 1 | 1 | 0 | 1 | 0 | NA | 0 | 0 | 10 |
| PDT_LUNG_2 | 2 | CUP_9 | CL2000080125AA | CL2000080822AA | 1 | 1 | 2 | 10 | 1 | 1 | 0 | 1 | 0 | NA | 0 | 0 | 10 |
| PDT_LUNG_3 | 2 | CUP_8 | CL2000081101AA | CL2000081501AA | 1 | 1 | 2 | 10 | 1 | 1 | 0 | 1 | 0 | NA | 0 | 0 | 10 |
| PDT_LUNG_4 | 2 | CUP_6 | CL2000080122AA | CL2000080819AA | 1 | 1 | 2 | 10 | 1 | 1 | 0 | 1 | 0 | NA | 0 | 0 | 10 |
| PDT_LUNG_5 | 2 | CUP_22 | CL2000081112AA | CL2000081512AA | 1 | 1 | 2 | 10 | 1 | 1 | 0 | 1 | 0 | NA | 0 | 0 | 10 |
| PDT_LUNG_6 | 2 | CUP_7 | CL2000080123AA | CL2000080820AA | 1 | 1 | 2 | 10 | 1 | 1 | 0 | 1 | 0 | NA | 0 | 0 | 10 |
| PDT_LUNG_7 | 2 | CUP_10 | CL2000080126AA | CL2000080823AA | 1 | 1 | 2 | 10 | 1 | 1 | 0 | 1 | 0 | NA | 0 | 0 | 10 |
| PDT_LUNG_8 | 2 | CUP_4 | CL2000080120AA | CL2000080817AA | 1 | 1 | 2 | 10 | 1 | 1 | 0 | 1 | 0 | NA | 0 | 0 | 10 |
| PDT_OVARY_1 | 2 | CUP_13 | CL2000081103AA | CL2000081503AA | 1 | 1 | 2 | 8 | 1 | 1 | 0 | 1 | 0 | NA | 0 | 0 | 10 |
| PDT_OVARY_2 | 2 | CUP_14 | CL2000081104AA | CL2000081504AA | 1 | 1 | 2 | 8 | 1 | 1 | 0 | 1 | 0 | NA | 0 | 0 | 10 |
| PDT_OVARY_3 | 2 | CUP_17 | CL2000081107AA | CL2000081507AA | 1 | 1 | 2 | 8 | 1 | 1 | 0 | 1 | 0 | NA | 0 | 0 | 10 |
| PDT_STOM_1 | 2 | | | | 1 | 1 | 2 | 1 | 1 | 2 | 0 | 1 | 1 | NA | 0 | 0 | 10 |
| N_MLUNG_1 | 3 | | | | 1 | 4 | 1 | 26 | 1 | 0 | 0 | 1 | 0 | NA | 0 | 0 | 5 |
| N_MLUNG_2 | 3 | | | | 1 | 4 | 1 | 26 | 1 | 0 | 0 | 1 | 0 | NA | 0 | 0 | 5 |
| N_MLUNG_3 | 3 | | | | 1 | 4 | 1 | 26 | 1 | 0 | 0 | 1 | 0 | NA | 0 | 0 | 5 |
| N_MLUNG_4 | 3 | | | | 1 | 4 | 1 | 26 | 1 | 0 | 0 | 1 | 0 | NA | 0 | 0 | 5 |
| N_MLUNG_5 | 3 | | | | 1 | 4 | 1 | 26 | 1 | 0 | 0 | 1 | 0 | NA | 0 | 0 | 5 |
| T_MLUNG_1 | 3 | | | | 1 | 4 | 2 | 26 | 1 | 0 | 0 | 1 | 0 | NA | 0 | 0 | 5 |
| T_MLUNG_2 | 3 | | | | 1 | 4 | 2 | 26 | 1 | 0 | 0 | 1 | 0 | NA | 0 | 0 | 5 |
| T_MLUNG_3 | 3 | | | | 1 | 4 | 2 | 26 | 1 | 0 | 0 | 1 | 0 | NA | 0 | 0 | 5 |
| T_MLUNG_4 | 3 | | | | 1 | 4 | 2 | 26 | 1 | 0 | 0 | 1 | 0 | NA | 0 | 0 | 5 |
| T_MLUNG_5 | 3 | | | | 1 | 4 | 2 | 26 | 1 | 0 | 0 | 1 | 0 | NA | 0 | 0 | 5 |
| T_MLUNG_6 | 3 | | | | 1 | 4 | 2 | 26 | 1 | 0 | 0 | 1 | 0 | NA | 0 | 0 | 5 |
| T_MLUNG_7 | 3 | | | | 1 | 4 | 2 | 26 | 1 | 0 | 0 | 1 | 0 | NA | 0 | 0 | 5 |
| T_SJ_ALL_1 | 4 | | | | 2 | 2 | 2 | 19 | 1 | 0 | 3 | 0 | 0 | NA | 0 | 0 | 5 |
| T_SJ_ALL_2 | 4 | | | | 2 | 2 | 2 | 19 | 1 | 0 | 9 | 0 | 0 | NA | 0 | 0 | 5 |
| T_SJ_ALL_3 | 4 | | | | 2 | 2 | 2 | 19 | 1 | 0 | 4 | 0 | 0 | NA | 0 | 0 | 5 |
| T_SJ_ALL_4 | 4 | | | | 2 | 2 | 2 | 19 | 1 | 0 | 3 | 0 | 0 | NA | 0 | 0 | 5 |
| T_SJ_ALL_5 | 4 | | | | 2 | 2 | 2 | 20 | 1 | 0 | 6 | 0 | 0 | NA | 0 | 0 | 5 |
| T_SJ_ALL_6 | 4 | | | | 2 | 2 | 2 | 19 | 1 | 0 | 1 | 0 | 0 | NA | 0 | 0 | 5 |
| T_SJ_ALL_7 | 4 | | | | 2 | 2 | 2 | 19 | 1 | 0 | 9 | 0 | 0 | NA | 0 | 0 | 5 |
| T_SJ_ALL_8 | 4 | | | | 2 | 2 | 2 | 20 | 1 | 0 | 6 | 0 | 0 | NA | 0 | 0 | 5 |
| T_SJ_ALL_9 | 4 | | | | 2 | 2 | 2 | 19 | 1 | 0 | 4 | 0 | 0 | NA | 0 | 0 | 5 |
| T_SJ_ALL_10 | 4 | | | | 2 | 2 | 2 | 19 | 1 | 0 | 5 | 0 | 0 | NA | 0 | 0 | 5 |
| T_SJ_ALL_11 | 4 | | | | 2 | 2 | 2 | 19 | 1 | 0 | 7 | 0 | 0 | NA | 0 | 0 | 5 |
| T_SJ_ALL_12 | 4 | | | | 2 | 2 | 2 | 19 | 1 | 0 | 7 | 0 | 0 | NA | 0 | 0 | 5 |
| T_SJ_ALL_13 | 4 | | | | 2 | 2 | 2 | 19 | 1 | 0 | 7 | 0 | 0 | NA | 0 | 0 | 5 |
| T_SJ_ALL_14 | 4 | | | | 2 | 2 | 2 | 20 | 1 | 0 | 6 | 0 | 0 | NA | 0 | 0 | 5 |
| T_SJ_ALL_15 | 4 | | | | 2 | 2 | 2 | 19 | 1 | 0 | 3 | 0 | 0 | NA | 0 | 0 | 5 |
| T_SJ_ALL_16 | 4 | | | | 2 | 2 | 2 | 19 | 1 | 0 | 4 | 0 | 0 | NA | 0 | 0 | 5 |
| T_SJ_ALL_17 | 4 | | | | 2 | 2 | 2 | 20 | 1 | 0 | 6 | 0 | 0 | NA | 0 | 0 | 5 |
| T_SJ_ALL_18 | 4 | | | | 2 | 2 | 2 | 20 | 1 | 0 | 6 | 0 | 0 | NA | 0 | 0 | 5 |
| T_SJ_ALL_19 | 4 | | | | 2 | 2 | 2 | 19 | 1 | 0 | 9 | 0 | 0 | NA | 0 | 0 | 5 |
| T_SJ_ALL_20 | 4 | | | | 2 | 2 | 2 | 19 | 1 | 0 | 7 | 0 | 0 | NA | 0 | 0 | 5 |
| T_SJ_ALL_21 | 4 | | | | 2 | 2 | 2 | 20 | 1 | 0 | 6 | 0 | 0 | NA | 0 | 0 | 5 |
| T_SJ_ALL_22 | 4 | | | | 2 | 2 | 2 | 20 | 1 | 0 | 6 | 0 | 0 | NA | 0 | 0 | 5 |
| T_SJ_ALL_23 | 4 | | | | 2 | 2 | 2 | 19 | 1 | 0 | 9 | 0 | 0 | NA | 0 | 0 | 5 |
| T_SJ_ALL_24 | 4 | | | | 2 | 2 | 2 | 20 | 1 | 0 | 6 | 0 | 0 | NA | 0 | 0 | 5 |
| T_SJ_ALL_25 | 4 | | | | 2 | 2 | 2 | 19 | 1 | 0 | 5 | 0 | 0 | NA | 0 | 0 | 5 |
| T_SJ_ALL_26 | 4 | | | | 2 | 2 | 2 | 20 | 1 | 0 | 6 | 0 | 0 | NA | 0 | 0 | 5 |
| T_SJ_ALL_27 | 4 | | | | 2 | 2 | 2 | 19 | 1 | 0 | 2 | 0 | 0 | NA | 0 | 0 | 5 |
| T_SJ_ALL_28 | 4 | | | | 2 | 2 | 2 | 19 | 1 | 0 | 4 | 0 | 0 | NA | 0 | 0 | 5 |
| T_SJ_ALL_29 | 4 | | | | 2 | 2 | 2 | 19 | 1 | 0 | 2 | 0 | 0 | NA | 0 | 0 | 5 |
| T_SJ_ALL_30 | 4 | | | | 2 | 2 | 2 | 19 | 1 | 0 | 1 | 0 | 0 | NA | 0 | 0 | 5 |
| T_SJ_ALL_31 | 4 | | | | 2 | 2 | 2 | 19 | 1 | 0 | 5 | 0 | 0 | NA | 0 | 0 | 5 |
| T_SJ_ALL_32 | 4 | | | | 2 | 2 | 2 | 19 | 1 | 0 | 5 | 0 | 0 | NA | 0 | 0 | 5 |
| T_SJ_ALL_33 | 4 | | | | 2 | 2 | 2 | 19 | 1 | 0 | 1 | 0 | 0 | NA | 0 | 0 | 5 |
| T_SJ_ALL_34 | 4 | | | | 2 | 2 | 2 | 20 | 1 | 0 | 6 | 0 | 0 | NA | 0 | 0 | 5 |
| T_SJ_ALL_35 | 4 | | | | 2 | 2 | 2 | 19 | 1 | 0 | 2 | 0 | 0 | NA | 0 | 0 | 5 |
| T_SJ_ALL_36 | 4 | | | | 2 | 2 | 2 | 20 | 1 | 0 | 6 | 0 | 0 | NA | 0 | 0 | 5 |
| T_SJ_ALL_37 | 4 | | | | 2 | 2 | 2 | 20 | 1 | 0 | 6 | 0 | 0 | NA | 0 | 0 | 5 |
| T_SJ_ALL_38 | 4 | | | | 2 | 2 | 2 | 19 | 1 | 0 | 3 | 0 | 0 | NA | 0 | 0 | 5 |
| T_SJ_ALL_39 | 4 | | | | 2 | 2 | 2 | 19 | 1 | 0 | 4 | 0 | 0 | NA | 0 | 0 | 5 |
| T_SJ_ALL_40 | 4 | | | | 2 | 2 | 2 | 20 | 1 | 0 | 6 | 0 | 0 | NA | 0 | 0 | 5 |
| T_SJ_ALL_41 | 4 | | | | 2 | 2 | 2 | 19 | 1 | 0 | 2 | 0 | 0 | NA | 0 | 0 | 5 |
| T_SJ_ALL_42 | 4 | | | | 2 | 2 | 2 | 19 | 1 | 0 | 7 | 0 | 0 | NA | 0 | 0 | 5 |
| T_SJ_ALL_43 | 4 | | | | 2 | 2 | 2 | 20 | 1 | 0 | 6 | 0 | 0 | NA | 0 | 0 | 5 |
| T_SJ_ALL_44 | 4 | | | | 2 | 2 | 2 | 19 | 1 | 0 | 3 | 0 | 0 | NA | 0 | 0 | 5 |
| T_SJ_ALL_45 | 4 | | | | 2 | 2 | 2 | 19 | 1 | 0 | 4 | 0 | 0 | NA | 0 | 0 | 5 |
| T_SJ_ALL_46 | 4 | | | | 2 | 2 | 2 | 19 | 1 | 0 | 7 | 0 | 0 | NA | 0 | 0 | 5 |
| T_SJ_ALL_47 | 4 | | | | 2 | 2 | 2 | 20 | 1 | 0 | 6 | 0 | 0 | NA | 0 | 0 | 5 |
| T_SJ_ALL_48 | 4 | | | | 2 | 2 | 2 | 19 | 1 | 0 | 1 | 0 | 0 | NA | 0 | 0 | 5 |
| T_SJ_ALL_49 | 4 | | | | 2 | 2 | 2 | 19 | 1 | 0 | 5 | 0 | 0 | NA | 0 | 0 | 5 |
| T_SJ_ALL_50 | 4 | | | | 2 | 2 | 2 | 19 | 1 | 0 | 4 | 0 | 0 | NA | 0 | 0 | 5 |
| T_SJ_ALL_51 | 4 | | | | 2 | 2 | 2 | 19 | 1 | 0 | 3 | 0 | 0 | NA | 0 | 0 | 5 |
| T_SJ_ALL_52 | 4 | | | | 2 | 2 | 2 | 19 | 1 | 0 | 3 | 0 | 0 | NA | 0 | 0 | 5 |
| T_SJ_ALL_53 | 4 | | | | 2 | 2 | 2 | 19 | 1 | 0 | 5 | 0 | 0 | NA | 0 | 0 | 5 |
| T_SJ_ALL_54 | 4 | | | | 2 | 2 | 2 | 20 | 1 | 0 | 6 | 0 | 0 | NA | 0 | 0 | 5 |
| T_SJ_ALL_55 | 4 | | | | 2 | 2 | 2 | 20 | 1 | 0 | 6 | 0 | 0 | NA | 0 | 0 | 5 |
| T_SJ_ALL_56 | 4 | | | | 2 | 2 | 2 | 19 | 1 | 0 | 4 | 0 | 0 | NA | 0 | 0 | 5 |
| T_SJ_ALL_57 | 4 | | | | 2 | 2 | 2 | 19 | 1 | 0 | 2 | 0 | 0 | NA | 0 | 0 | 5 |
| T_SJ_ALL_58 | 4 | | | | 2 | 2 | 2 | 20 | 1 | 0 | 6 | 0 | 0 | NA | 0 | 0 | 5 |
| T_SJ_ALL_59 | 4 | | | | 2 | 2 | 2 | 20 | 1 | 0 | 6 | 0 | 0 | NA | 0 | 0 | 5 |
| T_SJ_ALL_60 | 4 | | | | 2 | 2 | 2 | 19 | 1 | 0 | 7 | 0 | 0 | NA | 0 | 0 | 5 |
| T_SJ_ALL_61 | 4 | | | | 2 | 2 | 2 | 19 | 1 | 0 | 7 | 0 | 0 | NA | 0 | 0 | 5 |
| T_SJ_ALL_62 | 4 | | | | 2 | 2 | 2 | 19 | 1 | 0 | 3 | 0 | 0 | NA | 0 | 0 | 5 |
| T_SJ_ALL_63 | 4 | | | | 2 | 2 | 2 | 19 | 1 | 0 | 7 | 0 | 0 | NA | 0 | 0 | 5 |
| T_SJ_ALL_64 | 4 | | | | 2 | 2 | 2 | 19 | 1 | 0 | 2 | 0 | 0 | NA | 0 | 0 | 5 |
| T_SJ_ALL_65 | 4 | | | | 2 | 2 | 2 | 19 | 1 | 0 | 2 | 0 | 0 | NA | 0 | 0 | 5 |
| T_SJ_ALL_66 | 4 | | | | 2 | 2 | 2 | 19 | 1 | 0 | 2 | 0 | 0 | NA | 0 | 0 | 5 |
| T_SJ_ALL_67 | 4 | | | | 2 | 2 | 2 | 19 | 1 | 0 | 1 | 0 | 0 | NA | 0 | 0 | 5 |
| T_SJ_ALL_68 | 4 | | | | 2 | 2 | 2 | 19 | 1 | 0 | 7 | 0 | 0 | NA | 0 | 0 | 5 |
| T_SJ_ALL_69 | 4 | | | | 2 | 2 | 2 | 19 | 1 | 0 | 2 | 0 | 0 | NA | 0 | 0 | 5 |
| T_SJ_ALL_70 | 4 | | | | 2 | 2 | 2 | 19 | 1 | 0 | 4 | 0 | 0 | NA | 0 | 0 | 5 |
| T_SJ_ALL_71 | 4 | | | | 2 | 2 | 2 | 19 | 1 | 0 | 3 | 0 | 0 | NA | 0 | 0 | 5 |
| T_SJ_ALL_72 | 4 | | | | 2 | 2 | 2 | 19 | 1 | 0 | 7 | 0 | 0 | NA | 0 | 0 | 5 |
| T_SJ_ALL_73 | 4 | | | | 2 | 2 | 2 | 19 | 1 | 0 | 3 | 0 | 0 | NA | 0 | 0 | 5 |
| TCL_HL60_1 | 5 | | | | 1 | 4 | 3 | 24 | 9 | 0 | 0 | 0 | 0 | 1-Day | 0 | 0 | 5 |
| | | | | | | | | | | | | | | ATRA | | | |
| TCL_HL60_2 | 5 | | | | 1 | 4 | 3 | 24 | 9 | 0 | 0 | 0 | 0 | 3-Day | 0 | 0 | 5 |
| | | | | | | | | | | | | | | ATRA | | | |
| TCL_HL60_3 | 5 | | | | 1 | 4 | 3 | 24 | 9 | 0 | 0 | 0 | 0 | 5-Day | 0 | 0 | 5 |
| | | | | | | | | | | | | | | ATRA | | | |
| TCL_HL60_4 | 5 | | | | 1 | 4 | 3 | 24 | 9 | 0 | 0 | 0 | 0 | 1-Day + | 0 | 0 | 5 |
| | | | | | | | | | | | | | | ATRA | | | |
| TCL_HL60_5 | 5 | | | | 1 | 4 | 3 | 24 | 9 | 0 | 0 | 0 | 0 | 3-Day + | 0 | 0 | 5 |
| | | | | | | | | | | | | | | ATRA | | | |
| TCL_HL60_6 | 5 | | | | 1 | 4 | 3 | 24 | 9 | 0 | 0 | 0 | 0 | 5-Day + | 0 | 0 | 5 |
| | | | | | | | | | | | | | | ATRA | | | |
| N_ERYTH_1 | 6 | | | | 2 | 4 | 1 | 27 | 1 | 0 | 0 | 0 | 0 | 2-Day | 0 | 0 | 1.6 |
| N_ERYTH_2 | 6 | | | | 2 | 4 | 1 | 27 | 1 | 0 | 0 | 0 | 0 | 4-Day | 0 | 0 | 1.6 |
| N_ERYTH_3 | 6 | | | | 2 | 4 | 1 | 27 | 1 | 0 | 0 | 0 | 0 | 6-Day | 0 | 0 | 1.6 |
| N_ERYTH_4 | 6 | | | | 2 | 4 | 1 | 27 | 1 | 0 | 0 | 0 | 0 | 8-Day | 0 | 0 | 1.6 |
| N_ERYTH_{—} | 6 | | | | 2 | 4 | 1 | 27 | 1 | 0 | 0 | 0 | 0 | 10-Day | 0 | 0 | 1.6 |
| N_ERYTH_{—} | 6 | | | | 2 | 4 | 1 | 27 | 1 | 0 | 0 | 0 | 0 | 12-Day | 0 | 0 | 1.6 |

| | |
|---|---|
| Field | Description |
| Name | Sample name used in this study |
| Data Set | Data set that stores the miRNA expression data; 1 for miGCM, 2 for PDT_miRNA, 3 for mLung, 4 for ALL, 5 for HL60, 6 for erythroid |
| SR Name | Corresponding sample name in Ramaswamy et al, PNAS, 2001, 98: 15149-15154; empty entry for no match |
| HuFL Scan | Scan name for Affymetrix HuFL (Hu6800) chip, if available |
| Hu35KsubA | Scan name for Affymetrix Hu35KsubA chip, if available |
| Scan | |
| BV | Bead version that is used to detect the sample |
| SSC | Sample source code; 1 for Ramaswamy study, 2 for St Jude, 3 for Dana-Farber, 4 for MIT |
| MAL | Maliganancy status code; 1 for Normal, 2 for Tumor, 3 for cell line |
| TT | Tissue type code; 1 for stomach, 2 for colon, 3 for pancreas, 4 for liver, 5 for kidney, 6 for bladder, 7 for prostate, 8 for ovary, 9 for uterus, 10 |
| | for human lung, 11 for mesothelioma, 12 for melanoma, 13 for breast, 14 for brain, 19 for B cell ALL, 20 for T cell ALL, 21 for follicular |
| | cleaved lymphoma, 22 for large B cell lymphoma, 23 for mycosis fungoidis, 24 for acute myelogenous leukemia, 26 for mouse lung, 27 for |
| | erythrocytes |
| CLT | Cell line type code; 1 for non-cell-line/others, 2 for MCF-7, 3 for SKMEL-5, 4 for PC-3, 5 for K562, 6 for HEL, 7 for TF-1, 8 for 293, 9 for |
| | HL60, 10 for T-ALL cell lines |
| PDT | Poorly differentiated tumor (PDT) code; 0 for others, 1 for PDT used in prediction, 2 for PDT not used in prediction due to lack of successful |
| | Affymetrix scans |
| AS | ALL Subtype; 0 for others or unknowns, 1 for BCR/ABL, 2 for E2A/PBX1, 3 for Hyperdiploid 47 to 50, 4 for Hyperdiploid >50, 5 or MLL, 6 for |
| | T_ALL, 7 for TEL/AML1, 9 for Normal ploidy |
| EP | Epithelial code; 0 for others, 1 for epithelial sample |
| GI | Gastrointestinal tract code; 0 for others and cell lines, 1 for GI sample |
| Culture | Description of culture condition for HL-60 and erythrocyte differentiation experiments |
| N-T CLS | Sample used to build the normal/tumor classifier; 0 for others, 1 for used |
| MultiC CLS | Sample used to build the multi-cancer classifier; 0 for others, 1 for used |
| RNA | Sample quantity of total RNA for profiling, measured in micrograms |

TABLE 10a-10b
Probe Information+TZ,1/64

| | | |
|---|---|---|
| Field | Description+TZ,1/64 | |
| Probe ID | Probe name | |
| Seq Type | Biosequence type; oligo for deoxyoligonucleotides | |
| Probe Sequence | 5′ to 3′ capture probe sequence | |
| Target Sequence | 5′ to 3′ target or target mutant sequence; NA for not available | |
| Human | Human miRNA recognized by probe according to microRNA registry rfam 5.0 | |
| Mouse | Mouse miRNA recognized by probe according to microRNA registry rfam 5.0 | |
| Rat | Rat | miRNA recognized by probe according to microRNA registry rfam 5.0 |
| Other | Special note about recognition | |
| Control | Whether the feature is a control feature and what | type of control |
| Set Number (V1) | The set of beads this feature belongs to in version 1 | |
| Set Number (V2) | The set of beads this feature belongs to in version 2 | |
| Usage | Whether the feature is used in the final dataset for analyses and why not+TZ,1/64 | |

TABLE 10a+TZ,1/64

| | | | | | |
|---|---|---|---|---|---|
| Probe | Seq | +TL,12 | +TL,41 | +TL,56 | +TL,64 |
| ID | Type | Probe Sequence | Target Sequence+TZ,1/64 | | |

| | | | | | |
|---|---|---|---|---|---|
| EAM103 | Oligo | /5AmMC6/TGGCATTCACCGCGTGCCTTA | seq id no:286 | UUAAGGCACGCGGUGAAUGCCA | seq id no 568 |
| EAM105 | Oligo | /5AmMC6/TCACAAGTTAGGGTCTCAGGGA | seq id no:287 | UCCCUGAGACCCUAACUUGUGA | seq id no:569 |
| EAM109 | Oligo | /5AmMC6/AACAACAAAATCACTAGTCTTCCA | seq id no:288 | UGGAAGACUAGUGAUUUUGUU | seq id no:570 |
| EAM111 | Oligo | /5AmMC6/TAACTGTACAAACTACTACCTCA | seq id no:289 | UGAGGUAGUAGUUUGUACAGU | seq id no:571 |
| EAM115 | Oligo | /5AmMC6/CGCCAATATTTACGTGCTGCTA | seq id no:290 | UAGCAGCACGUAAAUAUUGGCG | seq id no:572 |
| EAM119 | Oligo | /5AmMC6/AACACTGATTTCAAATGGTGCTA | seq id no:291 | UAGCACCAUUUGAAAUCAGUGU | seq id no:573 |
| EAM121 | Oligo | /5AmMC6/CACAAGATCGGATCTACGGGT | seq id no:292 | AACCCGUAGAUCCGAUCUUGUG | seq id no:574 |
| EAM131 | Oligo | /5AmMC6/ACAGGCCGGGACAAGTGCAATAT | seq id no:293 | UAUUGCACUUGUCCCGGCCUGU | seq id no:575 |
| EAM139 | Oligo | /5AmMC6/TAACCCATGGAATTCAGTTCTCA | seq id no:294 | UGAGAACUGAAUUCCAUGGGUU | seq id no:576 |
| EAM145 | Oligo | /5AmMC6/AACCATACAACCTACTACCTCA | seq id no:295 | UGAGGUAGUAGGUUGUAUGGUU | seq id no:577 |
| EAM152 | Oligo | /5AmMC6/ACTTTCGGTTATCTAGCTTTAT | seq id no:296 | UAAAGCUAGAUAACCGAAAGU | seq id no:578 |
| EAM238 | Oligo | /5AmMC6/ATACATACTTCTTTACATTCCA | seq id no:297 | UGGAAUGUAAAGAAGUAUGUA | seq id no:579 |
| EAM270 | Oligo | /5AmMC6/GCTGAGTGTAGGATGTTTACA | seq id no:298 | UGUAAACAUCCUACACUCAGC | seq id no:580 |
| EAM159 | Oligo | /5AmMC6/ATGCCCTTTTAACATTGCACTG | seq id no:299 | CAGUGCAAUGUUAAAAGGGC | seq id no:581 |
| EAM163 | Oligo | /5AmMC6/TCCATAAAGTAGGAAACACTACA | seq id no:300 | UGUAGUGUUUCCUACUUUAUGGA | seq id no:582 |
| EAM171 | Oligo | /SAmMC6/CTACGCGTATTCTTAAGCAATAA | seq id no:301 | UAUUGCUUAAGAAUACGCGUAG | seq id no:583 |
| EAM183 | Oligo | /5AmMC6/AGCACAAACTACTACCTCA | seq id no:302 | UGAGGUAGUAGUUUGUGCU | seq id no:584 |
| EAM184 | Oiigo | /5AmMC6/CACAAGTTCGGATCTACGGGTT | seq id no:303 | AACCCGUAGAUCCGAACUUGUG | seq id no:585 |
| EAM186 | Oligo | /5AmMC6/GCTACCTGCACTGTAAGCACTTTT | seq id no:304 | AAAAGUGCUUACAGUGCAGGUAGC | seq id no:586 |
| EAM189 | Oligo | /5AmMC6/CACAAATTCGGATCTACAGGGTA | seq id no:305 | UACCCUGUAGAUCCGAAUUUGUG | seq id no:587 |
| EAM191 | Oligo | /5AmMC6/ACAAACACCATTGTCACACTCCA | seq id no:306 | UGGAGUGUGACAAUGGUGUUUGU | seq id no:588 |
| EAM192 | Oligo | /5AmMC6/CGCGTACCAAAAGTAATAATG | seq id no:307 | CAUUAUUACUUUUGGUACGCG | seq id no:589 |
| EAM198 | Oligo | /5AmMC6/GCCCTTTCATCATTGCACTG | seq id no:308 | CAGUGCAAUGAUGAAAGGGCAU | seq id no:590 |
| EAM202 | Oligo | /5AmMC6/TCCCTCTGGTCAACCAGTCACA | seq id no:309 | UGUGACUGGUUGACCAGAGGG | seq id no:591 |
| EAM209 | Oligo | /5AmMC6/GTAGTGCTTTCTACTTTATG | seq id no:310 | CAUAAAGUAGAAAGCACUAC | seq id no:592 |
| EAM221 | Oligo | /5AmMC6/CCCCTATCACAATTAGCATTAA | seq id no:311 | UUAAUGCUAAUUGUGAUAGGGG | seq id no:593 |
| EAM223 | Oligo | /5AmMC6/TGTAAACCATGATGTGCTGCTA | seq id no:312 | UAGCAGCACAUCAUGGUUUACA | seq id no:594 |
| EAM224 | Oligo | /5AmMC6/ACTACCTGCACTGTAAGCACTTTG | seq id no:313 | CAAAGUGCUUACAGUGCAGGUAGU | seq id no:595 |
| EAM225 | Oligo | /5AmMC6/TATCTGCACTAGATGCACCTTA | seq id no:314 | UAAGGUGCAUCUAGUGCAGAUA | seq id no:596 |
| EAM226 | Oligo | /5AmMC6/ACTCACCGACAGCGTTGAATGTT | seq id no:315 | AACAUUCAACGCUGUCGGUGAGU | seq id no:597 |
| EAM227 | Oligo | /5AmMC6/AACCCACCGACAGCAATGAATGTT | seq id no:316 | AACAUUCAUUGCUGUCGGUGGGUU | seq id no:598 |
| EAM234 | Oligo | /5AmMC6/GAACAGGTAGTCTGAACACTGGG | seq id no:317 | CCCAGUGUUCAGACUACCUGUUC | seq id no:599 |
| EAM235 | Oligo | /5AmMC6/GAACAGATAGTCTAAACACTGGG | seq id no:318 | CCCAGUGUUUAGACUAUCUGUUC | seq id no:600 |
| EAM236 | Oligo | /5AmMC6/TCAGTTTTGCATAGATTTGCACA | seq id no:319 | UGUGCAAAUCUAUGCAAAACUGA | seq id no:601 |
| EAM241 | Oligo | /5AmMC6/CTAGTGGTCCTAAACATTTCAC | seq id no:320 | GUGAAAUGUUUAGGACCACUAG | seq id no:602 |
| EAM242 | Oligo | /5AmMC6/AGGCATAGGATGACAAAGGGAA | seq id no:321 | UUCCCUUUGUCAUCCUAUGCCUG | seq id no:603 |
| EAM243 | Oligo | /5AmMC6/CAGACTCCGGTGGAATGAAGGA | seq id no:322 | UCCUUCAUUCCACCGGAGUCUG | seq id no:604 |
| EAM245 | Oligo | /5AmMC6/CAGCCGCTGTCACACGCACAG | seq id no:323 | CUGUGCGUGUGACAGCGGCUG | seq id no:605 |
| EAM249 | Oligo | /5AmMC6/CTGCCTGTCTGTGCCTGCTGT | seq id no:324 | ACAGCAGGCACAGACAGGCAG | seq id no:606 |
| EAM254 | Oligo | /5AmMC6/AGAATTGCGTTTGGACAATCA | seq id no:325 | UGAUUGUCCAAACGCAAUUCU | seq id no:607 |
| EAM257 | Oligo | /5AmMC6/GAAACCCAGCAGACAATGTAGCT | seq id no:326 | AGCUACAUUGUCUGCUGGGUUUC | seq id no:608 |
| EAM258 | Oligo | /5AmMC6/GAGACCCAGTAGCCAGATGTAGCT | seq id no:327 | AGCUACAUCUGGCUACUGGGUCUC | seq id no:609 |
| EAM259 | Oligo | /5AmMC6/GGGGTATTTGACAAACTGACA | seq id no:328 | UGUCAGUUUGUCAAAUACCCC | seq id no:610 |
| EAM273 | Oligo | /5AmMC6/CAATGCAACTACAATGCAC | seq id no:329 | GUGCAUUGUAGUUGCAUUG | seq id no:611 |
| EAM288 | Oligo | /5AmMC6/ACACAAATTCGGTTCTACAGGG | seq id no:330 | CCCUGUAGAACCGAAUUUGUGU | seq id no:612 |
| EAM293 | Oligo | /5AmMC6/ACCCTCCACCATGCAAGGGATG | seq id no:331 | CAUCCCUUGCAUGGUGGAGGGU | seq id no:613 |
| EAM297 | Oligo | /5AmMC6/CTGGGACTTTGTAGGCCAGTT | seq id no:332 | AACUGGCCUACAAAGUCCCAG | seq id no:614 |
| EAM301 | Oligo | /5AmMC6/CCTATCTCCCCTCTGGACC | seq id no:333 | GGUCCAGAGGGGAGAUAGG | seq id no:615 |
| EAM304 | Oligo | /5AmMC6/CATCGTTACCAGACAGTGTTA | seq id no:334 | UAACACUGUCUGGUAACGAUGU | seq id no:616 |
| EAM306 | Oligo | /5AmMC6/AGAACAATGCCTTAGTGAGTA | seq id no:335 | UACUCAGUAAGGCAUUGUUCU | seq id no:617 |
| EAM307 | Oligo | /5AmMC6/TCTTCCCATGCGCTATACCTCT | seq id no:336 | AGAGGUAUAGCGCAUGGGAAGA | seq id no:618 |
| EAM308 | Oligo | /5AmMC6/CCACACACTTCCTTACATTCCA | seq id no:337 | UGGAAUGUAAGGAAGUGUGUGG | seq id no:619 |
| EAM309 | Oligo | /5AmMC6/GAGGGAGGAGAGCCAGGAGAAGC | seq id no:338 | GCUUCUCCUGGCUCUCCUCCCUG | seq id no:620 |
| EAM310 | Oligo | /5AmMC6/ACAAGCTTTTTGCTCGTCTTAT | seq id no:339 | AUAAGACGAGCAAAAAGCUUGU | seq id no:621 |
| EAM247 | Oligo | /5AmMC6/GGCCGTGACTGGAGACTGTTA | seq id no:340 | UAACAGUCUCCAGUCACGGCC | seq id no:622 |
| EAM251 | Oligo | /5AmMC6/CACAGTTGCCAGCTGAGATTA | seq id no:341 | UAAUCUCAGCUGGCAACUGUG | seq id no:623 |
| EAM253 | Oligo | /5AmMC6/ACATGGTTAGATCAAGCACAA | seq id no:342 | UUGUGCUUGAUCUAACCAUGU | seq id no:624 |
| EAM275 | Oligo | /5AmMC6/ACAACCAGCTAAGACACTGCCA | seq id no:343 | UGGCAGUGUCUUAGCUGGUUGUU | seq id no:625 |
| EAM246 | Oligo | /5AmMC6/AGGCGAAGGATGACAAAGGGAA | seq id no:344 | UUCCCUUUGUCAUCCUUCGCCU | seq id no:626 |
| EAM250 | Oligo | /5AmMC6/GTCTGTCAATTCATAGGTCAT | seq id no:345 | AUGACCUAUGAAUUGACAGAC | seq id no:627 |
| EAM252 | Oligo | /5AmMC6/ATCCAATCAGTTCCTGATGCAGTA | seq id no:346 | UACUGCAUCAGGAACUGAUUGGAU | seq id no:628 |
| EAM305 | Oligo | /5AmMC6/GTCATCATTACCAGGCAGTATTA | seq id no:347 | UAAUACUGCCUGGUAAUGAUGAC | seq id no:629 |
| EAM303 | Oligo | /5AmMG6/AACCAATGTGCAGACTACTGTA | seq id no:348 | UACAGUAGUCUGCACAUUGGUU | seq id no:630 |
| EAM300 | Oligo | /5AmMG6/GCTGGGTGGAGAAGGTGGTGAA | seq id no:349 | UUCACCACCUUCUCCACCCAGC | seq id no:631 |
| EAM299 | Oligo | /5AmMC6/GCCAATATTTCTGTGCTGCTA | seq id no:350 | UAGCAGCACAGAAAUAUUGGC | seq id no:632 |
| EAM298 | Oligo | /5AmMC6/TCCACATGGAGTTGCTGTTACA | seq id no:351 | UGUAACAGCAACUCCAUGUGGA | seq id no:633 |
| EAM296 | Oligo | /5AmMC6/AGCTGCTTTTGGGATTCCGTTG | seq id no:352 | CAACGGAAUCCCAAAAGCAGCU | seq id no:634 |
| EAM295 | Oligo | /5AmMC6/ACCTAATATATCAAACATATCA | seq id no:353 | UGAUAUGUUUGAUAUAUUAGGU | seq id no:635 |
| EAM292 | Oligo | /5AmMC6/AAGCCCAAAAGGAGAATTCTTTG | seq id no:354 | CAAAGAAUUCUCCUUUUGGGCUU | seq id no:636 |
| EAM112 | Oligo | /5AmMC6/TAACTGTAGAAAGTACTACCTCA | seq id no:355 | TGAGGTAGTACTTTCTACAGTTA | seq id no:637 |
| EAM116 | Oligo | /5AmMC6/CGCCAATATTAAGGTGCTGCTA | seq id no:356 | TAGCAGCACCTTAATATTGGCG | seq id no:638 |
| EAM120 | Oligo | /5AmMC6/AACACTGATTTGAAAAGGTGCTA | seq id no:357 | TAGCACCTTTTCAAATCAGTGTT | seq id no:639 |
| EAM122 | Oligo | /5AmMC6/CACAAGATGGGATGTACGGGT | seq id no:358 | ACCCGTACATCCCATCTTGTG | seq id no:640 |
| EAM132 | Oligo | /5AmMC6/ACAGGCCGGGAGAAGAGCAATAT | seq id no:359 | ATATTGCTCTTCTCCCGGCCTGT | seq id no:641 |
| EAM140 | Oligo | /5AmMC6/TAACCCATGGAAATGAGTTCTCA | seq id no:360 | TGAGAACTCATTTCCATGGGUA | seq id no:642 |
| EAM282 | Oligo | /5AmMC6/GAACAGGTAGTCTAAACACTGGG | seq id no:361 | CCCAGUGUUUAGACUACCUGUUC | seq id no:643 |
| EAM281 | Oligo | /5AmMC6/atccagtcagttcctgatgcagta | seq id no:362 | UACUGCAUCAGGAACUGACUGGAU | seq id no:644 |
| EAM280 | Oligo | /5AmMC6/GCTGCAAACATCCGACTGAAAG | seq id no:363 | CUUUCAGUCGGAUGUUUGCAGC | seq id no:645 |
| EAM279 | Oligo | /5AmMC6/TAACCGATTTCAAATGGTGCTA | seq id no:364 | UAGCACCAUUUGAAAUCGGUUA | seq id no:646 |
| EAM278 | Oligo | /5AmMC6/AACAATACAACTTACTACCTCA | seq id no:365 | UGAGGUAGUAAGUUGUAUUGUU | seq id no:647 |
| EAM277 | Oligo | /5AmMC6/GCAAAAATGTGCTAGTGCCAAA | seq id no:366 | UUUGGCACUAGCACAUUUUUGCU | seq id no:648 |
| EAM276 | Oligo | /5AmMC6/TCATACAGCTAGATAACCAAAGA | seq id no:367 | UCUUUGGUUAUCUAGCUGUAUGA | seq id no:649 |
| EAM272 | Oligo | /5AmMC6/CTTCCAGTCGGGGATGTTTACA | seq id no:368 | UGUAAACAUCCCCGACUGGAAG | seq id no:650 |
| EAM271 | Oligo | /5AmMC6/GCTGAGAGTGTAGGATGTTTACA | seq id no:369 | UGUAAACAUCCUACACUCUCAGC | seq id no:651 |
| EAM268 | Oligo | /5AmMC6/AACCGATTTCAGATGGTGCTAG | seq id no:370 | CUAGCACCAUCUGAAAUCGGUU | seq id no:652 |
| EAM264 | Oligo | /5AmMC6/CAGAACTTAGCCACTGTGAA | seq id no:371 | UUCACAGUGGCUAAGUUCUG | seq id no:653 |
| EAM263 | Oligo | /5AmMC6/AGCCTATCCTGGATTACTTGAA | seq id no:372 | UUCAAGUAAUCCAGGAUAGGCU | seq id no:654 |
| EAM262 | Oligo | /5AmMC6/CTGTTCCTGCTGAACTGAGCCA | seq id no:373 | UGGCUCAGUUCAGCAGGAACAG | seq id no:655 |
| EAM261 | Oligo | /5AmMC6/GTGGTAATCCCTGGCAATGTGAT | seq id no:374 | AUCACAUUGCCAGGGAUUACCAC | seq id no:656 |
| EAM260 | Oligo | /5AmMC6/GGAAATCCCTGGCAATGTGAT | seq id no:375 | AUCACAUUGCCAGGGAUUUCC | seq id no:657 |
| EAM256 | Oligo | /5AmMC6/AAAGTGTCAGATACGGTGTGG | seq id no:376 | CCACACCGUAUCUGACACUUU | seq id no:658 |
| EAM255 | Oligo | /5AmMC6/ACAGTTCTTCAACTGGCAGCTT | seq id no:377 | AAGCUGCCAGUUGAAGAACUGU | seq id no:659 |
| EAM248 | Oligo | /5AmMC6/GGTACAATCAACGGTCGATGGT | seq id no:378 | ACCAUCGACCGUUGAUUGUACC | seq id no:660 |
| EAM244 | Oligo | /5AmMC6/TCAACATCAGTCTGATAAGCTA | seq id no:379 | UAGCUUAUCAGACUGAUGUUGA | seq id no:661 |
| EAM240 | Oligo | /5AmMC6/CTACCTGCACTATAAGCACTTTA | seq id no:380 | UAAAGUGCUUAUAGUGCAGGUAG | seq id no:662 |
| EAM237 | Oligo | /5AmMC6/TCAGTTTTGCATGGATTTGCACA | seq id no:381 | UGUGCAAAUCCAUGCAAAACUGA | seq id no:663 |
| EAM233 | Oligo | /5AmMC6/CCCAACAACATGAAACTACCTA | seq id no:382 | UAGGUAGUUUCAUGUUGUUGG | seq id no:664 |
| EAM232 | Oligo | /5AmMC6/GGCTGTCAATTCATAGGTCAG | seq id no:383 | CUGACCUAUGAAUUGACAGCC | seq id no:665 |
| EAM231 | Oligo | /5AmMC6/CGGCTGCAACACAAGACACGA | seq id no:384 | UCGUGUCUUGUGUUGCAGCCGG | seq id no:666 |
| EAM230 | Oligo | /5AmMC6/CAGTGAATTCTACCAGTGCCATA | seq id no:385 | UAUGGCACUGGUAGAAUUCACUG | seq id no:667 |
| EAM229 | Oligo | /5AmMC6/TGTGAGTTCTACCATTGCCAAA | seq id no:386 | UUUGGCAAUGGUAGAACUCACA | seq id no:668 |
| EAM228 | Oligo | /5AmMC6/ACTCACCGACAGGTTGAATGTT | seq id no:387 | AACAUUCAACCUGUCGGUGAGU | seq id no:669 |
| EAM222 | Oligo | /5AmMC6/CACAAACCATTATGTGCTGCTA | seq id no:388 | UAGCAGCACAUAAUGGUUUGUG | seq id no:670 |
| EAM220 | Oligo | /5AmMC6/CGAAGGCAACACGGATAACCTA | seq id no:389 | UAGGUUAUCCGUGUUGCCUUCG | seq id no:671 |
| EAM219 | Oligo | /5AmMC6/TCACTTTTGTGACTATGCAA | seq id no:390 | UUGCAUAGUCACAAAAGUGA | seq id no:672 |
| EAM218 | Oligo | /5AmMC6/CCAAGTTCTGTCATGCACTGA | seq id no:391 | UCAGUGCAUGACAGAACUUGG | seq id no:673 |
| EAM217 | Oligo | /5AmMC6/ACACTGGTACAAGGGTTGGGAGA | seq id no:392 | UCUCCCAACCCUUGUACCAGUG | seq id no:674 |
| EAM216 | Oligo | /5AmMC6/GGAGTGAAGACACGGAGCCAGA | seq id no:393 | UCUGGCUCCGUGUCUUCACUCC | seq id no:675 |
| EAM215 | Oligo | /5AmMC6/ACAAAGTTCTGTGATGCACTGA | seq id no:394 | UCAGUGCAUCACAGAACUUUGU | seq id no:676 |
| EAM214 | Oligo | /5AmMC6/ACAAAGTTCTGTAGTGCACTGA | seq id no:395 | UCAGUGCACUACAGAACUUUGU | seq id no:677 |
| EAM212 | Oligo | /5AmMC6/AAGGGATTCCTGGGAAAACTGGAC | seq id no:396 | GUCCAGUUUUCCCAGGAAUCCCUU | seq id no:678 |
| EAM211 | Oligo | /5AmMC6/CTAGTACATCATCTATACTGTA | seq id no:397 | UACAGUAUAGAUGAUGUACUAG | seq id no:679 |
| EAM210 | Oligo | /5AmMC6/tgAGCTACAGTGCTTCATCTCA | seq id no:398 | UGAGAUGAAGCACUGUAGCUCA | seq id no:680 |
| EAM208 | Oligo | /5AmMC6/CCATCTTTACCAGACAGTGTT | seq id no:399 | AACACUGUCUGGUAAAGAUGG | seq id no:681 |
| EAM207 | Oligo | /5AmMC6/CTACCATAGGGTAAAACCACT | seq id no:400 | AGUGGUUUUACCCUAUGGUAG | seq id no:682 |
| EAM206 | Oligo | /5AmMC6/AGACACGTGCACTGTAGA | seq id no:401 | UCUACAGUGCACGUGUCU | seq id no:683 |
| EAM205 | Oligo | /5AmMC6/GATTCACAACACCAGCT | seq id no:402 | AGCUGGUGUUGUGAAUC | seq id no:684 |
| EAM203 | Oligo | /5AmMC6/TTCACATAGGAATAAAAAGCCATA | seq id no:403 | UAUGGCUUUUUAUUCCUAUGUGA | seq id no:685 |
| EAM200 | Oligo | /5AmMC6/ACAGCTGGTTGAAGGGGACCAA | seq id no:404 | UUGGUCCCCUUCAACCAGCUGU | seq id no:686 |
| EAM195 | Oligo | /5AmMC6/GAAAGAGACCGGTTCACTGTGA | seq id no:405 | UCACAGUGAACCGGUCUCUUUC | seq id no:687 |
| EAM194 | Oligo | /5AmMG6/AAAAGAGACCGGTTCACTGTGA | seq id no:406 | UCACAGUGAACCGGUCUCUUUU | seq id no:688 |
| EAM193 | Oligo | /5AmMC6/CACAGGTTAAAGGGTCTCAGGGA | seq id no:407 | UCCCUGAGACCCUUUAACCUGUG | seq id no:689 |
| EAM190 | Oligo | /5AmMC6/ACAAATTCGGTTCTACAGGGTA | seq id no:408 | UACCCUGUAGAACCGAAUUUGU | seq id no:690 |
| EAM187 | Oligo | /5AmMC6/TGATAGCCCTGTACAATGCTGCT | seq id no:409 | AGCAGCAUUGUACAGGGCUAUCA | seq id no:691 |
| EAM185 | Oligo | /5AmMC6/TCATAGCCCTGTACAATGCTGCT | seq id no:410 | AGCAGCAUUGUACAGGGCUAUGA | seq id no:692 |
| EAM181 | Oligo | /5AmMC6/AACTATACAATCTACTACCTCA | seq id no:411 | UGAGGUAGUAGAUUGUAUAGUU | seq id no:693 |
| EAM179 | Oligo | /5AmMC6/ACTATGCAACCTACTACCTCT | seq id no:412 | AGAGGUAGUAGGUUGCAUAGU | seq id no:694 |
| EAM177 | Oligo | /5AmMC6/TTCAGCTATCACAGTACTGTA | seq id no:413 | UACAGUACUGUGAUAGCUGAAG | seq id no:695 |
| EAM175 | Oligo | /5AmMC6/TCGCCCTCTCAACCCAGCTTTT | seq id no:414 | AAAAGCUGGGUUGAGAGGGCGAA | seq id no:696 |
| EAM168 | Oligo | /5AmMC6/CTATACAACCTCCTACCTCA | seq id no:415 | UGAGGUAGGAGGUUGUAUAGU | seq id no:697 |
| EAM161 | Oligo | /5AmMC6/CTCAATAGACTGTGAGCTCCTT | seq id no:416 | AAGGAGCUCACAGUCUAUUGAG | seq id no:698 |
| EAM160 | Oligo | /5AmMC6/AACCTATCCTGAATTACTTGAA | seq id no:417 | UUCAAGUAAUUCAGGAUAGGUU | seq id no:699 |
| EAM155 | Oligo | /5AmMC6/TCCATCATCAAAACAAATGGAGT | seq id no:418 | ACUCCAUUUGUUUUGAUGAUGGA | seq id no:700 |
| EAM153 | Oligo | /5AmMC6/AACTATACAACCTACTACCTCA | seq id no:419 | UGAGGUAGUAGGUUGUAUAGUU | seq id no:701 |
| EAM147 | Oligo | /5AmMC6/AACCACACAACCTACTACCTCA | seq id no:420 | UGAGGUAGUAGGUUGUGUGGUU | seq id no:702 |
| EAM137 | Oligo | /5AmMG6/CGGACCATGGCTGTAGACTGTTA | seq id no:421 | UAACAGUCUACAGCCAUGGUCG | seq id no:703 |
| EAM133 | Oligo | /5AmMC6/ACACCAATGCCCTAGGGGATGCG | seq id no:422 | CGCAUCCCCUAGGGCAUUGGUGU | seq id no:704 |
| EAM311 | Oligo | /5AmMC6/CTTCAGTTATCACAGTACTGTA | seq id no:423 | UACAGUACUGUGAUAACUGAAG | seq id no:705 |
| EAM312 | Oligo | /5AmMC6/ACAGGAGTCTGAGCATTTGA | seq id no:424 | UCAAAUGCUCAGACUCCUGU | seq id no:706 |
| EAM313 | Oligo | /5AmMC6/ATCTGCACTGTCAGCACTTTA | seq id no:425 | UAAAGUGCUGACAGUGCAGAU | seq id no:707 |
| EAM314 | Oligo | /5AmMC6/GCATTATTACTCACGGTACGA | seq id no:426 | UCGUACCGUGAGUAAUAAUGC | seq id no:708 |
| EAM315 | Oligo | /5AmMC6/AGCCAAGCTCAGACGGATCCGA | seq id no:427 | UCGGAUCCGUCUGAGCUUGGCU | seq id no:709 |
| EAM316 | Oligo | /5AmMC6/GCAGAAGCATTTCCACACAC | seq id no:428 | GUGUGUGGAAAUGCUUCUGC | seq id no:710 |
| EAM317 | Oligo | /5AmMC6/CCCCTATCACGATTAGCATTAA | seq id no:429 | UUAAUGCUAAUCGUGAUAGGGG | seq id no:711 |
| EAM318 | Oligo | /5AmMC6/ACAAGTGCCTTCACTGCAGT | seq id no:430 | ACUGCAGUGAAGGCACUUGU | seq id no:712 |
| EAM319 | Oligo | /5AmMC6/TAGTTGGCAAGTCTAGAACCA | seq id no:431 | UGGUUCUAGACUUGCCAACUA | seq id no:713 |
| EAM320 | Oligo | /5AmMC6/ACTGATATCAGCTCAGTAGGCAC | seq id no:432 | GUGCCUACUGAGCUGAUAUCAGU | seq id no:714 |
| EAM321 | Oligo | /5AmMC6/CATCATTACCAGGCAGTATTAGA | seq id no:433 | CUCUAAUACUGCCUGGUAAUGAUG | seq id no:715 |
| EAM291 | Oligo | /5AmMC6/GAACTGCCTTTCTCTCCA | seq id no:434 | UGGAGAGAAAGGCAGUUC | seq id no:716 |
| EAM290 | Oligo | /5AmMC6/ACCCTTATCAGTTCTCCGTCCA | seq id no:435 | UGGACGGAGAACUGAUAAGGGU | seq id no:717 |
| EAM322 | Oligo | /5AmMC6/TCCATCATTACCCGGCAGTATT | seq id no:436 | AAUACUGCCGGGUAAUGAUGGA | seq id no:718 |
| EAM323 | Oligo | /5AmMC6/TAAACGGAACCACTAGTGACTTG | seq id no:437 | CAAGUCACUAGUGGUUCCGUUUA | seq id no:719 |
| EAM324 | Oligo | /5AmMC6/TCAGACCGAGACAAGTGCAATG | seq id no:438 | CAUUGCACUUGUCUCGGUCUGA | seq id no:720 |
| EAM325 | Oligo | /5AmMC6/GGCGGAACTTAGCCACTGTGAA | seq id no:439 | UUCACAGUGGCUAAGUUCCGCC | seq id no:721 |
| EAM326 | Oligo | /5AmMC6/AGAGGATTGAGGGGGGGCCCT | seq id no:440 | AGGGCCCCCCCUCAAUCCUGU | seq id no:722 |
| EAM327 | Oligo | /5AmMC6/ATGTATGTGGGACGGTAAACCA | seq id no:441 | UGGUUUACCGUCCCACAUACAU | seq id no:723 |
| EAM328 | Oligo | /5AmMC6/GCTTTGACAATACTATTGCACTG | seq id no:442 | CAGUGCAAUAGUAUUGUCAAAGC | seq id no:724 |
| EAM329 | Oligo | /5AmMC6/TCACCAAAACATGGAAGCACTTA | seq id no:443 | UAAGUGCUUCCAUGUUUUGGUGA | seq id no:725 |
| EAM330 | Oligo | /5AmMC6/GCTTCCAGTCGAGGATGTTTACA | seq id no:444 | UGUAAACAUCCUCGACUGGAAGC | seq id no:726 |
| EAM331 | Oligo | /5AmMC6/TCCAGTCAAGGATGTTTACA | seq id no:445 | UGUAAACAUCCUUGACUGGA | seq id no:727 |
| EAM332 | Oligo | /5AmMC6/CAGCTATGCCAGCATCTTGCCT | seq id no:446 | AGGCAAGAUGCUGGCAUAGCUG | seq id no:728 |
| EAM333 | Oligo | /5AmMC6/GCAACTTAGTAATGTGCAATA | seq id no:447 | UAUUGCACAUUACUAAGUUGC | seq id no:729 |
| EAM334 | Oligo | /5AmMC6/GAACCCACAATCCCTGGCTTA | seq id no:448 | UAAGCCAGGGAUUGUGGGUUC | seq id no:730 |
| EAM335 | Oligo | /5AmMC6/CAATCAGCTAATGACACTGCCT | seq id no:449 | AGGCAGUGUCAUUAGCUGAUUG | seq id no:731 |
| EAM336 | Oligo | /5AmMC6/GCAATCAGCTAACTACACTGCCT | seq id no:450 | AGGCAGUGUAGUUAGCUGAUUGC | seq id no:732 |
| EAM337 | Oligo | /5AmMC6/CTACCTGCACGAACAGCACTTTG | seq id no:451 | CAAAGUGCUGUUCGUGCAGGUAG | seq id no:733 |
| EAM338 | Oligo | /5AmMC6/TGCTCAATAAATACCCGTTGAA | seq id no:452 | UUCAACGGGUAUUUAUUGAGCA | seq id no:734 |
| EAM339 | Oligo | /5AmMC6/CGCTTGGTCGGTTCTTCGGGTG | seq id no:453 | CACCCGUAGAACCGACCUUGCG | seq id no:735 |
| EAM340 | Oligo | /5AmMC6/AGAAAGGCAGCAGGTCGTATAG | seq id no:454 | CUAUACGACCUGCUGCCUUUCU | seq id no:736 |
| EAM341 | Oligo | /5AmMC6/TACCTGCACTGTTAGCACTTTG | seq id no:455 | CAAAGUGCUAACAGUGCAGGUA | seq id no:737 |
| EAM342 | Oligo | /5AmMC6/CACATAGGAATGAAAAGCCATA | seq id no:456 | UAUGGCUUUUCAUUCCUAUGUG | seq id no:738 |
| EAM343 | Oligo | /5AmMC6/CCTCAAGGAGCCTCAGTCTAGT | seq id no:457 | ACUAGACUGAGGCUCCUUGAGG | seq id no:739 |
| EAM344 | Oligo | /5AmMC6/ACAAGTGCCCTCACTGCAGT | seq id no:458 | ACUGCAGUGAGGGCACUUGU | seq id no:740 |
| EAM345 | Oligo | /5AmMC6/TAAACGGAACCACTAGTGACTTA | seq id no:459 | UAAGUCACUAGUGGUUCCGUUUA | seq id no:741 |
| EAM346 | Oligo | /5AmMC6/AAAAAGTGCCCCCATAGTTTGAG | seq id no:460 | CUCAAACUAUGGGGGCACUUUUU | seq id no:742 |
| EAM347 | Oligo | /5AmMC6/GGCACACAAAGTGGAAGCACTTT | seq id no:461 | AAAGUGCUUCCACUUUGUGUGCC | seq id no:743 |
| EAM348 | Oligo | /5AmMC6/AGAGAGGGCCTCCACTTTGATG | seq id no:462 | CAUCAAAGUGGAGGCCCUCUCU | seq id no:744 |
| EAM349 | Oligo | /5AmMC6/ACACTCAAAACCTGGCGGCACTT | seq id no:463 | AAGUGCCGCCAGGUUUUGAGUGU | seq id no:745 |
| EAM350 | 0ligo | /5AmMC6/CAAAAGAGCCCCCAGTTTGAGT | seq id no:464 | ACUCAAACUGGGGGCUCUUUUG | seq id no:746 |
| EAM351 | Oligo | /5AmMC6/ACACTACAAACTCTGCGGCACT | seq id no:465 | AGUGCCGCAGAGUUUGUAGUGU | seq id no:747 |
| EAM352 | Oligo | /5AmMC6/ACACACAAAAGGGAAGCACTTT | seq id no:466 | AAAGUGCUUCCCUUUUGUGUGU | seq id no:748 |
| EAM353 | Oligo | /5AmMC6/AGACTCAAAAGTAGTAGCACTTT | seq id no:467 | AAAGUGCUACUACUUUUGAGUCU | seq id no:749 |
| EAM354 | Oligo | /5AmMC6/CATGCACATGCACACATACAT | seq id no:468 | AUGUAUGUGUGCAUGUGCAUG | seq id no:750 |
| EAM355 | Oligo | /5AmMC6/GGAAGAACAGCCCTCCTCTGCC | seq id no:469 | GGCAGAGGAGGGCUGUUCUUCC | seq id no:751 |
| EAM356 | Oligo | /5AmMC6/GAAGAGAGCTTGCCCTTGCATA | seq id no:470 | UAUGCAAGGGCAAGCUCUCUUC | seq id no:752 |
| EAM357 | Oligo | /5AmMC6/TGTTGCTGCGCTTCTTGTTT | seq id no:471 | AAACAUGAAGCGCUGCAACA | seq id no:753 |
| EAM358 | Oligo | /5AmMC6/AGAGGTCGACCGTGTAATGTGC | seq id no:472 | GCACAUUACACGGUCGACCUCU | seq id no:754 |
| EAM359 | Oligo | /5AmMC6/CCAGCAGCACCTGGGGCAGT | seq id no:473 | CCACUGCCCCAGGUGCUGCUGG | seq id no:755 |
| EAM360 | Oligo | /5AmMC6/ACACTTACTGAGCACCTACTAGG | seq id no:474 | CCUAGUAGGUGCUCAGUAAGUGU | seq id no:756 |
| EAM361 | Oligo | /5AmMC6/ACTGGAGGAAGGGCCCAGAGG | seq id no:475 | CCUCUGGGCCCUUCCUCCAGU | seq id no:757 |
| EAM362 | Oligo | /5AmMC6/ACGGAAGGGCAGAGAGGGCCAG | seq id no:476 | CUGGCCCUCUCUGCCCUUCCGU | seq id no:758 |
| EAM363 | Oligo | /5AmMC6/AAAAAGGTTAGCTGGGTGTGTT | seq id no:477 | AACACACCCAGCUAACCUUUUU | seq id no:759 |
| EAM364 | Oligo | /5AmMC6/TCTCTGCTGGCCCTGTGCTTTGC | seq id no:478 | GCAAAGCACAGGGCCUGCAGAGA | seq id no:760 |
| EAM365 | Oligo | /5AmMC6/TTCTAGGATAGGCCCAGGGGC | seq id no:479 | GCCCCUGGGCCUAUCCUAGAA | seq id no:761 |
| EAM366 | Oligo | /5AmMC6/AAAGGCATCATATAGGAGCTGAA | seq id no:480 | UUCAGCUCCUAUAUGAUGCCUUU | seq id no:762 |
| EAM367 | Oligo | /5AmMC6/TCAACAAAATCACTGATGCTGGA | seq id no:481 | UCCAGCAUCAGUGAUUUUGUUGA | seq id no:763 |
| EAM368 | Oligo | /5AmMC6/TGAGCTCCTGGAGGACAGGGA | seq id no:482 | UCCCUGUCCUCCAGGAGCUCA | seq id no:764 |
| EAM369 | Oligo | /5AmMC6/GGCTATAAAGTAACTGAGACGGA | seq id no:483 | UCCGUCUCAGUUACUUUAUAGCC | seq id no:765 |
| EAM370 | Oligo | /5AmMC6/ACTGACCGACCGACCGATCGA | seq id no:484 | UCGAUCGGUCGGUCGGUCAGU | seq id no:766 |
| EAM371 | Oligo | /5AmMC6/GACGGGTGCGATTTCTGTGTGAGA | seq id no:485 | UCUCACACAGAAAUCGCACCCGUC | seq id no:767 |
| EAM372 | Oligo | /5AmMC6/ACAGTCAGGCTTTGGCTAGATCA | seq id no:486 | UGAUCUAGCCAAAGCCUGACUGU | seq id no:768 |
| EAM373 | Oligo | /5AmMC6/GCACTGGACTAGGGGTCAGCA | seq id no:487 | UGCUGACCCCUAGUCCAGUGC | seq id no:769 |
| EAM374 | Oligo | /5AmMC6/AGAGGCAGGCACTCGGGCAGA | seq id no:488 | UGUCUGCCCGAGUGCCUGCCUCU | seq id no:770 |
| EAM375 | Oligo | /5AmMC6/CAATCAGCTAATTACACTGCCTA | seq id no:489 | UAGGCAGUGUAAUUAGCUGAUUG | seq id no:771 |
| EAM376 | Oligo | /5AmMC6/GTGAAAGTGTATGGGCTTTGTG | seq id no:490 | UUCACAAAGCCCAUACACUUUCAC | seq id no:772 |
| EAM377 | Oligo | /5AmMC6/CAGGCTCAAAGGGCTCCTCAGG | seq id no:491 | UCCCUGAGGAGCCCUUUGAGCCUG | seq id no:773 |
| EAM378 | Oligo | /5AmMC6/AACAAAATCACAAGTCTTCCA | seq id no:492 | UGGAAGACUUGUGAUUUUGUU | seq id no:774 |
| EAM379 | Oligo | /5AmMC6/TTGCTTTTTGGGGTTTGGGCTT | seq id no:493 | AAGCCCUUACCCCAAAAAGCAU | seq id no:775 |
| EAM380 | Oligo | /5AmMC6/TGTCCGTGGTTCTTCCCTGTG | seq id no:494 | UACCACAGGGUAGAACCACGGACA | seq id no:776 |
| EAM381 | Oligo | /5AmMC6/TACTAGACTGTGAGCTCCTCGA | seq id no:495 | UCGAGGAGCUCACAGUCUAGUA | seq id no:777 |
| EAM382 | Oligo | /5AmMC6/TGTAAGTGCTCGTAATGCAGT | seq id no:496 | ACUGCAUUACGAGCACUUACA | seq id no:778 |
| EAM383 | Oligo | /5AmMC6/ACCCTCATGCCCCTCAAGG | seq id no:497 | CCUUGAGGGGCAUGAGGGU | seq id no:779 |
| EAM384 | Oligo | /5AmMC6/AAAAGTAACTAGCACACCAC | seq id no:498 | GUGGUGUGCUAGUUACUUUU | seq id no:780 |
| EAM385 | Oligo | /5AmMC6/ACATTTTTCGTTATTGCTCTT | seq id no:499 | UCAAGAGCAAUAACGAAAAAUGU | seq id no:781 |
| EAM386 | Oligo | /5AmMC6/AGACTAGATATGGAAGGGTGA | seq id no:500 | UCACCCUUCCAUAUCUAGUCU | seq id no:782 |
| EAM387 | Oligo | /5AmMC6/ACTGGGCACACGGAGGGAGA | seq id no:501 | UCUCCCUCCGUGUGCCCAGU | seq id no:783 |
| EAM388 | Oligo | /5AmMC6/ACGGTCAGGCTTTGGCTAGAT | seq id no:502 | UGAUCUAGCCAAAGCCUGACCGU | seq id no:784 |
| EAM389 | Oligo | /5AmMC6/AGAGGCAGGCACTCAGGCAGA | seq id no:503 | UGUCUGCCUGAGUGCCUGCCUCU | seq id no:785 |
| EAM390 | Oligo | /5AmMC6/TGGGCGACCCAGAGGGACA | seq id no:504 | UGUCCCUCUGGGUCGCCCA | seq id no:786 |
| EAM391 | Oligo | /5AmMC6/AGAGGTTAAGACAGCAGGGCTG | seq id no:505 | CAGCCCUGCUGUCUUAACCUCU | seq id no:787 |
| EAM392 | Oligo | /5AmMC6/TACTATGCAACCTACTACTCT | seq id no:506 | AGAGUAGUAGGUUGCAUAGUA | seq id no:788 |
| EAM393 | Oligo | /5AmMC6/TATGGCAGACTGTGATTTGTTG | seq id no:507 | CAACAAAUCACAGUCUGCCAUA | seq id no:789 |
| emc139 | Oligo | /5AmMC6/CGAAATGCGTCTCATACAAAATC | seq id no:508 | NA | seq id no:790 |
| EAM289 | Oligo | /5AmMC6/AACAAGCCCAGACCGCAAAAAG | seq id no:509 | CUUUUUGCGGUCUGGGCUUGCU | seq id no:791 |
| EAM283 | Oligo | /5AmMC6/AGGCAAAGGATGACAAAGGGAA | seq id no:510 | UUCCCUUUGUCAUCCUUUGCCU | seq id no:792 |
| PTG20210 | Oligo | /5AmC12/CATTGAGGCTCGCTGAGAGT | seq id no:511 | GTGACTCTCAGCGAGCCTCAATGC | seq id no:793 |
| MRC677 | Oligo | /5AmC12/GATGAAATCGGCTCCCGCAG- | seq id no:512 | TGTCTGCGGGAGCCGATTTCATCA | seq id no:794 |
| FVR506 | Oligo | /5AmC12/TGTATTCCTCGCCTGTCCAG | seq id no:513 | TCCCTGGACAGGCGAGGAATACAG | seq id no:795 |
| EAM104 | Oligo | /5AmMC6/TGGCATTCAGCGGGTGCCTTA | seq id no:514 | TAAGGCACCCGCTGAATGCCA | seq id no:796 |
| EAM106 | Oligo | /5AmMC6/TCACAAGTAAGGGTGTCAGGGA | seq id no:515 | TCCCTGACACCCTTACTTGTGA | seq id no:797 |
| EAM110 | Oligo | /5AmMC6/AACAACAAAATGAGTAGTCTTCCA | seq id no:516 | TGGAAGACTACTCATTTTGTTGTT | seq id no:798 |
| EAM1101 | Oligo | /5AmMC6/GTGGTAGCGCAGTGCGTAGAA | seq id no:517 | TTCTACGCACTGCGCTACCAC | seq id no:799 |
| EAM1102 | Oligo | /5AmMC6/GGTGATGCCCTGAATGTTGTC | seq id no:518 | NA | seq id no:800 |
| EAM1103 | Oligo | /5AmMC6/TGTCATGGATGACCTTGGCCA | seq id no:519 | NA | seq id no:801 |
| EAM1104 | Oligo | /5AmMC6/CTTTTGACATTGAAGGGAGCT | seq id no:520 | NA | seq id no:802 |
| EAM146 | Oligo | /5AmMC6/AACCATACAAGCTAGTACCTCA | seq id no:521 | TGAGGTACTAGCTTGTATGGTT | seq id no:803 |
| emc130 | Oligo | /5AmMC6/CTTGTACCAGTTATCTGCAA | seq id no:522 | UUGCAGAUAACUGGUACAAG | seq id no:804 |
| emc115 | Oligo | /5AmMC6/TTGTACGTTTACATGGAGGTC | seq id no:523 | GACCUCCAUGUAAACGUACAA | seq id no:805 |
| EAM148 | Oligo | /5AmMC6/AACCACACAAGCTAGTACCTCA | seq id no:524 | TGAGGTACTAGCTTGTGTGGTT | seq id no:806 |
| EAM138 | Oligo | /5AmMC6/CCGACCATGGGTGAAGACTGTTA | seq id no:525 | TAACAGTCTTCACCCATGGTCGG | seq id no:807 |
| EAM134 | Oligo | /5AmMC6/ACACCAATGGCGTAGGGGATGCG | seq id no:526 | CGCATCCCCTACGCCATTGGTGT | seq id no:808 |
| EAM395 | Oligo | /5AmMC6/CTGACTGACTGACTGACTGACTG | seq id no:527 | CAGUCAGUCAGUCAGUCAGUCAG | seq id no:809 |
| EAM149I | Oligo | /5AmMC6/GTCACTATTGTTGAGAACGTTGGCC | seq id no:528 | NA | seq id no:810 |
| EAM150I | Oligo | /5AmMC6/GTCACTATTGTAGAGAAGGTTGGCC | seq id no:529 | NA | seq id no:811 |
| EAM399 | Oligo | /5AmMC6/TTCAATTTCTGCCGCAAAAG | seq id no:530 | UAUCUUUUGCGGCAGAAAUUGAA | seq id no:812 |
| EAM400 | Oligo | /5AmMC6/GCTATCTGCTGCAACAGAATTT | seq id no:531 | AAAUUCUGUUGCAGCAGAUAGC | seq id no:813 |
| EAM401 | Oligo | /5AmMC6/GTGTGCTTACACACTTCCCGTTA | seq id no:532 | UAACGGGAAGUGUGUAAGCACAC | seq id no:814 |
| EAM402 | Oligo | /5AmMC6/TAGCTGGTTGAAGGGGACCAA | seq id no:533 | UUGGUCCCCUUCAACCAGCUA | seq id no:815 |
| EAM403 | Oligo | /5AmMC6/CCTCAAGGAGCTTCAGTCTAGT | seq id no:534 | ACUAGACUGAAGCUCCUUGAGG | seq id no:816 |
| EAM404 | Oligo | /5AmMC6/CCAACAACAGGAAACTACCTA | seq id no:535 | UAGGUAGUUUCCUGUUGUUGG | seq id no:817 |
| EAM405 | Oligo | /5AmMC6/CTACTAAAACATGGAAGCACTTA | seq id no:536 | UAAGUGCUUCCAUGUUUUAGUAG | seq id no:818 |
| EAM406 | Oligo | /5AmMC6/AGAAAGCACTTCCATGTTAAAGT | seq id no:537 | ACUUUAAGAUGGAAGUGCUUUCU | seq id no:819 |
| EAM407 | Oligo | /5AmMC6/CCACTGAAACATGGAAGCACTTA | seq id no:538 | UAAGUGCUUCCAUGUUUCAGUGG | seq id no:820 |
| EAM408 | Oligo | /5AmMC6/CAGCAGGTACCCCCATGTTA | seq id no:539 | UUUAACAUGGGGGUACCUGCUG | seq id no:821 |
| EAM409 | Oligo | /5AmMC6/ACACTCAAACATGGAAGCACTTA | seq id no:540 | UAAGUGCUUCCAUGUUUGAGUGU | seq id no:822 |
| EAM410 | Oligo | /5AmMC6/ACTTACTGGACACCTACTAGG | seq id no:541 | CCUAGUAGGUGUCCAGUAAGU | seq id no:823 |
| EAM411 | Oligo | /5AmMC6/TCTCTGCTGGCCGTGTGCTT | seq id no:542 | GCAAAGCACACGGCCUGCAGAGA | seq id no:824 |
| EAM412 | Oligo | /5AmMC6/AAAGGCATCATATAGGAGCTGGA | seq id no:543 | UCCAGCUCCUAUAUGAUGCCUUU | seq id no:825 |
| EAM413 | Oligo | /5AmMC6/GCCCTGGACTAGGAGTCAGCA | seq id no:544 | UGCUGACUCCUAGUCCAGGGC | seq id no:826 |
| EAM414 | Oligo | /5AmMC6/AGAGGCAGGCATGCGGGCAG | seq id no:545 | UGUCUGCCCGCAUGCCUGCCUCU | seq id no:827 |
| EAM415 | Oligo | /5AmMC6/TCACCATTGCTAAAGTGCAATT | seq id no:546 | AAUUGCACUUUAGCAAUGGUGA | seq id no:828 |
| EAM416 | Oligo | /5AmMC6/AAACGTGGAATTTCCTCTATGT | seq id no:547 | ACAUAGAGGAAAUUCCACGUUU | seq id no:829 |
| EAM417 | Oligo | /5AmMC6/AAAGATCAACCATGTATTATT | seq id no:548 | AAUAAUACAUGGUUGAUCUUU | seq id no:830 |
| EAM418 | Oligo | /5AmMC6/CCAGGTTCCACCCCAGCAGG | seq id no:549 | GCCUGCUGGGGUGGAACCUGG | seq id no:831 |
| EAM419 | Oligo | /5AmMC6/ACACTCAAAAGATGGCGGCA | seq id no:550 | GUGCCGCCAUCUUUUGAGUGU | seq id no:832 |
| EAM420 | Oligo | /5AmMC6/ACGCTCAAATGTCGCAGCAC | seq id no:551 | AAAGUGCUGCGACAUUUGAGCGU | seq id no:833 |
| EAM421 | Oligo | /5AmMC6/ACACCCCAAAATCGAAGCAC | seq id no:552 | GAAGUGCUUCGAUUUUGGGGUGU | seq id no:834 |
| EAM422 | Oligo | /5AmMC6/GGAAAGCGCCCCCATTTTGA | seq id no:553 | ACUCAAAAUGGGGGCGCUUUCC | seq id no:835 |
| EAM423 | Oligo | /5AmMC6/CACTTATCAGGTTGTATTATAA | seq id no:554 | UUAUAAUACAACCUGAUAAGUG | seq id no:836 |
| EAM424 | Oligo | /5AmMC6/TAGCTGGTTGAAGGGGACCA | seq id no:555 | UUGGUCCCCUUCAACCAGCUA | seq id no:837 |
| EAM425 | Oligo | /5AmMC6/CCAACAACAGGAAACTACCTA | seq id no:556 | UAGGUAGUUUCCUGUUGUUGG | seq id no:838 |
| EAM426 | Oligo | /5AmMC6/GTCTGTCAAATCATAGGTCAT | seq id no:557 | AUGACCUAUGAUUUGACAGAC | seq id no:839 |
| EAM427 | Oligo | /5AmMC6/GGGGTTCACCGAGCAACATTC | seq id no:558 | GAAUGUUGCUCGGUGAACCCCUU | seq id no:840 |
| EAM428 | Oligo | /5AmMC6/CAGGCCATCTGTGTTATATT | seq id no:559 | AAUAUAACACAGAUGGCCUGUU | seq id no:841 |
| EAM429 | Oligo | /5AmMC6/AGTGGATGTTCCTCTATGAT | seq id no:560 | AUCAUAGAGGAACAUCCACUUU | seq id no:842 |
| EAM430 | Oligo | /5AmMC6/CGTGGATTTTCCTCTACGAT | seq id no:561 | AUCGUAGAGGAAAAUCCACGUU | seq id no:843 |
| EAM431 | Oligo | /5AmMC6/GAGGGTTAGTGGACCGTGTT | seq id no:562 | AACACGGUCCACUAACCCUCAGU | seq id no:844 |
| EAM432 | Oligo | /5AmMC6/GATGTGGACCATACTACATA | seq id no:563 | UAUGUAGUAUGGUCCACAUCUU | seq id no:845 |
| EAM433 | Oligo | /5AmMC6/GGCTAGTGGACCAGGTGAAG | seq id no:564 | CUUCACCUGGUCCACUAGCCGU | seq id no:846 |
| EAM396 | Oligo | /5AmMC6/AGCACGTCACTTCCACTAAGA | seq id no:565 | UCUUAGUGGAAGUGACGUGCU | seq id no:847 |
| EAM397 | Oligo | /5AmMC6/GCAAGGGCGAATGCAGAAAA | seq id no:566 | UAUUUUCUGCAUUCGCCCUUGC | seq id no:848 |
| EAM398 | Oligo | /5AmMC6/AACTCCGGGGCTGATCAGGT | seq id no:567 | UAACCUGAUCAGCCCCGGAGUU | seq id no:849+TZ,1/64 |

TABLE 10b

| | | | | | | Set | Set | |
|---|---|---|---|---|---|---|---|---|
| Probe | | | | | | No. | No. | |
| ID | Human | Mouse | Rat | Other | Control | (V1) | (V2) | Usage |
| EAM103 | hsa-miR-124a | mmu-miR-124a | rno-miR-124a | | | 1 | 1 | Used |
| EAM105 | hsa-miR-125b | mmu-miR-125b | rno-miR-125b | | | 1 | 1 | Used |
| EAM109 | hsa-miR-7 | mmu-miR-7 | rno-miR-7 | | | 1 | 1 | Used |
| EAM111 | hsa-let-7g | mmu-let-7g | | | | 1 | 1 | Used |
| EAM115 | hsa-miR-16 | mmu-miR-16 | rno-miR-16 | | | 1 | 1 | Used |
| EAM119 | hsa-miR-29b | mmu-miR-29b | rno-miR-29b | | | 1 | 1 | Used |
| EAM121 | hsa-miR-99a | mmu-miR-99a | rno-miR-99a | | | 1 | 1 | Used |
| EAM131 | hsa-miR-92 | mmu-miR-92 | rno-miR-92 | | | 1 | 1 | Used |
| EAM139 | hsa-miR-146 | mmu-miR-146 | rno-miR-146 | | | 1 | 1 | Used |
| EAM145 | hsa-let-7c | mmu-let-7c | rno-let-7c | | | 1 | 1 | Used |
| EAM152 | hsa-miR-9* | mmu-miR-9* | | | | 1 | 1 | Used |
| EAM238 | hsa-miR-1 | mmu-miR-1 | | | | 1 | 1 | Used |
| EAM270 | hsa-miR-30b | mmu-miR-30b | rno-miR-30b | | | 1 | 1 | Used |
| EAM159 | hsa-miR-130a | mmu-miR-130a | rno-miR-130a | | | 1 | 1 | Used |
| EAM163 | hsa-miR-142-3p | mmu-miR-142-3p | rno-miR-142-3p | | | 1 | 1 | Used |
| EAM171 | hsa-miR-137 | mmu-miR-137 | rno-miR-137 | | | 1 | 1 | Used |
| EAM183 | hsa-let-7i | mmu-let-7i | rno-let-7i | | | 1 | 1 | Used |
| EAM184 | hsa-miR-100 | mmu-miR-100 | rno-miR-100 | | | 1 | 1 | Used |
| EAM186 | hsa-miR-106a | | | | | 1 | 1 | Used |
| EAM189 | hsa-miR-10a | mmu-miR-10a | rno-miR-10a | | | 1 | 1 | Used |
| EAM191 | hsa-miR-122a | mmu-miR-122a | rno-miR-122a | | | 1 | 1 | Used |
| EAM192 | hsa-miR-126* | mmu-miR-126* | rno-miR-126* | | | 1 | 1 | Used |
| EAM198 | hsa-miR-130b | mmu-miR-130b | rno-miR-130b | | | 1 | 1 | Used |
| EAM202 | hsa-miR-134 | mmu-miR-134 | rno-miR-134 | | | 1 | 1 | Used |
| EAM209 | hsa-miR-142-5p | mmu-miR-142-5p | rno-miR-142-5p | | | 1 | 1 | Used |
| EAM221 | mmu-miR-155 | | | | | 1 | 1 | Used |
| EAM223 | hsa-miR-15b | mmu-miR-15b | rno-miR-15b | | | 1 | 1 | Used |
| EAM224 | hsa-miR-17-5p | mmu-miR-17-5p | rno-miR-17-5p | | | 1 | 1 | Used |
| EAM225 | hsa-miR-18 | mmu-miR-18 | rno-miR-18 | | | 1 | 1 | Used |
| EAM226 | hsa-miR-181a | mmu-miR-181a | rno-miR-181a | | | 1 | 1 | Used |
| EAM227 | hsa-miR-181b | mmu-miR-181b | rno-miR-181b | | | 1 | 1 | Used |
| EAM234 | hsa-miR-199a | mmu-miR-199a | rno-miR-199a | | | 1 | 1 | Used |
| EAM235 | hsa-miR-199b | | | | | 1 | 1 | Used |
| EAM236 | hsa-miR-19a | mmu-miR-19a | rno-miR-19a | | | 1 | 1 | Used |
| EAM241 | hsa-miR-203 | mmu-miR-203 | rno-miR-203 | | | 1 | 1 | Used |
| EAM242 | hsa-miR-204 | mmu-miR-204 | rno-miR-204 | | | 1 | 1 | Used |
| EAM243 | hsa-miR-205 | mmu-miR-205 | rno-miR-205 | | | 1 | 1 | Used |
| EAM245 | hsa-miR-210 | mmu-miR-210 | rno-miR-210 | | | 1 | 1 | Used |
| EAM249 | hsa-miR-214 | mmu-miR-214 | rno-miR-214 | | | 1 | 1 | Used |
| EAM254 | hsa-miR-219 | mmu-miR-219 | rno-miR-219 | | | 1 | 3 | Used |
| EAM257 | hsa-miR-221 | mmu-miR-221 | rno-miR-221 | | | 1 | 3 | Used |
| EAM258 | hsa-miR-222 | mmu-miR-222 | rno-miR-222 | | | 1 | 3 | Used |
| EAM259 | hsa-miR-223 | mmu-miR-223 | rno-miR-223 | | | 1 | 3 | Used |
| EAM273 | hsa-miR-33 | mmu-miR-33 | rno-miR-33 | | | 1 | 3 | Used |
| EAM288 | | mmu-miR-10b | | | | 1 | 3 | Used |
| EAM293 | hsa-miR-188 | mmu-miR-188 | | | | 1 | 3 | Used |
| EAM297 | hsa-miR-193 | mmu-miR-193 | rno-miR-193 | | | 1 | 3 | Used |
| EAM301 | hsa-miR-198 | | | | | 1 | 3 | Used |
| EAM304 | hsa-miR-200a | mmu-miR-200a | rno-miR-200a | | | 1 | 2 | Used |
| EAM306 | | mmu-miR-201 | | | | 1 | 1 | Used |
| EAM307 | | mmu-miR-202 | | | | 1 | 1 | Used |
| EAM308 | hsa-miR-206 | mmu-miR-206 | rno-miR-206 | | | 1 | 1 | Used |
| EAM309 | | mmu-miR-207 | | | | 1 | 1 | Used |
| EAM310 | hsa-miR-208 | mmu-miR-208 | rno-miR-208 | | | 1 | 1 | Used |
| EAM247 | hsa-miR-212 | mmu-miR-212 | rno-miR-202 | | | 1 | 1 | Used |
| EAM251 | hsa-miR-216 | mmu-miR-216 | rno-miR-216 | | | 1 | 1 | Used |
| EAM253 | hsa-miR-218 | mmu-miR-218 | rno-miR-218 | | | 1 | 1 | Used |
| EAM275 | hsa-miR-34a | mmu-miR-34a | rno-miR-34a | | | 1 | 1 | Used |
| EAM246 | hsa-miR-211 | | | | | 1 | 1 | Used |
| EAM250 | hsa-miR-215 | | | | | 1 | 1 | Used |
| EAM252 | hsa-miR-217 | | | | | 1 | 1 | Used |
| EAM305 | | mmu-miR-200b | | | | 1 | 3 | Used |
| EAM303 | hsa-miR-199a* | mmu-miR-199a* | | | | 1 | 3 | Used |
| EAM300 | hsa-miR-197 | | | | | 1 | 3 | Used |
| EAM299 | hsa-miR-195 | mmu-miR-195 | rno-miR-195 | | | 1 | 3 | Used |
| EAM298 | hsa-miR-194 | mmu-miR-194 | rno-miR-194 | | | 1 | 2 | Used |
| EAM296 | hsa-miR-191 | mmu-miR-191 | rno-miR-191 | | | 1 | 2 | Not Used, |
| | | | | | | | | high |
| | | | | | | | | background |
| EAM295 | hsa-miR-190 | mmu-miR-190 | rno-miR-190 | | | 1 | 2 | Used |
| EAM292 | hsa-miR-186 | mmu-miR-186 | rno-miR-186 | | | 1 | 2 | Used |
| EAM112 | | | | | Yes, | 1 | 1 | Not Used, |
| | | | | | Mismatch | | | control |
| | | | | | | | | feature |
| EAM116 | | | | | Yes, | 1 | 1 | Not Used, |
| | | | | | Mismatch | | | control |
| | | | | | | | | feature |
| EAM120 | | | | | Yes, | 1 | 1 | Not Used, |
| | | | | | Mismatch | | | control |
| | | | | | | | | feature |
| EAM122 | | | | | Yes, | 1 | 1 | Not Used, |
| | | | | | Mismatch | | | control |
| | | | | | | | | feature |
| EAM132 | | | | | Yes, | 1 | 1 | Not Used, |
| | | | | | Mismatch | | | control |
| | | | | | | | | feature |
| EAM140 | | | | | Yes, | 1 | 1 | Not Used, |
| | | | | | Mismatch | | | control |
| | | | | | | | | feature |
| EAM282 | | mmu-miR-199b | | | | 2 | 1 | Used |
| EAM281 | | mmu-miR-217 | rno-miR-217 | | | 2 | 1 | Used |
| EAM280 | hsa-miR-30a-3p | mmu-miR-30a-3p | rno-miR-30a-3p | | | 2 | 1 | Used |
| EAM279 | hsa-miR-29c | mmu-miR-29c | rno-miR-29c | | | 2 | 1 | Used |
| EAM278 | hsa-miR-98 | mmu-miR-98 | rno-miR-98 | | | 2 | 1 | Used |
| EAM277 | hsa-miR-96 | mmu-miR-96 | rno-miR-96 | | | 2 | 3 | Used |
| EAM276 | hsa-miR-9 | mmu-miR-9 | rno-miR-9 | | | 2 | 3 | Used |
| EAM272 | hsa-miR-30d | mmu-miR-30d | rno-miR-30d | | | 2 | 3 | Used |
| EAM271 | hsa-miR-30c | mmu-miR-30c | rno-miR-30c | | | 2 | 3 | Used |
| EAM268 | hsa-miR-29a | mmu-miR-29a | rno-miR-29a | | | 2 | 3 | Used |
| EAM264 | hsa-miR-27b | mmu-miR-27b | rno-miR-27b | | | 2 | 3 | Used |
| EAM263 | hsa-miR-26a | mmu-miR-26a | rno-miR-26a | | | 2 | 3 | Used |
| EAM262 | hsa-miR-24 | mmu-miR-24 | rno-miR-24 | | | 2 | 3 | Used |
| EAM261 | hsa-miR-23b | mmu-miR-23b | rno-miR-23b | | | 2 | 3 | Used |
| EAM260 | hsa-miR-23a | mmu-miR-23a | rno-miR-23a | | | 2 | 3 | Used |
| EAM256 | hsa-miR-220 | | | | | 2 | 3 | Used |
| EAM255 | hsa-miR-22 | mmu-miR-22 | rno-miR-22 | | | 2 | 3 | Used |
| EAM248 | hsa-miR-213 | mmu-miR-213 | rno-miR-213 | | | 2 | 3 | Used |
| EAM244 | hsa-miR-21 | mmu-miR-21 | rno-miR-21 | | | 2 | 3 | Used |
| EAM240 | hsa-miR-20 | mmu-miR-20 | rno-miR-20 | | | 2 | 3 | Used |
| EAM237 | hsa-miR-19b | mmu-miR-19b | rno-miR-19b | | | 2 | 3 | Used |
| EAM233 | hsa-miR-196a | mmu-miR-196a | rno-miR-196a | | | 2 | 3 | Used |
| EAM232 | hsa-miR-192 | mmu-miR-192 | rno-miR-192 | | | 2 | 3 | Used |
| EAM231 | hsa-miR-187 | mmu-miR-187 | rno-miR-187 | | | 2 | 3 | Used |
| EAM230 | hsa-miR-183 | mmu-miR-183 | rno-miR-183 | | | 2 | 3 | Used |
| EAM229 | hsa-miR-182 | mmu-miR-182 | | | | 2 | 3 | Used |
| EAM228 | hsa-miR-181c | mmu-miR-181c | rno-miR-181c | | | 2 | 1 | Used |
| EAM222 | hsa-miR-15a | mmu-miR-15a | | | | 2 | 1 | Used |
| EAM220 | hsa-miR-154 | mmu-miR-154 | rno-miR-154 | | | 2 | 3 | Used |
| EAM219 | hsa-miR-153 | mmu-miR-153 | rno-miR-153 | | | 2 | 3 | Used |
| EAM218 | hsa-miR-152 | mmu-miR-152 | rno-miR-152 | | | 2 | 3 | Used |
| EAM217 | hsa-miR-150 | mmu-miR-150 | rno-miR-150 | | | 2 | 3 | Used |
| EAM216 | hsa-miR-149 | mmu-miR-149 | | | | 2 | 3 | Used |
| EAM215 | hsa-miR-148b | mmu-miR-148b | rno-miR-148b | | | 2 | 3 | Used |
| EAM214 | hsa-miR-148a | mmu-miR-148a | | | | 2 | 3 | Used |
| EAM212 | hsa-miR-145 | mmu-miR-145 | rno-miR-145 | | | 2 | 3 | Used |
| EAM211 | hsa-miR-144 | mmu-miR-144 | rno-miR-144 | | | 2 | 3 | Used |
| EAM210 | hsa-miR-143 | mmu-miR-143 | rno-miR-143 | | | 2 | 3 | Used |
| EAM208 | hsa-miR-141 | mmu-miR-141 | rno-miR-141 | | | 2 | 3 | Used |
| EAM207 | hsa-miR-140 | mmu-miR-140 | rno-miR-140 | | | 2 | 3 | Used |
| EAM206 | hsa-miR-139 | mmu-miR-139 | rno-miR-139 | | | 2 | 3 | Used |
| EAM205 | hsa-miR-138 | mmu-miR-138 | rno-miR-138 | | | 2 | 3 | Used |
| EAM203 | hsa-miR-135a | mmu-miR-135a | rno-miR-135a | | | 2 | 3 | Used |
| EAM200 | hsa-miR-133a | mmu-miR-133a | rno-miR-133a | | | 2 | 3 | Used |
| EAM195 | hsa-miR-128b | mmu-miR-128b | rno-miR-128b | | | 2 | 3 | Used |
| EAM194 | hsa-miR-128a | mmu-miR-128a | rno-miR-128a | | | 2 | 3 | Used |
| EAM193 | hsa-miR-125a | mmu-miR-125a | rno-miR-125a | | | 2 | 1 | Used |
| EAM190 | hsa-miR-10b | | rno-miR-10b | | | 2 | 1 | Used |
| EAM187 | hsa-miR-107 | mmu-miR-107 | rno-miR-107 | | | 2 | 1 | Used |
| EAM185 | hsa-miR-103 | mmu-miR-103 | rno-miR-103 | | | 2 | 1 | Used |
| EAM181 | hsa-let-7f | mmu-let-7f | rno-let-7f | | | 2 | 1 | Used |
| EAM179 | hsa-let-7d | mmu-let-7d | rno-let-7d | | | 2 | 1 | Used |
| EAM177 | | mmu-miR-101b | rno-miR-101b | | | 2 | 1 | Used |
| EAM175 | hsa-miR-320 | mmu-miR-320 | rno-miR-320 | | | 2 | 1 | Used |
| EAM168 | hsa-let-7e | mmu-let-7e | rno-let-7e | | | 2 | 1 | Used |
| EAM161 | hsa-miR-28 | mmu-miR-28 | rno-miR-28 | | | 2 | 1 | Used |
| EAM160 | hsa-miR-26b | mmu-miR-26b | rno-miR-26b | | | 2 | 1 | Used |
| EAM155 | hsa-miR-136 | mmu-miR-136 | rno-miR-136 | | | 2 | 1 | Used |
| EAM153 | hsa-let-7a | mmu-let-7a | rno-let-7a | | | 2 | 1 | Used |
| EAM147 | hsa-let-7b | mmu-let-7b | rno-let-7b | | | 2 | 1 | Used |
| EAM137 | hsa-miR-132 | mmu-miR-132 | rno-miR-132 | | | 2 | 1 | Used |
| EAM133 | hsa-miR-324-5p | mmu-miR-324-5p | rno-miR-324-5p | | | 2 | 1 | Used |
| EAM311 | hsa-miR-101 | mmu-miR-101 | rno-miR-101 | | | 2 | 2 | Used |
| EAM312 | hsa-miR-105 | | | | | 2 | 2 | Used |
| EAM313 | hsa-miR-106b | mmu-miR-106b | rno-miR-106b | | | 2 | 2 | Used |
| EAM314 | hsa-miR-126 | mmu-miR-126 | rno-miR-126 | | | 2 | 2 | Used |
| EAM315 | hsa-miR-127 | mmu-miR-127 | rno-miR-127 | | | 2 | 2 | Used |
| EAM316 | hsa-miR-147 | | | | | 2 | 2 | Used |
| EAM317 | hsa-miR-155 | | | | | 2 | 2 | Used |
| EAM318 | hsa-miR-17-3p | | | | | 2 | 2 | Used |
| EAM319 | hsa-miR-182* | | | | | 2 | 2 | Used |
| EAM320 | hsa-miR-189 | mmu-miR-189 | | | | 2 | 2 | Used |
| EAM321 | hsa-miR-200b | | rno-miR-200b | | | 2 | 2 | Used |
| EAM291 | hsa-miR-185 | mmu-miR-185 | rno-miR-185 | | | 2 | 2 | Used |
| EAM290 | hsa-miR-184 | mmu-miR-184 | rno-miR-184 | | | 2 | 2 | Used |
| EAM322 | hsa-miR-200c | mmu-miR-200c | rno-miR-200c | | | 3 | 2 | Used |
| EAM323 | hsa-miR-224 | | | | | 3 | 2 | Used |
| EAM324 | hsa-miR-25 | mmu-miR-25 | rno-miR-25 | | | 3 | 2 | Used |
| EAM325 | hsa-miR-27a | mmu-miR-27a | rno-miR-27a | | | 3 | 2 | Used |
| EAM326 | hsa-miR-296 | mmu-miR-296 | rno-miR-296 | | | 3 | 2 | Used |
| EAM327 | hsa-miR-299 | mmu-miR-299 | rno-miR-299 | | | 3 | 2 | Used |
| EAM328 | hsa-miR-301 | mmu-miR-301 | rno-miR-301 | | | 3 | 2 | Used |
| EAM329 | hsa-miR-302a | mmu-miR-302 | | | | 3 | 2 | Used |
| EAM330 | hsa-miR-30a-5p | mmu-miR-30a-5p | rno-miR-30a-5p | | | 3 | 2 | Used |
| EAM331 | hsa-miR-30e | mmu-miR-30e | rno-miR-30e | | | 3 | 2 | Used |
| EAM332 | hsa-miR-31 | mmu-miR-31 | rno-miR-31 | | | 3 | 2 | Used |
| EAM333 | hsa-miR-32 | mmu-miR-32 | rno-miR-32 | | | 3 | 2 | Used |
| EAM334 | | | | OLD_miR-321, | | 3 | 2 | Used |
| | | | | ARG_tRNA_ | | | | |
| | | | | FRAGMENT | | | | |
| EAM335 | hsa-miR-34b | | | | | 3 | 2 | Used |
| EAM336 | hsa-miR-34c | mmu-miR-34c | rno-miR-34c | | | 3 | 2 | Used |
| EAM337 | hsa-miR-93 | mmu-miR-93 | rno-miR-93 | | | 3 | 2 | Used |
| EAM338 | hsa-miR-95 | | | | | 3 | 2 | Used |
| EAM339 | hsa-miR-99b | mmu-miR-99b | rno-miR-99b | | | 3 | 2 | Used |
| EAM340 | | mmu-let-7d* | rno-let-7d* | | | 3 | 2 | Used |
| EAM341 | | mmu-miR-106a | | | | 3 | 2 | Used |
| EAM342 | hsa-miR-135b | mmu-miR-135b | rno-miR-135b | | | 3 | 2 | Used |
| EAM343 | | mmu-miR-151 | rno-miR-151 | | | 3 | 2 | Used |
| EAM344 | | mmu-miR-17-3p | | | | 3 | 2 | Used |
| EAM345 | | mmu-miR-224 | | | | 3 | 2 | Used |
| EAM346 | | mmu-miR-290 | rno-miR-290 | | | 3 | 2 | Used |
| EAM347 | | mmu-miR-291-3p | rno-miR-291-3p | | | 3 | 2 | Used |
| EAM348 | | mmu-miR-291-5p | rno-miR-291-5p | | | 3 | 2 | Used |
| EAM349 | | mmu-miR-292-3p | rno-miR-292-3p | | | 3 | 2 | Used |
| EAM350 | | mmu-miR-292-5p | rno-miR-292-5p | | | 3 | 2 | Used |
| EAM351 | | mmu-miR-293 | | | | 3 | 2 | Used |
| EAM352 | | mmu-miR-294 | | | | 3 | 2 | Used |
| EAM353 | | mmu-miR-295 | | | | 3 | 2 | Used |
| EAM354 | | mmu-miR-297 | | | | 3 | 2 | Used |
| EAM355 | | mmu-miR-298 | rno-miR-298 | | | 3 | 2 | Used |
| EAM356 | | mmu-miR-300 | rno-miR-300 | | | 3 | 2 | Used |
| EAM357 | | mmu-miR-322 | rno-miR-322 | | | 3 | 2 | Used |
| EAM358 | hsa-miR-323 | mmu-miR-323 | rno-miR-323 | | | 3 | 2 | Used |
| EAM359 | hsa-miR-324-3p | mmu-miR-324-3p | rno-miR-324-3p | | | 3 | 2 | Used |
| EAM360 | | mmu-miR-325 | rno-miR-325 | | | 3 | 2 | Used |
| EAM361 | hsa-miR-326 | mmu-miR-326 | rno-miR-326 | | | 3 | 2 | Used |
| EAM362 | hsa-miR-328 | mmu-miR-328 | rno-miR-328 | | | 3 | 2 | Used |
| EAM363 | | mmu-miR-329 | rno-miR-329 | | | 3 | 2 | Used |
| EAM364 | | mmu-miR-330 | rno-miR-330 | | | 3 | 2 | Used |
| EAM365 | hsa-miR-331 | mmu-miR-331 | rno-miR-331 | | | 3 | 2 | Used |
| EAM366 | | mmu-miR-337 | rno-miR-337 | | | 3 | 2 | Used |
| EAM367 | hsa-miR-338 | mmu-miR-338 | rno-miR-338 | | | 3 | 2 | Used |
| EAM368 | hsa-miR-339 | mmu-miR-339 | rno-miR-339 | | | 3 | 2 | Used |
| EAM369 | hsa-miR-340 | mmu-miR-340 | rno-miR-340 | | | 3 | 2 | Used |
| EAM370 | | mmu-miR-341 | rno-miR-341 | | | 3 | 2 | Used |
| EAM371 | hsa-miR-342 | mmu-miR-342 | rno-miR-342 | | | 3 | 2 | Used |
| EAM372 | | mmu-miR-344 | | | | 3 | 2 | Used |
| EAM373 | | mmu-miR-345 | rno-miR-345 | | | 3 | 2 | Used |
| EAM374 | | mmu-miR-346 | | | | 3 | 2 | Used |
| EAM375 | | mmu-miR-34b | rno-miR-34b | | | 3 | 2 | Used |
| EAM376 | | mmu-miR-350 | rno-miR-350 | | | 3 | 2 | Used |
| EAM377 | | mmu-miR-351 | rno-miR-351 | | | 3 | 2 | Used |
| EAM378 | | mmu-miR-7b | rno-miR-7b | | | 3 | 2 | Used |
| EAM379 | | | rno-miR-129* | | | 3 | 2 | Used |
| EAM380 | | | rno-miR-140* | | | 3 | 2 | Used |
| EAM381 | | | rno-miR-151* | | | 3 | 2 | Used |
| EAM382 | | | rno-miR-20* | | | 3 | 2 | Used |
| EAM383 | | | rno-miR-327 | | | 3 | 2 | Used |
| EAM384 | | | rno-miR-333 | | | 3 | 2 | Used |
| EAM385 | hsa-miR-335 | mmu-miR-335 | rno-miR-335 | | | 3 | 2 | Used |
| EAM386 | | | rno-miR-336 | | | 3 | 2 | Used |
| EAM387 | | | rno-miR-343 | | | 3 | 2 | Used |
| EAM388 | | | rno-miR-344 | | | 3 | 2 | Used |
| EAM389 | | | rno-miR-346 | | | 3 | 2 | Used |
| EAM390 | | | rno-miR-347 | | | 3 | 2 | Used |
| EAM391 | | | rno-miR-349 | | | 3 | 2 | Used |
| EAM392 | | | rno-miR-352 | | | 3 | 2 | Used |
| EAM393 | | | rno-miR-7* | | | 3 | 2 | Used |
| emc139 | | | | | Yes, | 3 | Not | Not Used, |
| | | | | | Other | | Used | control |
| | | | | | | | | feature |
| EAM289 | hsa-miR-129 | mmu-miR-129 | rno-miR-129 | | | 3 | 1 | Used |
| EAM283 | | mmu-miR-211 | rno-miR-211 | | | 3 | 1 | Used |
| PTG20210 | | | | | Yes, | 1,2,3 | 1,2,3 | Not Used, |
| | | | | | post- | | | control |
| | | | | | ctrl | | | feature |
| MRC677 | | | | | Yes, | 1,2,3 | 1,2,3 | Not Used, |
| | | | | | Other | | | control |
| | | | | | | | | feature |
| FVR506 | | | | | Yes, | 1,2,3 | 1,2,3 | Not Used, |
| | | | | | post- | | | control |
| | | | | | ctrl | | | feature |
| EAM104 | | | | | Yes, | 1,2,3 | 1 | Not Used, |
| | | | | | Mismatch | | | control |
| | | | | | | | | feature |
| EAM106 | | | | | Yes, | 1,2,3 | 1 | Not Used, |
| | | | | | Mismatch | | | control |
| | | | | | | | | feature |
| EAM110 | | | | | Yes, | 1,2,3 | 1 | Not Used, |
| | | | | | Mismatch | | | control |
| | | | | | | | | feature |
| EAM1101 | | | | | Yes, | 1,2,3 | 1 | Not Used, |
| | | | | | Mismatch | | | control |
| | | | | | | | | feature |
| EAM1102 | | | | | Yes, | 1,2,3 | Not | Not Used, |
| | | | | | Other | | Used | control |
| | | | | | | | | feature |
| EAM1103 | | | | | Yes, | 1,2,3 | Not | Not Used, |
| | | | | | Other | | Used | control |
| | | | | | | | | feature |
| EAM1104 | | | | | Yes, | 1,2,3 | Not | Not Used, |
| | | | | | Other | | Used | control |
| | | | | | | | | feature |
| EAM146 | | | | | Yes, | 1,2,3 | 1 | Not Used, |
| | | | | | Mismatch | | | control |
| | | | | | | | | feature |
| emc130 | | | | | Yes, | 1,2,3 | 1,2,3 | Not Used, |
| | | | | | Other | | | control |
| | | | | | | | | feature |
| emc115 | | | | | Yes, | 1,2,3 | 1,2,3 | Not Used, |
| | | | | | pre- | | | control |
| | | | | | ctrl | | | feature |
| EAM148 | | | | | Yes, | 1,2,3 | 1 | Not Used, |
| | | | | | Mismatch | | | control |
| | | | | | | | | feature |
| EAM138 | | | | | Yes, | 1,2,3 | 1 | Not Used, |
| | | | | | Mismatch | | | control |
| | | | | | | | | feature |
| EAM134 | | | | | Yes, | 1,2,3 | 1 | Not Used, |
| | | | | | Mismatch | | | control |
| | | | | | | | | feature |
| EAM395 | | | | | Yes, | 1,2,3 | 1,2,3 | Not Used, |
| | | | | | Other | | | control |
| | | | | | | | | feature |
| EAM149I | | | | | Yes, | 1,2,3 | Not | Not Used, |
| | | | | | Other | | Used | control |
| | | | | | | | | feature |
| EAM150I | | | | | Yes, | 1,2,3 | Not | Not Used, |
| | | | | | Other | | Used | control |
| | | | | | | | | feature |
| EAM399 | | | | ebv-miR-BHRF1-2 | | Not | 3 | Used only |
| | | | | | | Used | | in ALL |
| | | | | | | | | study |
| EAM400 | | | | ebv-miR-BHRF1-2* | | Not | 3 | Used only |
| | | | | | | Used | | in ALL |
| | | | | | | | | study |
| EAM401 | | | | ebv-miR-BHRF1-3 | | Not | 3 | Used only |
| | | | | | | Used | | in ALL |
| | | | | | | | | study |
| EAM402 | hsa-miR-133b | mmu-miR-133b | | | | Not | 3 | Used only |
| | | | | | | Used | | in ALL |
| | | | | | | | | study |
| EAM403 | hsa-miR-151 | | | | | Not | 3 | Used only |
| | | | | | | Used | | in ALL |
| | | | | | | | | study |
| EAM404 | hsa-miR-196b | mmu-miR-196b | rno-miR-196b | | | Not | 3 | Used only |
| | | | | | | Used | | in ALL |
| | | | | | | | | study |
| EAM405 | hsa-miR-302b | | | | | Not | 3 | Used only |
| | | | | | | Used | | in ALL |
| | | | | | | | | study |
| EAM406 | hsa-miR-302b* | | | | | Not | 3 | Used only |
| | | | | | | Used | | in ALL |
| | | | | | | | | study |
| EAM407 | hsa-miR-302c | | | | | Not | 3 | Used only |
| | | | | | | Used | | in ALL |
| | | | | | | | | study |
| EAM408 | hsa-miR-302c* | | | | | Not | 3 | Used only |
| | | | | | | Used | | in ALL |
| | | | | | | | | study |
| EAM409 | hsa-miR-302d | | | | | Not | 3 | Used only |
| | | | | | | Used | | in ALL |
| | | | | | | | | study |
| EAM410 | hsa-miR-325 | | | | | Not | 3 | Used only |
| | | | | | | Used | | in ALL |
| | | | | | | | | study |
| EAM411 | hsa-miR-330 | | | | | Not | 3 | Used only |
| | | | | | | Used | | in ALL |
| | | | | | | | | study |
| EAM412 | hsa-miR-337 | | | | | Not | 3 | Used only |
| | | | | | | Used | | in ALL |
| | | | | | | | | study |
| EAM413 | hsa-miR-345 | | | | | Not | 3 | Used only |
| | | | | | | Used | | in ALL |
| | | | | | | | | study |
| EAM414 | hsa-miR-346 | | | | | Not | 3 | Used only |
| | | | | | | Used | | in ALL |
| | | | | | | | | study |
| EAM415 | hsa-miR-367 | | | | | Not | 3 | Used only |
| | | | | | | Used | | in ALL |
| | | | | | | | | study |
| EAM416 | hsa-miR-368 | | | | | Not | 3 | Used only |
| | | | | | | Used | | in ALL |
| | | | | | | | | study |
| EAM417 | hsa-miR-369 | | | | | Not | 3 | Used only |
| | | | | | | Used | | in ALL |
| | | | | | | | | study |
| EAM418 | hsa-miR-370 | mmu-miR-370 | | | | Not | 3 | Used only |
| | | | | | | Used | | in ALL |
| | | | | | | | | study |
| EAM419 | hsa-miR-371 | | | | | Not | 3 | Used only |
| | | | | | | Used | | in ALL |
| | | | | | | | | study |
| EAM420 | hsa-miR-372 | | | | | Not | 3 | Used only |
| | | | | | | Used | | in ALL |
| | | | | | | | | study |
| EAM421 | hsa-miR-373 | | | | | Not | 3 | Used only |
| | | | | | | Used | | in ALL |
| | | | | | | | | study |
| EAM422 | hsa-miR-373* | | | | | Not | 3 | Used only |
| | | | | | | Used | | in ALL |
| | | | | | | | | study |
| EAM423 | hsa-miR-374 | | | | | Not | 3 | Used only |
| | | | | | | Used | | in ALL |
| | | | | | | | | study |
| EAM424 | hsa-miR-133b | mmu-miR-133b | | | | Not | 3 | Used only |
| | | | | | | Used | | in ALL |
| | | | | | | | | study |
| EAM425 | hsa-miR-196b | mmu-miR-196b | rno-miR-196b | | | Not | 3 | Used only |
| | | | | | | Used | | in ALL |
| | | | | | | | | study |
| EAM426 | | mmu-miR-215 | | | | Not | 3 | Used only |
| | | | | | | Used | | in ALL |
| | | | | | | | | study |
| EAM427 | | mmu-miR-409 | | | | Not | 3 | Used only |
| | | | | | | Used | | in ALL |
| | | | | | | | | study |
| EAM428 | | mmu-miR-410 | | | | Not | 3 | Used only |
| | | | | | | Used | | in ALL |
| | | | | | | | | study |
| EAM429 | | mmu-miR-376b | | | | Not | 3 | Used only |
| | | | | | | Used | | in ALL |
| | | | | | | | | study |
| EAM430 | | mmu-miR-376a | | | | Not | 3 | Used only |
| | | | | | | Used | | in ALL |
| | | | | | | | | study |
| EAM431 | | mmu-miR-411 | | | | Not | 3 | Used only |
| | | | | | | Used | | in ALL |
| | | | | | | | | study |
| EAM432 | | mmu-miR-380-3p | | | | Not | 3 | Used only |
| | | | | | | Used | | in ALL |
| | | | | | | | | study |
| EAM433 | | mmu-miR-412 | | | | Not | 3 | Used only |
| | | | | | | Used | | in ALL |
| | | | | | | | | study |
| EAM396 | | | | ebv-miR-BART1 | | Not | 3 | Used only |
| | | | | | | Used | | in ALL |
| | | | | | | | | study |
| EAM397 | | | | ebv-miR-BART2 | | Not | 3 | Used only |
| | | | | | | Used | | in ALL |
| | | | | | | | | study |
| EAM398 | | | | ebv-miR-BHRF1-1 | | Not | 3 | Used only |
| | | | | | | Used | | in ALL |
| | | | | | | | | study |

TABLE 11

Oligonucleotide Sequences for Detection
Specificity Experiment

| miRNA or | |
|---|---|
| Mutant Name | Oligonucleotide Sequence (5′ to 3′) |
| hsa-let-7g | CTGGAATTCGCGGTTAAAACTGTACAAACTACTACCTCA |
| | TTTAGTGAGGAATTCCGT |
| | (Seq ID No:850) |
| let-7-mut1 | CTGGAATTCGCGGTTAAATAACTGTAGAAAGTACTACCT |
| | CATTTAGTGAGGAATTCCGT |
| | (Seq ID No:851) |
| hsa-let-7c | CTGGAATTCGCGGTITAAAAACCATACAACCTACTACCT |
| | CATTTTAGTGAGGAATTCCGT |
| | (Seq ID No:852) |
| let-7-mut2 | CTGGAATTCGCGGTTAAAAACCATACAAGCTAGTACCTC |
| | ATTTAGTGAGGAATTCCGT |
| | (Seq ID No:853) |
| hsa-let-7b | CTGGAATTCGCGGTTAAAAACCACACAACCTACTACCTC |
| | ATTTAGTGAGGAATTCCGT |
| | (Seq ID No:854) |
| let-7-mut3 | CTGGAATTCGCGGTTAAAAACCACACAAGCTAGTACCTC |
| | ATTTAGTGAGGAATTCCGT |
| | (Seq ID No:855) |
| hsa-let-7a | CTGGAATTCGCGGTTAAAAACTATACAACCTACTACCTC |
| | ATTTAGTGAGGAATTCCGT |
| | (Seq ID No:856) |
| hsa-let-7e | CTGGAATTCGCGGTTAAAACTATACAACCTCCTACCTCA |
| | TTTAGTGAGGAATTCCGT |
| | (Seq ID No:857) |
| hsa-let-7d | CTGGAATTCGCGGTTAAAACTATGCAACCTACTACCTCT |
| | TTTAGTGAGGAATTCCGT |
| | (Seq ID No:858) |
| hsa-let-7f | CTGGAATTCGCGGTTAAAAACTATACAATCTACTACCTC |
| | ATTTAGTGAGGAATTCCGT |
| | (Seq ID No:858) |
| hsa-let-7i | CTGGAATTCGCGGTTAAAAGCACAAACTACTACCTCATT |
| | TAGTGAGGAATTCCGT |
| | (Seq ID No:860) |

TABLE 12

Alignment of Human let-7 miRNAs
and Mutant Sequences

| | | |
|---|---|---|
| UGAGGUAGUAGUUUGUACAGU | (Seq ID No:861) | hsa-let-7g |
| UGAGGUAGUACUUUCUACAGUUA | (Seq ID No:862) | let-7-mut1 |
| UGAGGUAGUAGGUUGUAUGGUU | (Seq ID No:863) | hsa-let-7c |
| UGAGGUACUAGCUUGUAUGGUU | (Seq ID No:864) | let-7-mut2 |
| UGAGGUAGUAGGUUGUGUGGUU | (Seq ID No:865) | hsa-let-7b |
| UGAGGUACUAGCUUGUGUGGUU | (Seq ID No:866) | let-7-mut3 |
| UGAGGUAGUAGGUUGUAUAGUU | (Seq ID No:867) | hsa-let-7a |
| UGAGGUAGGAGGUUGUAUAGU | (Seq ID No:868) | hsa-let-7e |
| AGAGGUAGUAGGUUGCAUAGU | (Seq ID No:869) | hsa-let-7d |
| UGAGGUAGUAGAUUGUAUAGUU | (Seq ID No:870) | hsa-let-7f |
| UGAGGUAGUAGUUUGUGCU | (Seq ID No:871) | hsa-let-7i |

TABLE 13
220 mRNA genes with transcription factor activity annotation

| Chip | Probe Set ID | Gene Title |
|---|---|---|
| Hu6800 | AB000468_at | ring finger protein 4 |
| Hu6800 | D43642_at | transcription factor-like 1 |
| Hu6800 | D83784_at | pleiomorphic adenoma gene-like 2 |
| Hu6800 | D86479_at | AE binding protein 1 |
| Hu6800 | D87673_at | heat shock transcription factor 4 |
| Hu6800 | J03161_at | serum response factor (c-fos serum response element- |
| | | binding transcription factor) |
| Hu6800 | J03827_at | nuclease sensitive element binding protein 1 |
| Hu6800 | L02785_at | solute carrier family 26, member 3 |
| Hu6800 | L11672_at | zinc finger protein 91 (HPF7, HTF10) |
| Hu6800 | L11672_r_at | zinc finger protein 91 (HPF7, HTF10) |
| Hu6800 | L13203_at | forkhead box I1 |
| Hu6800 | L13740_at | nuclear receptor subfamily 4, group A, member 1 |
| Hu6800 | L17131_rna1_at | high mobility group AT-hook 1 |
| Hu6800 | L20298_at | core-binding factor, beta subunit |
| Hu6800 | L22342_at | SP110 nuclear body protein |
| Hu6800 | L22454_at | nuclear respiratory factor 1 |
| Hu6800 | L40904_at | peroxisome proliferative activated receptor, gamma |
| Hu6800 | M14328_s_at | enolase 1, (alpha) |
| Hu6800 | M16938_s_at | homeo box C6 |
| Hu6800 | M19720_rna1_at | v-myc myelocytomatosis viral oncogene homolog 1, lung |
| | | carcinoma derived (avian) |
| Hu6800 | M23263_at | androgen receptor (dihydrotestosterone receptor; testicular |
| | | feminization; spinal and bulbar muscular atrophy; Kennedy |
| | | disease) |
| Hu6800 | M24900_at | thyroid hormone receptor, alpha (erythroblastic leukemia |
| | | viral (v-erb-a) oncogene homolog, avian) /// nuclear |
| | | receptor subfamily 1, group D, member 1 |
| Hu6800 | M25269_at | ELK1, member of ETS oncogene family |
| Hu6800 | M31627_at | X-box binding protein 1 |
| Hu6800 | M36542_s_at | POU domain, class 2, transcription factor 2 |
| Hu6800 | M38258_at | retinoic acid receptor, gamma |
| Hu6800 | M64673_at | heat shock transcription factor 1 |
| Hu6800 | M65214_s_at | transcription factor 3 (E2A immunoglobulin enhancer |
| | | binding factors E12/E47) |
| Hu6800 | M68891_at | GATA binding protein 2 |
| Hu6800 | M76732_s_at | msh homeo box homolog 1 (Drosophila) |
| Hu6800 | M77698_at | YY1 transcription factor |
| Hu6800 | M79462_at | promyelocytic leukemia |
| Hu6800 | M79463_s_at | promyelocytic leukemia |
| Hu6800 | M93650_at | paired box gene 6 (aniridia, keratitis) |
| Hu6800 | M95929_at | sideroflexin 3 |
| Hu6800 | M97676_at | msh homeo box homolog 1 (Drosophila) |
| Hu6800 | M97935_s_at | signal transducer and activator of transcription 1, 91 kDa |
| Hu6800 | M97936_at | signal transducer and activator of transcription 1, 91 kDa |
| Hu6800 | M99701_at | transcription elongation factor A (SII)-like 1 |
| Hu6800 | S81264_s_at | T-box 2 |
| Hu6800 | U00968_at | sterol regulatory element binding transcription factor 1 |
| Hu6800 | U11861_at | maternal G10 transcript |
| Hu6800 | U18018_at | ets variant gene 4 (E1A enhancer binding protein, E1AF) |
| Hu6800 | U20734_s_at | jun B proto-oncogene |
| Hu6800 | U28687_at | zinc finger protein 157 (HZF22) |
| Hu6800 | U29175_at | SWI/SNF related, matrix associated, actin dependent |
| | | regulator of chromatin, subfamily a, member 4 |
| Hu6800 | U35048_at | transforming growth factor beta 1 induced transcript 4 |
| Hu6800 | U36922_at | forkhead box O1A (rhabdomyosarcoma) |
| Hu6800 | U39840_at | forkhead box A1 |
| Hu6800 | U44755_at | small nuclear RNA activating complex, polypeptide 2, |
| | | 45 kDa |
| Hu6800 | U51003_s_at | distal-less homeo box 2 |
| Hu6800 | U51127_at | interferon regulatory factor 5 |
| Hu6800 | U53830_at | interferon regulatory factor 7 |
| Hu6800 | U58681_at | neurogenic differentiation 2 |
| Hu6800 | U63842_at | neurogenin 1 |
| Hu6800 | U69126_s_at | KH-type splicing regulatory protein (FUSE binding protein |
| | | 2) |
| Hu6800 | U72649_at | BTG family, member 2 |
| Hu6800 | U73843_at | E74-like factor 3 (ets domain transcription factor, epithelial- |
| | | specific) |
| Hu6800 | U76388_at | nuclear receptor subfamily 5, group A, member 1 |
| Hu6800 | U81599_at | homeo box B13 |
| Hu6800 | U81600_at | paired related homeobox 2 |
| Hu6800 | U82759_at | homeo box A9 |
| Hu6800 | U85193_at | nuclear factor I/B |
| Hu6800 | U85658_at | transcription factor AP-2 gamma (activating enhancer |
| | | binding protein 2 gamma) |
| Hu6800 | U95040_at | tripartite motif-containing 28 |
| Hu6800 | X03635_at | estrogen receptor 1 |
| Hu6800 | X06614_at | retinoic acid receptor, alpha |
| Hu6800 | X12794_at | nuclear receptor subfamily 2, group F, member 6 |
| Hu6800 | X13293_at | v-myb myeloblastosis viral oncogene homolog (avian)-like 2 |
| Hu6800 | X13810_s_at | POU domain, class 2, transcription factor 2 |
| Hu6800 | X16316_at | vav 1 oncogene |
| Hu6800 | X16665_at | homeo box B2 |
| Hu6800 | X16706_at | FOS-like antigen 2 |
| Hu6800 | X17360_rna1_at | homeo box D4 |
| Hu6800 | X17651_at | myogenin (myogenic factor 4) |
| Hu6800 | X51345_at | jun B proto-oncogene |
| Hu6800 | X52541_at | early growth response 1 |
| Hu6800 | X55005_rna1_at | thyroid hormone receptor, alpha (erythroblastic leukemia |
| | | viral (v-erb-a) oncogene homolog, avian) |
| Hu6800 | X55037_s_at | GATA binding protein 3 |
| Hu6800 | X56681_s_at | jun D proto-oncogene |
| Hu6800 | X58072_at | GATA binding protein 3 |
| Hu6800 | X60003_s_at | cAMP responsive element binding protein 1 |
| Hu6800 | X61755_rna1_s_at | homeo box C5 |
| Hu6800 | X65463_at | retinoid X receptor, beta |
| Hu6800 | X66079_at | Spi-B transcription factor (Spi-1/PU.1 related) |
| Hu6800 | X68688_rna1_s_at | zinc finger protein 11b (KOX 2) /// zinc finger protein 33a |
| | | (KOX 31) |
| Hu6800 | X69699_at | paired box gene 8 |
| Hu6800 | X70683_at | SRY (sex determining region Y)-box 4 |
| Hu6800 | X72632_s_at | thyroid hormone receptor, alpha (erythroblastic leukemia |
| | | viral (v-erb-a) oncogene homolog, avian) /// nuclear |
| | | receptor subfamily 1, group D, member 1 |
| Hu6800 | X78992_at | zinc finger protein 36, C3H type-like 2 |
| Hu6800 | X85786_at | regulatory factor X, 5 (influences HLA class II expression) |
| Hu6800 | X90824_s_at | upstream transcription factor 2, c-fos interacting |
| Hu6800 | X93996_rna1_at | myeloid/lymphoid or mixed-lineage leukemia (trithorax |
| | | homolog, Drosophila); translocated to, 7 |
| Hu6800 | X96401_at | MAX binding protein |
| Hu6800 | X96506_s_at | DR1-associated protein 1 (negative cofactor 2 alpha) |
| Hu6800 | X99101_at | estrogen receptor 2 (ER beta) |
| Hu6800 | Y08976_at | FEV (ETS oncogene family) |
| Hu6800 | Z11899_s_at | POU domain, class 5, transcription factor 1 |
| Hu6800 | Z17240_at | high-mobility group box 2 |
| Hu6800 | Z22951_rna1_s_at | — |
| Hu6800 | Z49825_s_at | hepatocyte nuclear factor 4, alpha |
| Hu6800 | Z50781_at | delta sleep inducing peptide, immunoreactor |
| Hu6800 | Z56281_at | interferon regulatory factor 3 |
| Hu35KsubA | AA010750_at | LAG1 longevity assurance homolog 2 (S. cerevisiae) |
| Hu35KsubA | AA036900_at | FOS-like antigen 2 |
| Hu35KsubA | AA091017_at | nuclear factor of activated T-cells 5, tonicity-responsive |
| Hu35KsubA | AA099501_at | p66 alpha |
| Hu35KsubA | AA127183_s_at | serologically defined colon cancer antigen 33 |
| Hu35KsubA | AA157520_at | signal transducer and activator of transcription 5B |
| Hu35KsubA | AA287840_at | Runt-related transcription factor 2 |
| Hu35KsubA | AA328684_at | SLC2A4 regulator |
| Hu35KsubA | AA347664_at | lymphoid enhancer-binding factor 1 |
| Hu35KsubA | AA355201_at | SRY (sex determining region Y)-box 4 |
| Hu35KsubA | AA418098_at | cAMP responsive element binding protein-like 2 |
| Hu35KsubA | AA424381_s_at | Forkhead box C1 |
| Hu35KsubA | AA431268_at | — |
| Hu35KsubA | AA436315_at | forkhead box O3A |
| Hu35KsubA | AA456687_at | nuclear factor I/A |
| Hu35KsubA | AA459542_s_at | regulatory factor X-associated ankyrin-containing protein |
| Hu35KsubA | AA489299_at | transcriptional adaptor 3 (NGG1 homolog, yeast)-like |
| Hu35KsubA | AA504413_at | Solute carrier family 25, member 29 |
| Hu35KsubA | AB002302_at | myeloid/lymphoid or mixed-lineage leukemia 4 |
| Hu35KsubA | AB002305_at | aryl-hydrocarbon receptor nuclear translocator 2 |
| Hu35KsubA | AB004066_at | basic helix-loop-helix domain containing, class B, 2 |
| Hu35KsubA | C02099_s_at | methionine sulfoxide reductase B2 |
| Hu35KsubA | D45333_at | prefoldin 1 |
| Hu35KsubA | D61676_at | Pre-B-cell leukemia transcription factor 1 |
| Hu35KsubA | D82636_at | CCR4-NOT transcription complex, subunit 7 |
| Hu35KsubA | H45647_at | hairy/enhancer-of-split related with YRPW motif 1 |
| Hu35KsubA | IKAROS_at | zinc finger protein, subfamily 1A, 1 (Ikaros) |
| Hu35KsubA | L07592_at | peroxisome proliferative activated receptor, delta |
| Hu35KsubA | L13203_at | forkhead box I1 |
| Hu35KsubA | L16794_s_at | MADS box transcription enhancer factor 2, polypeptide D |
| | | (myocyte enhancer factor 2D) |
| Hu35KsubA | L40904_at | peroxisome proliferative activated receptor, gamma |
| Hu35KsubA | L41067_at | nuclear factor of activated T-cells, cytoplasmic, calcineurin- |
| | | dependent 3 |
| Hu35KsubA | M23263_at | androgen receptor (dihydrotestosterone receptor; testicular |
| | | feminization; spinal and bulbar muscular atrophy; Kennedy |
| | | disease) |
| Hu35KsubA | M62626_s_at | T-cell leukemia, homeobox 1 |
| Hu35KsubA | M79462_at | promyelocytic leukemia |
| Hu35KsubA | M92299_s_at | homeo box B5 |
| Hu35KsubA | M93650_at | paired box gene 6 (aniridia, keratitis) |
| Hu35KsubA | M96577_s_at | E2F transcription factor 1 |
| Hu35KsubA | M97676_at | msh homeo box homolog 1 (Drosophila) |
| Hu35KsubA | N32724_at | high-mobility group 20B |
| Hu35KsubA | N83192_at | KIAA0669 gene product |
| Hu35KsubA | RC_AA029288_at | zinc finger protein 83 (HPF1) |
| Hu35KsubA | RC_AA040699_at | ELK3, ETS-domain protein (SRF accessory protein 2) |
| Hu35KsubA | RC_AA045545_at | glucocorticoid modulatory element binding protein 2 |
| Hu35KsubA | RC_AA055932_at | TAF5-like RNA polymerase II, p300/CBP-associated factor |
| | | (PCAF)-associated factor, 65 kDa |
| Hu35KsubA | RC_AA065094_at | trinucleotide repeat containing 4 |
| Hu35KsubA | RC_AA069549_at | zinc finger protein 37a (KOX 21) |
| Hu35KsubA | RC_AA114866_s_at | homeo box A11 |
| Hu35KsubA | RC_AA121121_at | Huntingtin interacting protein 2 |
| Hu35KsubA | RC_AA135095_at | high-mobility group 20B |
| Hu35KsubA | RC_AA136474_at | Meis1, myeloid ecotropic viral integration site 1 homolog 2 |
| | | (mouse) |
| Hu35KsubA | RC_AA150205_at | Kruppel-like factor 7 (ubiquitous) |
| Hu35KsubA | RC_AA156112_at | Krueppel-related zinc finger protein |
| Hu35KsubA | RC_AA156359_at | TAR DNA binding protein |
| Hu35KsubA | RC_AA156792_at | hairy/enhancer-of-split related with YRPW motif-like |
| Hu35KsubA | RC_AA235980_at | transcription factor EB |
| Hu35KsubA | RC_AA252161_at | p66 alpha |
| Hu35KsubA | RC_AA253429_at | zinc finger protein 175 |
| Hu35KsubA | RC_AA256678_at | CCR4-NOT transcription complex, subunit 7 |
| Hu35KsubA | RC_AA256680_at | Nuclear factor I/B |
| Hu35KsubA | RC_AA280130_at | checkpoint suppressor 1 |
| Hu35KsubA | RC_AA284143_at | arginine-glutamic acid dipeptide (RE) repeats |
| Hu35KsubA | RC_AA286809_at | upstream binding protein 1 (LBP-1a) |
| Hu35KsubA | RC_AA292717_at | forkhead box P1 |
| Hu35KsubA | RC_AA347288_at | growth arrest-specific 7 |
| Hu35KsubA | RC_AA379087_s_at | apoptosis antagonizing transcription factor |
| Hu35KsubA | RC_AA393876_s_at | nuclear receptor subfamily 2, group F, member 2 |
| Hu35KsubA | RC_AA419547_at | E74-like factor 5 (ets domain transcription factor) |
| Hu35KsubA | RC_AA421050_at | zinc finger protein 444 |
| Hu35KsubA | RC_AA425309_at | Nuclear factor I/B |
| Hu35KsubA | RC_AA428024_at | ubinuclein 1 |
| Hu35KsubA | RC_AA430032_at | pituitary tumor-transforming 1 |
| Hu35KsubA | RC_AA431399_at | arginine-glutamic acid dipeptide (RE) repeats |
| Hu35KsubA | RC_AA436608_at | SATB family member 2 |
| Hu35KsubA | RC_AA443090_s_at | interferon regulatory factor 7 |
| Hu35KsubA | RC_AA443962_at | MYST histone acetyltransferase 2 |
| Hu35KsubA | RC_AA452256_at | zinc finger protein 265 |
| Hu35KsubA | RC_AA456289_at | nuclear factor I/A |
| Hu35KsubA | RC_AA456677_at | zinc finger protein, subfamily 1A, 4 (Eos) |
| Hu35KsubA | RC_AA464251_at | LOC440448 |
| Hu35KsubA | RC_AA476720_at | nuclear factor of activated T-cells, cytoplasmic, calcineurin- |
| | | dependent 1 |
| Hu35KsubA | RC_AA478590_at | forkhead box O3A |
| Hu35KsubA | RC_AA478596_at | zinc fingers and homeoboxes 2 |
| Hu35KsubA | RC_AA504110_at | v-ets erythroblastosis virus E26 oncogene homolog 1 |
| | | (avian) |
| Hu35KsubA | RC_AA504144_at | CAMP responsive element binding protein 1 |
| Hu35KsubA | RC_AA504147_s_at | Solute carrier family 25, member 29 |
| Hu35KsubA | RC_AA609017_s_at | forkhead box O1A (rhabdomyosarcoma) |
| Hu35KsubA | RC_AA621179_at | forkhead box J2 |
| Hu35KsubA | RC_AA621680_at | Kruppel-like factor 4 (gut) |
| Hu35KsubA | RC_D59299_i_at | myeloid/lymphoid or mixed-lineage leukemia (trithorax |
| | | homolog, Drosophila); translocated to, 10 |
| Hu35KsubA | U09366_at | zinc finger protein 133 (clone pHZ-13) |
| Hu35KsubA | U17163_at | ets variant gene 1 |
| Hu35KsubA | U28687_at | zinc finger protein 157 (HZF22) |
| Hu35KsubA | U33749_s_at | thyroid transcription factor 1 |
| Hu35KsubA | U53831_s_at | interferon regulatory factor 7 |
| Hu35KsubA | U62392_at | zinc finger protein 193 |
| Hu35KsubA | U63824_at | TEA domain family member 4 |
| Hu35KsubA | U76388_at | nuclear receptor subfamily 5, group A, member 1 |
| Hu35KsubA | U81600_at | paired related homeobox 2 |
| Hu35KsubA | U85707_at | Meis1, myeloid ecotropic viral integration site 1 homolog |
| | | (mouse) |
| Hu35KsubA | U88047_at | AT rich interactive domain 3A (BRIGHT-like) |
| Hu35KsubA | U89995_at | forkhead box E1 (thyroid transcription factor 2) |
| Hu35KsubA | W20276_f_at | CG9886-like |
| Hu35KsubA | W26259_at | forkhead box O3A |
| Hu35KsubA | W55861_at | Myeloid/lymphoid or mixed-lineage leukemia (trithorax |
| | | homolog, Drosophila) |
| Hu35KsubA | W67850_s_at | TGFB-induced factor 2 (TALE family homeobox) |
| Hu35KsubA | X13403_s_at | POU domain, class 2, transcription factor 1 |
| Hu35KsubA | X16666_s_at | homeo box B1 |
| Hu35KsubA | X52402_s_at | homeo box C5 |
| Hu35KsubA | X52560_s_at | CCAAT/enhancer binding protein (C/EBP), beta |
| Hu35KsubA | X58431_rna2_s_at | homeo box B6 |
| Hu35KsubA | X68688_rna1_s_at | zinc finger protein 11b (KOX 2) /// zinc finger protein 33a |
| | | (KOX 31) |
| Hu35KsubA | X70683_at | SRY (sex determining region Y)-box 4 |
| Hu35KsubA | X99101_at | estrogen receptor 2 (ER beta) |
| Hu35KsubA | X99350_rna1_at | forkhead box J1 |
| Hu35KsubA | Y10746_at | methyl-CpG binding domain protein 1 |
| Hu35KsubA | Z14077_s_at | YY1 transcription factor |

TABLE 14
Number of Training Samples Used to Build
the Normal/Tumor Classifier

| Tissue | Number of Normal | Number of Tumor |
|---|---|---|
| Colon | 5 | 10 |
| Kidney | 3 | 5 |
| Prostate | 8 | 6 |
| Uterus | 9 | 10 |
| Lung | 4 | 6 |
| Breast | 3 | 6 |

TABLE 15

Normal/Tumor Makers Selected
On the Training Set

| | | | |
|---|---|---|---|
| | | Bonferroni- | Variance- |
| | | corrected | thresholded |
| Probe | Description | p-value | t-test score |

| | | | |
|---|---|---|---|
| EAM159 | hmr_miR-130a | 0 | 10.984 |
| EAM331 | hmr_miR-30e | 0 | 10.756 |
| EAM311 | hmr_miR-101 | 0 | 10.392 |
| EAM299 | hmr_miR-195 | 0 | 9.957 |
| EAM314 | hmr_miR-126 | 0 | 9.498 |
| EAM300 | h_miR-197 | 0 | 8.762 |
| EAM181 | hmr_let-7f | 0 | 8.299 |
| EAM380 | r_miR-140* | 0 | 8.238 |
| EAM111 | hm_let-7g | 0 | 8.235 |
| EAM381 | r_miR-151* | 0 | 8.198 |
| EAM218 | hmr_miR-152 | 0 | 8.180 |
| EAM183 | hmr_let-7i | 0 | 8.098 |
| EAM253 | hmr_miR-218 | 0 | 8.077 |
| EAM155 | hmr_miR-136 | 0 | 8.058 |
| EAM192 | hmr_miR-126* | 0 | 7.991 |
| EAM222 | hm_miR-15a | 0 | 7.970 |
| EAM161 | hmr_miR-28 | 0 | 7.949 |
| EAM184 | hmr_miR-100 | 0 | 7.894 |
| EAM271 | hmr_miR-30c | 0 | 7.848 |
| EAM270 | hmr_miR-30b | 0 | 7.731 |
| EAM303 | hm_miR-199a* | 0 | 7.519 |
| EAM121 | hmr_miR-99a | 0 | 7.515 |
| EAM392 | r_miR-352 | 0 | 7.476 |
| EAM255 | hmr_miR-22 | 0 | 7.465 |
| EAM249 | hmr_miR-214 | 0 | 7.338 |
| EAM160 | hmr_miR-26b | 0 | 7.313 |
| EAM133 | hmr_miR-324-5p | 0 | 7.266 |
| EAM238 | hm_miR-1 | 0 | 7.259 |
| EAM179 | hmr_let-7d | 0 | 7.235 |
| EAM339 | hmr_miR-99b | 0 | 7.225 |
| EAM185 | hmr_miR-103 | 0 | 7.047 |
| EAM168 | hmr_let-7e | 0 | 7.034 |
| EAM200 | hmr_miR-133a | 0 | 6.959 |
| EAM278 | hmr_miR-98 | 0 | 6.952 |
| EAM333 | hmr_miR-32 | 0 | 6.951 |
| EAM291 | hmr_miR-185 | 0 | 6.910 |
| EAM187 | hmr_miR-107 | 0 | 6.879 |
| EAM263 | hmr_miR-26a | 0 | 6.818 |
| EAM261 | hmr_miR-23b | 0 | 6.814 |
| EAM371 | hmr_miR-342 | 0 | 6.743 |
| EAM330 | hmr_miR-30a-5p | 0 | 6.717 |
| EAM280 | hmr_miR-30a-3p | 0 | 6.662 |
| EAM233 | hmr_miR-196a | 0 | 6.630 |
| EAM292 | hmr_miR-186 | 0 | 6.602 |
| EAM115 | hmr_miR-16 | 0 | 6.558 |
| EAM272 | hmr_miR-30d | 0 | 6.516 |
| EAM367 | hmr_miR-338 | 0 | 6.428 |
| EAM379 | r_mIR-129* | 0 | 6.323 |
| EAM193 | hmr_miR-125a | 0 | 6.222 |
| EAM273 | hmr_miR-33 | 0 | 6.209 |
| EAM223 | hmr_miR-15b | 0 | 6.148 |
| EAM105 | hmr_miR-125b | 0 | 6.111 |
| EAM385 | hmr_miR-335 | 0 | 6.011 |
| EAM237 | hmr_miR-19b | 0 | 5.981 |
| EAM320 | hm_miR-189 | 0 | 5.938 |
| EAM262 | hmr_miR-24 | 0 | 5.909 |
| EAM240 | hmr_miR-20 | 0 | 5.908 |
| EAM260 | hmr_miR-23a | 0 | 5.901 |
| EAM297 | hmr_miR-193 | 0 | 5.856 |
| EAM236 | hmr_miR-19a | 0 | 5.789 |
| EAM264 | hmr_miR-27b | 0 | 5.780 |
| EAM205 | hmr_miR-138 | 0 | 5.721 |
| EAM234 | hmr_miR-199a | 0 | 5.718 |
| EAM207 | hmr_miR-140 | 0 | 5.561 |
| EAM217 | hmr_miR-150 | 0 | 5.531 |
| EAM235 | h_miR-199b | 0 | 5.516 |
| EAM190 | hr_miR-10b | 0 | 5.511 |
| EAM282 | m_miR-199b | 0 | 5.483 |
| EAM335 | h_miR-34b | 0 | 5.315 |
| EAM288 | m_miR-10b | 0 | 5.291 |
| EAM275 | hmr_miR-34a | 0 | 5.287 |
| EAM195 | hmr_miR-128b | 0 | 5.253 |
| EAM328 | hmr_miR-301 | 0 | 5.203 |
| EAM365 | hmr_miR-331 | 0 | 5.191 |
| EAM131 | hmr_miR-92 | 0 | 5.155 |
| EAM215 | hmr_miR-148b | 0 | 5.091 |
| EAM325 | hmr_miR-27a | 0 | 5.090 |
| EAM279 | hmr_miR-29c | 0 | 5.025 |
| EAM369 | hmr_miR-340 | 0 | 4.959 |
| EAM354 | m_miR-297 | 0 | 4.953 |
| EAM119 | hmr_miR-29b | 0 | 4.937 |
| EAM210 | hmr_miR-143 | 0 | 4.908 |
| EAM361 | hmr_miR-326 | 0 | 4.790 |
| EAM324 | hmr_miR-25 | 0 | 4.764 |
| EAM226 | hmr_miR-181a | 0 | 4.742 |
| EAM343 | mr_miR-151 | 0 | 4.740 |
| EAM228 | hmr_miR-181c | 0 | 4.675 |
| EAM366 | mr_miR-337 | 0 | 4.661 |
| EAM349 | mr_miR-292-3p | 0 | 4.652 |
| EAM189 | hmr_miR-10a | 0 | 4.494 |
| EAM355 | mr_miR-298 | 0 | 4.446 |
| EAM318 | h_miR-17-3p | 0 | 4.324 |
| EAM387 | r_miR-343 | 0 | 4.140 |
| EAM363 | mr_miR-329 | 0 | 4.118 |
| EAM268 | hmr_miR-29a | 0 | 4.044 |
| EAM175 | hmr_miR-320 | 0 | 3.875 |
| EAM212 | hmr_miR-145 | 0 | 3.869 |
| EAM378 | mr_miR-7b | 0 | 3.853 |
| EAM281 | mr_miR-217 | 0 | 3.670 |
| EAM307 | m_miR-202 | 0 | 3.625 |
| EAM209 | hmr_miR-142-5p | 0 | 3.594 |
| EAM163 | hmr_miR-142-3p | 0 | 3.545 |
| EAM384 | r_miR-333 | 0 | 3.410 |
| EAM362 | hmr_miR-328 | 0 | 3.356 |
| EAM329 | hm_miR-302a | 0 | 3.348 |
| EAM368 | hmr_miR-339 | 0 | 3.007 |
| EAM351 | m_miR-293 | 0 | 2.852 |
| EAM153 | hmr_let-7a | 0 | 2.818 |
| EAM360 | mr_miR-325 | 0 | 2.753 |
| EAM145 | hmr_let-7c | 0 | 2.393 |
| EAM348 | mr_miR-291-5p | 0 | 2.092 |
| EAM298 | hmr_miR-194 | 0 | 2.068 |
| EAM250 | h_miR-215 | 0 | 1.746 |
| EAM229 | hm_miR-182 | 0.005 | −4.074 |
| EAM224 | hmr_miR-17-5p | 0.005 | 4.875 |
| EAM341 | m_miR-106a | 0.005 | 4.185 |
| EAM242 | hmr_miR-204 | 0.005 | 3.457 |
| EAM295 | hmr_miR-190 | 0.005 | 3.186 |
| EAM353 | m_miR-295 | 0.005 | 2.916 |
| EAM246 | h_miR-211 | 0.005 | 2.663 |
| EAM248 | hmr_miR-213 | 0.01 | 3.369 |
| EAM186 | h_miR-106a | 0.01 | 4.650 |
| EAM137 | hmr_miR-132 | 0.01 | 3.388 |
| EAM258 | hmr_miR-222 | 0.015 | 4.257 |
| EAM230 | hmr_miR-183 | 0.02 | −3.977 |
| EAM364 | mr_miR-330 | 0.02 | 3.982 |
| EAM206 | hmr_miR-139 | 0.02 | 3.761 |
| EAM327 | hmr_miR-299 | 0.025 | 2.353 |
| EAM232 | hmr_miR-192 | 0.04 | 1.065 |
| EAM257 | hmr_miR-221 | 0.04 | 4.321 |
| EAM216 | hm_miR-149 | 0.04 | 3.711 |

TABLE 16
Prediction results of mouse lung samples
Test set: 12 mouse lung samples

| SAMPLE | MAL | PRED-MAL | CORRECT? |
|---|---|---|---|
| N_MLUNG_1 | Normal | Normal | Yes |
| N_MLUNG_2 | Normal | Normal | Yes |
| N_MLUNG_3 | Normal | Normal | Yes |
| N_MLUNG_4 | Normal | Normal | Yes |
| N_MLUNG_5 | Normal | Normal | Yes |
| T_MLUNG_1 | Tumor | Tumor | Yes |
| T_MLUNG_2 | Tumor | Tumor | Yes |
| T_MLUNG_3 | Tumor | Tumor | Yes |
| T_MLUNG_4 | Tumor | Tumor | Yes |
| T_MLUNG_5 | Tumor | Tumor | Yes |
| T_MLUNG_6 | Tumor | Tumor | Yes |
| T_MLUNG_7 | Tumor | Tumor | Yes |

| | |
|---|---|
| Field Description | |
| SAMPLE | Sample name |
| MAL | Malignancy status (Normal/Tumor) |
| PRED-MAL | Predicted Malignancy status (Normal/Tumor). |
| | Prediction performed by kNN (k = 3) using a |
| | training set of 75 samples |
| CORRECT? | Is the prediction correct? |

TABLE 17
59 miRNAs Detected in HL-60 Cells

| | |
|---|---|
| Probe | miRNA |

| | |
|---|---|
| EAM103 | Hmr_miR-124a |
| EAM111 | Hm_let-7g |
| EAM115 | Hmr_miR-16 |
| EAM119 | Hmr_miR-29b |
| EAM131 | Hmr_miR-92 |
| EAM145 | Hmr_let-7c |
| EAM270 | hmr_miR-30b |
| EAM163 | hmr_miR-142-3p |
| EAM186 | h_miR-106a |
| EAM209 | hmr_miR-142-5p |
| EAM223 | hmr_miR-15b |
| EAM224 | hmr_miR-17-5p |
| EAM226 | hmr_miR-181a |
| EAM227 | hmr_miR-181b |
| EAM236 | hmr_miR-19a |
| EAM257 | hmr_miR-221 |
| EAM258 | hmr_miR-222 |
| EAM259 | hmr_miR-223 |
| EAM273 | hmr_miR-33 |
| EAM297 | hmr_miR-193 |
| EAM282 | m_miR-199b |
| EAM279 | hmr_miR-29c |
| EAM278 | hmr_miR-98 |
| EAM272 | hmr_miR-30d |
| EAM264 | hmr_miR-27b |
| EAM263 | hmr_miR-26a |
| EAM262 | hmr_miR-24 |
| EAM261 | hmr_miR-23b |
| EAM260 | hmr_miR-23a |
| EAM244 | hmr_miR-21 |
| EAM240 | hmr_miR-20 |
| EAM237 | hmr_miR-19b |
| EAM228 | hmr_miR-181c |
| EAM222 | hm_miR-15a |
| EAM219 | hmr_miR-153 |
| EAM218 | hmr_miR-152 |
| EAM206 | hmr_miR-139 |
| EAM193 | hmr_miR-125a |
| EAM187 | hmr_miR-107 |
| EAM185 | hmr_miR-103 |
| EAM181 | hmr_let-7f |
| EAM179 | hmr_let-7d |
| EAM175 | hmr_miR-320 |
| EAM160 | hmr_miR-26b |
| EAM153 | hmr_let-7a |
| EAM147 | hmr_let-7b |
| EAM311 | hmr_miR-101 |
| EAM313 | hmr_miR-106b |
| EAM318 | h_miR-17-3p |
| EAM324 | hmr_miR-25 |
| EAM329 | hm_miR-302a |
| EAM331 | hmr_miR-30e |
| EAM337 | hmr_miR-93 |
| EAM341 | m_miR-106a |
| EAM352 | m_miR-294 |
| EAM364 | mr_miR-330 |
| EAM368 | hmr_miR-339 |
| EAM380 | r_miR-140* |
| EAM392 | r_miR-352 |

TABLE 18
mRNAs used to estimate proliferation rates

| Chip | Probe Set ID | Gene Title |
|---|---|---|
| Hu6800 | AB003698_at | CDC7 cell division cycle 7 (S. cerevisiae) |
| Hu6800 | D00596_at | thymidylate synthetase |
| Hu6800 | D14134_at | RAD51 homolog (RecA homolog, E. coli) (S. cerevisiae) |
| Hu6800 | D21063_at | MCM2 minichromosome maintenance deficient 2, mitotin (S. cerevisiae) |
| Hu6800 | D38073_at | MCM3 minichromosome maintenance deficient 3 (S. cerevisiae) |
| Hu6800 | D38550_at | E2F transcription factor 3 |
| Hu6800 | D84557_at | MCM6 minichromosome maintenance deficient 6 (MIS5 |
| | | homolog, S. pombe) (S. cerevisiae) |
| Hu6800 | J00139_s_at | dihydrofolate reductase pseudogene 1 /// dihydrofolate |
| | | reductase |
| Hu6800 | J04088_at | topoisomerase (DNA) II alpha 170 kDa |
| Hu6800 | J05614_at | proliferating cell nuclear antigen |
| Hu6800 | L07493_at | replication protein A3, 14 kDa |
| Hu6800 | L25876_at | cyclin-dependent kinase inhibitor 3 (CDK2-associated dual |
| | | specificity phosphatase) |
| Hu6800 | L32866_at | baculoviral IAP repeat-containing 5 (survivin) |
| Hu6800 | L47276_s_at | topoisomerase (DNA) II alpha 170 kDa |
| Hu6800 | M15796_at | proliferating cell nuclear antigen |
| Hu6800 | M25753_at | cyclin B1 |
| Hu6800 | M34065_at | cell division cycle 25C |
| Hu6800 | M74093_at | cyclin E1 |
| Hu6800 | M87339_at | replication factor C (activator 1) 4, 37 kDa |
| Hu6800 | M94362_at | lamin B2 |
| Hu6800 | S49592_s_at | E2F transcription factor 1 |
| Hu6800 | S78187_at | cell division cycle 25B |
| Hu6800 | U04810_at | trophinin associated protein (tastin) |
| Hu6800 | U05340_at | CDC20 cell division cycle 20 homolog (S. cerevisiae) |
| Hu6800 | U14518_at | centromere protein A, 17 kDa |
| Hu6800 | U20979_at | chromatin assembly factor 1, subunit A (p150) |
| Hu6800 | U22398_at | cyclin-dependent kinase inhibitor 1C (p57, Kip2) |
| Hu6800 | U26727_at | cyclin-dependent kinase inhibitor 2A (melanoma, p16, inhibits |
| | | CDK4) |
| Hu6800 | U28386_at | karyopherin alpha 2 (RAG cohort 1, importin alpha 1) |
| Hu6800 | U30872_at | centromere protein F, 350/400 ka (mitosin) |
| Hu6800 | U37022_rna1_at | cyclin-dependent kinase 4 |
| Hu6800 | U47677_at | E2F transcription factor 1 |
| Hu6800 | U56816_at | membrane-associated tyrosine- and threonine-specific cdc2- |
| | | inhibitory kinase |
| Hu6800 | U65410_at | MAD2 mitotic arrest deficient-like 1 (yeast) |
| Hu6800 | U74612_at | forkhead box M1 |
| Hu6800 | U77949_at | CDC6 cell division cycle 6 homolog (S. cerevisiae) |
| Hu6800 | X05360_at | cell division cycle 2, G1 to S and G2 to M |
| Hu6800 | X13293_at | v-myb myeloblastosis viral oncogene homolog (avian)-like 2 |
| Hu6800 | X51688_at | cyclin A2 |
| Hu6800 | X54942_at | CDC28 protein kinase regulatory subunit 2 |
| Hu6800 | X59543_at | ribonucleotide reductase M1 polypeptide |
| Hu6800 | X59618_at | ribonucleotide reductase M2 polypeptide |
| Hu6800 | X62153_s_at | MCM3 minichromosome maintenance deficient 3 (S. cerevisiae) |
| Hu6800 | X65550_at | antigen identified by monoclonal antibody Ki-67 |
| Hu6800 | X74330_at | primase, polypeptide 1, 49 kDa |
| Hu6800 | X74794_at | MCM4 minichromosome maintenance deficient 4 (S. cerevisiae) |
| Hu6800 | X74795_at | MCM5 minichromosome maintenance deficient 5, cell division |
| | | cycle 46 (S. cerevisiae) |
| Hu6800 | X87843_at | menage a trois 1 (CAK assembly factor) |
| Hu6800 | X89398_cds2_at | uracil-DNA glycosylase |
| Hu6800 | X95406_at | cyclin E1 |
| Hu6800 | X97795_at | RAD54-like (S. cerevisiae) |
| Hu6800 | Z15005_at | centromere protein E, 312 kDa |
| Hu6800 | Z29066_s_at | NIMA (never in mitosis gene a)-related kinase 2 |
| Hu6800 | Z29077_xpt1_at | cell division cycle 25C |
| Hu6800 | Z36714_at | cyclin F |
| Hu35KsubA | AA436304_at | RAN, member RAS oncogene family |
| Hu35KsubA | AF004709_at | mitogen-activated protein kinase 13 |
| Hu35KsubA | M96577_s_at | E2F transcription factor 1 |
| Hu35KsubA | RC_AA599859_at | Cyclin B1 |
| Hu35KsubA | RC_AA620553_s_at | flap structure-specific endonuclease 1 |
| Hu35KsubA | U75285_rna1_at | baculoviral IAP repeat-containing 5 (survivin) |
| Hu35KsubA | U78310_at | pescadillo homolog 1, containing BRCT domain (zebrafish) |
| Hu35KsubA | W28391_at | proliferation-associated 2G4, 38 kDa |
| Hu35KsubA | X74794_at | MCM4 minichromosome maintenance deficient 4 (S. cerevisiae) |
| Hu35KsubA | Z68092_s_at | cell division cycle 25B |

TABLE 19
Information on Poorly Differentiated Tumor Samples

| | Sample of | | |
|---|---|---|---|
| | Primary | | |
| | or Metastatic | | |
| Sample Name | Origin | Primary Site | Metastatic Site |
| PDT_BRST_1 | Primary | Breast | |
| PDT_BRST_2 | Primary | Breast | |
| PDT_BRST_3 | Primary | Breast | |
| PDT_BRST_4 | Primary | Breast | |
| PDT_BRST_5 | Metastatic | Breast | Lymph node/ |
| | | | supraclavic |
| PDT_COLON_1 | Primary | Colon | |
| PDT_LBL_1 | Primary | Lymph node | Groin |
| PDT_LUNG_1 | Metastatic | Lung | Kidney |
| PDT_LUNG_2 | Primary | Lung | |
| PDT_LUNG_3 | Primary | Lung | |
| PDT_LUNG_4 | Primary | Lung | |
| PDT_LUNG_5 | Metastatic | Lung | Adrenal |
| PDT_LUNG_6 | Primary | Lung | |
| PDT_LUNG_7 | Primary | Lung | |
| PDT_LUNG_8 | Primary | Lung | |
| PDT_OVARY_1 | Primary | Ovary | |
| PDT_OVARY_2 | Metastatic | Ovary | Omentum |
| PDT_OVARY_3 | Primary | Ovary | |
| PDT_STOM_1 | Primary | Stomach/GE_Jct | |

TABLE 20
Training and prediction results of poorly differentiated tumors

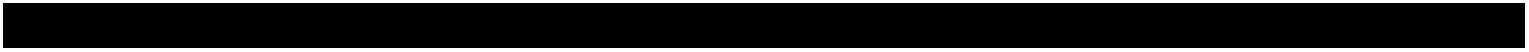

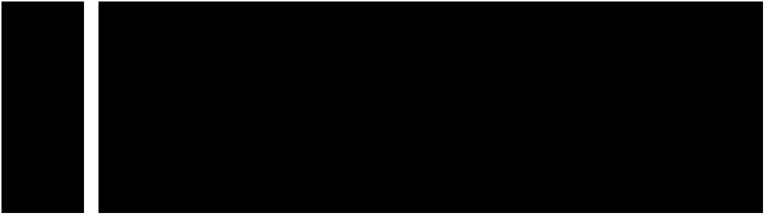

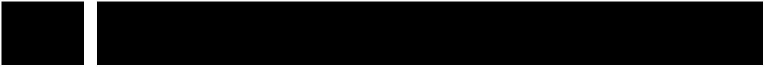

miRNA Data
Training set: 68 samples, 11 tissue-types

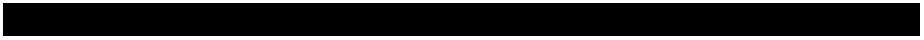

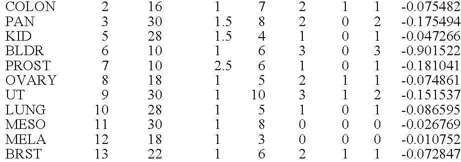

Test set: 17 samples, 4 tissue-types

| SAMPLE | PDT_COLON_1 | PDT_OVARY_1 | PDT_OVARY_2 | PDT_OVARY_3 | PDT_LUNG_1 |
|---|---|---|---|---|---|
| TRUE | 2 | 8 | 8 | 8 | 10 |
| PRED | 2 | 8 | 8 | 8 | 2 |
| PROB | 0.95 | 0.838 | 0.823 | 0.929 | 0.312 |
| CORR | 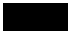 | 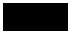 | 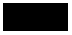 | 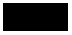 | 0 |

| SAMPLE | PDT_LUNG_2 | PDT_LUNG_3 | PDT_LUNG_4 | PDT_LUNG_5 | PDT_LUNG_6 | PDT_LUNG_7 |
|---|---|---|---|---|---|---|
| TRUE | 10 | 10 | 10 | 10 | 10 | 10 |
| PRED | 10 | 13 | 7 | 10 | 10 | 13 |
| PROB | 0.207 | 0.161 | 0.128 | 0.229 | 0.345 | 0.377 |
| CORR | 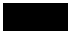 | 0 | 0 | 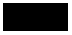 | 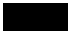 | 0 |
| SAMPLE | PDT_LUNG_8 | PDT_BRST_1 | PDT_BRST_2 | PDT_BRST_3 | PDT_BRST_4 | PDT_BRST_5 |
| TRUE | 10 | 13 | 13 | 13 | 13 | 13 |
| PRED | 10 | 13 | 13 | 13 | 9 | 13 |
| PROB | 0.299 | 0.905 | 0.479 | 0.552 | 0.476 | 0.773 |
| CORR | 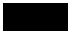 | 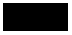 | 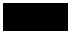 | 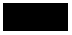 | 0 | 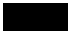 |

Test set: Posterior probability matrix

| Tissue | | | | | |
|---|---|---|---|---|---|
| Type\ | | | | | |
| SAMPLE | PDT_COLON_1 | PDT_OVARY_1 | PDT_OVARY_2 | PDT_OVARY_3 | PDT_LUNG_1 |
| COLON | 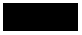 | 0 | 0 | 0 | 0.242 |
| PAN | 0.069 | 0.012 | 0.011 | 0.004 | 0.034 |
| KID | 0 | 0 | 0 | 0 | 0.02 |
| BLDR | 0 | 0 | 0 | 0 | 0 |
| PROST | 0 | 0.003 | 0.001 | 0 | 0 |
| OVARY | 0 | 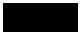 | 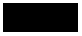 | 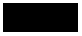 | 0.03 |
| UT | 0 | 0.342 | 0.193 | 0.225 | 0.312 |
| LUNG | 0 | 0 | 0 | 0 | 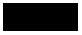 |
| MESO | 0 | 0 | 0 | 0 | 0 |
| MELA | 0 | 0 | 0 | 0 | 0 |
| BRST | 0 | 0.001 | 0 | 0 | 0.001 |

| Tissue | | | | | | |
|---|---|---|---|---|---|---|
| Type\ | | | | | | |
| SAMPLE | PDT_LUNG_2 | PDT_LUNG_3 | PDT_LUNG_4 | PDT_LUNG_5 | PDT_LUNG_6 | PDT_LUNG_7 |
| COLON | 0 | 0 | 0 | 0 | 0.247 | 0 |
| PAN | 0.003 | 0 | 0.001 | 0.004 | 0.152 | 0.006 |
| KID | 0 | 0 | 0 | 0 | 0 | 0 |
| BLDR | 0 | 0.01 | 0 | 0 | 0 | 0 |
| PROST | 0.078 | 0 | 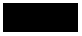 | 0.011 | 0 | 0.048 |
| OVARY | 0 | 0.001 | 0.121 | 0.025 | 0 | 0.003 |
| UT | 0 | 0.029 | 0 | 0.012 | 0.001 | 0 |
| LUNG | 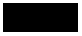 | 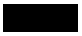 | 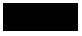 | 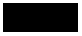 | 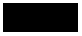 | 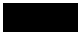 |
| MESO | 0.002 | 0 | 0 | 0 | 0 | 0 |
| MELA | 0 | 0 | 0 | 0 | 0 | 0 |
| BRST | 0 | 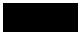 | 0.074 | 0 | 0.02 | 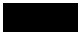 |
| Tissue | | | | | | |
| Type\ | | | | | | |
| SAMPLE | PDT_LUNG_8 | PDT_BRST_1 | PDT_BRST_2 | PDT_BRST_3 | PDT_BRST_4 | PDT_BRST_5 |
| COLON | 0 | 0 | 0 | 0 | 0 | 0 |
| PAN | 0 | 0.003 | 0.011 | 0 | 0.004 | 0.007 |
| KID | 0 | 0 | 0 | 0 | 0 | 0 |
| BLDR | 0 | 0.002 | 0.001 | 0.077 | 0.006 | 0 |
| PROST | 0.03 | 0.001 | 0.003 | 0.001 | 0 | 0.003 |
| OVARY | 0.001 | 0 | 0 | 0.13 | 0.009 | 0 |
| UT | 0.002 | 0.003 | 0 | 0.004 | 0.476 | 0.005 |
| LUNG | 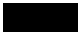 | 0.017 | 0.035 | 0 | 0 | 0.277 |
| MESO | 0 | 0 | 0 | 0 | 0 | 0 |
| MELA | 0 | 0 | 0 | 0 | 0 | 0 |
| BRST | 0.149 | 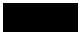 | 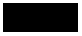 | 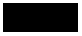 | 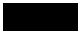 | 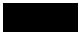 |

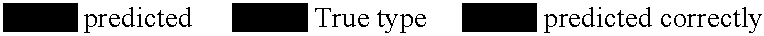

mRNA Data
Training set: 68 samples, 11 tissue-types

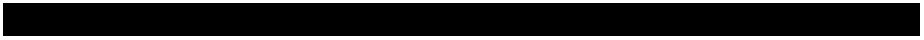

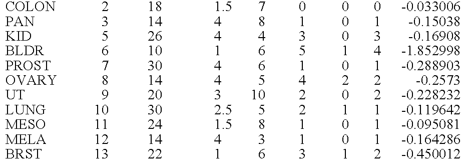

Test set: 17 samples, 4 tissue-types

| SAMPLE | PDT_COLON_1 | PDT_OVARY_1 | PDT_OVARY_2 | PDT_OVARY_3 | PDT_LUNG_1 |
|---|---|---|---|---|---|
| TRUE | 2 | 8 | 8 | 8 | 10 |
| PRED | 7 | 5 | 9 | 8 | 8 |
| PROB | 0.013 | 1 | 0.376 | 0.76 | 0.229 |
| CORR | 0 | 0 | 0 | 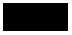 | 0 |

| SAMPLE | PDT_LUNG_2 | PDT_LUNG_3 | PDT_LUNG_4 | PDT_LUNG_5 | PDT_LUNG_6 | PDT_LUNG_7 |
|---|---|---|---|---|---|---|
| TRUE | 10 | 10 | 10 | 10 | 10 | 10 |
| PRED | 6 | 6 | 3 | 8 | 8 | 8 |
| PROB | 0.128 | 0.022 | 0.102 | 0.305 | 0.014 | 0.091 |
| CORR | 0 | 0 | 0 | 0 | 0 | 0 |
| SAMPLE | PDT_LUNG_8 | PDT_BRST_1 | PDT_BRST_2 | PDT_BRST_3 | PDT_BRST_4 | PDT_BRST_5 |
| TRUE | 10 | 13 | 13 | 13 | 13 | 13 |
| PRED | 6 | 9 | 8 | 8 | 6 | 3 |
| PROB | 0.173 | 0.133 | 0.362 | 0.301 | 0.05 | 0.027 |
| CORR | 0 | 0 | 0 | 0 | 0 | 0 |

Test set: Posterior probability matrix

| Tissue | | | | | |
|---|---|---|---|---|---|
| Type\ | | | | | |
| SAMPLE | PDT_COLON_1 | PDT_OVARY_1 | PDT_OVARY_2 | PDT_OVARY_3 | PDT_LUNG_1 |
| COLON | 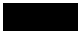 | 0 | 0 | 0 | 0 |
| PAN | 0.012 | 0.019 | 0.005 | 0.002 | 0.027 |
| KID | 0 | 1 | 0 | 0 | 0 |
| BLDR | 0.001 | 0.166 | 0.001 | 0.003 | 0.191 |
| PROST | 0.013 | 0.006 | 0.012 | 0.081 | 0.006 |
| OVARY | 0 | 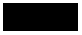 | 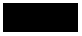 | 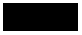 | 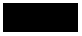 |
| UT | 0 | 0 | 0.376 | 0.084 | 0.074 |
| LUNG | 0.001 | 0 | 0.261 | 0 | 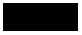 |
| MESO | 0 | 0.01 | 0.007 | 0.001 | 0.004 |
| MELA | 0 | 0 | 0 | 0 | 0 |
| BRST | 0 | 0.142 | 0 | 0 | 0.018 |

| Tissue | | | | | | |
|---|---|---|---|---|---|---|
| Type\ | | | | | | |
| SAMPLE | PDT_LUNG_2 | PDT_LUNG_3 | PDT_LUNG_4 | PDT_LUNG_5 | PDT_LUNG_6 | PDT_LUNG_7 |
| COLON | 0 | 0 | 0 | 0 | 0 | 0 |
| PAN | 0.024 | 0.004 | 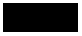 | 0.016 | 0.009 | 0.011 |
| KID | 0 | 0 | 0 | 0 | 0 | 0 |
| BLDR | 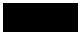 | 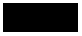 | 0.041 | 0.059 | 0.001 | 0.057 |
| PROST | 0.007 | 0.015 | 0.002 | 0.028 | 0.005 | 0.005 |
| OVARY | 0.072 | 0.006 | 0.062 | 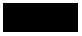 | 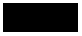 | 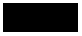 |
| UT | 0.007 | 0.013 | 0.038 | 0.05 | 0.002 | 0.009 |
| LUNG | 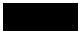 | 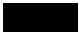 | 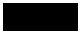 | 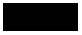 | 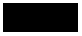 | 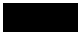 |
| MESO | 0 | 0.003 | 0.006 | 0.024 | 0.01 | 0.002 |
| MELA | 0 | 0 | 0 | 0 | 0 | 0 |
| BRST | 0 | 0 | 0 | 0 | 0 | 0 |
| Tissue | | | | | | |
| Type\ | | | | | | |
| SAMPLE | PDT_LUNG_8 | PDT_BRST_1 | PDT_BRST_2 | PDT_BRST_3 | PDT_BRST_4 | PDT_BRST_5 |
| COLON | 0 | 0 | 0 | 0 | 0 | 0 |
| PAN | 0.03 | 0.016 | 0.026 | 0.019 | 0.021 | 0.027 |
| KID | 0 | 0 | 0 | 0 | 0 | 0 |
| BLDR | 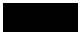 | 0.014 | 0.044 | 0.237 | 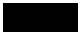 | 0.003 |
| PROST | 0.005 | 0.006 | 0.025 | 0.001 | 0.003 | 0.021 |
| OVARY | 0.055 | 0.01 | 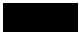 | 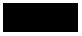 | 0 | 0 |
| UT | 0.012 | 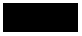 | 0.01 | 0.036 | 0.011 | 0.001 |
| LUNG | 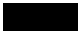 | 0 | 0.001 | 0.002 | 0 | 0 |
| MESO | 0.001 | 0.044 | 0 | 0.002 | 0.007 | 0.01 |
| MELA | 0 | 0 | 0 | 0 | 0 | 0 |
| BRST | 0 | 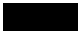 | 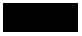 | 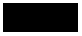 | 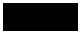 | 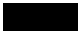 |

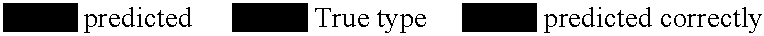

We claim:

1. A solution-based method for determining the expression level of a population of target nucleic acids, comprising:
a) providing in solution a population of target-specific bead sets, wherein each target-specific bead set is individually detectable and comprises a capture probe which corresponds to an individual target nucleic acid referred to as an individual bead set;
b) hybridizing in solution the population of target-specific bead sets with a population of molecules that can contain a population of detectable target molecules, wherein each target nucleic acid has been transformed into a corresponding detectable target molecule which will specifically bind to its corresponding individual target-specific bead set; and
c) screening in solution for detectable target molecules hybridized to target-specific beads to determine the expression level of the population of target nucleic acids.

2. The method of claim 1, wherein the population of target-specific bead sets comprises at least 5 individual bead sets that can bind with a corresponding set of target nucleic acids.

3. The method of claim 1, wherein the population of target-specific beads comprises at least 100 individual bead sets that can bind with a corresponding set of target nucleic acids.

4. The method of claim 1, wherein the population of target nucleic acids is a population of mRNAs.

5. The method of claim 1, wherein the population of target nucleic acids is a population of mRNAs and wherein each mRNA has been transformed into a corresponding detectable target molecule by a process comprising:
a) reverse transcribing the mRNA target nucleic acid to generate a cDNA;
b) contacting the cDNA with an upstream probe and a downstream probe, wherein the upstream probe comprises a universal upstream sequence and an upstream target-specific sequence, and the downstream probe comprises a universal downstream sequence and a downstream target-specific sequence, such that when the upstream probe and the downstream probe are both hybridized to the cDNA the two probes are capable of being ligated;
c) ligating said cDNA contacted with said upstream and downstream probes to generate ligation complexes; and
d) amplifying said ligation complexes with a pair of universal primers comprising a universal upstream primer and a universal downstream primer, wherein the universal upstream primer is complementary to the universal upstream sequence and the universal downstream primer is complementary to the universal downstream sequence, wherein at least one of the pair of universal primers is detectably labeled, wherein the product of the amplification is detectably labeled, thereby generating a detectable target molecule which corresponds to the target nucleic acid.

6. The method of claim 1, wherein the population of target nucleic acids is a population of mRNAs, wherein each mRNA has been transformed into a corresponding detectable target molecule by a process comprising:
a) reverse transcribing the mRNA target nucleic acid to generate a cDNA;
b) contacting the cDNA with an upstream probe and a downstream probe, wherein the upstream probe comprises a universal upstream sequence and an upstream target-specific sequence, and the downstream probe comprises a universal downstream sequence and a downstream target-specific sequence, such that when the upstream probe and the downstream probe are both hybridized to the cDNA the two probes are capable of being ligated;
c) ligating said cDNA contacted with said upstream and downstream probes to generate ligation complexes; and
d) amplifying said ligation complexes with a pair of universal primers comprising a universal upstream primer and a universal downstream primer, wherein the universal upstream primer is complementary to the universal upstream sequence and the universal downstream primer is complementary to the universal downstream sequence, wherein at least one of the pair of universal primers is detectably labeled, wherein the product of the amplification is detectably labeled, thereby generating a detectable target molecule which corresponds to the target nucleic acid, wherein either the upstream probe further comprises an amplicon tag between the universal sequence and the target-specific sequence or the downstream probe further comprises an amplicon tag between the universal sequence and the target-specific sequence, wherein the amplicon tag comprises a nucleic acid sequence that is complementary to the sequence of the capture probe of the bead set.

7. A method of identifying an expression signature associated with the presence or risk of cancer, infection, cellular disorder, or response to treatment comprising:
a) isolating cells from a group of individuals with said cancer, infection, cellular disorder, or response to treatment, and determining the expression levels of a group of genes;
b) isolating cells from a group of individuals without said cancer, infection, cellular disorder, or response to treatment, and determining the expression levels of said group of genes; and
c) identifying differentially expressed genes from said group of genes which are together indicative of the presence or risk of cancer, infection, cellular disorder, or response to treatment in an individual, thereby identifying an expression signature associated with the presence or risk of cancer, infection, cellular disorder, or response to treatment, wherein the expression levels of the group of genes is determined using the method of claim 1 and the population of target nucleic acids are mRNAs.

8. The method of claim 1, wherein the population of target nucleic acids is a population of microRNAs.

9. A method of identifying an expression signature associated with the presence or risk of cancer, infection, cellular disorder, or response to treatment comprising:
a) isolating cells from a group of individuals with said cancer, infection, cellular disorder, or response to treatment, and determining the expression levels of a group of genes;
b) isolating cells from a group of individuals without said cancer, infection, cellular disorder, or response to treatment, and determining the expression levels of said group of genes; and
c) identifying differentially expressed genes from said group of genes which are together indicative of the presence or risk of cancer, infection, cellular disorder, or response to treatment in an individual, thereby identifying an expression signature associated with the presence or risk of cancer, infection, cellular disorder, or response to treatment, wherein the expression levels of the group of genes is determined using the method of claim 1, wherein the population of target nucleic acids is a population of microRNAs and, wherein the expression signature comprises at least 5 genes.

10. The method of claim 1, wherein the population of target nucleic acids is a population of microRNAs and wherein each microRNA has been transformed into a corresponding detectable target molecule by a process comprising:
a) ligating at least one adaptor to the microRNA, generating an adaptor-microRNA molecule;
b) detectably labeling said adaptor-microRNA molecule, thereby generating a detectable target molecule which corresponds to the target nucleic acid.

11. The method of claim 1, wherein the population of target nucleic acids is a population of microRNAs and wherein each microRNA has been transformed into a corresponding detectable target molecule by a process comprising:
a) ligating at least one adaptor to the microRNA, generating an adaptor-microRNA molecule;
b) detectably labeling said adaptor-microRNA molecule, thereby generating a detectable target molecule which corresponds to the target nucleic acid, wherein the adaptor-microRNA is detectably labeled by reverse transcription using the adaptor-microRNA as a template for polymerase chain reaction, wherein a pair of primers is used in said polymerase chain reaction, and wherein at least one of said primers is detectably labeled.

12. A method of screening for the presence of malignant cells in a test sample comprising:
a) determining the level of expression of a group of microRNAs in the test sample, and
b) comparing the level of expression of a group of microRNAs between the test sample and a corresponding reference sample, wherein a lower level of expression of the group of microRNAs in the test sample compared to the reference sample is indicative of the test sample containing malignant cells.

13. The method of claim 12, wherein the reference sample is known to express a predetermined expression signature indicative of the presence of malignancy, infection, or cellular disorder, and the similarity of the expression signature of the test sample to the predetermined expression signature of the reference sample indicates the presence of malignant cells, infected cells, or cellular disorder, in the test sample.

14. The method of claim 12, wherein the group of microRNAs comprises at least 5 microRNAs.

15. The method of claim 12, wherein the test sample is isolated from an individual at risk of or suspected of having cancer.

16. A method of classifying a tumor sample comprising:
a) determining the expression pattern of a group of microRNAs in a tumor sample of unknown tissue origin, generating a tumor sample profile;
b) providing a model of tumor origin microRNA expression patterns based on a dataset of the expression of microRNAs of tumors of known origin; and
c) comparing the tumor sample profile to the model to determine which tumors of known origin the sample most closely resembles, thereby classifying the tissue origin of the tumor sample.

17. A method of classifying a tumor sample comprising:
a) determining the expression pattern of a group of microRNAs in a tumor sample of unknown tissue origin, generating a tumor sample profile;
b) providing a model of tumor origin microRNA expression patterns based on a dataset of the expression of microRNAs of tumors of known origin; and
c) comparing the tumor sample profile to the model to determine which tumors of known origin the sample most closely resembles, thereby classifying the tissue origin of the tumor sample, wherein the expression pattern of the group of microRNAs is determined using the methods of claim 1, wherein each target nucleic acid is a microRNA which has been transformed into a corresponding detectable target molecule by a process comprising:
d) ligating at least one adaptor to the microRNA, generating an adaptor-microRNA molecule;
e) detectably labeling said adaptor-microRNA molecule, thereby generating a detectable target molecule which corresponds to the target nucleic acid.

18. A method for identifying an active compound or molecule, comprising: contacting cells with a plurality of compounds or molecules, determining the expression of a set of marker genes present in the cells using the method of claim 1, and scoring the expression of the marker genes to identify a cellular phenotype, the presence of a specific cellular phenotype being indicative of an active compound or molecule.

19. A method for identifying an active compound or molecule, comprising: contacting cells with a plurality of compounds or molecules, determining the expression of a set of marker genes present in the cells using the method of claim 1, and scoring the expression of the marker genes to identify a cellular phenotype, the presence of a specific cellular phenotype being indicative of an active compound or molecule, wherein the set of marker genes comprises genes which encode microRNAs.

20. A method for identifying an active compound or molecule, comprising: contacting cells with a plurality of compounds or molecules, determining the expression of a set of marker genes present in the cells using the method of claim 1, and scoring the expression of the marker genes to identify a cellular phenotype, the presence of a specific cellular phenotype being indicative of an active compound or molecule, wherein the set of marker genes comprises genes which encode messenger RNAs.

21. A kit for determining in solution the expression level of a population of target nucleic acids, wherein said kit comprises:
a) a population of detectable bead sets, wherein each target-specific bead set is individually detectable and is capable of being coupled to a capture probe which corresponds to an individual target nucleic acid of interest;
b) components for transforming a target nucleic acid of interest into a corresponding detectable target molecule which will specifically bind to its corresponding individual target-specific bead set c) capture probes capable of specifically hybridizing to at least 10 different microRNAs or at least 10 different mRNAs.

22. The kit of claim 21, wherein the population of target nucleic acids comprises mRNAs, wherein the kit further comprises
a) components for reverse transcribing the mRNA to generate cDNA;
b) upstream and downstream probes, wherein the upstream probe comprises a universal upstream sequence and an upstream target-specific sequence, and the downstream probe comprises a universal downstream sequence and a downstream target-specific sequence, such that when the upstream probe and the downstream probe are both hybridized to the cDNA the two probes are capable of being ligated;
c) components for ligating DNA;
d) a pair of universal primers; and
e) components for amplifying DNA.

23. The kit of claim 21, wherein the population of target nucleic acids comprises microRNAs, wherein the kit further comprises
a) adaptors;
b) components for ligating the microRNAs to the adaptors;
c) components for reverse transcribing the microRNA to generate cDNA;
d) a pair of universal primers; and
e) components for amplifying DNA.

24. The kit of claim 21, further comprising a polymerase and nucleotide bases.

25. The kit of claim 21, further comprising a plurality of detectable labels.
